(19) United States
(12) Patent Application Publication
(10) Pub. No.: US 2020/0270618 A1
(43) Pub. Date: Aug. 27, 2020

(54) COMPOSITIONS AND METHODS FOR TREATING ALPHA-1 ANTITRYPSIN DEFICIENCY

(71) Applicant: Intellia Therapeutics, Inc., Cambridge, MA (US)

(72) Inventors: John Finn, Cambridge, MA (US); Hon-Ren Huang, Cambridge, MA (US); Anthony Forget, Cambridge, MA (US); Xin Xie, Cambridge, MA (US)

(21) Appl. No.: 16/657,967

(22) Filed: Oct. 18, 2019

Related U.S. Application Data
(60) Provisional application No. 62/747,522, filed on Oct. 18, 2018.

(51) Int. Cl. C12N 15/62; C12N 15/10; C12N 15/113; C07K 14/81

(52) U.S. Cl. CPC C12N 15/62; C07K 14/8125; C12N 15/113; C12N 15/102

(57) ABSTRACT

Compositions and methods for expressing alpha 1 antitrypsin (AAT) in a host cell are provided. Also provided are compositions and methods for treating subjects having alpha 1 antitrypsin deficiency (AATD).

This application claims the benefit of priority from U.S. Provisional Application No. 62/747,522, filed on Oct. 18, 2018. The specification of the foreigoing application is incorporated herein by reference in its entirety.

### SEQUENCE LISTING

The instant application contains a Sequence Listing which has been filed electronically in ASCII format and is hereby incorporated by reference in its entirety. Said ASCII copy, created on Nov. 21, 2019, is named 1861884-0002-005-101_SL.txt and is 218,587 bytes in size.

Alpha-1 antitrypsin (AAT or A1AT) or serum trypsin inhibitor is a type of serine protease inhibitor (also termed a serpin) encoded by the SERPINA1 gene. AAT is primarily synthesized and secreted by hepatocytes, and functions to inhibit the activity of neutrophil elastase in the lung. Without sufficient quantities of functioning AAT, neutrophil elastase is uncontrolled and damages alveoli in the lung. Thus, mutations in SERPINA1 that result in decreased levels of AAT, or decreased levels of properly functioning AAT, lead to lung pathology. Moreover, mutations in SERPINA1 that lead to production of misformed AAT leads to liver pathology due to accumulation of AAT in the hepatocytes. Thus, insufficient and improperly formed AAT caused by SERPINA1 mutation can lead to lung and liver pathology.

More than one hundred allelic variants have been described for the SERPINA1 gene. Variants are generally classified according to their effect on serum levels of AAT. For example, M alleles are normal variants associated with normal serum AAT levels, whereas Z and S alleles are mutant variants associated with decreased AAT levels. The presence of Z and S alleles is associated with al-antitrypsin deficiency (AATD or A1AD), a genetic disorder characterized by mutations in the SERPINA1 gene that leads to the production of abnormal AAT.

There are many forms and degrees of AATD. The “Z-variant” is the most common, causing severe clinical disease in both liver and lung. The Z-variant is characterized by a single nucleotide change in the 5′ end of the 5^{th} exon that results in a missense mutation of glutamic acid to lysine at amino acid position 342 (E342K). Symptoms arise in patients that are both homozygous (ZZ) and heterozygous (MZ or SZ) at the Z allele. The presence of one or two Z alleles results in SERPINA1 mRNA instability, and AAT protein polymerization and aggregation in liver hepatocytes. Patients having at least one Z allele have an increased incidence of liver cancer due to the accumulation of aggregated AAT protein in the liver. In addition to liver pathology, AATD characterized by at least one Z allele is also characterized by lung disease due to the decrease in AAT in the alveoli and the resulting decrease in inhibition of neutrophil elastase. The prevalence of the severe ZZ-form (i.e., homozygous expression of the Z-variant) is 1: 2,000 in northern European populations, and 1: 4,500 in the United States. The other common mutation is the S-variant, which results in a protein that is degraded intracellularly before secretion. Compared to the Z-variant, the S-variant causes milder reduction in serum AAT and lower risk for lung disease. A need exists to ameliorate the negative effects of AATD in both the liver and lung.

The present disclosure provides compositions and methods for expressing heterologous AAT at a human genomic locus, such as an albumin safe harbor site, thereby allowing secretion of heterologous AAT and alleviating the negative effects of AATD in the lung. The present disclosure also provides compositions and methods to knock out the endogenous SERPINA1 gene thereby eliminating the production of mutant forms of AAT that are associated with liver symptoms in patients with AATD. The invention combines knock out of an endogenous SERPINA1 allelle with insertion of heterologous AAT at a safe harbor site to restore AAT function in a cell or an organism.

In particular, provided herein are guide RNAs for use in targeted insertion of a nucleic acid sequence encoding AAT into a human safe harbor site, such as intron 1 of an albumin safe harbor site. Also provided are donor constructs (e.g., bidirectional constructs), comprising a sequence encoding AAT, for use in targeted insertion into a human safe harbor site, such as intron 1 of an albumin safe harbor site. In some embodiments, the guide RNA disclosed herein can be used in combination with a RNA-guided DNA binding agent (e.g., Cas nuclease) and a donor construct comprising a sequence encoding AAT (e.g., bidirectional construct). In some embodiments, the donor construct (e.g., bidirectional construct) can be used with any one or more gene editing systems (e.g., CRISPR/Cas system; zinc finger nuclease (ZFN) system; transcription activator-like effector nuclease (TALEN) system). The following embodiments are provided.

In some embodiments, the present disclosure provides a method of introducing a SERPINA1 nucleic acid to a cell or population of cells, comprising administering: i) a nucleic acid construct comprising a heterologous AAT protein coding sequence; ii) a RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID NOs: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33, thereby introducing the SERPINA1 nucleic acid to the cell or population of cells.

In some embodiments, the present disclosure provides a method of expressing AAT in a subject in need thereof, comprising administering: i) a nucleic acid construct comprising a heterologous AAT protein coding sequence; ii) a RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33, thereby expressing AAT in a subject in need thereof.

In some embodiments, the present disclosure provides a method of treating alpha-1 antitrypsin deficiency (AATD) in a subject in need of AAT protein, comprising administering: i) a nucleic acid construct comprising a heterologous AAT protein coding sequence; ii) a RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33, thereby treating AATD in the subject.

In some embodiments, the present disclosure provides a method of increasing AAT secretion from a liver cell or population of cells, comprising administering: i) a nucleic acid construct comprising a heterologous AAT protein coding sequence; ii) a RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33, thereby increasing AAT secretion from the liver cell or the population of cells.

### BRIEF DESCRIPTION OF THE DRAWINGS

FIGS. 1A-1C show results of in vitro screening of a bidirectional construct across target sites in primary mouse hepatocytes. FIG. 1A represents a schematic of the vector tested. FIG. 1B shows varied levels of editing using various gRNAs tested (guide IDs indicated on x-axis). FIG. 1C shows high level of editing did not necessarily result in more efficient expression of the transgene.

FIGS. 2A-2C show results from in vitro screening of bidirectional constructs across target sites in primary cynomolgus hepatocytes. FIG. 2A shows varied levels of editing detected for each of the combinations tested. FIG. 2B and FIG. 2C show that significant levels of editing (as indel formation at a specific target site) did not necessarily result in more efficient insertion or expression of the transgenes.

FIGS. 3A-3C show results from in vitro screening of bidirectional constructs across target sites in primary human hepatocytes. FIG. 3A shows varied levels of editing detected for each of the combinations tests. FIG. 3B and FIG. 3C show that significant levels of editing (as indel formation at a specific target site) did not necessarily result in more efficient insertion or expression of the transgenes.

FIGS. 4A-4C show results from in vivo studies of hSERPINA1 insertion into the mAlbumin locus. FIG. 4A shows editing results using the gRNAs tested (indicated on the x-axis). FIG. 4B shows serum hA1AT levels at 1, 2, and 4 weeks post dose. FIG. 4C shows a positive correlation between the levels of expression as measured in RLU for a given guide from in vitro experiments and hA1AT transgene expression levels in vivo.

FIGS. 5A-5D show results from in vivo knockdown of hSERPINA1 PiZ transgene and insertion of hSERPINA1 into mAlbumin locus. FIG. 5A outlines the editing conditions used for each test group. FIG. 5B shows indel formation in the hSERPINA1 PiZ variant that was targeted in Stage 1. FIG. 5C shows indel formation in the albumin locus targeted in Stage 2. FIG. 5D shows hA1AT protein levels in serum at various time points as measured by ELISA, as well as hA1AT levels as measured in human plasma.

FIG. 6 shows relative lucferase units from a luciferase-based fluorescence detection assay.

FIG. 7 shows the results from in vitro screening of a bidirectional construct across target sites using various sgRNAs in primary mouse hepatocytes. FIG. 7 shows that varied levels of expression were detected using various sgRNAs.

FIG. 8 shows the results from in vitro screening of a bidirectional construct across target sites in primary rat hepatocytes. FIG. 8 shows insertion using certain guide RNAs (relative luciferase units).

FIG. 9 shows insertion using various concentrations of guide RNAs.

FIG. 10 shows AAT levels using various AAV constructs.

FIG. 11 shows AAT levels at various time points as measured by ELISA.

FIG. 12 shows indel formation in the albumin locus.

FIG. 13A shows AAT levels at various time points as measured by ELISA. FIG. 13B shows AAT levels at various time points as measured by LC-MS/MS.

FIGS. 14A and FIG. 14B show expression levels of AAT with various concentrations of LNP or AAV. FIG. 14C and FIG. 14D show indel formation using various concentrations of LNP and AAV.

FIG. 15 shows a schematic of two bidirectional AAT AAV constructs.

### DETAILED DESCRIPTION

Reference will now be made in detail to certain embodiments of the invention, examples of which are illustrated in the accompanying drawings. While the present teachings are described in conjunction with various embodiments, it is not intended to limit the invention to those embodiments. On the contrary, the present teachings encompass various alternatives, modifications, and equivalents, as will be appreciated by those of skill in the art.

Before describing the present teachings in detail, it is to be understood that the disclosure is not limited to specific compositions or process steps, as such may vary. It should be noted that, as used in this specification and the appended embodiments, the singular form “a”, “an” and “the” include plural references unless the context dictates otherwise. Thus, for example, reference to “a conjugate” includes a plurality of conjugates and reference to “a cell” includes a plurality of cells and the like. As used herein, the term “include” and its grammatical variants are intended to be non-limiting, such that recitation of items in a list is not to the exclusion of other like items that can be substituted or added to the listed items.

Numeric ranges are inclusive of the numbers defining the range. Measured and measureable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the embodiments). The term “or” is used in an inclusive sense, i.e., equivalent to “and/or,” unless the context clearly indicates otherwise.

The term “about”, when used before a list, modifies each member of the list. The term “about” or “approximately” means an acceptable error for a particular value as determined by one of ordinary skill in the art, which depends in part on how the value is measured or determined.

The section headings used herein are for organizational purposes only and are not to be construed as limiting the desired subject matter in any way. In the event that any material incorporated by reference contradicts any term defined in this specification or any other express content of this specification, this specification controls.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with optional substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleosides and one or more nucleoside analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to either a guide that comprises a guide sequence, e.g. either a crRNA (also known as CRISPR RNA), or the combination of a crRNA and a trRNA (also known as tracrRNA crRNA (also known as CRISPR RNA), or the combination of a crRNA and a trRNA (also known as tracrRNA). The crRNA and trRNA may be associated as a single RNA molecule (single guide RNA, sgRNA) or, for example, in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs, such as sgRNAs or dgRNAs, can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 15, 16, 17, 18, 19, or 20 contiguous nucleotides of an albumin guide sequence selected from SEQ ID NOs: 2-33 or SERPINA1 guide sequence selected from SEQ ID Nos: 1000-1128. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 15, 16, 17, 18, 19, or 20 contiguous nucleotides of an albumin guide sequence selected from SEQ ID NOs: 2-33 or SERPINA1 guide sequence selected from SEQ ID Nos: 1000-1128. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 15, 16, 17, 18, 19, 20 or more base pairs. In some embodiments, the guide sequence and the target region may contain 1-4 mismatches where the guide sequence comprises at least 15, 16, 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse complement), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the sense or antisense strand (e.g. reverse complement) of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA-binding agent” means a polypeptide or complex of polypeptides having RNA and DNA binding activity, or a DNA-binding subunit of such a complex, wherein the DNA binding activity is sequence-specific and depends on the sequence of the RNA. The term RNA-guided DNA binding-agent also includes nucleic acids encoding such polypeptides. Exemplary RNA-guided DNA-binding agents include Cas cleavases/nickases. Exemplary RNA-guided DNA-binding agents may include inactivated forms thereof (“dCas DNA-binding agents”), e.g. if those agents are modified to permit DNA cleavage, e.g. via fusion with a FokI cleavase domain. “Cas nuclease”, as used herein, encompasses Cas cleavases and Cas nickases. Cas cleavases and Cas nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated”), if those agents are modified to permit DNA cleavage. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015) also contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015). As used herein, delivery of an RNA-guided DNA-binding agent (e.g. a Cas nuclease, a Cas9 nuclease, or an S. pyogenes Cas9 nuclease) includes delivery of the polypeptide or mRNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm inteace provided by the EBI at the www.ebi.ac.uk web server is generally appropriate.

As used herein, a first sequence is considered to be “X % complementary to” a second sequence if X % of the bases of the first sequence base pairs with the second sequence. For example, a first sequence 5′AAGA3′ is 100% complementary to a second sequence 3′TTCT5′, and the second sequence is 100% complementary to the first sequence. In some embodiments, a first sequence 5′AAGA3′ is 100% complementary to a second sequence 3′TTCTGTGA5′, whereas the second sequence is 50% complementary to the first sequence.

As used herein, “mRNA” is used herein to refer to a polynucleotide that is entirely or predominantly RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Exemplary guide sequences useful in the guide RNA compositions and methods described herein are shown in Table 1, Table 2, and throughout the application.

As used herein, “indels” refer to insertion/deletion mutations consisting of a number of nucleotides that are either inserted or deleted at the site of double-stranded breaks (DSBs) in a target nucleic acid.

As used herein, “heterologous alpha-1 antitrypsin” is used interchangeably with “heterologous AAT” or “heterologous A1AT” or “AAT/A1AT transgene”, which is the gene product of a SERPINA1 gene that is heterologous with respect to its insertion site. In some embodiments, the SERPINA1 gene is exogenous. The human wild-type AAT protein sequence is available at NCBI NP_000286; gene sequence is available at NCBI NM_000295. The human wild-type AAT cDNA has been sequenced (see, e.g., Long et al., “Complete sequence of the cDNA for human alpha 1-antitrypsin and the gene for the S variant,” Biochemistry 1984) and encodes a precursor molecule containing a signal peptide and a mature AAT peptide. Domains of the peptide responsible for intracellular targeting, carbohydrate attachment, catalytic function, protease inhibitory activity, etc., have been characterized (see, e.g., Kalsheker, “Alpha 1-antitrypsin: structure, function and molecular biology of the gene,” Biosci Rep. 1989; Matamala et al., “Identification of Novel Short C-Terminal Transcripts of Human SERPINA1 Gene,” PLoS One 2017; Niemann et al., “Isolation and serine protease inhibitory activity of the 44-residue, C-terminal fragment of alpha 1-antitrypsin from human placenta,” Matrix 1992). As used herein, heterologous AAT encompasses precursor AAT, mature AAT, and variants and fragments thereof, e.g., functional fragements, e.g., fragments that retain protease inhibitory activity (e.g., at least 60%, 70%, 80%, 85%, 90%, 92%, 94%, 96%, 98%, 99%, or 100%, compared to wild-type AAT, e.g., as assayed by a commercially available protease inhibition assay or human neutrophil elastase (HNE) inhibition assay). In some embodiments, the functional fragment is naturally occurring, e.g., a short C-terminal fragment. In some embodiments, the functional fragment is genetically engineered, e.g., a hyperactive functional fragment. Examples of the AAT protein sequence are described herein (e.g. SEQ ID NO: 700 or SEQ ID NO: 702). As used herein, heterologous AAT also encompasses a variant of AAT, e.g., a variant that possesses increased protease inhibitor activity as compared to wild type AAT. As used herein, heterologous AAT also encompasses a variant that is 80%, 85%, 90%, 93%, 95%, 97%, 99% identical to SEQ ID NO: 700, having functional activity—e.g., at least 60%, 70%, 80%, 85%, 90%, 92%, 94%, 96%, 98%, 99%, 100%, or more, activity as compared to wild type AAT, e.g., as assayed by HNE inhibition. As used herein, heterologous AAT also encompasses a fragment that possesses functional activity—e.g., at least 80%, 85%, 90%, 92%, 94%, 96%, 98%, 99%, 100%, or more, activity as compared to wild type AAT, e.g., as assayed by HNE inhibition. As used herein, heterologous AAT refers to an AAT, e.g. a functional AAT, useful in treating AATD, which may be wild-type AAT or a variant thereof useful in treating AATD.

As used herein, a “heterologous gene” refers to a gene that has been introduced as an exogenous source to a site within a host cell genome (e.g., at a genomic locus such as a safe harbor locus, including an albumin intron 1 site). A polypeptide expressed from such heterologous gene is referred to as a “heterologous polypeptide.” The heterologous gene can be naturally-occuring or engineered, and can be wild type or a variant. The heterologous gene may include nucleotide sequences other than the sequence that encodes the heterologous polypeptide. The heterologous gene can be a gene that occurs naturally in the host genome, as a wild type or a variant (e.g., mutant). For example, although the host cell contains the gene of interest (as a wild type or as a variant), the same gene or variant thereof can be introduced as an exogenous source for, e.g., expression at a locus that is highly expressed. The heterologous gene can also be a gene that is not naturally occurring in the host genome, or that expresses a heterologous polypeptide that does not naturally occur in the host genome. “Heterologous gene”, “exogenous gene”, and “transgene” are used interchangeably. In some embodiments, the heterologous gene or transgene includes an exogenous nucleic acid sequence, e.g. a nucleic acid sequence is not endogenous to the recipient cell. In certain embodiments, the heterologous gene can include an AAT nucleic acid sequence that does not naturally ocurr in the recipient cell. For example, heterologous AAT may be heterologous with respect to its insertion site and with respect to its recipient cell.

As used herein, “mutant SERPINA1” or “mutant SERPINA1 allele” refers to a SERPINA1 sequence having a change in the nucleotide sequence of SERPINA1 compared to the wildtype sequence (NCBI Gene ID: 5265; NCBI NM_000295; Ensembl: Ensembl:ENSG00000197249). In some embodiments, a mutant SERPINA1 allele encodes a non-functional and/or non-secreted AAT protein.

As used herein, “AATD” or “A1AD” refers to alpha-1 antitrypsin deficiency. AATD comprises diseases and disorders caused by a variety of different genetic mutations in SERPINA1. AATD may refer to a disease where decreased levels of functional AAT are expressed (e.g., less than 100%, 90%, 80%, 70%, 60%, 50%, 40%, 30%, 20%, 10%, or 5% AAT gene or protein expression as compared to a control sample, e.g., by nephelometry or immunoturbidimetry, e.g., AAT less than about 100 mg/dL, 90 mg/dL, 80 mg/dL, 70 mg/dL, 60 mg/dL, 50 mg/dL, 40 mg/dL, 30 mg/dL, 20 mg/dL, 10 mg/dL, or 5 mg/dL in serum), functional AAT is not expressed, or a mutant or non-functional AAT is expressed (e.g., forms aggregates and/or is not capable of being secreted and/or has decreased protease inhibitor activity). See, e.g., Greulich and Vogelmeier, Ther Adv Respir Dis 2016. In some embodiments, AATD refers to a disease where AAT is aggregated and/or accumulated intracellularly, e.g., in a hepatocyte, and not secreted, e.g., into circulation where it may be delivered to the lungs to function as a protease inhibitor. In some embodiments, AATD may be detected by PASD staining of liver tissue sections, e.g., to measure aggregation. In some embodiments, AATD may be detected by decreased inhibition of neutrophil elastase, e.g., in the lung.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “normal” or “healthy” individuals include those individuals that do not have the AATD-associated alleles—e.g., AATD-associated alleles are ZZ, MZ, or SZ.

As used herein, “treatment” refers to any administration or application of a therapeutic for disease or disorder in a subject, and includes inhibiting the disease, arresting its development, relieving one or more symptoms of the disease, curing the disease, or preventing reoccurrence of one or more symptoms of the disease. AATD may be associated with lung disease and/or liver disease; wheezing or shortness of breath; increased risk of lung infections; chronic obstructive pulmonary disease (COPD); bronchitis, asthma, dyspnea; cirrhosis; neonatal jaundice; panniculitis; chronic cough and/or phlegm; recurring chest colds; yellowing of the skin or the white part of the eyes; swelling of the belly or legs. For example, treatment of AATD may comprise alleviating symptoms of AATD, e.g., liver and/or lung symptoms. In some embodiments, treatment refers to increasing serum AAT levels, e.g., to protective levels. In some embodiments, treatment refers to increasing serum AAT levels, e.g., within the normal range. In some embodiments, treatment refers to increasing serum AAT levels, e.g., above 40, 50, 60, 70, 80, 90, or 100 mg/dL, e.g., as measured using nephelometry or immunoturbidimetry and a purified standard. In some embodiments, treatment refers to improvement in baseline serum AAT as compared to control, e.g., before and after treatment. In some embodiments, treatment refers to a improvement in histologic grading of AATD associated liver disease, e.g., by 1, 2, 3, or more points, as compared to control, e.g., before and after treatment. In some embodiments, treatment refers to improvement in Ishak fibrosis score as compared to control, e.g., before and after treatment. In some embodiments, treatment refers to improvement in genotype serum level, AAT lung function, spirometry test, chest X-ray of lung, CT scan of lung, blood testing of liver function, and/or ultrasound of liver.

As used herein, “knockdown” refers to a decrease in expression of a particular gene product (e.g., protein, mRNA, or both). Knockdown of a protein can be measured by, for example, detecting protein secreted by tissue or population of cells (e.g., in serum or cell media) or by detecting total cellular amount of the protein from a tissue or cell population of interest. Methods for measuring knockdown of mRNA are known, and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues). In some embodiments, the methods of the disclosure “knockdown” endogenous AAT in one or more cells (e.g., in a population of cells including in vivo populations such as those found in tissues). Relevant cells include cells that are capable of producing AAT. In some embodiments, the methods of the invention knockdown an endogenous mutant SERPINA1 allele, and/or an endogenous wildtype SERPINA1 allele (e.g., in a heterozygous MZ individual).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. Relevant cells include cells that are capable of producing AAT. In some embodiments, the methods of the invention “knockout” endogenous AAT in one or more cells (e.g., in a population of cells including in vivo populations such as those found in tissues). In some embodiments, the methods of the of the disclosure knockout an endogenous mutant SERPINA1 allele, and/or an endogenous wildtype SERPINA1 allele (e.g., in a heterozygous MZ individual). In some embodiments, a knockout is the complete loss of expression of endogenous AAT protein in a cell.

As used herein, “polypeptide” refers to a wild-type or variant protein (e.g., mutant, fragment, fusion, or combinations thereof). A variant polypeptide may possess at least or about 5%, 10%, 15%, 20%, 30%, 35%, 40%, 45%, 50%, 55%, 60%, 65%, 70%, 75%, 80%, 85%, 90%, 95%, or 100% functional activity of the wild-type polypeptide. In some embodiments, the variant is at least 70%, 75%, 80%, 85%, 90%, 92%, 93%, 94%, 95%, 96%, 97%, 98%, or 99% identical to the sequence of the wild-type polypeptide. In some embodiments, a variant polypeptide may be a hyperactive variant. In certain instances, the variant possesses between about 80% and about 120%, 140%, 160%, 180%, 200% of the functional activity of the wild-type polypeptide.

As used herein, a “bidirectional nucleic acid construct” (interchangeably referred to herein as “bidirectional construct”) comprises at least two nucleic acid segments, wherein one segment (the first segment) comprises a coding sequence that encodes a polypeptide of interest (the coding sequence may be referred to herein as “transgene” or a first transgene), while the other segment (the second segment) comprises a sequence wherein the complement of the sequence encodes a polypeptide of interest, or a second transgene. That is, the at least two segments can encode identical or different polypeptides. When the two segments encode the identical polypeptide, the coding sequence of the first segment need not be identical to the complement of the sequence of the second segment. In some embodiments, the sequence of the second segment is a reverse complement of the coding sequence of the first segment. A bidirectional construct can be single-stranded or double-stranded. The bidirectional construct disclosed herein encompasses a construct that is capable of expressing any polypeptide of interest.

As used herein, a “reverse complement” refers to a sequence that is a complement sequence of a reference sequence, wherein the complement sequence is written in the reverse orientation. For example, for a hypothetical sequence 5′ CTGGACCGA 3′ (SEQ ID NO: 500), the “perfect” complement sequence is 3′ GACCTGGCT 5′ (SEQ ID NO: 501), and the “perfect” reverse complement is written 5′ TCGGTCCAG 3′ (SEQ ID NO: 502). A reverse complement sequence need not be “perfect” and may still encode the same polypeptide or a similar polypeptide as the reference sequence. Due to codon usage redundancy, a reverse complement can diverge from a reference sequence that encodes the same polypeptide. As used herein, “reverse complement” also includes sequences that are, e.g., 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence.

In some embodiments, a bidirectional nucleic acid construct comprises a first segment that comprises a coding sequence that encodes a first polypeptide (a first transgene), and a second segment that comprises a sequence wherein the complement of the sequence encodes a second polypeptide (a second transgene). In some embodiments, the first and the second polypeptides are at least 50%, 55%, 60%, 65%, 70%, 75%, 80%, 85%, 90%, 91%, 92%, 93%, 94%, 95%, 96%, 97%, 98%, 99%, or 100% identical. In some embodiments, the first and the second polypeptides comprise an amino acid sequence that is at least 80%, 85%, 90%, 91%, 92%, 93%, 94%, 95%, 96%, 97%, 98%, 99%, or 100% identical, e.g. across 50, 100, 200, 500, 1000 or more amino acid residues.

A “safe harbor” locus is a locus within the genome wherein a gene may be inserted without significant deleterious effects on the host cell, e.g. hepatocyte, e.g., without causing apoptosis, necrosis, and/or senescence, or without causing more than 5%, 10%, 15%, 20%, 25%, 30%, or 40% apoptosis, necrosis, and/or senescence as compared to a control cell. See, e.g., Hsin et al., “Hepatocyte death in liver inflammation, fibrosis, and tumorigenesis,” 2017. In some embodiments, a safe harbor locus allows overexpression of an exogenous gene without significant deleterious effects on the host cell, e.g. hepatocyte, without causing apoptosis, necrosis, and/or senescence, or without causing more than 5%, 10%, 15%, 20%, 25%, 30%, or 40% apoptosis, necrosis, and/or senescence as compared to a control cell. In some embodiments, a desirable safe harbor locus may be one in which expression of the inserted gene sequence is not perturbed by read-through expression from neighboring genes. The safe harbor may be within an albumin gene, such as a human albumin gene. The safe harbor may be within an albumin intron 1 region, e.g., human albumin intron 1. The safe harbor may be a human safe harbor, e.g., for a liver tissue or hepatocyte host cell. In some embodiments, a safe harbor allows overexpression of an exogenous gene without significant deleterious effects on the host cell or cell population, such as hepatocytes or liver cells, e.g. without causing apoptosis, necrosis, and/or senescence, or without causing more than 5%, 10%, 15%, 20%, 25%, 30%, or 40% apoptosis, necrosis, and/or senescence as compared to a control cell.

In some embodiments, the gene may be inserted into a safe harbor locus and use the safe harbor locus's endogenous signal sequence, e.g., the albumin signal sequence encoded by exon 1. For example, an AAT coding sequence may be inserted into human albumin intron 1 such that it is downstream of and fuses to the signal sequence of human albumin exon 1.

In some embodiments, the gene may comprise its own signal sequence, may be inserted into the safe harbor locus, and may further use the safe habor locus's endogenous signal sequence. For example, an AAT coding sequence comprising an AAT signal sequence may be inserted into human albumin intron 1 such that it is downstream of and and fuses to the signal sequence of human albumin encoded by exon 1.

In some embodiments, the gene may comprise its own signal sequence and an internal ribosomal entry site (IRES), may be inserted into the safe harbor locus, and may further use the safe habor locus's endogenous signal sequence. For example, an AAT coding sequence comprising an AAT signal sequence and an IRES sequence may be inserted into human albumin intron 1 such that it is downstream of and fuses to the signal sequence of human albumin encoded by exon 1.

In some embodiments, the gene may comprise its own signal sequence and IRES, may be inserted into the safe harbor locus, and does not use the safe habor locus's endogenous signal sequence. For example, an AAT coding sequence comprising an AAT signal sequence and an IRES sequence may be inserted into human albumin intron 1 such that it does not fuse to the signal sequence of human albumin encoded by exon 1. In these embodiments, the protein is translated from the IRES site and is not chimeric (e.g., albumin signal peptide fused to AAT protein), which may be advantageously non- or low-immunogenic. In some embodiments, the protein is not secreted and/or transported extracellularly.

In some embodiments, the gene may be inserted into the safe harbor locus and may comprise an IRES and does not not use any signal sequence. For example, an AAT coding sequence comprising an IRES sequence and no AAT signal sequence may be inserted into human albumin intron 1 such that it does not fuse to the signal sequence of human albumin encoded by exon 1. In some embodiments, the proteins is translated from the IRES site without the need for any signal sequence. In some embodiments, the proteins is not transported extracellularly.

As used herein, a cell that is not undergoing mitotic cell division is referred to as a “non-dividing” cell. A “non-dividing” cell encompasses cell types that never or rarely undergo mitotic cell division, e.g., many types of neurons. A “non-dividing” cell also encompasses cells that are capable of, but not undergoing or about to undergo, mitotic cell division, e.g., a quiescent cell. Liver cells, for example, retain the ability to divide (e.g., when injured or resected), but do not typically divide. During mitotic cell division, homologous recombination is a mechanism by which the genome is protected and double-stranded breaks are repaired. In some embodiments, a “non-dividing” cell refers to a cell in which homologous recombination (HR) is not the primary mechanism by which double-stranded DNA breaks are repaired in the cell, e.g., as compared to a control dividing cell. In some embodiments, a “non-dividing” cell refers to a cell in which non-homologous end joining (NHEJ) is the primary mechanism by which double-stranded DNA breaks are repaired in the cell, e.g., as compared to a control dividing cell.

Non-dividing cell types have been described in the literature, e.g. by active NHEJ double-stranded DNA break repair mechanisms. See, e.g. Iyama, DNA Repair (Amst.) 2013, 12(8): 620-636. In some embodiments, the host cell includes, but is not limited to, a liver cell, a muscle cell, or a neuronal cell. In some embodiments, the host cell is a hepatocyte, such as a mouse, cyno, or human hepatocyte. In some embodiments, the host cell is a myocyte, such as a mouse, cyno, or human myocyte. In some embodiments, provided herein is a host cell, described above, that comprises the bidirectional construct disclosed herein. In some embodiments the host cell expresses the transgene polypeptide encoded by the bidirectional construct disclosed herein. In some embodiments, provided herein is a host cell made by a method disclosed herein. In certain embodiments, the host cell is made by administering or delivering to a host cell a bidirectional nucleic acid construct described herein, and a gene editing system such as a ZFN, TALEN, or CRISPR/Cas9 system.

### II. Compositions

### A. Compositions Comprising Safe Harbor Albumin Guide RNA (gRNAs) and/or SERPINA1 Guide RNA (gRNAs)

Provided herein are albumin guide RNA compositions, AAT template compositions, and methods useful for inserting and expressing a heterologous AAT gene (e.g., a functional or wild-type AAT) within a genomic locus such as a safe harbor gene of a host cell. In particular, as exemplified herein, targeting and inserting a heterologous AAT gene at the albumin locus (e.g., at intron 1) allows the use of albumin's endogenous promoter to drive robust expression of the heterologous AAT gene. The present disclosure is based, in part, on the identification of albumin guide RNAs that specifically target sites within intron 1 of the albumin gene, and which provide efficient insertion and expression of the heterologous AAT gene. As shown in the Examples and further described herein, the ability of identified gRNAs to mediate high levels of editing as measured through indel forming activity, unexpectedly does not necessarily correlate with use of the same gRNAs to mediate efficient insertion of heterologous genes as measured through, e.g., expression of the AAT transgene. That is, certain gRNAs that are able to achieve a high level of editing are not necessarily able to mediate efficient insertion, and conversely, some gRNAs shown to achieve low levels of editing may mediate efficient insertion and expression of a transgene.

In some embodiments, disclosed herein are compositions useful for introducing or inserting a heterologous AAT gene (e.g., a functional or wild-type AAT) within a locus such as an albumin locus (e.g., intron 1) of a host cell, e.g., using an albumin guide RNA disclosed herein with an RNA-guided DNA binding agent (e.g., Cas nuclease), and a construct (e.g., donor construct or template) comprising a heterologous AAT nucleic acid (“AAT transgene”). In some embodiments, disclosed herein are compositions useful for expressing a heterologous AAT gene at an albumin locus of a host cell, e.g., using an albumin guide RNA disclosed herein with an RNA-guided DNA binding agent and a construct (e.g., donor) comprising a heterologous AAT nucleic acid. In some embodiments, disclosed herein are compositions useful for expressing a heterologous AAT at an albumin locus of a host cell, e.g., using an albumin guide RNA disclosed herein with an RNA-guided DNA binding agent and a bidirectional construct comprising a heterologous AAT nucleic acid. In some embodiments, disclosed herein are compositions useful for inducing a break (e.g., double-stranded break (DSB) or single-stranded break (SSB or nick)) within the albumin gene of a host cell, e.g., using an albumin guide RNA disclosed herein with an RNA-guided DNA binding agent (e.g., a CRISPR/Cas system). The compositions may be used in vitro or in vivo for, e.g., treating AATD.

In some embodiments, the albumin guide RNAs disclosed herein comprise a guide sequence that binds, or is capable of binding, within an intron of an albumin locus. In some embodiments, the albumin guide RNAs disclosed herein bind within a region of intron 1 of the human albumin gene of SEQ ID NO: 1. It will be appreciated that not every base of the albumin guide sequence must bind within the recited regions. For example, in some embodiments, 15, 16, 17, 18, 19, 20, or more, bases of the albumin guide RNA sequence bind within the recited regions. For example, in some embodiments, 15, 16, 17, 18, 19, 20, or more contiguous bases of the guide RNA sequence bind with the recited regions.

In some embodiments, the albumin guide RNAs disclosed herein mediate a target-specific cutting by a RNA-guided DNA binding agent (e.g., Cas nuclease) at a site within intron 1 of human albumin (SEQ ID NO: 1). It will be appreciated that, in some embodiments, the guide RNAs comprise guide sequences that bind to, or are capable of binding to, said regions.

In some embodiments, the albumin guide RNAs disclosed herein comprise a guide sequence that is at least 99%, 98%, 97%, 96%, 95%, 94%, 93%, 92%, 91%, 90%, 89% or 88% identical to a sequence selected from the group consisting of SEQ ID NOs: 2-33.

In some embodiments, the albumin guide RNAs disclosed herein comprise a guide sequence having at least 15, 16, 17, 18, 19, or 20 contiguous nucleotides of a sequence selected from the group consisting of SEQ ID NOs: 2-33.

In some embodiments, the albumin guide RNA (gRNA) comprises a guide sequence chosen from: a) a sequence that is at least 95%, 90%, 85%, 80%, or 75% identical to a sequence selected from the group consisting of SEQ ID NOs: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33. In some embodiments, the albumin guide RNA comprises a sequence selected from the group consisting of SEQ ID NO:

2, 8, 13, 19, 28, 29, 31, 32, 33. See Table 1.

TABLE 1
Albumin targeted human guide RNA sequences and chromosomal
coordinates

| | | | SEQ |
|---|---|---|---|
| Guide | | | ID |
| ID | Guide Sequence | Genomic Coordinates | NO: |
| G009844 | GAGCAACCUCACUCUUGUCU | chr4:73405113-73405133 | 2 |
| G009851 | AUGCAUUUGUUUCAAAAUAU | chr4:73405000-73405020 | 3 |
| G009852 | UGCAUUUGUUUCAAAAUAUU | chr4:73404999-73405019 | 4 |
| G009857 | AUUUAUGAGAUCAACAGCAC | chr4:73404761-73404781 | 5 |
| G009858 | GAUCAACAGCACAGGUUUUG | chr4:73404753-73404773 | 6 |
| G009859 | UUAAAUAAAGCAUAGUGCAA | chr4:73404727-73404747 | 7 |
| G009860 | UAAAGCAUAGUGCAAUGGAU | chr4:73404722-73404742 | 8 |
| G009861 | UAGUGCAAUGGAUAGGUCUU | chr4:73404715-73404735 | 9 |
| G009866 | UACUAAAACUUUAUUUUACU | chr4:73404452-73404472 | 10 |
| G009867 | AAAGUUGAACAAUAGAAAAA | chr4:73404418-73404438 | 11 |
| G009868 | AAUGCAUAAUCUAAGUCAAA | chr4:73405013-73405033 | 12 |
| G009874 | UAAUAAAAUUCAAACAUCCU | chr4:73404561-73404581 | 13 |
| G012747 | GCAUCUUUAAAGAAUUAUUU | chr4:73404478-73404498 | 14 |
| G012748 | UUUGGCAUUUAUUUCUAAAA | chr4:73404496-73404516 | 15 |
| G012749 | UGUAUUUGUGAAGUCUUACA | chr4:73404529-73404549 | 16 |
| G012750 | UCCUAGGUAAAAAAAAAAAA | chr4:73404577-73404597 | 17 |
| G012751 | UAAUUUUCUUUUGCGCACUA | chr4:73404620-73404640 | 18 |
| G012752 | UGACUGAAACUUCACAGAAU | chr4:73404664-73404684 | 19 |
| G012753 | GACUGAAACUUCACAGAAUA | chr4:73404665-73404685 | 20 |
| G012754 | UUCAUUUUAGUCUGUCUUCU | chr4:73404803-73404823 | 21 |
| G012755 | AUUAUCUAAGUUUGAAUAUA | chr4:73404859-73404879 | 22 |
| G012756 | AAUUUUUAAAAUAGUAUUCU | chr4:73404897-73404917 | 23 |
| G012757 | UGAAUUAUUCUUCUGUUUAA | chr4:73404924-73404944 | 24 |
| G012758 | AUCAUCCUGAGUUUUUCUGU | chr4:73404965-73404985 | 25 |
| G012759 | UUACUAAAACUUUAUUUUAC | chr4:73404453-73404473 | 26 |
| G012760 | ACCUUUUUUUUUUUUUACCU | chr4:73404581-73404601 | 27 |
| G012761 | AGUGCAAUGGAUAGGUCUUU | chr4:73404714-73404734 | 28 |
| G012762 | UGAUUCCUACAGAAAAACUC | chr4:73404973-73404993 | 29 |
| G012763 | UGGGCAAGGGAAGAAAAAAA | chr4:73405094-73405114 | 30 |
| G012764 | CCUCACUCUUGUCUGGGCAA | chr4:73405107-73405127 | 31 |
| G012765 | ACCUCACUCUUGUCUGGGCA | chr4:73405108-73405128 | 32 |
| G012766 | UGAGCAACCUCACUCUUGUC | chr4:73405114-73405134 | 33 |

The albumin guide RNAs disclosed herein mediate a target-specific cutting resulting in a double-stranded break (DSB). The albumin guide RNAs disclosed herein mediate a target-specific cutting resulting in a single-stranded break (SSB or nick).

In some embodiments, the albumin guide RNAs disclosed herein bind to a region upstream of a propospacer adjacent motif (PAM). As would be understood by those of skill in the art, the PAM sequence occurs on the strand opposite to the strand that contains the target sequence. That is, the PAM sequence is on the complement strand of the target strand (the strand that contains the target sequence to which the guide RNA binds). In some embodiments, the PAM is selected from the group consisting of NGG, NNGRRT, NNGRR(N), NNAGAAW, NNNNG(A/C)TT, and NNNNRYAC. In some embodiments, the PAM is NGG.

In some embodiments, the guide RNA sequences provided herein are complementary to a sequence adjacent to a PAM sequence.

In some embodiments, the guide RNA sequence comprises a sequence that is complementary to a sequence within a genomic region selected from the tables herein according to coordinates in human reference genome hg38. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides from within a genomic region selected from the tables herein. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides spanning a genomic region selected from the tables herein.

The guide RNAs disclosed herein mediate a target-specific cutting resulting in a double-stranded break (DSB). The guide RNAs disclosed herein mediate a target-specific cutting resulting in a single-stranded break (SSB or nick).

In some embodiments, the albumin guide RNAs disclosed herein mediates target-specific cutting by a RNA-guided DNA binding agent (e.g., a Cas nuclease, as disclosed herein), wherein a resultant cut site allows insertion of a heterologous AAT nucleic acid (e.g., a functional or wild-type AAT) within intron 1 of an albumin gene. In some embodiments, the guide RNA and/or cut site allows between 1 and 5%, 5 and 10%, 15 and 20%, 20 and 25%, 25 and 30%, 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% insertion of a heterologous AAT gene. In some embodiments, the guide RNA and/or cut site allows at least 1%, at least 5%, at least 10%, at least 15%, at least 20%, at least 30%, at least 40%, at least 50%, at least 60%, at least 70%, at least 80%, at least 90% insertion of a heterologous AAT nucleic acid. Insertion rates can be measured in vitro or in vivo. For example, in some embodiments, rate of insertion can be determined by detecting and measuring the inserted heterologous AAT nucleic acid within a population of cells, and calculating a percentage of the population that contains the inserted heterologous AAT nucleic acid. Methods of measuring insertion rates are known and available in the art. Such methods include, e.g., sequencing of the insertion site or sequencing mRNA isolated from a tissue or cell population of interest.

In some embodiments, the guide RNA allows between 5 and 10%, 10 and 15%, 15 and 20%, 20 and 25%, 25 and 30%, 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, 95 and 99% or more increased expression and/or secretion of a heterologous AAT gene. For example, in some embodiments, increased expression and/or secretion can be determined by detecting and measuring the AAT polypeptide level and comparing the level against the AAT polypeptide level before, e.g., treating the cells or administration to a subject. Increased expression and/or secretion of a heterologous AAT gene can be measured in vitro or in vivo. In some embodiments, secretion and/or expression of AAT is measured either by detecting protein secreted by tissue or population of cells (e.g., in serum or cell media) or by detecting total cellular amount of the protein from a tissue or cell population of interest, using, e.g., an enzyme-linked immunosorbent assay (ELISA), HPLC, mass spectrometry (e.g., liquid mass spectrometry (e.g., LC-MS, LC-MS/MS), or western blot assay with culture media and/or cell or tissue (e.g., liver) extract. In some embodiments, secretion and/or expression of AAT is measured in primary human hepatocytes, e.g. media or cellular samples. In some embodiments, secretion of AAT is measured in HUH7 cells, e.g. media samples. In some embodiments, the cell used is HUH7 cells. In some embodiments, the amount of AAT is compared to the amount of glyceraldehyde 3-phosphate dehydrogenase GAPDH (a housekeeping gene) to control for changes in cell number. In some embodiments, AAT may be assessed by PASD staining of liver tissue sections, e.g., to measure aggregation. In some embodiments, AAT may be assessed by measuring inhibition of neutrophil elastase, e.g., in the lung.

In some embodiments, the guide RNA allows between 5 and 10%, 10 and 15%, 15 and 20%, 20 and 25%, 25 and 30%, 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, 95 and 99% or more increased activity that results from expression of a heterologous AAT gene (e.g., a functional or wild-type AAT). For example, increased activity can be determined by detecting and measuring the protease inhibitor activity level and comparing the level against a level of activity before, e.g., treating the cells or administration to a subject. Such methods are available and known in the art. See, e.g., Mullins et al., “Standardized automated assay for functional alpha 1-antitrypsin,” 1984; Eckfeldt et al., “Automated assay for alpha-1-antitiypsin with N-a-benzoyl-DL-arginine-p-nitroanilide astrypsin substrate and standardized with p-nitrophenyl-p′-guanidinobenzoateastitrant fortrypsinactivesites,” 1982.

In some embodiments, the target sequence or region within intron 1 of a human albumin locus (of SEQ ID NO: 1) may be complementary to the guide sequence of the albumin guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is about 20, or 20. In some embodiments, the target sequence and the guide sequence of the gRNA may contain 1-4 mismatches where the guide sequence is about 20, or 20 nucleotides.

As described and exemplified herein, the albumin guide RNAs can be used to insert and express a heterologous AAT gene (e.g., a functional or wild-type AAT) at intron 1 of an albumin gene, in combination with a SERPINA1 guide RNA to knockdown or knockout an endogenous SERPINA1 gene (e.g., a mutant SERPINA1 gene). Thus, in some embodiments, the present disclosure includes compositions comprising one or more SERPINA1 guide RNA (gRNA) comprising guide sequences that direct a RNA-guided DNA binding agent (e.g., Cas9) to a target DNA sequence in SERPINA1. The gRNA may comprise one or more of the guide sequences shown in Table 2. In some embodiments, provided herein are one or more SERPINA1 guide RNAs comprising a guide sequence of any one of SEQ ID NOs: 1000-1128.

In one aspect, the disclosure provides a SERPINA1 gRNA that comprises a guide sequence that is at least 99%, 98%, 97%, 96%, 95%, 94%, 93%, 92%, 91%, 90%, 89%, or 88% identical to a sequence selected from SEQ ID NOs: 1000-1128.

In other embodiments, the composition comprises at least two SERPINA1 gRNA's comprising guide sequences selected from any two or more of the guide sequences of SEQ ID NOs: 1000-1128. In some embodiments, the composition comprises at least two gRNA's that each are at least 99%, 98%, 97%, 96%, 95%, 94%, 93%, 92%, 91%, 90%, 89%, or 88% identical to any of the nucleic acids of SEQ ID NOs: 1000-1128.

The SERPINA1 guide RNA compositions of the present invention are designed to recognize a target sequence in the SERPINA1 gene. For example, the SERPINA1 target sequence may be recognized and cleaved by the provided RNA-guided DNA binding agent. In some embodiments, a Cas protein may be directed by a SERPINA1 guide RNA to a target sequence of the SERPINA1 gene, where the guide sequence of the guide RNA hybridizes with the target sequence and the Cas protein cleaves the target sequence.

In some embodiments, the selection of the one or more SERPINA1 guide RNAs is determined based on target sequences within the SERPINA1 gene.

Without being bound by any particular theory, mutations in critical regions of the gene may be less tolerable than mutations in non-critical regions of the gene, thus the location of a DSB is an important factor in the amount or type of protein knockdown or knockout that may result. In some embodiments, a SERPINA1 gRNA complementary or having complementarity to a target sequence within SERPINA1 is used to direct the Cas protein to a particular location in the SERPINA1 gene. In some embodiments, SERPINA1 gRNAs are designed to have guide sequences that are complementary or have complementarity to target sequences in exons 2, 3, 4, or 5 of SERPINA1.

In some embodiments, SERPINA1 gRNAs are designed to be complementary or have complementarity to target sequences in exons of SERPINA1 that code for the N-terminal region of AAT.

TABLE 2
SERPINA1 targeted and control guide sequence nomenclature, chromosomal
coordinates, and sequence

| SEQ | | | | |
|---|---|---|---|---|
| ID | | | Human Chromosomal | |
| No | Guide ID | Description | coordinates (hg38) | Guide Sequences |
| 1000 | CR001261 | Control 1 | Chr1:55039269- | GCCAGACUCCAAGUUCUGCC |
| | | | 55039291 | |
| 1001 | CR001262 | Control 2 | Chr1:55039155- | UAAGGCCAGUGGAAAGAAUU |
| | | | 55039177 | |
| 1002 | CR001263 | Control 3 | Chr1:55039180- | GGCAGCGAGGAGUCCACAGU |
| | | | 55039202 | |
| 1003 | CR001264 | Control 4 | Chr1:55039149- | UCUUUCCACUGGCCUUAACC |
| | | | 55039171 | |
| 1004 | CR001367 | Exon 2 | Chr14:94383211- | CAAUGCCGUCUUCUGUCUCG |
| | | | 94383233 | |
| 1005 | CR001368 | Exon 2 | Chr14:94383210- | AAUGCCGUCUUCUGUCUCGU |
| | | | 94383232 | |
| 1006 | CR001369 | Exon 2 | Chr14:94383209- | AUGCCGUCUUCUGUCUCGUG |
| | | | 94383231 | |
| 1007 | CR001370 | Exon 2 | Chr14:94383206- | AUGCCCCACGAGACAGAAGA |
| | | | 94383228 | |
| 1008 | CR001371 | Exon 2 | Chr14:94383195- | CUCGUGGGGCAUCCUCCUGC |
| | | | 94383217 | |
| 1009 | CR001372 | Exon 2 | Chr14:94383152- | GGAUCCUCAGCCAGGGAGAC |
| | | | 94383174 | |
| 1010 | CR001373 | Exon 2 | Chr14:94383146- | UCCCUGGCUGAGGAUCCCCA |
| | | | 94383168 | |
| 1011 | CR001374 | Exon 2 | Chr14:94383145- | UCCCUGGGGAUCCUCAGCCA |
| | | | 94383167 | |
| 1012 | CR001375 | Exon 2 | Chr14:94383144- | CUCCCUGGGGAUCCUCAGCC |
| | | | 94383166 | |
| 1013 | CR001376 | Exon 2 | Chr14:94383115- | GUGGGAUGUAUCUGUCUUCU |
| | | | 94383137 | |
| 1014 | CR001377 | Exon 2 | Chr14:94383114- | GGUGGGAUGUAUCUGUCUUC |
| | | | 94383136 | |
| 1015 | CR001378 | Exon 2 | Chr14:94383105- | AGAUACAUCCCACCAUGAUC |
| | | | 94383127 | |
| 1016 | CR001379 | Exon 2 | Chr14:94383097- | UGGGUGAUCCUGAUCAUGGU |
| | | | 94383119 | |
| 1017 | CR001380 | Exon 2 | Chr14:94383096- | UUGGGUGAUCCUGAUCAUGG |
| | | | 94383118 | |
| 1018 | CR001381 | Exon 2 | Chr14:94383093- | AGGUUGGGUGAUCCUGAUCA |
| | | | 94383115 | |
| 1019 | CR001382 | Exon 2 | Chr14:94383078- | GGGUGAUCUUGUUGAAGGUU |
| | | | 94383100 | |
| 1020 | CR001383 | Exon 2 | Chr14:94383077- | GGGGUGAUCUUGUUGAAGGU |
| | | | 94383099 | |
| 1021 | CR001384 | Exon 2 | Chr14:94383069- | CAACAAGAUCACCCCCAACC |
| | | | 94383091 | |
| 1022 | CR001385 | Exon 2 | Chr14:94383057- | AGGCGAACUCAGCCAGGUUG |
| | | | 94383079 | |
| 1023 | CR001386 | Exon 2 | Chr14:94383055- | GAAGGCGAACUCAGCCAGGU |
| | | | 94383077 | |
| 1024 | CR001387 | Exon 2 | Chr14:94383051- | GGCUGAAGGCGAACUCAGCC |
| | | | 94383073 | |
| 1025 | CR001388 | Exon 2 | Chr14:94383037- | CAGCUGGCGGUAUAGGCUGA |
| | | | 94383059 | |
| 1026 | CR001389 | Exon 2 | Chr14:94383036- | CUUCAGCCUAUACCGCCAGC |
| | | | 94383058 | |
| 1027 | CR001390 | Exon 2 | Chr14:94383030- | GGUGUGCCAGCUGGCGGUAU |
| | | | 94383052 | |
| 1028 | CR001391 | Exon 2 | Chr14:94383021- | UGUUGGACUGGUGUGCCAGC |
| | | | 94383043 | |
| 1029 | CR001392 | Exon 2 | Chr14:94383009- | AGAUAUUGGUGCUGUUGGAC |
| | | | 94383031 | |
| 1030 | CR001393 | Exon 2 | Chr14:94383004- | GAAGAAGAUAUUGGUGCUGU |
| | | | 94383026 | |
| 1031 | CR001394 | Exon 2 | Chr14:94382995- | CACUGGGGAGAAGAAGAUAU |
| | | | 94383017 | |
| 1032 | CR001395 | Exon 2 | Chr14:94382980- | GGCUGUAGCGAUGCUCACUG |
| | | | 94383002 | |
| 1033 | CR001396 | Exon 2 | Chr14:94382979- | AGGCUGUAGCGAUGCUCACU |
| | | | 94383001 | |
| 1034 | CR001397 | Exon 2 | Chr14:94382978- | AAGGCUGUAGCGAUGCUCAC |
| | | | 94383000 | |
| 1035 | CR001398 | Exon 2 | Chr14:94382928- | UGACACUCACGAUGAAAUCC |
| | | | 94382950 | |
| 1036 | CR001399 | Exon 2 | Chr14:94382925- | CACUCACGAUGAAAUCCUGG |
| | | | 94382947 | |
| 1037 | CR001400 | Exon 2 | Chr14:94382924- | ACUCACGAUGAAAUCCUGGA |
| | | | 94382946 | |
| 1038 | CR001401 | Exon 2 | Chr14:94382910- | GGUUGAAAUUCAGGCCCUCC |
| | | | 94382932 | |
| 1039 | CR001402 | Exon 2 | Chr14:94382904- | GGGCCUGAAUUUCAACCUCA |
| | | | 94382926 | |
| 1040 | CR001403 | Exon 2 | Chr14:94382895- | UUUCAACCUCACGGAGAUUC |
| | | | 94382917 | |
| 1041 | CR001404 | Exon 2 | Chr14:94382892- | CAACCUCACGGAGAUUCCGG |
| | | | 94382914 | |
| 1042 | CR001405 | Exon 2 | Chr14:94382889- | GAGCCUCCGGAAUCUCCGUG |
| | | | 94382911 | |
| 1043 | CR001406 | Exon 2 | Chr14:94382876- | CCGGAGGCUCAGAUCCAUGA |
| | | | 94382898 | |
| 1044 | CR001407 | Exon 2 | Chr14:94382850- | UGAGGGUACGGAGGAGUUCC |
| | | | 94382872 | |
| 1045 | CR001408 | Exon 2 | Chr14:94382841- | CUGGCUGGUUGAGGGUACGG |
| | | | 94382863 | |
| 1046 | CR001409 | Exon 2 | Chr14:94382833- | CUGGCUGUCUGGCUGGUUGA |
| | | | 94382855 | |
| 1047 | CR001410 | Exon 2 | Chr14:94382810- | CUCCAGCUGACCACCGGCAA |
| | | | 94382832 | |
| 1048 | CR001411 | Exon 2 | Chr14:94382808- | GGCCAUUGCCGGUGGUCAGC |
| | | | 94382830 | |
| 1049 | CR001412 | Exon 2 | Chr14:94382800- | GAGGAACAGGCCAUUGCCGG |
| | | | 94382822 | |
| 1050 | CR001413 | Exon 2 | Chr14:94382797- | GCUGAGGAACAGGCCAUUGC |
| | | | 94382819 | |
| 1051 | CR001414 | Exon 2 | Chr14:94382793- | CAAUGGCCUGUUCCUCAGCG |
| | | | 94382815 | |
| 1052 | CR001415 | Exon 2 | Chr14:94382792- | AAUGGCCUGUUCCUCAGCGA |
| | | | 94382814 | |
| 1053 | CR001416 | Exon 2 | Chr14:94382787- | UCAGGCCCUCGCUGAGGAAC |
| | | | 94382809 | |
| 1054 | CR001417 | Exon 2 | Chr14:94382781- | CUAGCUUCAGGCCCUCGCUG |
| | | | 94382803 | |
| 1055 | CR001418 | Exon 2 | Chr14:94382778- | CAGCGAGGGCCUGAAGCUAG |
| | | | 94382800 | |
| 1056 | CR001419 | Exon 2 | Chr14:94382769- | AAAACUUAUCCACUAGCUUC |
| | | | 94382791 | |
| 1057 | CR001420 | Exon 2 | Chr14:94382766- | GAAGCUAGUGGAUAAGUUUU |
| | | | 94382788 | |
| 1058 | CR001421 | Exon 2 | Chr14:94382763- | GCUAGUGGAUAAGUUUUUGG |
| | | | 94382785 | |
| 1059 | CR001422 | Exon 2 | Chr14:94382724- | UGACAGUGAAGGCUUCUGAG |
| | | | 94382746 | |
| 1060 | CR001423 | Exon 2 | Chr14:94382716- | AAGCCUUCACUGUCAACUUC |
| | | | 94382738 | |
| 1061 | CR001424 | Exon 2 | Chr14:94382715- | AGCCUUCACUGUCAACUUCG |
| | | | 94382737 | |
| 1062 | CR001425 | Exon 2 | Chr14:94382713- | GUCCCCGAAGUUGACAGUGA |
| | | | 94382735 | |
| 1063 | CR001426 | Exon 2 | Chr14:94382703- | CAACUUCGGGGACACCGAAG |
| | | | 94382725 | |
| 1064 | CR001427 | Exon 2 | Chr14:94382689- | GAUCUGUUUCUUGGCCUCUU |
| | | | 94382711 | |
| 1065 | CR001428 | Exon 2 | Chr14:94382680- | GUAAUCGUUGAUCUGUUUCU |
| | | | 94382702 | |
| 1066 | CR001429 | Exon 2 | Chr14:94382676- | GAAACAGAUCAACGAUUACG |
| | | | 94382698 | |
| 1067 | CR001430 | Exon 2 | Chr14:94382670- | GAUCAACGAUUACGUGGAGA |
| | | | 94382692 | |
| 1068 | CR001431 | Exon 2 | Chr14:94382669- | AUCAACGAUUACGUGGAGAA |
| | | | 94382691 | |
| 1069 | CR001432 | Exon 2 | Chr14:94382660- | UACGUGGAGAAGGGUACUCA |
| | | | 94382682 | |
| 1070 | CR001433 | Exon 2 | Chr14:94382659- | ACGUGGAGAAGGGUACUCAA |
| | | | 94382681 | |
| 1071 | CR001434 | Exon 2 | Chr14:94382643- | UCAAGGGAAAAUUGUGGAUU |
| | | | 94382665 | |
| 1072 | CR001435 | Exon 2 | Chr14:94382637- | GAAAAUUGUGGAUUUGGUCA |
| | | | 94382659 | |
| 1073 | CR001436 | Exon 2 | Chr14:94382607- | CAGAGACACAGUUUUUGCUC |
| | | | 94382629 | |
| 1074 | CR001437 | Exon 3 | Chr14:94381127- | UCCCCUCUCUCCAGGCAAAU |
| | | | 94381149 | |
| 1075 | CR001438 | Exon 3 | Chr14:94381098- | CUCGGUGUCCUUGACUUCAA |
| | | | 94381120 | |
| 1076 | CR001439 | Exon 3 | Chr14:94381097- | CUUUGAAGUCAAGGACACCG |
| | | | 94381119 | |
| 1077 | CR001440 | Exon 3 | Chr14:94381080- | CACGUGGAAGUCCUCUUCCU |
| | | | 94381102 | |
| 1078 | CR001441 | Exon 3 | Chr14:94381079- | CGAGGAAGAGGACUUCCACG |
| | | | 94381101 | |
| 1079 | CR001442 | Exon 3 | Chr14:94381073- | AGAGGACUUCCACGUGGACC |
| | | | 94381095 | |
| 1080 | CR001443 | Exon 3 | Chr14:94381064- | CGGUGGUCACCUGGUCCACG |
| | | | 94381086 | |
| 1081 | CR001444 | Exon 3 | Chr14:94381058- | GGACCAGGUGACCACCGUGA |
| | | | 94381080 | |
| 1082 | CR001445 | Exon 3 | Chr14:94381055- | GCACCUUCACGGUGGUCACC |
| | | | 94381077 | |
| 1083 | CR001446 | Exon 3 | Chr14:94381047- | CAUCAUAGGCACCUUCACGG |
| | | | 94381069 | |
| 1084 | CR001447 | Exon 3 | Chr14:94381036- | GUGCCUAUGAUGAAGCGUUU |
| | | | 94381058 | |
| 1085 | CR001448 | Exon 3 | Chr14:94381033- | AUGCCUAAACGCUUCAUCAU |
| | | | 94381055 | |
| 1086 | CR001449 | Exon 3 | Chr14:94381001- | UGGACAGCUUCUUACAGUGC |
| | | | 94381023 | |
| 1087 | CR001450 | Exon 3 | Chr14:94380995- | CUGUAAGAAGCUGUCCAGCU |
| | | | 94381017 | |
| 1088 | CR001451 | Exon 3 | Chr14:94380974- | GGUGCUGCUGAUGAAAUACC |
| | | | 94380996 | |
| 1089 | CR001452 | Exon 3 | Chr14:94380973- | GUGCUGCUGAUGAAAUACCU |
| | | | 94380995 | |
| 1090 | CR001453 | Exon 3 | Chr14:94380956- | AGAUGGCGGUGGCAUUGCCC |
| | | | 94380978 | |
| 1091 | CR001454 | Exon 3 | Chr14:94380945- | AGGCAGGAAGAAGAUGGCGG |
| | | | 94380967 | |
| 1092 | CR001474 | Exon 5 | Chr14:94378611- | GGUCAGCACAGCCUUAUGCA |
| | | | 94378633 | |
| 1093 | CR001475 | Exon 5 | Chr14:94378581- | AGAAAGGGACUGAAGCUGCU |
| | | | 94378603 | |
| 1094 | CR001476 | Exon 5 | Chr14:94378580- | GAAAGGGACUGAAGCUGCUG |
| | | | 94378602 | |
| 1095 | CR001477 | Exon 5 | Chr14:94378565- | UGCUGGGGCCAUGUUUUUAG |
| | | | 94378587 | |
| 1096 | CR001478 | Exon 5 | Chr14:94378557- | GGGUAUGGCCUCUAAAAACA |
| | | | 94378579 | |
| 1097 | CR001483 | Exon 5 | Chr14:94378526- | UGUUGAACUUGACCUCGGGG |
| | | | 94378548 | |
| 1098 | CR001484 | Exon 5 | Chr14:94378521- | GGGUUUGUUGAACUUGACCU |
| | | | 94378543 | |
| 1099 | CR003190 | Exon 2 | Chr14:94383131- | UUCUGGGCAGCAUCUCCCUG |
| | | | 94383153 | |
| 1100 | CR003191 | Exon 2 | Chr14:94383129- | UCUUCUGGGCAGCAUCUCCC |
| | | | 94383151 | |
| 1101 | CR003196 | Exon 2 | Chr14:94383024- | UGGACUGGUGUGCCAGCUGG |
| | | | 94383046 | |
| 1102 | CR003204 | Exon 2 | Chr14:94382961- | AGCCUUUGCAAUGCUCUCCC |
| | | | 94382983 | |
| 1103 | CR003205 | Exon 2 | Chr14:94382935- | UUCAUCGUGAGUGUCAGCCU |
| | | | 94382957 | |
| 1104 | CR003206 | Exon 2 | Chr14:94382901- | UCUCCGUGAGGUUGAAAUUC |
| | | | 94382923 | |
| 1105 | CR003207 | Exon 2 | Chr14:94382822- | GUCAGCUGGAGCUGGCUGUC |
| | | | 94382844 | |
| 1106 | CR003208 | Exon 2 | Chr14:94382816- | AGCCAGCUCCAGCUGACCAC |
| | | | 94382838 | |
| 1107 | CR003217 | Exon 3 | Chr14:94380942- | AUCAGGCAGGAAGAAGAUGG |
| | | | 94380964 | |
| 1108 | CR003218 | Exon 3 | Chr14:94380938- | CAUCUUCUUCCUGCCUGAUG |
| | | | 94380960 | |
| 1109 | CR003219 | Exon 3 | Chr14:94380937- | AUCUUCUUCCUGCCUGAUGA |
| | | | 94380959 | |
| 1110 | CR003220 | Exon 3 | Chr14:94380881- | CGAUAUCAUCACCAAGUUCC |
| | | | 94380903 | |
| 1111 | CR003221 | Exon 4 | Chr14:94379554- | CAGAUCAUAGGUUCCAGUAA |
| | | | 94379576 | |
| 1112 | CR003222 | Exon 4 | Chr14:94379507- | AUCACUAAGGUCUUCAGCAA |
| | | | 94379529 | |
| 1113 | CR003223 | Exon 4 | Chr14:94379506- | UCACUAAGGUCUUCAGCAAU |
| | | | 94379528 | |
| 1114 | CR003224 | Exon 4 | Chr14:94379505- | CACUAAGGUCUUCAGCAAUG |
| | | | 94379527 | |
| 1115 | CR003225 | Exon 4 | Chr14:94379453- | CUCACCUUGGAGAGCUUCAG |
| | | | 94379475 | |
| 1116 | CR003226 | Exon 4 | Chr14:94379452- | UCUCACCUUGGAGAGCUUCA |
| | | | 94379474 | |
| 1117 | CR003227 | Exon 4 | Chr14:94379451- | AUCUCACCUUGGAGAGCUUC |
| | | | 94379473 | |
| 1118 | CR003235 | Exon 5 | Chr14:94378525- | UUGUUGAACUUGACCUCGGG |
| | | | 94378547 | |
| 1119 | CR003236 | Exon 5 | Chr14:94378524- | UUUGUUGAACUUGACCUCGG |
| | | | 94378546 | |
| 1120 | CR003237 | Exon 5 | Chr14:94378523- | GUUUGUUGAACUUGACCUCG |
| | | | 94378545 | |
| 1121 | CR003238 | Exon 5 | Chr14:94378522- | GGUUUGUUGAACUUGACCUC |
| | | | 94378544 | |
| 1122 | CR003240 | Exon 5 | Chr14:94378501- | UCAAUCAUUAAGAAGACAAA |
| | | | 94378523 | |
| 1123 | CR003241 | Exon 5 | Chr14:94378500- | UUCAAUCAUUAAGAAGACAA |
| | | | 94378522 | |
| 1124 | CR003242 | Exon 5 | Chr14:94378472- | UACCAAGUCUCCCCUCUUCA |
| | | | 94378494 | |
| 1125 | CR003243 | Exon 5 | Chr14:94378471- | ACCAAGUCUCCCCUCUUCAU |
| | | | 94378493 | |
| 1126 | CR003244 | Exon 5 | Chr14:94378463- | UCCCCUCUUCAUGGGAAAAG |
| | | | 94378485 | |
| 1127 | CR003245 | Exon 5 | Chr14:94378461- | CACCACUUUUCCCAUGAAGA |
| | | | 94378483 | |
| 1128 | CR003246 | Exon 5 | Chr14:94378460- | UCACCACUUUUCCCAUGAAG |
| | | | 94378482 | |

Each of the albumin guide sequences and SERPINA1 guide sequences shown in Table 1 at SEQ ID NOs: 2-33 and Table 2 at SEQ ID Nos: 1000-1128, respectively, may further comprise additional nucleotides to form a crRNA and/or guide RNA, e.g., with the following exemplary nucleotide sequence following the guide sequence at its 3′ end: GUUUUAGAGCUAUGCUGUUUUG (SEQ ID NO: 900) in 5′ to 3′ orientation. In the case of a sgRNA, the above guide sequences (the albumin guide sequences and SERPINA1 guide sequences shown in Table 1 at SEQ ID NOs:2-33 and Table 2 at SEQ ID Nos: 1000-1128, respectively) may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUUGA AAAAGUGGCACCGAGUCGGUGCUUUU (SEQ ID NO: 901) in 5′ to 3′ orientation.

The albumin and/or SERPINA1 guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, the sgRNA comprises one or more linkages between nucleotides that is not a phosphodiester linkage.

In each of the composition, use, and method embodiments described herein, the guide RNA may comprise two RNA molecules as a “dual guide RNA” or “dgRNA”. The dgRNA comprises a first RNA molecule comprising a crRNA comprising, e.g., a guide sequence shown in Table 1 and/or Table 2, and a second RNA molecule comprising a trRNA. The first and second RNA molecules may not be covalently linked, but may form a RNA duplex via the base pairing between portions of the crRNA and the trRNA.

In each of the composition, use, and method embodiments described herein, the guide RNA (albumin gRNA and/or SERPINA1 gRNA) may comprise a single RNA molecule as a “single guide RNA” or “sgRNA”. The sgRNA may comprise a crRNA (or a portion thereof) comprising a guide sequence shown in Table 1 or Table 2 covalently linked to a trRNA. The sgRNA may comprise 15, 16, 17, 18, 19, or 20 contiguous nucleotides of a guide sequence shown in Table 1 or Table 2. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond. In some embodiments, the guide RNA comprises a sgRNA shown in any one of SEQ ID No: 34-67 or 120-163. In some embodiments, the guide RNA comprises a sgRNA comprising any one of the guide sequences of SEQ ID No: 2-33, 98-119, 165-170, 172, 174-176, 182-185, 189-193, 195-193, 195, or 196 and the nucleotides of SEQ ID No: 901 , wherein the nucleotides of SEQ ID No: 901 are on the 3′ end of the guide sequence, and wherein the sgRNA may be modified as shown in Tables 9, 11, or 13 or SEQ ID NO: 300.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

### C. Modified gRNAs and mRNAs

In some embodiments, the gRNA disclosed herein (e.g., albumin or SERPINA1 gRNA) is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs and/or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

In some embodiments, the guide RNAs disclosed herein comprise one of the modification patterns disclosed in WO2018/107028 A1, filed Dec. 8, 2017, titled “Chemically Modified Guide RNAs,” the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, WO2017004279, US2018187186, US2019048338, the contents of which are hereby incorporated by reference in their entirety.

In some embodiments, the modified sgRNA comprises the following sequence: mN*mN*mN* GUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 300), where “N” may be any natural or non-natural nucleotide, and wherein the totality of N's comprise an albumin intron 1 guide sequence as described in Tables 1, 10, and 12; and SERPINA1 guide sequences as described in Table 2. For example, encompassed herein is SEQ ID NO: 300, where the N's are replaced with any of the guide sequences disclosed herein in Table 1 (SEQ ID Nos: 2-33) and/or Table 2 (SEQ ID Nos: 1000-1128).

Any of the modififications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

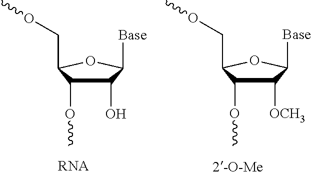

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

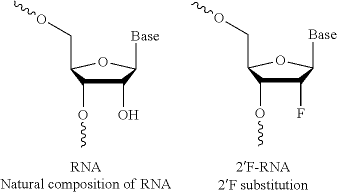

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S-into a nonbridging phosphate oxygen, generating a PS bond in lieu of a phosphodiester bond:

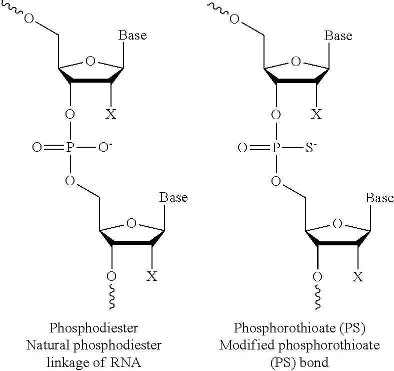

Abasic nucleotides refer to those which lack nitrogenous bases. The figure below depicts an oligonucleotide with an abasic (also known as apurinic) site that lacks a base:

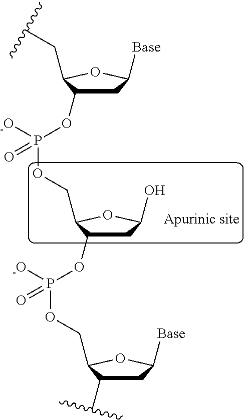

Inverted bases refer to those with linkages that are inverted from the normal 5′ to 3′ linkage (i.e., either a 5′ to 5′ linkage or a 3′ to 3′ linkage). For example:

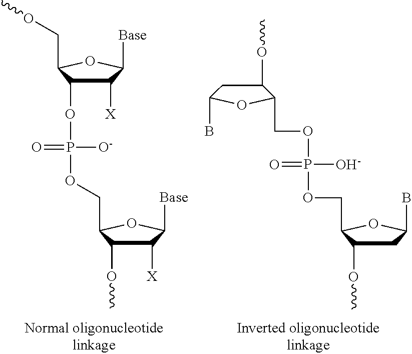

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, any of the guide RNAs disclosed herein comprises a modified sgRNA. In some embodiments, the sgRNA comprises the modification pattern shown in SEQ ID NO: 200, where N is any natural or non-natural nucleotide, and where the totality of the N's comprise a guide sequence (e.g., as shown in Table 1 or Table 2) that directs a nuclease to a target sequence (e.g., in human albumin intron 1 or SERPINA1).

As noted above, in some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered. As described below, the mRNA comprising a Cas nuclease may comprise a Cas9 nuclease, such as an S. pyogenes Cas9 nuclease having cleavase, nickase, and/or site-specific DNA binding activity. In some embodiments, the ORF encoding an RNA-guided DNA nuclease is a “modified RNA-guided DNA binding agent ORF” or simply a “modified ORF,” which is used as shorthand to indicate that the ORF is modified.

Cas9 ORFs, including modified Cas9 ORFs, are provided herein and are known in the art. As one example, the Cas9 ORF can be codon optimized, such that coding sequence includes one or more alternative codons for one or more amino acids. An “alternative codon” as used herein refers to variations in codon usage for a given amino acid, and may or may not be a preferred or optimized codon (codon optimized) for a given expression system. Preferred codon usage, or codons that are well-tolerated in a given system of expression, is known in the art. The Cas9 coding sequences, Cas9 mRNAs, and Cas9 protein sequences of WO2013/176772, WO2014/065596, WO2016/106121, and WO2019/067910 are hereby incorporated by reference. In particular, the ORFs and Cas9 amino acid sequences of the table at paragraph [0449] WO2019/067910, and the Cas9 mRNAs and ORFs of paragraphs [0214]-[0234] of WO2019/067910 are hereby incorporated by reference.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0 , Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

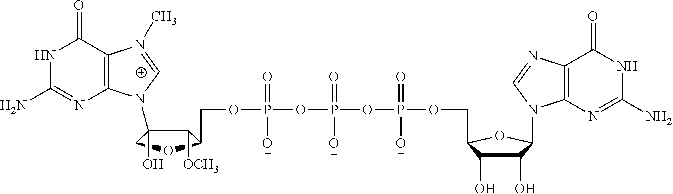

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

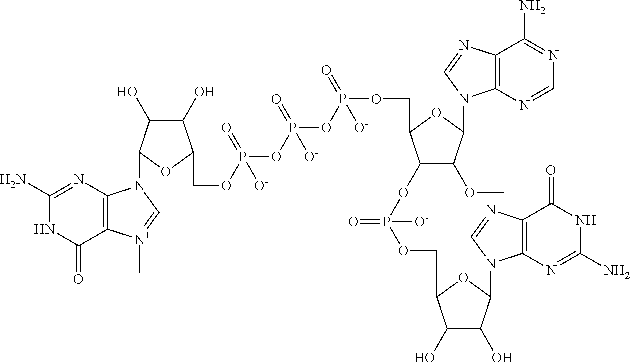 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0 , in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides.

### D. Donor Constructs

The compositions and methods described herein include the use of a nucleic acid construct that comprises a sequence encoding a heterologous AAT gene (e.g., a functional or wild-type AAT) to be inserted into a cut site created by a guide RNA of the present disclosure and a RNA-guided DNA binding agent. As used herein, such a construct is sometimes referred to as a “donor construct/template”. In some embodiments, the construct is a DNA construct. Methods of designing and making various functional/structural modifications to donor constructs are known in the art. In some embodiments, the construct may comprise any one or more of a polyadenylation tail sequence, a polyadenylation signal sequence, splice acceptor site, or selectable marker. In some embodiments, the polyadenylation tail sequence is encoded, e.g., as a “poly-A” stretch, at the 3′ end of the coding sequence. Methods of designing a suitable polyadenylation tail sequence and/or polyadenylation signal sequence are well known in the art. For example, the polyadenylation signal sequence AAUAAA (SEQ ID NO: 800) is commonly used in mammalian systems, although variants such as UAUAAA (SEQ ID NO: 801) or AU/GUAAA (SEQ ID NO: 802) have been identified. See, e.g., NJ Proudfoot, Genes & Dev. 25(17):1770-82, 2011.

The length of the construct can vary, depending on the size of the gene to be inserted, and can be, for example, from 200 base pairs (bp) to about 5000 bp, such as about 200 bp to about 2000 bp, such as about 500 bp to about 1500 bp. In some embodiments, the length of the DNA donor template is about 200 bp, or is about 500 bp, or is about 800 bp, or is about 1000 base pairs, or is about 1500 base pairs. In other embodiments, the length of the donor template is at least 200 bp, or is at least 500 bp, or is at least 800 bp, or is at least 1000 bp, or is at least 1500 bp, or at least 2000, or at least 2500, or at least 3000, or at least 3500, or at least 4000, or at least 4500, or at least 5000.

The construct can be DNA or RNA, single-stranded, double-stranded or partially single- and partially double-stranded and can be introduced into a host cell in linear or circular (e.g., minicircle) form. See, e.g., U.S. Patent Publication Nos. 2010/0047805, 2011/0281361, 2011/0207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters and genes encoding antibiotic resistance. A construct may omit viral elements. Moreover, donor constructs can be introduced as naked nucleic acid, as nucleic acid complexed with an agent such as a liposome or poloxamer, or can be delivered by viruses (e.g., adenovirus, AAV, herpesvirus, retrovirus, lentivirus).

In some embodiments, the construct may be inserted so that its expression is driven by the endogenous promoter at the insertion site (e.g., the endogenous albumin promoter when the donor is integrated into the host cell's albumin locus). In such cases, the transgene may lack control elements (e.g., promoter and/or enhancer) that drive its expression (e.g., a promoterless construct). Nonetheless, it will be apparent that in other cases the construct may comprise a promoter and/or enhancer, for example a constitutive promoter or an inducible or tissue specific (e.g., liver- or platelet-specific) promoter that drives expression of the functional protein upon integration. The construct may comprise a sequence encoding a heterologous AAT protein downstream of and operably linked to a signal sequence encoding a signal peptide. In some embodiments, the signal peptide is a signal peptide from a hepatocyte secreted protein. In some embodiments, the signal peptide is an AAT signal peptide. In some embodiments, the signal peptide is an albumin signal peptide. In some embodiments, the signal peptide is an Factor IX signal peptide. The construct may comprise a sequence encoding a heterologous AAT protein downstream of and operably linked to a signal sequence encoding an AAT signal peptide, e.g. SEQ ID NO: 700. The construct may comprise a sequence encoding a heterologous AAT protein downstream of and operably linked to a signal sequence encoding a heterologous signal peptide. In various embodiments, the methods comprise a sequence encoding a heterologous AAT protein downstream of and operably linked to a signal sequence encoding an albumin signal peptide (SEQ ID NO: 2000). In some embodiments, the nucleic acid construct works in homology-independent insertion of a nucleic acid that encodes an AAT protein. In some embodiments, the nucleic acid construct works in non-dividing cells, e.g., cells in which NHEJ, not HR, is the primary mechanism by which double-stranded DNA breaks are repaired. The nucleic acid may be a homology-independent donor construct.

In some embodiments, the donor construct comprises a heterologous AAT gene that encodes a functional AAT protein. In some embodiments, the functional AAT protein is a human wild-type AAT protein sequence according to SEQ ID NO: 700. In some embodiments, the functional AAT protein is a human wild-type AAT protein sequence according to SEQ ID NO: 702. Nucleic acid encoding AAT are also exemplified and disclosed herein. In some embodiments, the construct comprises a heterologous AAT gene that encodes a functional variant of AAT, e.g., a variant that possesses increased protease inhibitor activity as compared to wild type AAT. In some embodiments, the construct comprises a heterologous AAT gene that encodes a functional variant that is 80%, 85%, 90%, 93%, 95%, 97%, 99% identical to SEQ ID NO: 700, having a functional activity that is at least 80%, 85%, 90%, 92%, 94%, 96%, 98%, 99%, 100%, or more, activity as compared to wild type AAT. In some embodiments, the construct comprises a heterologous AAT gene that encodes a functional variant that is 80%, 85%, 90%, 93%, 95%, 97%, 99% identical to SEQ ID NO: 702, having a functional activity that is at least 80%, 85%, 90%, 92%, 94%, 96%, 98%, 99%, 100%, or more, activity as compared to wild type AAT. In some embodiments, the construct comprises a heterologous AAT gene that encodes a fragment of AAT protein that possesses functional activity that is at least 80%, 85%, 90%, 92%, 94%, 96%, 98%, 99%, 100%, or more, activity as compared to wild type AAT.

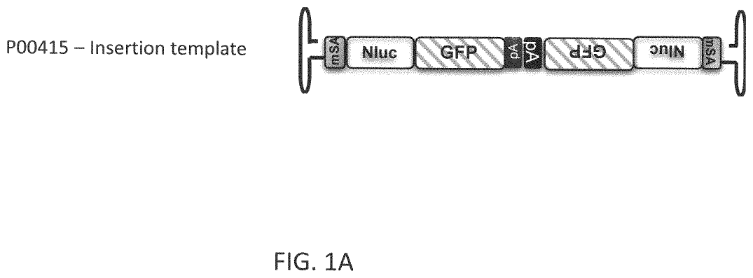

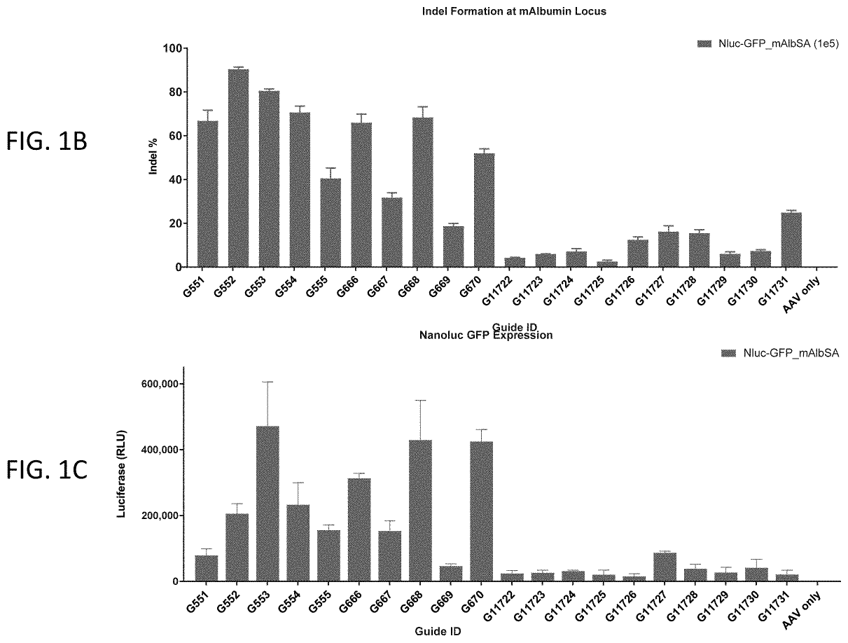

Also described herein are bidirectional nucleic acid constructs that allow enhanced insertion and expression of a heterologous AAT gene. Briefly, variousbidirectional constructs disclosed herein comprise at least two nucleic acid segments, wherein one segment (the first segment) comprises a coding sequence that encodes a heterologous AAT (sometimes interchangeably referred to herein as “transgene”), while the other segment (the second segment) comprises a sequence wherein the complement of the sequence encodes a heterologous AAT. The bidirectional constructs may comprise at least two nucleic acid segments in cis, wherein one segment (the first segment) comprises a coding sequence that encodes a heterologous AAT in one orientation, while the other segment (the second segment) comprises a sequence wherein its complement encodes a heterologous AAT in the other orientation. That is, first segment is a complement of the second segment (not necessarily a perfect complement); the complement of the second segment is the reverse complement of the first segment (not necessarily a perfect reverse complement as long as both encode a heterologous AAT). A bidirectional construct may comprise a first coding sequence that encodes a heterologous AAT linked to a splice acceptor and a second coding sequence wherein the complement encodes a heterologous AAT in the other orientation, also linked to a splice acceptor. When used in combination with a gene editing system (e.g., CRISPR/Cas system; zinc finger nuclease (ZFN) system; transcription activator-like effector nuclease (TALEN) system) as described herein, the bidirectionality of the nucleic acid constructs allows the construct to be inserted in either direction (is not limited to insertion in one direction) within a target insertion site, allowing the expression of a heterologous AAT from either a) a coding sequence of one segment (e.g., the left segment encoding “GFP” of FIG. 1 upper left ssAAV construct), or 2) a complement of the other segment (e.g., the complement of the right segment encoding “GFP” indicated upside down in the upper left ssAAV construct FIG. 1), thereby enhancing insertion and expression efficiency, as exemplified herein. Various known gene editing systems can be used in the practice of the present disclosure, including, e.g., CRISPR/Cas system; zinc finger nuclease (ZFN) system; transcription activator-like effector nuclease (TALEN) system.

The bidirectional constructs disclosed herein can be modified to include any suitable structural feature as needed for any particular use and/or that confers one or more desired function. In some embodiments, the bidirectional nucleic acid construct disclosed herein does not comprise a homology arm. In some embodiments, the bidirectional nucleic acid construct disclosed herein is a homology-independent donor construct. In some embodiments, owing in part to the bidirectional function of the nucleic acid construct, the bidirectional construct can be inserted into a genomic locus in either direction (orientation) as described herein to allow for efficient insertion and/or expression of a polypeptide of interest (e.g., a heterologous AAT).

In some embodiments, the bidirectional nucleic acid construct does not comprise a promoter that drives the expression of a heterologous AAT gene. For example, the expression of the polypeptide is driven by a promoter of the host cell (e.g., the endogenous albumin promoter when the transgene is integrated into a host cell's albumin locus). In some embodiments, the bidirectional nucleic acid construct includes a first segment and a second segment, each having a splice acceptor upstream of a transgene. In certain embodiments, the splice acceptor is compatible with the splice donor sequence of the host cell's safe harbor site, e.g. the splice donor of intron 1 of a human albumin gene.

In some embodiments, the bidirectional nucleic acid construct comprises a first segment comprising a coding sequence for heterologous AAT and a second segment comprising a reverse complement of a coding sequence of heterologous AAT. Thus, the coding sequence in the first segment is capable of expressing heterologous AAT, while the complement of the reverse complement in the second segment is also capable of expressing heterologous AAT. As used herein, “coding sequence” when referring to the second segment comprising a reverse complement sequence refers to the complementary (coding) strand of the second segment (i.e., the complement coding sequence of the reverse complement sequence in the second segment).

In some embodiments, the coding sequence that encodes a heterologous AAT in the first segment is less than 100% complementary to the reverse complement of a coding sequence that also encodes heterologous AAT. That is, in some embodiments, the first segment comprises a coding sequence (1) for heterologous AAT, and the second segment is a reverse complement of a coding sequence (2) for heterologous AAT, wherein the coding sequence (1) is not identical to the coding sequence (2). For example, coding sequence (1) and/or coding sequence (2) that encodes for heterologous AAT can be codon optimized, such that coding sequence (1) and the reverse complement of coding sequence (2) possess less than 100% complementarity. In some embodiments, the coding sequence of the second segment encodes heterologous AAT using one or more alternative codons for one or more amino acids of the same (i.e., same amino acid sequence) heterologous AAT encoded by the coding sequence in the first segment. An “alternative codon” as used herein refers to variations in codon usage for a given amino acid, and may or may not be a preferred or optimized codon (codon optimized) for a given expression system. Preferred codon usage, or codons that are well-tolerated in a given system of expression is known in the art.

In some embodiments, the second segment comprises a reverse complement sequence that adopts different codon usage from that of the coding sequence of the first segment in order to reduce hairpin formation. Such a reverse complement forms base pairs with fewer than all nucleotides of the coding sequence in the first segment, yet it optionally encodes the same polypeptide. In such cases, the coding sequence, e.g. for Polypeptide A, of the first segment many be homologous to, but not identical to, the coding sequence, e.g. for Polypeptide A of the second half of the bidirectional construct. In some embodiments, the second segment comprises a reverse complement sequence that is not substantially complementary (e.g., not more than 70% complementary) to the coding sequence in the first segment. In some embodiments, the second segment comprises a reverse complement sequence that is highly complementary (e.g., at least 90% complementary) to the coding sequence in the first segment. In some embodiments, the second segment comprises a reverse complement sequence having at least about 30%, about 35%, about 40%, about 45%, about 50%, about 55%, about 60%, about 65%, about 70%, about 75%, about 80%, about 85%, about 90%, about 95%, about 97%, or about 99% complementarity to the coding sequence in the first segment.

In some embodiments, the second segment comprises a reverse complement sequence having 100% complementarity to the coding sequence in the first segment. That is, the sequence in the second segment is a perfect reverse complement of the coding sequence in the first segment. By way of example, the first segment comprises a hypothetical sequence 5′ CTGGACCGA 3′ (SEQ ID NO: 500) and the second segment comprises the reverse complement of SEQ ID NO: 500—i.e., 5′ TCGGTCCAG 3′ (SEQ ID NO: 502).

In some embodiments, the bidirectional nucleic acid construct comprises a first segment comprising a coding sequence for a polypeptide or agent (e.g. a first polypeptide) and a second segment comprising a reverse complement of a coding sequence of a polypeptide or agent (e.g. a second polypeptide). In some embodiments, the first polypeptide and the second polypeptide are the same, as described above. In some embodiments, the first therapeutic agent and the second therapeutic agent are the same, as described above. In some embodiments, the first polypeptide and the second polypeptide are different. In some embodiments, the first therapeutic agent and the second therapeutic agent are different. For example, the first polypeptide is Polypeptide A and the second polypeptide is Polypeptide B. As a further example, the first polypeptide is Polypeptide A and the second polypeptide is a variant (e.g., a fragment (such as a functional fragment), mutant, fusion (including addition of as few as one amino acid at a polypeptide terminus), or combinations thereof) of Polypeptide A.

A coding sequence that encodes a polypeptide may optionally comprise one or more additional sequences, such as sequences encoding amino- or carboxy-terminal amino acid sequences such as a signal sequence, label sequence, or heterologous functional sequence (e.g. nuclear localization sequence (NLS)) linked to the polypeptide. A coding sequence that encodes a polypeptide may optionally comprise sequences encoding one or more amino-terminal signal peptide sequences. Each of these additional sequences can be the same or different in the first segment and second segment of the construct.

The bidirectional construct described herein can be used to express AAT as described herein.

In some embodiments, the bidirectional nucleic acid construct is linear. For example, the first and second segments are joined in a linear manner through a linker sequence. In some embodiments, the 5′ end of the second segment that comprises a reverse complement sequence is linked to the 3′ end of the first segment. In some embodiments, the 5′ end of the first segment is linked to the 3′ end of the second segment that comprises a reverse complement sequence. In some embodiments, the linker sequence is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 500, 1000, 1500, 2000 or more nucleotides in length. As would be appreciated by those of skill in the art, other structural elements in addition to, or instead of a linker sequence, can be inserted between the first and second segments.

The constructs disclosed herein can be modified to include any suitable structural feature as needed for any particular use and/or that confers one or more desired function. In some embodiments, the bidirectional nucleic acid construct disclosed herein does not comprise a homology arm. In some embodiments, owing in part to the bidirectional function of the nucleic acid construct, the bidirectional construct can be inserted into a genomic locus in either direction as described herein to allow for efficient insertion and/or expression of a polypeptide of interest.

In some embodiments, one or both of the first and second segment comprises a polyadenylation tail sequence and/or a polyadenylation signal sequence downstream of an open reading frame. In some embodiments, the polyadenylation tail sequence is encoded, e.g., as a “poly-A” stretch, at the 3′ end of the first and/or second segment. In some embodiments, a polyadenylation tail sequence is provided co-transcriptionally as a result of a polyadenylation signal sequence that is encoded at or near the 3′ end of the first and/or second segment. Methods of designing a suitable polyadenylation tail sequence and/or polyadenylation signal sequence are well known in the art. Suitable splice acceptor sequences are disclosed and exemplified herein, including mouse albumin and human FIX splice acceptor sites. In some embodiments, the polyadenylation signal sequence AAUAAA (SEQ ID NO: 800) is commonly used in mammalian systems, although variants such as UAUAAA (SEQ ID NO: 801) or AU/GUAAA (SEQ ID NO: 802) have been identified. See, e.g., NJ Proudfoot, Genes & Dev. 25(17):1770-82, 2011. In some embodiments, a polyA tail sequence is included.

In some embodiments, the constructs disclosed herein can be DNA or RNA, single-stranded, double-stranded, or partially single- and partially double-stranded. For example, the constructs can be single- or double-stranded DNA. In some embodiments, the nucleic acid can be modified (e.g., using nucleoside analogs), as described herein.

In some embodiments, the constructs disclosed herein comprise a splice acceptor site on either or both ends of the construct, e.g., 5′ of an open reading frame in the first and/or second segments, or 5′ of one or both transgene sequences. In some embodiments, the splice acceptor site comprises NAG. In further embodiments, the splice acceptor site consists of NAG. In some embodiments, the splice acceptor is an albumin splice acceptor, e.g., an albumin splice acceptor used in the splicing together of exons 1 and 2 of albumin. In some embodiments, the splice acceptor is derived from the human albumin gene. In some embodiments, the splice acceptor is derived from the mouse albumin gene. In some embodiments, the splice acceptor is a mouse albumin splice acceptor, e.g., the mouse albumin splice acceptor used in the splicing together of exons 1 and 2 of albumin. In some embodiments, the splice acceptor is derived from the human albumin gene. Additional suitable splice acceptor sites useful in eukaryotes, including artificial splice acceptors are known and can be derived from the art. See, e.g., Shapiro, et al., 1987, Nucleic Acids Res., 15, 7155-7174, Burset, et al., 2001, Nucleic Acids Res., 29, 255-259.

In some embodiments, the constructs disclosed herein can be modified on either or both ends to include one or more suitable structural features as needed, and/or to confer one or more functional benefit. For example, structural modifications can vary depending on the method(s) used to deliver the constructs disclosed herein to a host cell—e.g., use of viral vector delivery or packaging into lipid nanoparticles for delivery. Such modifications include, without limitation, e.g., terminal structures such as inverted terminal repeats (ITR), hairpin, loops, and other structures such as toroid. In some embodiments, the constructs disclosed herein comprise one, two, or three ITRs. In some embodiments, the constructs disclosed herein comprise no more than two ITRs. Various methods of structural modifications are known in the art.

In some embodiments, one or both ends of the construct can be protected (e.g., from exonucleolytic degradation) by methods known in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

In some embodiments, the constructs disclosed herein can be introduced into a cell as part of a vector having additional sequences such as, for example, replication origins, promoters and genes encoding antibiotic resistance. In some embodiments, the constructs can be introduced as naked nucleic acid, as nucleic acid complexed with an agent such as a liposome, polymer, or poloxamer, or can be delivered by viral vectors (e.g., adenovirus, AAV, herpesvirus, retrovirus, lentivirus).

In some embodiments, although not required for expression, the constructs disclosed herein may also include transcriptional or translational regulatory sequences, for example, promoters, enhancers, insulators, internal ribosome entry sites, sequences encoding peptides, and/or polyadenylation signals.

In some embodiments, the constructs comprising a coding sequence for a polypeptide of interest may include one or more of the following modifications: codon optimization (e.g., to human codons) and/or addition of one or more glycosylation sites. See, e.g., McIntosh et al. (2013) Blood (17):3335-44.

### E. Gene Editing System

Various known gene editing systems can be used for targeted insertion of a heterologous AAT gene in the practice of the present disclosure, including, e.g., CRISPR/Cas system; zinc finger nuclease (ZFN) system; and transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases have been, and additional nucleases are being, for example developed based on the Argonaute system (e.g., from T thermophilus, known as ‘TtAgo’, see Swarts et al (2014) Nature 507(7491): 258-261), which also may have the potential for uses in genome editing and gene therapy.

It will be appreciated that for methods that use the guide RNAs for a Cas nuclease, such as a Cas9 nuclease disclosed herein, the methods include the use of the CRISPR/Cas system (and any of the donor construct disclosed herein that comprises a sequence encoding a heterologous AAT). It will also be appreciated that the present disclosure contemplates methods of targeted insertion and expression of a heterologous AAT using the bidirectional constructs disclosed herein, which can be performed with or without the albumin guide RNAs disclosed herein (e.g., using a ZFN system to cause a break in a target DNA sequence, creating a site for insertion of the bidirectional construct).

In some embodiments, a CRISPR/Cas system (e.g., a guide RNA and RNA-guided DNA binding agent) can be used to create a site of insertion at a desired locus within a host genome, at which site a donor construct (e.g., bidirectional construct) comprising a sequence encoding a heterologous AAT disclosed herein can be inserted to express a heterologous AAT. In some embodiments, the heterologous AAT transgene may be heterologous with respect to its insertion site, for example inserted to a safe harbor locus, as described herein. In some embodiments, a guide RNA described herein (SEQ ID NO: 2-33) that targets a human albumin locus (e.g., intron 1) can be used according to the present methods with a RNA-guided DNA binding agent (e.g., Cas nuclease) to create a site of insertion, at which site a donor construct (e.g., bidirectional construct) comprising a sequence encoding a heterologous AAT can be inserted to express a heterologous AAT. The guide RNAs comprising guide sequences for targeted insertion of a heterologous AAT gene into intron 1 of the human albumin locus are exemplified and described herein (see, e.g., Table 1).

Methods of using various RNA-guided DNA-binding agents, e.g., a nuclease, such as a Cas nuclease, e.g., Cas9, are also well known in the art. It will be appreciated that, depending on the context, the RNA-guided DNA-binding agent can be provided as a nucleic acid (e.g., DNA or mRNA) or as a protein. In some embodiments, the present method can be practiced in a host cell that already expresses a RNA-guided DNA-binding agent.

In some embodiments, the RNA-guided DNA-binding agent, such as a Cas9 nuclease, has cleavase activity, which can also be referred to as double-strand endonuclease activity. In some embodiments, the RNA-guided DNA-binding agent, such as a Cas9 nuclease, has nickase activity, which can also be referred to as single-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see, e.g., the list in the next paragraph), and mutant (e.g., engineered or other variant) versions thereof. See, e.g., US2016/0312198 A1; US 2016/0312199 A1.

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA-binding agent is called a ribonucleoprotein complex (RNP). In some embodiments, the RNA-guided DNA-binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as FokI. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22: 163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)).

In some embodiments, a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA-binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be linked at the C-terminus of the RNA-guided DNA-binding agent. One or more linkers are optionally included at the fusion site.

### III. Delivery Methods

The guide RNA (albumin gRNA; SERPINA1 gRNA), RNA-guided DNA binding agents (e.g., Cas nuclease), and nucleic acid constructs (e.g., bidirectional construct) disclosed herein can be delivered to a host cell or subject, in vivo or ex vivo, using various known and suitable methods available in the art. The guide RNA, RNA-guided DNA binding agents, and nucleic acid constructs can be delivered individually or together in any combination, using the same or different delivery methods as appropriate.

Conventional viral and non-viral based gene delivery methods can be used to introduce the guide RNA disclosed herein as well as the RNA-guided DNA binding agent and donor construct in cells (e.g., mammalian cells) and target tissues. As further provided herein, non-viral vector delivery systems nucleic acids such as non-viral vectors, plasmid vectors, and, e.g naked nucleic acid, and nucleic acid complexed with a delivery vehicle such as a liposome, lipid nanoparticle (LNP), or poloxamer. Viral vector delivery systems include DNA and RNA viruses.

Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by AmaxaBiosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™) The preparation of lipid:nucleic acid complexes, including targeted liposomes such as immunolipid complexes, is well known in the art, and as described herein.

Various delivery systems (e.g., vectors, liposomes, LNPs) containing the guide RNAs, RNA-guided DNA binding agent, and donor construct, singly or in combination, can also be administered to an organism for delivery to cells in vivo or administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such nucleic acids are available and well known to those of skill in the art.

In certain embodiments, the present disclosure provides DNA or RNA vectors encoding any one or more of the compositions disclosed herein—e.g., a guide RNA (albumin gRNA; and/or SERPINA1 gRNA) comprising any one or more of the guide sequences described herein; a construct (e.g., bidirectional construct) comprising a sequence encoding heterologous AAT; or a sequence encoding a RNA-guided DNA binding agent. In certain embodiments, the invention comprises DNA or RNA vectors encoding any one or more of the compositions described herein, or in any combination. In some embodiments, the vectors further comprise, e.g., promoters, enhancers, and regulatory sequences. In some embodiments, the vector that comprises a bidirectional construct comprising a sequence that encodes a heterologous AAT does not comprise a promoter that drives heterologous AAT expression. In some embodiments, the vector that comprises a guide RNA comprising any one or more of the guide sequences described herein (albumin gRNA; and/or SERPINA1 gRNA) also comprises one or more nucleotide sequence(s) encoding a crRNA, a trRNA, or a crRNA and trRNA, as disclosed herein.

In some embodiments, the vector comprises a nucleotide sequence encoding a guide RNA (albumin gRNA; and/or SERPINA1 gRNA) described herein. In some embodiments, the vector comprises one copy of a guide RNA. In other embodiments, the vector comprises more than one copy of a guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with an RNA-guided DNA nuclease, such as a Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA (sgRNA). In other embodiments, the crRNA and the trRNA may be driven by their corresponding promoters on the same vector. In yet other embodiments, the crRNA and the trRNA may be encoded by different vectors.

In some embodiments, the nucleotide sequence encoding the guide RNA (albumin gRNA; and/or SERPINA1 gRNA) may be located on the same vector comprising the nucleotide sequence encoding an RNA-guided DNA binding agent such as a Cas protein. In some embodiments, one or more albumin gRNA and/or one or more SERPINA1 gRNA may be located on the same vector. In some embodiments, one or more albumin gRNA and/or one or more SERPINA1 gRNA may be located on the same vector with the nucleotide sequence encoding an RNA-guided DNA binding agent such as a Cas protein. In some embodiments, expression of the guide RNA and of the RNA-guided DNA binding agent such as a Cas protein may be driven by their own corresponding promoters. In some embodiments, expression of the guide RNA may be driven by the same promoter that drives expression of the RNA-guided DNA binding agent such as a Cas protein. In some embodiments, the guide RNA and the RNA-guided DNA binding agent such as a Cas protein transcript may be contained within a single transcript. For example, the guide RNA may be within an untranslated region (UTR) of the RNA-guided DNA binding agent such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript. In some embodiments, expression of the RNA-guided DNA binding agent such as a Cas protein and the guide RNA from the same vector in close temporal proximity may facilitate more efficient formation of the CRISPR RNP complex.

In some embodiments, the nucleotide sequence encoding the guide RNA (albumin gRNA; and/or SERPINA1 gRNA) and/or RNA-guided DNA binding agent may be located on the same vector comprising the construct that comprises a heterologous AAT gene. In some embodiments, proximity of the construct comprising the AAT gene and the guide RNA (and/or the RNA-guided DNA binding agent) on the same vector may facilitate more efficient insertion of the construct into a site of insertion created by the guide RNA/RNA-guided DNA binding agent.

In some embodiments, the vector comprises one or more nucleotide sequence(s) encoding a sgRNA (albumin gRNA; and/or SERPINA1 gRNA) and an mRNA encoding an RNA-guided DNA binding agent, which can be a Cas protein, such as Cas9 or Cpf1. In some embodiments, the vector comprises one or more nucleotide sequence(s) encoding a crRNA, a trRNA, and an mRNA encoding an RNA-guided DNA binding agent, which can be a Cas protein, such as, Cas9 or Cpf1. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the vector comprises a donor construct (e.g., the bidirectional nucleic acid construct) comprising a sequence that encodes a heterologous AAT, as disclosed herein. In some embodiments, in addition to the donor construct (e.g., bidirectional nucleic acid construct) disclosed herein, the vector may further comprise nucleic acids that encode the albumin guide RNAs described herein and/or nucleic acid encoding a RNA-guided DNA-binding agent (e.g., a Cas nuclease such as Cas9). In some embodiments, a nucleic acid encoding an albumin guide RNA and/or a nucleic acid encoding a RNA-guided DNA-binding agent are each or both on a separate vector from a vector that comprises the donor construct (e.g., bidirectional construct) disclosed herein. In any of the embodiments, the vector may include other sequences that include, but are not limited to, promoters, enhancers, regulatory sequences, as described herein. In some embodiments, the promoter does not drive the expression of the heterologous AAT of the donor construct (e.g., bidirectional construct). In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease (e.g., Cas9). In some embodiments, the vector comprises one or more nucleotide sequence(s) encoding a crRNA, a trRNA, and an mRNA encoding an RNA-guided DNA nuclease, which can be a Cas nuclease, such as, Cas9. In some embodiments, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the vector may be circular. In other embodiments, the vector may be linear. In some embodiments, the vector may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without a helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector.

In some embodiments, “AAV” refers all serotypes, subtypes, and naturally-occuring AAV as well as recombinant AAV. “AAV” may be used to refer to the virus itself or a derivative thereof. The term “AAV” includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest (e.g., AAT). The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh 10, AAVLK03, AV 10, AAV 11, AAV12, rh10, and hybrids thereof, avian AAV, bovine AAV, canine AAV, equine AAV, primate AAV, nonprimate AAV, and ovine AAV capside sequence. In general, the heterologous nucleic acid sequence (the transgene) is flanked by at least one, at least two, or at least three AAV inverted terminal repeat sequences (ITRs). An AAV vector may either be single-stranded (ssAAV) or self-complementary (scAAV).

In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding an RNA-guided DNA binding agent such as a Cas protein (e.g., Cas9), while a second AAV vector may contain one or more guide sequences.

In some embodiments, the vector system may be capable of driving expression of one or more coding sequences in a cell. In some embodiments, the vector does not comprise a promoter that drives expression of one or more coding sequences once it is integrated in a cell (e.g., uses the host cell's endogenous promoter such as when inserted at intron 1 of an albumin locus, as exempflied herein). In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding an RNA-guided DNA binding agent such as a Cas protein (e.g., Cas9) described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the vector may comprise any one or more of the constructs comprising a heterologous AAT gene described herein. In some embodiments, the heterologous AAT gene may be operably linked to at least one transcriptional or translational control sequence. In some embodiments, the heterologous AAT gene may be operably linked to at least one promoter. In some embodiments, the heterologous gene is not linked to a promoter that drives the expression of the heterologous gene.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the TetOn® promoter (Clontech).

In some embodiments, the promoter may be a tissue-specific promoter, e.g., a promoter specific for expression in the liver.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the TetOn® promoter (Clontech).

In additional embodiments, the vector system may comprise tissue-specific promoters to start expression only after it is delivered into a specific tissue.

The vector comprising: one or more guide RNA (albumin gRNA and/or SERPINA1 gRNA), RNA-binding DNA binding agent, or donor construct comprising a sequence encoding a heterologous AAT protein, individually or in any combination, may be delivered by liposome, a nanoparticle, an exosome, or a microvesicle. The vector may also be delivered by a lipid nanoparticle (LNP). One or more guide RNA (albumin gRNA and/or SERPINA1 gRNA), RNA-binding DNA binding agent (e.g. mRNA), or donor construct comprising a sequence encoding a heterologous AAT protein, individually or in any combination, may be delivered by liposome, a nanoparticle, an exosome, or a microvesicle. One or more guide RNA (albumin gRNA and/or SERPINA1 gRNA), RNA-binding DNA binding agent (e.g. mRNA), or donor construct comprising a sequence encoding a heterologous AAT protein, individually or in any combination, may be delivered by LNP.

Lipid nanoparticles (LNPs) are a well-known means for delivery of nucleotide and protein cargo, and may be used for delivery of any of the guide RNAs (e.g., albumin gRNA; and/or SERPINA1 gRNA), RNA-guided DNA binding agent, and/or donor construct (e.g., bidirectional construct) disclosed herein. In some embodiments, the LNPs deliver the compositions in the form of nucleic acid (e.g., DNA or mRNA), or protein (e.g., Cas nuclease), or nucleic acid together with protein, as appropriate.

In some embodiments, provided herein is a method for delivering any of the guide RNAs described herein (albumin gRNA; and/or SERPINA1 gRNA) and/or donor construct (e.g., bidirectional construct) disclosed herein, alone or in combination, to a host cell or subject, wherein any one or more of the components is associated with an LNP. In some embodiments, the method further comprises a RNA-guided DNA binding agent (e.g., Cas9 or a sequence encoding Cas9).

In some embodiments, provided herein is a composition comprising any of the guide RNAs described herein (albumin gRNA; and/or SERPINA1 gRNA) and/or donor construct (e.g., bidirectional construct) disclosed herein, alone or in combination, with an LNP. In some embodiments, the composition further comprises a RNA-guided DNA binding agent (e.g., Cas9 or a nucleic acid sequence encoding Cas9).

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of PCT/US2018/053559 (filed Sep. 28, 2018), WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, LNPs associated with the bidirectional construct disclosed herein are for use in preparing a medicament for treating a disease or disorder. The disease or disorder may be a disease associated with al-antitrypsin deficiency (AATD).

In some embodiments, any of the guide RNAs described herein, RNA-guided DNA binding agents described herein, and/or donor construct (e.g., bidirectional construct) disclosed herein, alone or in combination, whether naked or as part of a vector, is formulated in or administered via a lipid nanoparticle; see e.g., WO/2017/173054, the contents of which are hereby incorporated by reference in their entirety.

It will be apparent that any one or more guide RNA disclosed herein (albumin gRNA; and/or SERPINA1 gRNA), a RNA-guided DNA binding agent (e.g., Cas nuclease or a nucleic acid encoding a Cas nuclease), and a donor construct (e.g., bidirectional construct) comprising a sequence encoding a heterologous AAT can be delivered using the same or different systems. For example, the guide RNA, RNA-guided DNA binding agent (e.g., Cas nuclease), and construct can be carried by the same vector (e.g., AAV). Alternatively, the RNA-guided DNA binding agent such as a Cas nuclease (as a protein or mRNA) and/or gRNA (albumin gRNA; and/or SERPINA1 gRNA) can be carried by a plasmid or LNP, while the donor construct can be carried by a vector such as AAV. The use of any of the variety of combinations will be guided by, e.g., the practicality and efficiency of their use. Furthermore, the different delivery systems can be administered by the same or different routes (e.g. by infusion; by injection, such as intramuscular injection, tail vein injection, or other intravenous injection; by intraperitoneal administration and/or intramuscular inj ection).

The different delivery systems can be delivered in vitro or in vivo simultaneously or in any sequential order. In some embodiments, the donor construct, guide RNA (albumin gRNA; and/or SERPINA1 gRNA), and Cas nuclease can be delivered in vitro or in vivo simultaneously, e.g., in one vector, two vectors, three vectors, individual vectors, one LNP, two LNPs, three LNPs, individual LNPs, or a combination thereof. In some embodiments, the donor construct can be delivered in vivo or in vitro, as a vector and/or associated with a LNP, prior to (e.g., about 1, 2, 3, 4, 5, 6, 7, 8, 9, 10, 11, 12, 13, 14, or more days) delivering the albumin guide RNA and/or Cas nuclease, as a vector and/or associated with a LNP singly or together as a ribonucleoprotein (RNP). In some embodiments, the donor construct can be delivered in multiple administerations, e.g., every day, every two days, every three days, every four days, every week, every two weeks, every three weeks, or every four weeks. In some embodiments, the donor construct can be delivered at one-week intervals, e.g., at week 1, week 2, and week 3, etc. As a further example, the albumin guide RNA and Cas nuclease, as a vector and/or associated with a LNP singly or together as a ribonucleoprotein (RNP), can be delivered in vivo or in vitro, prior to (e.g., about 1, 2, 3, 4, 5, 6, 7, 8, 9, 10, 11, 12, 13, 14, or more days) delivering the construct, as a vector and/or associated with a LNP. In some embodiments, the albumin guide RNA can be delivered in multiple administerations, e.g., every day, every two days, every three days, every four days, every week, every two weeks, every three weeks, or every four weeks. In some embodiments, the the albumin guide RNA can be delivered at one-week intervals, e.g., at week 1, week 2, and week 3, etc. In some embodiments, the Cas nuclease can be delivered in multiple administerations, e.g., can be delivered every day, every two days, every three days, every four days, every week, every two weeks, every three weeks, or every four weeks. In some embodiments, the Cas nuclease can be delivered at one-week intervals, e.g., at week 1, week 2, and week 3, etc.

In some embodiments, the present disclosure also provides pharmaceutical formulations for administering any of the guide RNAs (albumin gRNA; and/or SERPINA1 gRNA) disclosed herein. In some embodiments, the pharmaceutical formulation includes a RNA-guided DNA binding agent (e.g., Cas nuclease) and a donor construct comprising a coding sequence of a heterologous AAT, as disclosed herein. Pharmaceutical formulations suitable for delivery into a subject (e.g., human subject) are well known in the art.

### IV. Methods of Use

The gene encoding AAT is located on chromosome 14q32.1 and part of the Protease

Inhibitor (Pi) locus. Normal AAT may be referred to as PiM. The PiZ mutation can cause liver and/or lung symptoms, including in homozygous (ZZ) and heterozygous (MZ or SZ) individuals. The PiS mutation can cause milder reduction in serum AAT and lower risk for lung disease. Numerous other allelic mutations are known in the art. See, e.g., Greulich et al. “Alpha-1-antitrypsin deficiency: increasing awareness and improving diagnosis,” Ther Adv Respir Dis. 2016.

AATD may be diagnosed by methods known in the art, e.g., by the presence of one or more physiologic symptoms, blood tests, and/or genetic tests for one or more of the 150+ known AAT mutations reported to date. See, e.g., id. Examples of blood and/or tests include, but are not limited to, assaying for serum AAT levels, detecting mutations by polymerase chain reaction (PCR) and/or next generation sequencing (NGS), isoelectric focusing (IEF) with or without immunoblotting, AAT gene locus sequencing, and serum separator cards (lateral flow assay to detect the Z protein).

In some embodiments, AAT serum levels may be considered normal within the 150-350 mg/dL range using immunodiffusion methods (which may overestimate serum levels). In these embodiments, a level of 80 mg/dL may be regarded as protective, e.g., decreased risk of one or more symptoms, e.g., emphysema, despite being lower than the normal range.

In some embodiments, AAT serum levels may be considered normal within the 90-200 mg/dL range using nephelometry or immunoturbidimetry and a purified standard. In these embodiments, a level of 50 mg/dL may be regarded as protective, e.g., decreased risk of decreased risk of one or more symptoms, e.g., emphysema, despite being lower than the normal range.

In some embodiments, AAT serum levels of less than about 130 mg/dL, 125 mg/dL, 120 mg/dL, 115 mg/dL, 110 mg/dL, 105 mg/dL, or 100 mg/dL indicates low likelihood of a homozygous AAT mutation and further genetic testing may not be necessary. In some embodiments, AAT serum levels of about 104 mg/dL indicates low likelihood of homozygous PiS, and 113 mg/dL indicates low likelihood of homozygous PiZ. In some embodiments, AAT serum levels may provide limited exclusion information for heterozygous carriers, and further genetic testing may be necessary, because AAT serum levels of about 150 mg/dL indicates low likelihood of heterozygous carrier PiMZ, and AAT serum levels of about 220 mg/dL indicates low likelihood of heterozygous carrier piMS.

Examples of detectable physiologic symptoms include, but are not limited to lung disease and/or liver disease; wheezing or shortness of breath; increased risk of lung infections; chronic obstructive pulmonary disease (COPD); bronchitis, asthma, dyspnea; cirrhosis; neonatal jaundice; panniculitis; chronic cough and/or phlegm; recurring chest colds; yellowing of the skin or the white part of the eyes; swelling of the belly or legs. In some embodiments, individuals may be subject to blood and/or genetic tests if they are COPD patients, nonresponsive asthmatic patients, patients with bronchiectasis of unknown etiology, individuals with cryptogenic cirrhosis/liver disease, granulomatosis with polyangiitis, necrotizing panniculitis, and/or first-degree relatives of patients/carriers with AATD. In some embodiments, pulmonary function testing (PFT), functional residual capacity (RFC), and/or lung density loss at total lung capacity (TLC) may be performed.

In some embodiments, subjects to be treated include individuals with AAT serum below the normal range. In some embodiments, subjects to be treated include individuals with any allelic mutation combination, e.g., ZZ, MZ, MS. In some embodiments, subjects to be treated include individuals with post-bronchodilator FEV1 of at least 30%, 40%, 50%, 60% of predicted normal value. In some embodiments, subjects to be treated include individuals eligible for bronchoscopy. In some embodiments, subjects to be treated include individuals with adequate hepatic and renal function, nonsmokers, individuals who have not had lung or liver lobectomy, transplant, individuals who have not had lung volume reduction surgery, individuals who have not had acute respiratory tract infection or COPD exacerbation immediately prior to treatment, and/or individuals who do not have unstable cor pulmonale.

As described herein, the present disclosure provides compositions and methods for expressing heterologous AAT (e.g., a functional or wild-type AAT) at a human safe harbor site, such as an albumin safe harbor site to allow secretion of the protein. In some embodiments, the methods thereby alleviate the negative effects of AATD in the lung. The present disclosure also provides compositions and methods to knock out the endogenous SERPINA1 gene thereby eliminating the production of mutant forms of AAT associated with AAT protein polymerization and aggregation in liver hepatocytes, which lead to liver symptoms in patients with AATD. See WO/2018/119182, incorporated by reference in its entirety. Accordingly, the compositions and methods disclosed herein treat AATD by alleviating the negative effects of the disorder in the lung as well as in the liver.

AAT is primarily synthesized and secreted by hepatocytes, and functions to inhibit the activity of neutrophil elastase in the lung. Without sufficient quantities of functioning AAT, neutrophil elastase is uncontrolled and damages alveoli in the lung. Thus, mutations in SERPINA1 that result in decreased levels of AAT, or decreased levels of properly functioning AAT, lead to lung pathology, including, e.g., chronic obstructive pulmonary disease (COPD), bronchitis, or asthma.

The albumin gRNAs, donor construct (e.g., bidirectional construct comprising a sequence encoding a functional heterologous AAT), and RNA-guided DNA binding agents described herein are useful for introducing a heterologous AAT nucleic acid to a host cell, in vivo or in vitro. In some embodiments, the albumin gRNAs, donor construct (e.g., bidirectional construct comprising a sequence encoding a heterologous AAT), and RNA-guided DNA binding agents described herein are useful for expressing a functional heterologous AAT in a host cell, or in a subject in need thereof. In some embodiments, the albumin gRNAs, donor construct (e.g., bidirectional construct comprising a sequence encoding a heterologous AAT), and RNA-guided DNA binding agents described herein are useful for treating AATD in a subject in need thereof. In some embodiments, treatment of AATD by expressing heterologous AAT at an albumin locus enhances secretion of functional (e.g., wild type) AAT, and alleviates one or more symptoms of AATD, e.g., negative effects on the lungs. For example, heterologous AAT expression may alleviate lung disease and/or liver disease; wheezing or shortness of breath; increased risk of lung infections; COPD; bronchitis, asthma, dyspnea; cirrhosis; neonatal jaundice; panniculitis; chronic cough and/or phlegm; recurring chest colds; yellowing of the skin or the white part of the eyes; swelling of the belly or legs. Administration of any one or more of the albumin gRNAs, donor construct (e.g., bidirectional construct comprising a sequence encoding heterologous AAT), and RNA-guided DNA binding agents described herein leads to an increase in functional (e.g., wild type) AAT gene expression, AAT protein levels (e.g. circulating, serum, or plasma levels) and/or AAT activity levels (e.g., trypsin inhibition) (e.g., greater than 10%, 20%, 30%, 40%, 50%, 60%, 70%, 80%, or 90% AAT gene expression or protein levels as compared to an untreated control, e.g., by nephelometry or immunoturbidimetry, e.g., AAT greater than about 40 mg/dL, 45 mg/dL, 50 mg/dL, 60 mg/dL, 70 mg/dL, 80 mg/dL, 90 mg/dL, 100 mg/dL, or 110 mg/dL in serum). In some embodiments, the effectiveness of the treatment can be assessed by measuring serum or plasma AAT activity, wherein an increase in the subject's serum or plasma level and/or activity of AAT indicates effectiveness of the treatment. In some embodiments, the effectiveness of the treatment can be assessed by measuring serum or plasma AAT protein and/or activity levels, wherein an increase in the subject's serum or plasma level and/or activity of AAT indicates effectiveness of the treatment. In some embodiments, effectiveness of the treatment can be assessed by PASD staining of liver tissue sections, e.g., to measure aggregation. In some embodiments, effectiveness of the treatment can be assessed by measuring inhibition of neutrophil elastase, e.g., in the lung. In some embodiments, effectiveness of the treatment can be assessed by genotype serum level, AAT lung function, spirometry test, chest X-ray of lung, CT scan of lung, blood testing of liver function, and/or ultrasound of liver.

In some embodiments, treatment refers to increasing serum AAT levels, e.g., to protective levels. In some embodiments, treatment refers to increasing serum AAT levels, e.g., within the normal range. In some embodiments, treatment refers to increasing serum AAT levels, e.g., above 40, 50, 60, 70, 80, 90, or 100 mg/dL, e.g., as measured using nephelometry or immunoturbidimetry and a purified standard.

In some embodiments, treatment refers to increasing serum AAT levels, e.g., to protective levels. In some embodiments, treatment refers to increasing serum AAT levels, e.g., within the normal range. In some embodiments, treatment refers to increasing serum AAT levels, e.g., above 40, 50, 60, 70, 80, 90, or 100 mg/dL, e.g., as measured using nephelometry or immunoturbidimetry and a purified standard. In some embodiments, treatment refers to improvement in baseline serum AAT as compared to control, e.g., before and after treatment. In some embodiments, treatment refers to a improvement in histologic grading of AATD associated liver disease, e.g., by 1, 2, 3, or more points, as compared to control, e.g., before and after treatment. In some embodiments, treatment refers to improvement in Ishak fibrosis score as compared to control, e.g., before and after treatment.

In normal or healthy individuals (e.g., individuals that do not possess the ZZ, MZ, or SZ allele), AAT levels vary between about 500 μg/ml to about 3000 μg/ml in the serum. Clinically, the level of circulating AAT can be measured by enzymologic and/or immunologic assay (e.g., ELISA), which methods are well known in the art. See, e.g., Stoller, J. and Aboussouan, L. (2005) Alphal-antitrypsin deficiency. Lancet 365: 2225-2236; Kanakoudi F, Drossou V, Tzimouli V, et al: Serum concentrations of 10 acute-phase proteins in healthy term and pre-term infants from birth to age 6 months. Clin Chem 1995;41:605-608; Morse J O: Alpha-1-antitrypsin deficiency. N Engl J Med 1978;299:1045-1048, 1099-1105; Cox D W: Alpha-1-antitrypsin deficiency. In The Metabolic and Molecular Basis of Inherited Disease. Vol 3. Seventh edition. Edited by C R Scriver, A L Beaudet, W S Sly, D Valle. New York, McGraw-Hill Book Company, 1995, pp 4125-4158.

Accordingly, in some embodiments, the compositions and methods disclosed herein are useful for increasing serum or plasma levels of AAT (e.g., functional AAT or wild type AAT) in a subject having AATD (e.g., individuals that possess the ZZ, MZ, or SZ allele) or at risk of developing AATD (e.g., individuals that possess the ZZ, MZ, or SZ allele) to about 500 μg/ml, or more. In some embodiments, the compositions and methods disclosed herein are useful for increasing AAT protein levels to about 1500 μg/ml. In some embodiments, the compositions and methods disclosed herein are useful for increasing AAT protein levels to about 1000 μg/ml to about 1500 μg/ml, about 1500 μg/ml to about 2000 μg/ml, about 2000 μg/ml to about 2500 μg/ml, about 2500 μg/ml to about 3000 μg/ml, or more. For example, the compositions and methods disclosed herein are useful for increasing serum or plasma levels of AAT in a subject having an AATD to about 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000μg/ml, or more.

In some embodiments, the compositions and methods disclosed herein are useful for increasing serum or plasma levels of AAT in a subject having AATD (e.g., individuals that possess the ZZ, MZ, or SZ allele) or at risk of developing AATD (e.g., individuals that possess the ZZ, MZ, or SZ allele) by about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150%, 160%, 170%, 180%, 190%, 200%, or more, as compared to the subject's serum or plasma level of AAT before administration.

In some embodiments, the compositions and methods disclosed herein are useful for increasing heterologous functional AAT protein and/or AAT activity in a host cell by about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150%, 160%, 170%, 180%, 190%, 200%, or more, as compared to an AAT level before administration to the host cell, e.g. a normal level. In some embodiments, the cell is a liver cell.

In some embodiments, the cell (host cell) or population of cells is capable of expressing AAT, e.g., cells that originate from tissue of any one or more of liver, lung, gastric organ, kidney, stomach, proximal and distal small intestine, pancreas, adrenal glands, or brain.

In some embodiments, the method comprises administering a guide RNA and an RNA-guided DNA binding agent (such as an mRNA encoding a Cas9 nuclease) in an LNP. In further embodiments, the method comprises administering an AAV nucleic acid construct encoding a AAT protein, such as an bidirectional AAT construct. CRISPR/Cas9 LNP, comprising guide RNA and an mRNA encoding a Cas9, can be administered intravenously. AAV AAT donor construct can be administered intravenously. Exemplary dosing of CRISPR/Cas9 LNP includes about 0.1, 0.25, 0.3, 0.5, 1, 2, 3, 4, 5, 6, 8, or 10 mpk (RNA). The units mg/kg and mpk are being used interchangably herein. Exemplary dosing of AAV comprising a nucleic acid encoding a AAT protein includes an MOI of about 10¹¹, 10¹², 10¹³, and 10¹⁴ vg/kg, optionally the MOI may be about 1×10¹³ to 1×10¹⁴vg/kg.

In some embodiments, the method comprises expressing a therapeutically effective amount of the AAT protein. In some embodiments, the method comprises achieving a therapeutically effective level of circulating AAT activity in an individual. In particular embodiments, the method comprises achieving AAT activity of at least about 5% to about 50% of normal. The method may comprise achieving AAT activity of at least about 50% to about 150% of normal. In certain embodiments, the method comprises achieving an increase in AAT activity over the patient's baseline AAT activity of at least about 1% to about 50% of normal AAT activity, or at least about 5% to about 50% of normal AAT activity, or at least about 50% to about 150% of normal AAT activity.

In some embodiments, the method further comprises achieving a durable effect, e.g. at least 1 month, 2 months, 6 months, 1 year, or 2 year effect. In some embodiments, the method further comprises achieving the therapeutic effect in a durable and sustained manner, e.g. at least 1 month, 2 months, 6 months, 1 year, or 2 year effect. In some embodiments, the level of circulating AAT activity and/or level is stable for at least 1 month, 2 months, 6 months, 1 year, or more. In some embodiments a steady-state activity and/or level of AAT protein is achieved by at least 7 days, at least 14 days, or at least 28 days. In additional embodiments, the method comprises maintaining AAT activity and/or levels after a single dose for at least 1, 2, 4, or 6 months, or at least 1, 2, 3, 4, or 5 years.

In additional embodiments involving insertion into the albumin locus, the individual's circulating albumin levels are normal. The method may comprise maintaining the individual's circulating albumin levels within ±5%, ±10%, ±15%, ±20%, or ±50% of normal circulating albumin levels. In certain embodiments, the individual's albumin levels are unchanged as compared to the albumin levels of untreated individuals by at least week 4, week 8, week 12, or week 20. In certain embodiments, the individual's albumin levels transiently drop then return to normal levels. In particular, the methods may comprise detecting no significant alterations in levels of plasma albumin.

In some embodiments, the invention comprises a method or use of modifying (e.g., creating a double strand break in) an albumin gene, such as a human albumin gene, comprising, administering or delivering to a host cell or population of host cells any one or more of the gRNAs, donor construct (e.g., bidirectional construct comprising a sequence encoding AAT), and RNA-guided DNA binding agents (e.g., Cas nuclease) described herein. In some embodiments, the invention comprises a method or use of modifying (e.g., creating a double strand break in) an albumin intron 1 region, such as a human albumin intron 1, comprising, administering or delivering to a host cell or population of host cells any one or more of the gRNAs, donor construct (e.g., bidirectional construct comprising a sequence encoding AAT), and RNA-guided DNA binding agents (e.g., Cas nuclease) described herein. In some embodiments, the invention comprises a method or use of modifying (e.g., creating a double strand break in) a human safe harbor, such as liver tissue or hepatocyte host cell, comprising, administering or delivering to a host cell or population of host cells any one or more of the gRNAs, donor construct (e.g., bidirectional construct comprising a sequence encoding AAT), and RNA-guided DNA binding agents (e.g., Cas nuclease) described herein. Insertion within a safe harbor locus, such as an albumin locus, allows overexpression of the SERPINA1 gene without significant deleterious effects on the host cell or cell population, such as hepatocytes or liver cells.

In some embodiments, the present disclosure provides a method or use of modifying (e.g., creating a double strand break in) intron 1 of a human albumin locus comprising, administering or delivering to a host cell any one or more of the albumin gRNAs, donor construct (e.g., bidirectional construct comprising a sequence encoding a heterologous AAT), and RNA-guided DNA binding agents (e.g., Cas nuclease) described herein. In some embodiments, the albumin guide RNA comprises a guide sequence that contains at least 15, 16, 17, 18, 19, or 20 contiguous nucleotides that are capable of binding to a region within intron 1 of a a human albumin locus (SEQ ID NO: 1). In some embodiments, the albumin guide RNA comprises at least 15, 16, 17, 18, 19, or 20 contiguous nucleotides of a sequence selected from the group consisting of SEQ ID NOs: 2-33. In some embodiments, the albumin guide RNA comprises a sequence that is at least 99%, 98%, 97%, 96%, 95%, 94%, 93%, 92%, 91%, 90%, 89% or 88% identical to a sequence selected from the group consisting of SEQ ID NOs: 2-33. In some embodiments, the albumin gRNA comprises a guide sequence comprising a sequence of any one of SEQ ID NOs.: 4, 13, 17, 19, 27, 28, 30, or 31. In some embodiments, the administration is in vitro. In some embodiments, the administration is in vivo. In some embodiments, the donor construct is a bidirectional construct that comprises a sequence encoding a heterologous AAT. In some embodiments, the host cell is a liver cell.

In some embodiments, the present disclosure provides a method or use of introducing a heterologous AAT nucleic acid (e.g., functional or wilde type AAT) to a host cell comprising, administering or delivering any one or more of the albumin gRNAs, donor construct (e.g., bidirectional construct comprising a sequence encoding a heterologous AAT), and RNA-guided DNA binding agents (e.g., Cas nuclease) described herein. In some embodiments, the albumin gRNA comprises a guide sequence that contains at least 15, 16, 17, 18, 19, or 20 contiguous nucleotides that are capable of binding to a region within intron 1 of a human albumin locus (SEQ ID NO: 1). In some embodiments, the albumin guide RNA comprises at least 15, 16, 17, 18, 19, or 20 contiguous nucleotides of a sequence selected from the group consisting of SEQ ID NOs: 2-33. In some embodiments, the albumin guide RNA comprises a sequence that is at least 99%, 98%, 97%, 96%, 95%, 94%, 93%, 92%, 91%, 90%, 89% or 88% identical to a sequence selected from the group consisting of SEQ ID NOs: 2-33. In some embodiments, the albumin gRNA comprises a guide sequence comprising a sequence of any one of SEQ ID NOs.: 4, 13, 17, 19, 27, 28, 30, or 31. In some embodiments, the albumin gRNA comprising a sequence chosen from: a) a sequence that is at least 95%, 90%, 85%, 80%, or 75% identical to a sequence selected from the group consisting of SEQ ID NOs: 2, 8, 13, 19, 28, 29, 31, 32, or 33; b) at least 17, 18, 19, or 20 contiguous nucleotides of a sequence selected from the group consisting of SEQ ID NOs: 2, 8, 13, 19, 28, 29, 31, 32, or 33; c) a sequence selected from the group consisting of SEQ ID NOs: 34, 40, 45, 51, 60, 61, 63, 64, 65, 66, 72, 77, 83, 92, 93, 95, 96, or 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33. In some embodiments, the administration is in vitro. In some embodiments, the administration is in vivo. In some embodiments, the donor construct is a bidirectional construct that comprises a sequence encoding a heterologous AAT (e.g., functional or wild type AAT). In some embodiments, the host cell is a liver cell.

In some embodiments, the present disclosure provides a method or use of expressing a heterologous AAT (e.g., functional or wild type AAT) in a host cell comprising, administering or delivering any one or more of the albumin gRNAs, donor construct (e.g., bidirectional construct comprising a sequence encoding a heterologous AAT), and RNA-guided DNA binding agents (e.g., Cas nuclease) described herein. In some embodiments, the subject in need thereof is between birth and 2 years of age; between 2 to 12 years of age; or between 12 to 21 years of age. In some embodiments, the albumin gRNA comprises a guide sequence that contains at least 15, 16, 17, 18, 19, or 20 contiguous nucleotides that are capable of binding to a region within intron 1 of a human albumin locus (SEQ ID NO: 1). In some embodiments, the albumin gRNA comprises at least 15, 16, 17, 18, 19, or 20 contiguous nucleotides of a sequence selected from the group consisting of SEQ ID NOs: 2-33. In some embodiments, the albumin gRNA comprises a sequence that is at least 99%, 98%, 97%, 96%, 95%, 94%, 93%, 92%, 91%, 90%, 89% or 88% identical to a sequence selected from the group consisting of SEQ ID NOs: 2-33. In some embodiments, the albumin gRNA comprises a guide sequence comprising a sequence of any one of SEQ ID NOs: 4, 13, 17, 19, 27, 28, 30, or 31. In some embodiments, the albumin gRNA comprising a sequence chosen from: a) a sequence that is at least 95%, 90%, 85%, 80%, or 75% identical to a sequence selected from the group consisting of SEQ ID Nos: 2, 8, 13, 19, 28, 29, 31, 32, or 33; b) at least 17, 18, 19, or 20 contiguous nucleotides of a sequence selected from the group consisting of SEQ ID NOs: 2, 8, 13, 19, 28, 29, 31, 32, or 33; c) a sequence selected from the group consisting of SEQ ID NOs: 34, 40, 45, 51, 60, 61, 63, 64, 65, 66, 72, 77, 83, 92, 93, 95, 96, or 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 5, 6, 7, 8, 9, 10, 11, 12, 13, 14, 15, 16, 17, 18, 19, 20, 21, 22, 23, 24, or 25 consecutive nucleotides within or spanning the genomic coordinates listed for SEQ ID NOs: 2-33. In some embodiments, the administration is in vitro. In some embodiments, the administration is in vivo. In some embodiments, the donor construct is a bidirectional construct that comprises a sequence encoding a heterologous AAT. In some embodiments, the host cell is a liver cell.

In some embodiments, the present disclosure provides a method or use of treating AATD comprising, administering or delivering any one or more of the albumin gRNAs, donor construct (e.g., bidirectional construct comprising a sequence encoding a heterologous AAT), and RNA-guided DNA binding agents (e.g., Cas nuclease) described herein to a subject in need thereof. In some embodiments, the albumin gRNA comprises a guide sequence that contains at least 15, 16, 17, 18, 19, or 20 contiguous nucleotides that are capable of binding to a region within intron 1 of a mouse or a human albumin locus (SEQ ID NO: 1). In some embodiments, the albumin gRNA comprises at least 15, 16, 17, 18, 19, or 20 contiguous nucleotides of a sequence selected from the group consisting of SEQ ID NOs: 2-33. In some embodiments, the albumin gRNA comprises a sequence that is at least 99%, 98%, 97%, 96%, 95%, 94%, 93%, 92%, 91%, 90%, 89% or 88% identical to a sequence selected from the group consisting of SEQ ID NOs: 2-33. In some embodiments, the albumin gRNA comprises a guide sequence comprising a sequence of any one of SEQ ID NO: 4, 13, 17, 19, 27, 28, 30, or 31. In some embodiments, the albumin gRNA comprising a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides +/−10 nucleotides of the genomic coordinates listed for SEQ ID NOs: 2-33. In some embodiments, the donor construct is a bidirectional construct that comprises a sequence encoding a heterologous AAT. In some embodiments, the host cell is a liver cell.

In some embodiments, the present disclosure provides a method or use of increasing functional AAT secretion from a liver cell comprising, administering or delivering any one or more of the albumin gRNAs, donor construct (e.g., bidirectional construct comprising a sequence encoding a heterologous AAT), and RNA-guided DNA binding agents (e.g., Cas nuclease) described herein. In some embodiments, the albumin gRNA comprises a guide sequence that contains at least 15, 16, 17, 18, 19, or 20 contiguous nucleotides that are capable of binding to a region within intron 1 of a mouse or a human albumin locus (SEQ ID NO: 1). In some embodiments, the albumin gRNA comprises at least 15, 16, 17, 18, 19, or 20 contiguous nucleotides of a sequence selected from the group consisting of SEQ ID NOs: 2-33. In some embodiments, the albumin gRNA comprises a sequence that is at least 99%, 98%, 97%, 96%, 95%, 94%, 93%, 92%, 91%, 90%, 89% or 88% identical to a sequence selected from the group consisting of SEQ ID NOs: 2-33. In some embodiments, the albumin gRNA comprises a guide sequence comprising a sequence of any one of SEQ ID NO.: 4, 13, 17, 19, 27, 28, 30, or 31. In some embodiments, the administration is in vitro. In some embodiments, the administration is in vivo. In some embodiments, the donor construct is a bidirectional construct that comprises a sequence encoding a heterologous AAT. In some embodiments, the host cell is a liver cell.

As described herein, the donor construct (e.g., bidirectional construct) comprising a sequence encoding a heterologous AAT, albumin gRNA, and RNA-guided DNA binding agent can be delivered using any suitable delivery system and method known in the art. The compositions can be delivered in vitro or in vivo simultaneously or in any sequential order. In some embodiments, the donor construct, albumin gRNA, and Cas nuclease can be delivered in vitro or in vivo simultaneously, e.g., in one vector, two vectors, individual vectors, one LNP, two LNPs, individual LNPs, or a combination thereof. In some embodiments, the donor construct can be delivered in vivo or in vitro, as a vector and/or associated with a LNP, prior to (e.g., about 1, 2, 3, 4, 5, 6, 7, 8, 9, 10, 11, 12, 13, 14, or more days) delivering the albumin gRNA and/or Cas nuclease, as a vector and/or associated with a LNP singly or together as a ribonucleoprotein (RNP).

As a further example, the guide RNA and Cas nuclease, as a vector and/or associated with a LNP singly or together as a ribonucleoprotein (RNP), can be delivered in vivo or in vitro, prior to (e.g., about 1, 2, 3, 4, 5, 6, 7, 8, 9, 10, 11, 12, 13, 14, or more days) delivering the construct, as a vector and/or associated with a LNP. In some embodiments, the guide RNA and Cas nuclease are associated with an LNP and delivered to the host cell prior to delivering the donor construct.

In some embodiments, the donor construct comprises a sequence encoding a heterologous AAT, wherein the AAT sequence is wild type AAT, e.g., SEQ ID NO: 700 or 702. In some embodiments, the sequence encodes a functional variant of AAT. For example, the variant possesses increased trypsin inhibition activity than wild type AAT. In some embodiments, the sequence encodes an AAT variant that is 80%, 85%, 90%, 93%, 95%, 97%, 99% identical to SEQ ID NO: 702, having at least 80%, 85%, 90%, 92%, 94%, 96%, 98%, 99%, 100%, or more, activity as compared to wild type AAT. In some embodiments, the sequence encodes a functional fragment of AAT, wherein the fragment possesses at least 80%, 85%, 90%, 92%, 94%, 96%, 98%, 99%, 100%, or more, activity as compared to wild type AAT.

In some embodiments, the donor construct (e.g., bidirectional construct) is administered in a nucleic acid vector, such as an AAV vector, e.g., AAV8. In some embodiments, the donor construct does not comprise a homology arm.

In some embodiments, the subject is a mammal. In some embodiments, the subject is human. In some embodiments, the subject is cow, pig, monkey, sheep, dog, cat, fish, or poultry.

In some embodiments, the donor construct (e.g., bidirectional construct) comprising a sequence encoding a heterologous AAT, albumin gRNA, and RNA-guided DNA binding agent are administered intravenously. In some embodiments, the donor construct (e.g., bidirectional construct) comprising a sequence encoding a heterologous AAT, albumin gRNA, and RNA-guided DNA binding agent are administered into the hepatic circulation.

In some embodiments, a single administration of a donor construct (e.g., bidirectional construct) comprising a sequence encoding a heterologous AAT, albumin gRNA, and RNA-guided DNA binding agent is sufficient to increase expression and secretion of AAT to a desirable level. In other embodiments, more than one administration of a composition comprising a donor construct (e.g., bidirectional construct) comprising a sequence encoding a heterologous AAT, albumin gRNA, and RNA-guided DNA binding agent may be beneficial to maximize therapeutic effects.

In some embodiments, multiple administrations of a donor construct (e.g., bidirectional construct) comprising a sequence encoding a heterologous AAT, albumin gRNA, and RNA-guided DNA binding agent are used to increase expression and secretion of AAT to a desirable level and/or maximize editing via cumulative effects. In some embodiments, multiple administrations of an albumin guide RNA are used to increase expression and secretion of AAT to a desirable level and/or maximize editing via cumulative effects. In some embodiments, multiple administrations of a Cas nuclease are used to increase expression and secretion of AAT to a desirable level and/or maximize editing via cumulative effects. In some embodiments, the donor construct, albumin guide RNA, and/or Cas nuclease can be delivered every day, every two days, every three days, every four days, every week, every two weeks, every three weeks, or every four weeks. In some embodiments, a method of treating AATD further includes administering a SERPINA1 guide RNA comprising any one or more of the guide sequences of SEQ ID Nos: 1000-1128. In some embodiments, SERPINA1 gRNAs comprising any one or more of the guide sequences of SEQ ID Nos: 1000-1128 administered to treat AATD. The SERPINA1 guide RNAs may be administered together with a Cas protein or an mRNA or vector encoding a Cas protein, such as, for example, Cas9.

In some embodiments, a method of treating AATD includes reducing or preventing the accumulation of AAT (e.g., mutant, non-functional AAT) in the serum, liver, liver tissue, liver cells, and/or hepatocytes of a subject is provided comprising administering a SERPINA1 guide RNA comprising any one or more of the guide sequences of SEQ ID NOs: 1000-1128. In some embodiments, SERPINA1 gRNAs comprising any one or more of the guide sequences of SEQ ID NOs: 1000-1128 are administered to reduce or prevent the accumulation of AAT (e.g., mutant, non-functional AAT) in the liver, liver tissue, liver cells, and/or hepatocytes. The gRNAs may be administered together with an RNA-guided DNA binding agent such as a Cas protein or an mRNA or vector encoding a Cas protein, such as, for example, Cas9.

In some embodiments, the SERPINA1 gRNAs comprising the guide sequences of Table 3 together with a Cas protein induce DSBs, and non-homologous ending joining (NHEJ) during repair leads to a mutation in the SERPINA1 gene. In some embodiments, NHEJ leads to a deletion or insertion of a nucleotide(s), which induces a frame shift or nonsense mutation in the SERPINA1 gene. In some embodiments, the gRNAs comprising the guide sequences of Table 2 together with a Cas protein induce DSBs, and NHEJ repair mediates insertion of the template nucleic acid construct. In some embodiments, insertion of the template nucleic acid increases secreted AAT protein levels. In some embodiments, insertion of the template nucleic acid increases secreted heterologous AAT protein levels. In some embodiments, insertion of the template nucleic acid increases blood, serum, and/or plasma AAT protein levels.

In some embodiments, administering the SERPINA1 guide RNAs disclosed herein reduces levels of mutated alpha-1 antitrypsin (AAT) produced by the subject, and therefore prevents accumulation and aggregation of AAT in the liver.

In some embodiments, a single administration of the SERPINA1 guide RNA disclosed herein is sufficient to knock down expression of the mutant protein. In some embodiments, a single administration of the SERPINA1 guide RNA disclosed herein is sufficient to knock down or knock out expression of the mutant protein. In other embodiments, more than one administration of the SERPINA1 guide RNA disclosed herein may be beneficial to maximize editing via cumulative effects.

In some embodiments, administering the insertion guide RNAs disclosed herein increases levels of circulating alpha-1 antitrypsin (AAT) produced by the subject, and therefore prevents damage associated with high neutrophil elastase activity.

In some embodiments, a single administration or multiple administerations of an insertion guide RNA disclosed herein is sufficient to increase expression of a functional AAT protein. In some embodiments, a single administration or multiple administerations of the insertion guide RNA disclosed herein is sufficient to supplement or restore expression of the AAT protein activity. In some embodiments, the insertion guide RNA results in increased AAT serum levels, e.g., to protective levels (e.g., at or above 80 mg/dL as measured by immunodiffusion, at or above 50 mg/dL as measured using nephelometry or immunoturbidimetry and a purified standard). In some embodiments, the insertion guide RNA results in increased AAT serum levels, e.g., to normal levels (e.g., 150-350 mg/dL as measured by immunodiffusion, 90-200 mg/dL as measured using nephelometry or immunoturbidimetry and a purified standard). In some embodiments, the insertion guide RNA results in improvement in histologic grading of AATD associated liver disease, e.g., by 1, 2, 3, or more points, as compared to control, e.g., before and after treatment. In some embodiments, the insertion guide RNA results in improvement in Ishak fibrosis score as compared to control, e.g., before and after treatment. In some embodiments, a single administration improves lung disease measures, e.g., as assayed by pulmonary function testing (PFT), functional residual capacity (RFC), and/or lung density loss at total lung capacity (TLC). In other embodiments, more than one administration of the insertion guide RNA disclosed herein may be beneficial to maximize editing via cumulative effects.

In some embodiments, the efficacy of treatment with the compositions provided herein is seen at 1 year, 2 years, 3 years, 4 years, 5 years, or 10 years after delivery.

In some embodiments, treatment slow or halts lung disease progression associated with AATD. In some embodiments, treatment improves lung disease measures. In some embodiments, lung disease is measured by changes in lung structure, lung function, or symptoms in the subject. In some embodiments, efficacy of treatment is measured by increased survival time of the subject.

In some embodiments, efficacy of treatment is measured by the slowing of development of pulmonary indications. In some embodiments, efficacy of treatment is measured by clinical improvement in any one or more COPD, emphysema, or dyspnea. In some embodiments, efficacy of treatment is measured by improvement in any one or more of cough, sputum production, or wheezing.

In some embodiments, treatment slows or halts liver disease progression. In some embodiments, treatment improves liver disease measures. In some embodiments, liver disease is measured by changes in liver structure, liver function, or symptoms in the subject.

In some embodiments, efficacy of treatment is measured by the ability to delay or avoid a liver transplantation in the subject. In some embodiments, efficacy of treatment is measured by increased survival time of the subject.

In some embodiments, efficacy of treatment is measured by reduction in liver enzymes in blood. In some embodiments, the liver enzymes are alanine transaminase (ALT) or aspartate transaminase (AST).

In some embodiments, efficacy of treatment is measured by the slowing of development of scar tissue or decrease in scar tissue in the liver based on biopsy results.

In some embodiments, efficacy of treatment is measured using patient-reported results such as fatigue, weakness, itching, loss of appetite, loss of appetite, weight loss, nausea, or bloating. In some embodiments, efficacy of treatment is measured by decreases in edema, ascites, or jaundice. In some embodiments, efficacy of treatment is measured by decreases in portal hypertension. In some embodiments, efficacy of treatment is measured by decreases in rates of liver cancer.

In some embodiments, efficacy of treatment is measured using imaging methods. In some embodiments, the imaging methods are ultrasound, computerized tomography, magnetic resonance imagery, or elastography.

In some embodiments, the serum and/or liver AAT levels (e.g., mutant, non-functional AAT) are reduced by 40-50%, 50-60%, 60-70%, 70-80%, 80-90%, 90-95%, 95-98%, 98-99%, or 99-100% as compared to serum and/or liver AAT levels (e.g., mutant, non-functional AAT) before administration of the composition.

In some embodiments, the percent editing of the SERPINA1 gene is between 30 and 99%. In some embodiments, the percent editing is between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99%.

In some embodiments, the use of any one or more guide RNAs (albumin gRNA; and/or SERPINA1 gRNA) comprising any one or more of the guide sequences in Table 1 or Table 2, or Table 3 (e.g., in a composition provided herein) is provided for the preparation of a medicament for treating a human subject having AATD.

In some embodiments, the present disclosure provides combination therapies comprising any one or more of the gRNAs comprising any one or more of the guide sequences disclosed in Table 1 or Table 2 together with an augmentation therapy suitable for alleviating the lung symptoms of AATD. In some embodiments, the augmentation therapy for lung disease is intravenous therapy with AAT purified from human plasma, as described in Turner, BioDrugs 2013 December; 27(6):547-58. In some embodiments, the augmentation therapy is with Prolastin® , Zemaira®, Aralast®, or Kamada®.

In some embodiments, the combination therapy comprises any one or more of the gRNAs comprising any one or more of the guide sequences disclosed in Table 1 or Table 2, together with a siRNA that targets ATT or mutant ATT. In some embodiments, the siRNA is any siRNA capable of further reducing or eliminating the expression of wild type or mutant AAT. In some embodiments, the siRNA is administered after any one or more of the gRNAs comprising any one or more of the guide sequences disclosed in Table 1 or Table 2. In some embodiments, the siRNA is administered on a regular basis following treatment with any of the gRNA compositions provided herein

In some embodiments, the combination therapy comprises any one or more of the gRNAs comprising any one or more of the guide sequences disclosed in Table 1 or Table 2 together with one or more treatment for smoking cessation, preventive vaccinations, bronchodilators, supplemental oxygen when indicated, and physical rehabilitation in a program similar to that designed for patients with smoking-related COPD.

This description and exemplary embodiments should not be taken as limiting. For the purposes of this specification and appended embodiments, unless otherwise indicated, all numbers expressing quantities, percentages, or proportions, and other numerical values used in the specification and embodiments, are to be understood as being modified in all instances by the term “about,” to the extent they are not already so modified. Accordingly, unless indicated to the contrary, the numerical parameters set forth in the following specification and attached embodiments are approximations that may vary depending upon the desired properties sought to be obtained. At the very least, and not as an attempt to limit the application of the doctrine of equivalents to the scope of the embodiments, each numerical parameter should at least be construed in light of the number of reported significant digits and by applying ordinary rounding techniques.

Human AAT Protein Sequence (SEQ ID NO: 700) NCBI
Ref: NP_000286:
MPSSVSWGILLLAGLCCLVPVSLAEDPQGDAAQKTDTSHHDQDHPTFNKI
TPNLAEFAFSLYRQLAHQSNSTNIFFSPVSIATAFAMLSLGTKADTHDEI
LEGLNFNLTEIPEAQIHEGFQELLRTLNQPDSQLQLTTGNGLFLSEGLKL
VDKFLEDVKKLYHSEAFTVNFGDTEEAKKQINDYVEKGTQGKIVDLVKEL
DRDTVFALVNYIFFKGKWERPFEVKDTEEEDFHVDQVTTVKVPMMKRLGM
FNIQHCKKLSSWVLLMKYLGNATAIFFLPDEGKLQHLENELTHDIITKFL
ENEDRRSASLHLPKLSITGTYDLKSVLGQLGITKVFSNGADLSGVTEEAP
LKLSKAVHKAVLTIDEKGTEAAGAMFLEAIPMSIPPEVKFNKPFVFLMIE
QNTKSPLFMGKVVNPTQK
Human AAT Nucleotide Sequence (SEQ ID NO: 701)
NCBI Ref: NM_000295):
ACAATGACTCCTTTCGGTAAGTGCAGTGGAAGCTGTACACTGCCCAGGCA
AAGCGTCCGGGCAGCGTAGGCGGGCGACTCAGATCCCAGCCAGTGGACTT
AGCCCCTGTTTGCTCCTCCGATAACTGGGGTGACCTTGGTTAATATTCAC
CAGCAGCCTCCCCCGTTGCCCCTCTGGATCCACTGCTTAAATACGGACGA
GGACAGGGCCCTGTCTCCTCAGCTTCAGGCACCACCACTGACCTGGGACA
GTGAATCGACAATGCCGTCTTCTGTCTCGTGGGGCATCCTCCTGCTGGCA
GGCCTGTGCTGCCTGGTCCCTGTCTCCCTGGCTGAGGATCCCCAGGGAGA
TGCTGCCCAGAAGACAGATACATCCCACCATGATCAGGATCACCCAACCT
TCAACAAGATCACCCCCAACCTGGCTGAGTTCGCCTTCAGCCTATACCGC
CAGCTGGCACACCAGTCCAACAGCACCAATATCTTCTTCTCCCCAGTGAG
CATCGCTACAGCCTTTGCAATGCTCTCCCTGGGGACCAAGGCTGACACTC
ACGATGAAATCCTGGAGGGCCTGAATTTCAACCTCACGGAGATTCCGGAG
GCTCAGATCCATGAAGGCTTCCAGGAACTCCTCCGTACCCTCAACCAGCC
AGACAGCCAGCTCCAGCTGACCACCGGCAATGGCCTGTTCCTCAGCGAGG
GCCTGAAGCTAGTGGATAAGTTTTTGGAGGATGTTAAAAAGTTGTACCAC
TCAGAAGCCTTCACTGTCAACTTCGGGGACACCGAAGAGGCCAAGAAACA
GATCAACGATTACGTGGAGAAGGGTACTCAAGGGAAAATTGTGGATTTGG
TCAAGGAGCTTGACAGAGACACAGTTTTTGCTCTGGTGAATTACATCTTC
TTTAAAGGCAAATGGGAGAGACCCTTTGAAGTCAAGGACACCGAGGAAGA
GGACTTCCACGTGGACCAGGTGACCACCGTGAAGGTGCCTATGATGAAGC
GTTTAGGCATGTTTAACATCCAGCACTGTAAGAAGCTGTCCAGCTGGGTG
CTGCTGATGAAATACCTGGGCAATGCCACCGCCATCTTCTTCCTGCCTGA
TGAGGGGAAACTACAGCACCTGGAAAATGAACTCACCCACGATATCATCA
CCAAGTTCCTGGAAAATGAAGACAGAAGGTCTGCCAGCTTACATTTACCC
AAACTGTCCATTACTGGAACCTATGATCTGAAGAGCGTCCTGGGTCAACT
GGGCATCACTAAGGTCTTCAGCAATGGGGCTGACCTCTCCGGGGTCACAG
AGGAGGCACCCCTGAAGCTCTCCAAGGCCGTGCATAAGGCTGTGCTGACC
ATCGACGAGAAAGGGACTGAAGCTGCTGGGGCCATGTTTTTAGAGGCCAT
ACCCATGTCTATCCCCCCCGAGGTCAAGTTCAACAAACCCTTTGTCTTCT
TAATGATTGAACAAAATACCAAGTCTCCCCTCTTCATGGGAAAAGTGGTG
AATCCCACCCAAAAATAACTGCCTCTCGCTCCTCAACCCCTCCCCTCCAT
CCCTGGCCCCCTCCCTGGATGACATTAAAGAAGGGTTGAGCTGGTCCCTG
CCTGCATGTGACTGTAAATCCCTCCCATGTTTTCTCTGAGTCTCCCTTTG
CCTGCTGAGGCTGTATGTGGGCTCCAGGTAACAGTGCTGTCTTCGGGCCC
CCTGAACTGTGTTCATGGAGCATCTGGCTGGGTAGGCACATGCTGGGCTT
GAATCCAGGGGGGACTGAATCCTCAGCTTACGGACCTGGGCCCATCTGTT
TCTGGAGGGCTCCAGTCTTCCTTGTCCTGTCTTGGAGTCCCCAAGAAGGA
ATCACAGGGGAGGAACCAGATACCAGCCATGACCCCAGGCTCCACCAAGC
ATCTTCATGTCCCCCTGCTCATCCCCCACTCCCCCCCACCCAGAGTTGCT
CATCCTGCCAGGGCTGGCTGTGCCCACCCCAAGGCTGCCCTCCTGGGGGC
CCCAGAACTGCCTGATCGTGCCGTGGCCCAGTTTTGTGGCATCTGCAGCA
ACACAAGAGAGAGGACAATGTCCTCCTCTTGACCCGCTGTCACCTAACCA
GACTCGGGCCCTGCACCTCTCAGGCACTTCTGGAAAATGACTGAGGCAGA
TTCTTCCTGAAGCCCATTCTCCATGGGGCAACAAGGACACCTATTCTGTC
CTTGTCCTTCCATCGCTGCCCCAGAAAGCCTCACATATCTCCGTTTAGAA
TCAGGTCCCTTCTCCCCAGATGAAGAGGAGGGTCTCTGCTTTGTTTTCTC
TATCTCCTCCTCAGACTTGACCAGGCCCAGCAGGCCCCAGAAGACCATTA
CCCTATATCCCTTCTCCTCCCTAGTCACATGGCCATAGGCCTGCTGATGG
CTCAGGAAGGCCATTGCAAGGACTCCTCAGCTATGGGAGAGGAAGCACAT
CACCCATTGACCCCCGCAACCCCTCCCTTTCCTCCTCTGAGTCCCGACTG
GGGCCACATGCAGCCTGACTTCTTTGTGCCTGTTGCTGTCCCTGCAGTCT
TCAGAGGGCCACCGCAGCTCCAGTGCCACGGCAGGAGGCTGTTCCTGAAT
AGCCCCTGTGGTAAGGGCCAGGAGAGTCCTTCCATCCTCCAAGGCCCTGC
TAAAGGACACAGCAGCCAGGAAGTCCCCTGGGCCCCTAGCTGAAGGACAG
CCTGCTCCCTCCGTCTCTACCAGGAATGGCCTTGTCCTATGGAAGGCACT
GCCCCATCCCAAACTAATCTAGGAATCACTGTCTAACCACTCACTGTCAT
GAATGTGTACTTAAAGGATGAGGTTGAGTCATACCAAATAGTGATTTCGA
TAGTTCAAAATGGTGAAATTAGCAATTCTACATGATTCAGTCTAATCAAT
GGATACCGACTGTTTCCCACACAAGTCTCCTGTTCTCTTAAGCTTACTCA
CTGACAGCCTTTCACTCTCCACAAATACATTAAAGATATGGCCATCACCA
AGCCCCCTAGGATGACACCAGACCTGAGAGTCTGAAGACCTGGATCCAAG
TTCTGACTTTTCCCCCTGACAGCTGTGTGACCTTCGTGAAGTCGCCAAAC
CTCTCTGAGCCCCAGTCATTGCTAGTAAGACCTGCCTTTGAGTTGGTATG
ATGTTCAAGTTAGATAACAAAATGTTTATACCCATTAGAACAGAGAATAA
ATAGAACTACATTTCTTGCA
Alpha 1-antitrypsin polypeptide encoded by P00450
(SEQ ID NO: 702):
EDPQGDAAQKTDTSHHDQDHPTFNKITPNLAEFAFSLYRQLAHQSNSTNI
FFSPVSIATAFAMLSLGTKADTHDEILEGLNFNLTEIPEAQIHEGFQELL
RTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SVLGQLGITKVFSNGADLSGVTEEAPLKLSKAVHKAVLTIDEKGTEAAGA
MFLEAIPMSIPPEVKFNKPFVFLMIEQNTKSPLFMGKVVNPTQK
Human AAT Protein Signal Sequence (SEQ ID NO:
1129)
MPSSVSWGILLLAGLCCLVPVSLA

### EXAMPLES

The following examples are provided to illustrate certain disclosed embodiments and are not to be construed as limiting the scope of this disclosure in any way.

### Example 1

### Materials and Methods

### Cloning and Plasmid Preparation

A bidirectional insertion construct flanked by AAV2 ITRs was synthesized and cloned into pUC57-Kan by a commercial vendor. The resulting construct (P00147) was used as the parental cloning vector for other vectors. The other insertion constructs (without ITRs) were also commercially synthesized and cloned into pUC57. Purified plasmid was digested with BglII restriction enzyme (New England BioLabs, cat# R0144S), and the insertion constructs were cloned into the parental vector. Plasmid was propagated in Stb13™ Chemically Competent E. coli (Thermo Fisher, Cat# C737303).

### AAV Production

Triple transfection in HEK293 cells was used to package genomes with constructs of interest for AAV8 and AAV-DJ production and resulting vectors were purified from both lysed cells and culture media through iodixanol gradient ultracentrifugation method (See, e.g., Lock et al., Hum Gene Ther. 2010 October; 21(10):1259-71). The plasmids used in the triple transfection that contained the genome with constructs of interest are referenced in the Examples by a “PXXXX” number, see also e.g., Table 14. Isolated AAV was dialyzed in storage buffer (PBS with 0.001% Pluronic F68). AAV titer was determined by qPCR using primers/probe located within the ITR region.

In Vitro Transcription (“IVT”) of Nuclease mRNA

Capped and polyadenylated Streptococcus pyogenes (“Spy”) Cas9 mRNA containing N1-methyl pseudo-U was generated by in vitro transcription using a linearized plasmid DNA template and T7 RNA polymerase. Generally, plasmid DNA containing a T7 promoter and a 100 nt poly (A/T) region was linearized by incubating at 37° C. with XbaI to complete digestion followed by heat inactivation of XbaI at 65° C. for 20 min. The linearized plasmid was purified from enzyme and buffer salts. The IVT reaction to generate Cas9 modified mRNA was incubated at 37° C. for 4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine RNase inhibitor (NEB); 0.004 U/μInorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The Cas9 mRNA was purified using a MegaClear Transcription Clean-up kit according to the manufacturer's protocol (ThermoFisher). Alternatively, the Cas9 mRNA was purified using LiCl precipitation, ammonium acetate precipitation, and sodium acetate precipitation or using a LiCl precipitation method, followed by further purification by tangential flow filtration. The transcript concentration was determined by measuring the light absorbance at 260 nm (Nanodrop), and the transcript was analyzed by capillary electrophoresis by Bioanlayzer (Agilent).

The Cas9 mRNAs below comprise Cas9 ORF SEQ ID NO: 288 or a sequence of Table 24 of PCT/US2019/053423 (which is hereby incorporated by reference).

SEQ ID NO: 288:
ATGGATAAGAAGTACTCAATCGGGCTGGATATCGGAACTAATTCCGTGGG
TTGGGCAGTGATCACGGATGAATACAAAGTGCCGTCCAAGAAGTTCAAGG
TCCTGGGGAACACCGATAGACACAGCATCAAGAAAAATCTCATCGGAGCC
CTGCTGTTTGACTCCGGCGAAACCGCAGAAGCGACCCGGCTCAAACGTAC
CGCGAGGCGACGCTACACCCGGCGGAAGAATCGCATCTGCTATCTGCAAG
AGATCTTTTCGAACGAAATGGCAAAGGTCGACGACAGCTTCTTCCACCGC
CTGGAAGAATCTTTCCTGGTGGAGGAGGACAAGAAGCATGAACGGCATCC
TATCTTTGGAAACATCGTCGACGAAGTGGCGTACCACGAAAAGTACCCGA
CCATCTACCATCTGCGGAAGAAGTTGGTTGACTCAACTGACAAGGCCGAC
CTCAGATTGATCTACTTGGCCCTCGCCCATATGATCAAATTCCGCGGACA
CTTCCTGATCGAAGGCGATCTGAACCCTGATAACTCCGACGTGGATAAGC
TTTTCATTCAACTGGTGCAGACCTACAACCAACTGTTCGAAGAAAACCCA
ATCAATGCTAGCGGCGTCGATGCCAAGGCCATCCTGTCCGCCCGGCTGTC
GAAGTCGCGGCGCCTCGAAAACCTGATCGCACAGCTGCCGGGAGAGAAAA
AGAACGGACTTTTCGGCAACTTGATCGCTCTCTCACTGGGACTCACTCCC
AATTTCAAGTCCAATTTTGACCTGGCCGAGGACGCGAAGCTGCAACTCTC
AAAGGACACCTACGACGACGACTTGGACAATTTGCTGGCACAAATTGGCG
ATCAGTACGCGGATCTGTTCCTTGCCGCTAAGAACCTTTCGGACGCAATC
TTGCTGTCCGATATCCTGCGCGTGAACACCGAAATAACCAAAGCGCCGCT
TAGCGCCTCGATGATTAAGCGGTACGACGAGCATCACCAGGATCTCACGC
TGCTCAAAGCGCTCGTGAGACAGCAACTGCCTGAAAAGTACAAGGAGATC
TTCTTCGACCAGTCCAAGAATGGGTACGCAGGGTACATCGATGGAGGCGC
TAGCCAGGAAGAGTTCTATAAGTTCATCAAGCCAATCCTGGAAAAGATGG
ACGGAACCGAAGAACTGCTGGTCAAGCTGAACAGGGAGGATCTGCTCCGG
AAACAGAGAACCTTTGACAACGGATCCATTCCCCACCAGATCCATCTGGG
TGAGCTGCACGCCATCTTGCGGCGCCAGGAGGACTTTTACCCATTCCTCA
AGGACAACCGGGAAAAGATCGAGAAAATTCTGACGTTCCGCATCCCGTAT
TACGTGGGCCCACTGGCGCGCGGCAATTCGCGCTTCGCGTGGATGACTAG
AAAATCAGAGGAAACCATCACTCCTTGGAATTTCGAGGAAGTTGTGGATA
AGGGAGCTTCGGCACAAAGCTTCATCGAACGAATGACCAACTTCGACAAG
AATCTCCCAAACGAGAAGGTGCTTCCTAAGCACAGCCTCCTTTACGAATA
CTTCACTGTCTACAACGAACTGACTAAAGTGAAATACGTTACTGAAGGAA
TGAGGAAGCCGGCCTTTCTGTCCGGAGAACAGAAGAAAGCAATTGTCGAT
CTGCTGTTCAAGACCAACCGCAAGGTGACCGTCAAGCAGCTTAAAGAGGA
CTACTTCAAGAAGATCGAGTGTTTCGACTCAGTGGAAATCAGCGGGGTGG
AGGACAGATTCAACGCTTCGCTGGGAACCTATCATGATCTCCTGAAGATC
ATCAAGGACAAGGACTTCCTTGACAACGAGGAGAACGAGGACATCCTGGA
AGATATCGTCCTGACCTTGACCCTTTTCGAGGATCGCGAGATGATCGAGG
AGAGGCTTAAGACCTACGCTCATCTCTTCGACGATAAGGTCATGAAACAA
CTCAAGCGCCGCCGGTACACTGGTTGGGGCCGCCTCTCCCGCAAGCTGAT
CAACGGTATTCGCGATAAACAGAGCGGTAAAACTATCCTGGATTTCCTCA
AATCGGATGGCTTCGCTAATCGTAACTTCATGCAATTGATCCACGACGAC
AGCCTGACCTTTAAGGAGGACATCCAAAAAGCACAAGTGTCCGGACAGGG
AGACTCACTCCATGAACACATCGCGAATCTGGCCGGTTCGCCGGCGATTA
AGAAGGGAATTCTGCAAACTGTGAAGGTGGTCGACGAGCTGGTGAAGGTC
ATGGGACGGCACAAACCGGAGAATATCGTGATTGAAATGGCCCGAGAAAA
CCAGACTACCCAGAAGGGCCAGAAAAACTCCCGCGAAAGGATGAAGCGGA
TCGAAGAAGGAATCAAGGAGCTGGGCAGCCAGATCCTGAAAGAGCACCCG
GTGGAAAACACGCAGCTGCAGAACGAGAAGCTCTACCTGTACTATTTGCA
AAATGGACGGGACATGTACGTGGACCAAGAGCTGGACATCAATCGGTTGT
CTGATTACGACGTGGACCACATCGTTCCACAGTCCTTTCTGAAGGATGAC
TCGATCGATAACAAGGTGTTGACTCGCAGCGACAAGAACAGAGGGAAGTC
AGATAATGTGCCATCGGAGGAGGTCGTGAAGAAGATGAAGAATTACTGGC
GGCAGCTCCTGAATGCGAAGCTGATTACCCAGAGAAAGTTTGACAATCTC
ACTAAAGCCGAGCGCGGCGGACTCTCAGAGCTGGATAAGGCTGGATTCAT
CAAACGGCAGCTGGTCGAGACTCGGCAGATTACCAAGCACGTGGCGCAGA
TCTTGGACTCCCGCATGAACACTAAATACGACGAGAACGATAAGCTCATC
CGGGAAGTGAAGGTGATTACCCTGAAAAGCAAACTTGTGTCGGACTTTCG
GAAGGACTTTCAGTTTTACAAAGTGAGAGAAATCAACAACTACCATCACG
CGCATGACGCATACCTCAACGCTGTGGTCGGTACCGCCCTGATCAAAAAG
TACCCTAAACTTGAATCGGAGTTTGTGTACGGAGACTACAAGGTCTACGA
CGTGAGGAAGATGATAGCCAAGTCCGAACAGGAAATCGGGAAAGCAACTG
CGAAATACTTCTTTTACTCAAACATCATGAACTTTTTCAAGACTGAAATT
ACGCTGGCCAATGGAGAAATCAGGAAGAGGCCACTGATCGAAACTAACGG
AGAAACGGGCGAAATCGTGTGGGACAAGGGCAGGGACTTCGCAACTGTTC
GCAAAGTGCTCTCTATGCCGCAAGTCAATATTGTGAAGAAAACCGAAGTG
CAAACCGGCGGATTTTCAAAGGAATCGATCCTCCCAAAGAGAAATAGCGA
CAAGCTCATTGCACGCAAGAAAGACTGGGACCCGAAGAAGTACGGAGGAT
TCGATTCGCCGACTGTCGCATACTCCGTCCTCGTGGTGGCCAAGGTGGAG
AAGGGAAAGAGCAAAAAGCTCAAATCCGTCAAAGAGCTGCTGGGGATTAC
CATCATGGAACGATCCTCGTTCGAGAAGAACCCGATTGATTTCCTCGAGG
CGAAGGGTTACAAGGAGGTGAAGAAGGATCTGATCATCAAACTCCCCAAG
TACTCACTGTTCGAACTGGAAAATGGTCGGAAGCGCATGCTGGCTTCGGC
CGGAGAACTCCAAAAAGGAAATGAGCTGGCCTTGCCTAGCAAGTACGTCA
ACTTCCTCTATCTTGCTTCGCACTACGAAAAACTCAAAGGGTCACCGGAA
GATAACGAACAGAAGCAGCTTTTCGTGGAGCAGCACAAGCATTATCTGGA
TGAAATCATCGAACAAATCTCCGAGTTTTCAAAGCGCGTGATCCTCGCCG
ACGCCAACCTCGACAAAGTCCTGTCGGCCTACAATAAGCATAGAGATAAG
CCGATCAGAGAACAGGCCGAGAACATTATCCACTTGTTCACCCTGACTAA
CCTGGGAGCCCCAGCCGCCTTCAAGTACTTCGATACTACTATCGATCGCA
AAAGATACACGTCCACCAAGGAAGTTCTGGACGCGACCCTGATCCACCAA
AGCATCACTGGACTCTACGAAACTAGGATCGATCTGTCGCAGCTGGGTGG
CGAT

Lipid Formulations for Delivery of Cas9 mRNA and gRNA

Cas9 mRNA and gRNA were delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG.

For experiments utilizing pre-mixed lipid formulations (referred to herein as “lipid packets”), the components were reconstituted in 100% ethanol at a molar ratio of ionizable lipid:cholesterol:DSPC:PEG2k-DMG of 50:38:9:3, prior to being mixed with RNA cargos (e.g., Cas9 mRNA and gRNA) at a lipid amine to RNA phosphate (N:P) molar ratio of about 6.0, as further described herein.

For experiments utilizing the components formulated as lipid nanoparticles (LNPs), the components were dissolved in 100% ethanol at various molar ratios. The RNA cargos (e.g., Cas9 mRNA and gRNA) were dissolved in 25 mM citrate, 100 mM NaCl, pH 5.0, resulting in a concentration of RNA cargo of approximately 0.45 mg/mL.

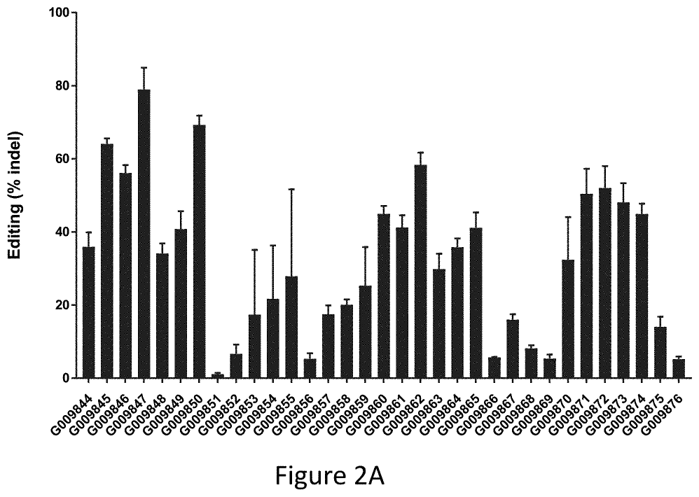

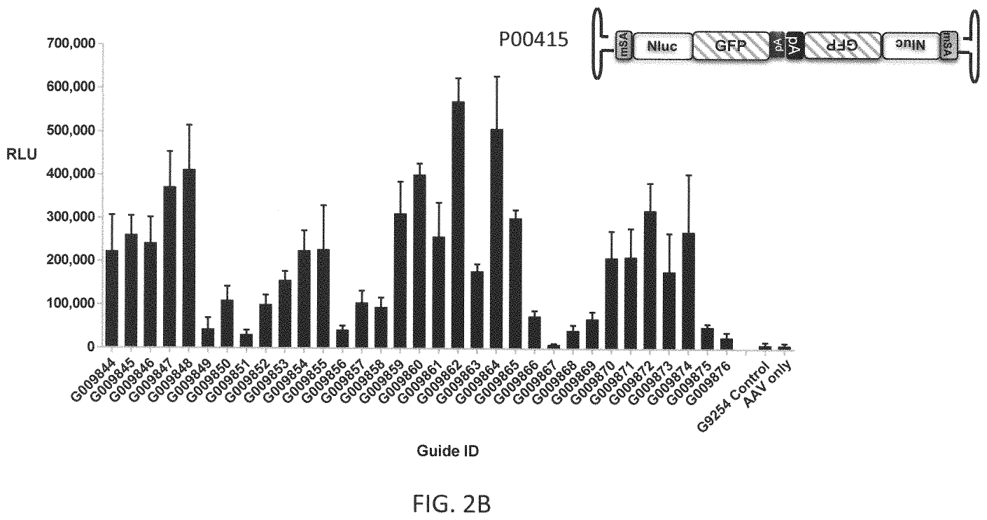

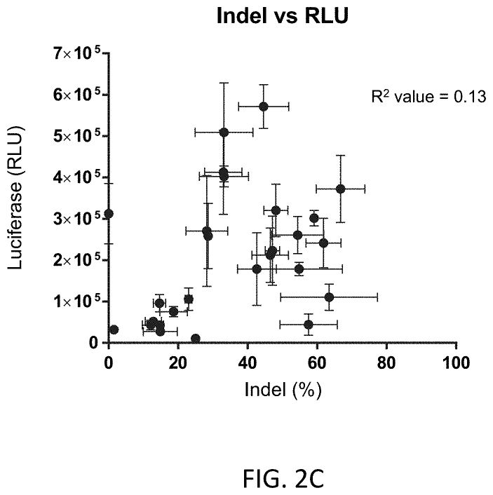

LNPs were prepared using a cross-flow technique utilizing impinging jet mixing of the lipid in ethanol with two volumes of RNA solutions and one volume of water. The lipid in ethanol was mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). Diluted LNPs were concentrated using tangential flow filtration on a flat sheet cartridge (Sartorius, 100kD MWCO) and then buffer exchanged by diafiltration into 50 mM Tris, 45 mM NaCl, 5% (w/v) sucrose, pH 7.5 (TSS). Alternatively, the final buffer exchange into TSS was completed with PD-10 desalting columns (GE). If required, formulations were concentrated by centrifugation with Amicon 100 kDa centrifugal filters (Millipore). The resulting mixture was then filtered using a 0.2 μm sterile filter. The final LNP was stored at 4° C. or −80° C. until further use. The LNPs were formulated at a molar ratio of ionizable lipid:cholesterol:DSPC:PEG2k-DMG of 50:38:9:3, with a lipid amine to RNA phosphate (N:P) molar ratio of about 6.0, and a ratio of gRNA to mRNA of 1:1 by weight.

Cell Culture and in Vitro Delivery of Cas9 mRNA, gRNA, and Insertion Constructs Primary Hepatocytes

Primary mouse hepatocytes (PMH), primary rat hepatocytes (PRH), and primary human hepatocytes (PHH) were thawed and resuspended in hepatocyte thawing medium with supplements (ThermoFisher) followed by centrifugation. The supernatant was discarded, and the pelleted cells resuspended in hepatocyte plating medium plus supplement pack (ThermoFisher). Cells were counted and plated on Bio-coat collagen I coated 96-well plates at a density of 33,000 cells/well for PHH, 50,000 cells/well for PCH, 35,000 cell/well for PRH, and 15,000 cells/well for PMH. Plated cells were allowed to settle and adhere for 6 hours in a tissue culture incubator at 37° C. and 5% CO₂ atmosphere. After incubation cells were checked for monolayer formation and were washed twice with hepatocyte maintenance prior and incubated at 37° C.

For experiments utilizing MessengerMax delivery, 100 ng Cas9 mRNA and 25 nM gRNA were each separately diluted in Opti-MEM medium. MessengerMAX reagent were diluted in Opti-MEM and incubated for 10min before adding to each tube containing Cas9 mRNA or gRNA. Incubated for 5min before adjusted to 50 ul with hepatocyte maintenance media. Media was aspirated from the cells prior to transfection of 50 ul of MessengerMAX/Cas9 mRNA mixture and MessengerMAX/gRNA mixture to the cells, followed by addition of AAV (diluted in maintenance media) at an MOI of 1e5 for PMH or 1e6 for PRH. Media was collected 72 hours post-treatment for analysis and cells were harvested for further analysis, as described herein.

For experiments utilizing LNP delivery, various volume of LNP containing desired concentration of Cas9/sgRNA were diluted with hepatocyte maintenance media supplemented with 3% FBS and incubated at 37 degree for 10min. Media was aspirated from the cells prior to addition of 100 ul of LNP/media mixture to the cells, followed by addition of AAV (diluted in maintenance media) at an MOI of 1e5 for PMH or 1e6 for PRH. Media was collected 72 hours post-treatment for analysis and cells were harvested for further analysis, as described herein. For experiments utilizing lipid packet delivery, Cas9 mRNA and gRNA were each separately diluted to 2 mg/ml in maintenance media and 2.9 μl of each were added to wells (in a 96-well Eppendorf plate) containing 12.5 μl of 50 mM sodium citrate, 200 mM sodium chloride at pH 5 and 6.9 μl of water. 12.5 μl of lipid packet formulation was then added, followed by 12.5 μl of water and 150 μl of TSS. Each well was diluted to 20 ng/μl (with respect to total RNA content) using hepatocyte maintenance media, and then diluted to 10 ng/μl (with respect to total RNA content) with 6% fresh mouse serum. Media was aspirated from the cells prior to transfection and 40 μl of the lipid packet/RNA mixtures were added to the cells, followed by addition of AAV (diluted in maintenance media) at an MOI of 1e5. Media was collected 72 hours post-treatment for analysis and cells were harvested for further analysis, as described herein.

### Luciferase Assays

For experiments involving NanoLuc detection in cell media, one volume of Nano-Glo® Luciferase Assay Substrate was combined with 50 volumes of Nano-Glo® Luciferase Assay Buffer. The assay was run on a Promega Glomax runner at an integration time of 0.5 sec using 50 ul of samples or 1:10 dilution of samples (50 μl of reagent+40 μl water+10 μl cell media).For experiments involving detection of the HiBit tag in cell media, LgBiT Protein and Nano-GloR HiBiT Extracellular Substrate were diluted 1:100 and 1:50, respectively, in room temperature Nano-GloR HiBiT Extracellular Buffer. The assay was run on a Promega Glomax runner at an integration time of 1.0 sec using 1:10 dilution of samples (50 μl of reagent+40 μl water+10 μl cell media).

In Vivo Delivery of LNP and/or AAV

Mice and rats were dosed with AAV, LNP, both AAV and LNP, or vehicle (PBS +0.001% Pluronic for AAV vehicle, TSS for LNP vehicle) via the lateral tail vein. AAV were administered in a volume of 0.1 mL per animal with amounts (vector genomes/mouse, “vg/ms”) as described herein. LNPs were diluted in TSS and administered at amounts as indicated herein, at about 5 μl/gram body weight. Typically, mice were injected first with AAV and then with LNP, if applicable. At various times points post-treatment, serum and/or liver tissue was collected for certain analyses as described further below.

### Next-Generation Sequencing (“NGS”) and Analysis for On-Target Cleavage Efficiency

Deep sequencing was utilized to identify the presence of insertions and deletions introduced by gene editing, e.g., within intron 1 of albumin. PCR primers were designed around the target site and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

Human Alpha 1-Antitrypsin (hA1AT) ELISA Analysis

For in vivo studies, blood was collected and the serum was isolated as indicated. The total human alpha 1-antitripsin levels were determined using an Alpha 1-Antitrypsin ELISA Kit (Human) (Aviva Biosystems, Cat# OKIA00048 or Abcam, Cat # ab108799) according to manufacturer's protocol. Serum hA1AT levels were quantitated off a standard curve using 4 parameter logistic fit and expressed as μg/mL of serum.

Human Alpha 1-Antitrypsin (hA1AT) LC-MS/MS Analysis

For in vivo studies, blood was collected and the serum was isolated as indicated. The total hA1AT levels were determined using liquid chromatography-tandem mass spectrometry (LC-MS/MS). Purified lyophilized native hA1AT derived from human plasma was obtained from Athens Research & Technology. Lyophilized hA1AT was dissolved in fetal calf serum at the appropriate concentration for standards and quality controls. Serum samples were diluted 10 fold into fetal calf serum. 10 μL of 1900 ng/mL stable labeled internal standards were added to 10 μL of the fetal calf serum diluted samples, standards, and quality controls. Samples were then denatured with 25 μL trifluoroethanol, diluted with 25 μL 50 mM ammonium bicarbonate immediately before 5 μL of 200 mM DTT was added and incubated for 30 min at 55° C. The reduced samples were treated with 10 μL of 200 mM iodacetamide and incubated for one hour at room temperature in the dark with shaking. The samples were diluted with 400 μL of 50 mM ammonium bicarbonate and treated with 20 μL of 1 g/L trypsin, and incubated overnight at 37° C. Digestion was terminated with 10 μL of formic acid.

Identification of Wild-Type or Mutant hA1AT Peptides:

The pure A1AT digest was analyzed by LC-MS/MS and signature peptides that contained the mutant and wild-type alleles were identified. Specifically, the mutant hA1AT (Glu342Lys) was detected using heavy labeled mutant specific peptide (AVLTIDK (SEQ ID NO: 1130)), and the wild-type hA1AT was detected using a different heavy labeled wild-type specific peptide (AVLTIDEK (SEQ ID NO: 1131)). The combined wild-type and mutant hA1AT concentration was detected using a third heavy labeled peptide (SASLHLPK (SEQ ID NO: 1132)). Each of these peptides were synthesized by incorporation of a single ¹³C₆¹⁵N-leucine at the position noted by bold underline in the SEQ ID NOs: 1130-1132).

Determining Levels of Serum hA1AT Using Mass Spectrometry:

Serum was digested according to the methods described above. After digestion, the digested serum was loaded onto the column and analyzed by LC-MS/MS as described below. Identification of wild-type and combined wild-type plus mutant hA1AT levels were obtained by comparison to a calibration curve. Mutant hA1AT levels were obtained by single point internal calibration.

### LC-MS/MS Conditions:

LC-MS/MS analysis was performed with a 2.1×50 mm C8 column. Mobile phase A consisted of 0.1% formic acid in water and mobile phase B consisted of 0.1% formic acid in acetonitrile. A needle wash consisted of 0.1% Formic Acid, 1% dimethylsulfoxide in Methanol: Water (35:65). Analysis of the A1AT digest was performed on a mass spectrometer with the following parameters: (a) Ion Source: Turbo Spray IonDrive; (b) Curtain Gas: 35.0; (c) Collision Gas: Medium; (d) IonSpray Voltage: 5500; (e) Temperature: 500° C.; (f) Ion Source Gas 1: 50; and (g) Ion Source Gas 2: 50.

### Example 2

### In Vitro Screening of a Bidirectional Construct Across Target Sites in Primary Mouse Hepatocytes

The experiment described in this Example tested the insertion of hSERPINA1 at a panel of target sites utilizing 20 different gRNAs targeting intron 1 of murine albumin in primary mouse hepatocytes (PMH).

The ssAAV and lipid packet delivery materials tested in this Example were prepared and delivered to PMH as described in Example 1, with the AAV at an MOI of 1e5. Following treatment, isolated genomic DNA and cell media was collected for editing and transgene expression analysis, respectively. The reporter vector comprised a NanoLuc ORF (in addition to GFP) that can be measured through luciferase-based fluorescence detection as described in Example 1, plotted in FIG. 1C as relative luciferase units (“RLU”). A schematic of the vector tested is provided in FIG. 1A. The gRNAs tested are shown in FIGS. 1B and 1C, using a shortened number for those listed in Table 11 (e.g., where the leading zeros are omitted, for example where “G551” corresponds to “G000551” in Table 11).

As shown in FIG. 1B and Table 31, varied levels of editing were detected. However, as shown in FIG. 1C and Table 31, high levels of editing did not necessarily result in more efficient expression of the transgenes.

TABLE 31
Indel Formation at mAlbumin Locus and NanoLuc GFP Expression

| | P00415 | | P00415 | |
|---|---|---|---|---|
| | Average | St.Dev | Average | St.Dev |
| | Indel | Indel | Luciferase | Luciferase |
| Guide ID | (%) | (%) | (RLU) | (RLU) |
| G000551 | 66.73 | 4.90 | 78633.33 | 20274.70 |
| G000552 | 90.37 | 1.01 | 205333.33 | 30664.86 |
| G000553 | 80.50 | 0.85 | 471666.67 | 134001.00 |
| G000554 | 70.60 | 2.91 | 232666.67 | 67002.49 |
| G000555 | 40.47 | 4.75 | 155666.67 | 15947.83 |
| G000666 | 65.90 | 3.99 | 313000.00 | 15394.80 |
| G000667 | 31.67 | 2.29 | 153000.00 | 30805.84 |
| G000668 | 68.30 | 4.90 | 429000.00 | 120751.80 |
| G000669 | 18.70 | 1.25 | 46466.67 | 6543.19 |
| G000670 | 51.97 | 2.06 | 424666.67 | 36473.73 |
| G011722 | 4.20 | 0.26 | 24000.00 | 8915.16 |
| G011723 | 5.93 | 0.15 | 26100.00 | 8109.87 |
| G011724 | 7.13 | 1.27 | 30933.33 | 3365.02 |
| G011725 | 2.53 | 0.65 | 20366.67 | 13955.05 |
| G011726 | 12.43 | 1.33 | 14950.00 | 8176.03 |
| G011727 | 16.20 | 2.69 | 86700.00 | 5023.94 |
| G011728 | 15.50 | 1.56 | 38166.67 | 13829.08 |
| G011729 | 6.00 | 1.01 | 26966.67 | 16085.50 |
| G011730 | 7.33 | 0.59 | 41233.33 | 25687.03 |
| G011731 | 24.87 | 1.01 | 20756.67 | 13096.20 |
| AAV Only | 0.10 | 0.00 | 207.00 | 1.41 |

### Example 3

### In Vitro Screening of Bidirectional Constructs Across Target Sites in Primary Cynomolgus and Primary Human Hepatocytes

In this Example, ssAAV vectors comprising a bidirectional construct were tested across a panel of target sites utilizing gRNAs targeting intron 1 of cynomolgus (“cyno”) and human albumin in primary cyno (PCH) and primary human hepatocytes (PHH), respectively.

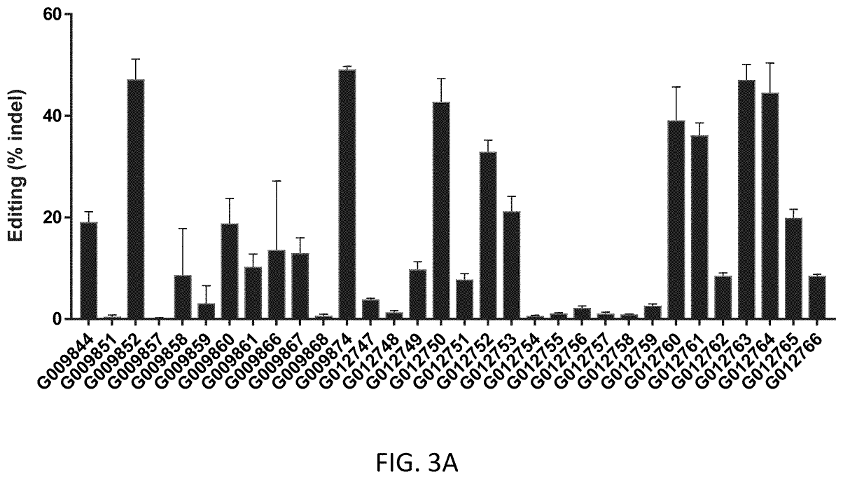

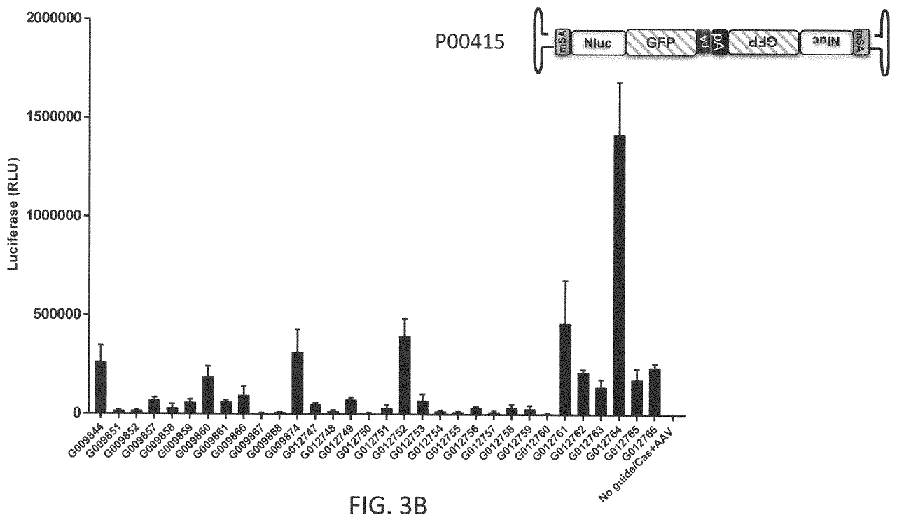

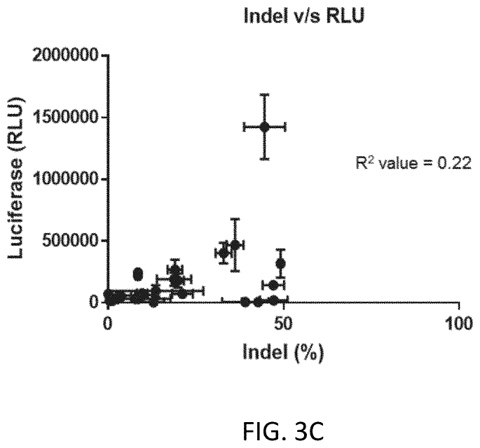

The ssAAV and lipid packet delivery materials tested in this Example were prepared and delivered to PCH and PHH as described in Example 1. Following treatment, isolated genomic DNA and cell media was collected for editing and transgene expression analysis, respectively. Each of the vectors comprised a reporter that can be measured through luciferase-based fluorescence detection as described in Example 1 (derived from plasmid P00415), plotted in FIGS. 2B and 3B as relative luciferase units (“RLU”). For example, the AAV vectors contained the NanoLuc ORF (in addition to GFP). Schematics of the vectors tested are provided in FIGS. 2B and 3B. The gRNAs tested are shown in each of the Figures using a shortened number for those listed in Table 9 and Table 13 .

As shown in FIG. 2A for PCH and FIG. 3A for PHH, varied levels of editing were detected for each of the combinations tested (editing data for some combinations tested in the PCH experiment are not reported in FIG. 2A and Table 3 due to failure of certain primer pairs used for the amplicon based sequencing). The editing data shown in FIGS. 2A and 3A graphically, are reproduced numerically in Table 3 and Table 4 below. However, as shown in FIGS. 2B, 2C and FIGS. 3B and 3C, high levels of editing did not necessarily result in more efficient expression of the transgenes, indicating little correlation between editing and insertion/expression of the bidirectional constructs in PCH and PHH, respectively.

TABLE 3
Albumin intron 1 editing data for sgRNAs
delivered to primary cynomolgus hepatocytes

| GUIDE ID | Avg % Edit | Std Dev % Edit |
|---|---|---|
| G009867 | 25.05 | 0.21 |
| G009866 | 18.7 | 3.96 |
| G009876 | 14.85 | 4.88 |
| G009875 | 12.85 | 2.33 |
| G009874 | 28.25 | 6.01 |
| G009873 | 42.65 | 5.59 |
| G009865 | 59.15 | 0.21 |
| G009872 | 48.15 | 3.46 |
| G009871 | 46.5 | 5.23 |
| G009864 | 33.2 | 8.34 |
| G009863 | 54.8 | 12.45 |
| G009862 | 44.6 | 7.21 |
| G009861 | 28.65 | 0.21 |
| G009860 | 33.2 | 7.07 |
| G009859 | 0.05 | 0.07 |
| G009858 | 14.65 | 1.77 |
| G009857 | 23 | 0.99 |
| G009856 | 14.8 | 0.99 |
| G009851 | 1.5 | 0.42 |
| G009868 | 12.15 | 2.47 |
| G009850 | 63.45 | 13.93 |
| G009849 | 57.55 | 8.27 |
| G009848 | 33 | 5.37 |
| G009847 | 66.75 | 7 |
| G009846 | 61.85 | 5.02 |
| G009845 | 54.4 | 7.5 |
| G009844 | 47.15 | 2.05 |

TABLE 4
Albumin intron 1 editing data for sgRNAs
delivered to primary human hepatocytes

| GUIDE ID | Avg % Edit | Std Dev % Edit |
|---|---|---|
| G009844 | 19.07 | 2.07 |
| G009851 | 0.43 | 0.35 |
| G009852 | 47.20 | 3.96 |
| G009857 | 0.10 | 0.14 |
| G009858 | 8.63 | 9.16 |
| G009859 | 3.07 | 3.50 |
| G009860 | 18.80 | 4.90 |
| G009861 | 10.27 | 2.51 |
| G009866 | 13.60 | 13.55 |
| G009867 | 12.97 | 3.04 |
| G009868 | 0.63 | 0.32 |
| G009874 | 49.13 | 0.60 |
| G012747 | 3.83 | 0.23 |
| G012748 | 1.30 | 0.35 |
| G012749 | 9.77 | 1.50 |
| G012750 | 42.73 | 4.58 |
| G012751 | 7.77 | 1.16 |
| G012752 | 32.93 | 2.27 |
| G012753 | 21.20 | 2.95 |
| G012754 | 0.60 | 0.10 |
| G012755 | 1.10 | 0.10 |
| G012756 | 2.17 | 0.40 |
| G012757 | 1.07 | 0.25 |
| G012758 | 0.90 | 0.10 |
| G012759 | 2.60 | 0.35 |
| G012760 | 39.10 | 6.58 |
| G012761 | 36.17 | 2.43 |
| G012762 | 8.50 | 0.57 |
| G012763 | 47.07 | 3.07 |
| G012764 | 44.57 | 5.83 |
| G012765 | 19.90 | 1.68 |
| G012766 | 8.50 | 0.28 |

### Example 4

### In Vivo Insertion of hSERPINA1 into the mAlbumin Locus

The effectiveness of various guide sequences in facilitating the insertion of hSERPINA1 into the mouse albumin locus was tested. The ssAAV and LNPs tested in this Example were prepared and delivered to mice as described in Example 1. Sera collected at weeks 1 and 2 post-dose were taken to measure human alphal antitrypsin (hA1AT) serum expression. Four weeks post dose, the animals were euthanized and liver tissue and sera were collected for editing and hA1AT serum expression, respectively. Human A1AT levels in the serum were determined by ELISA (Aviva Biosystems, Cat# OKIA00048).

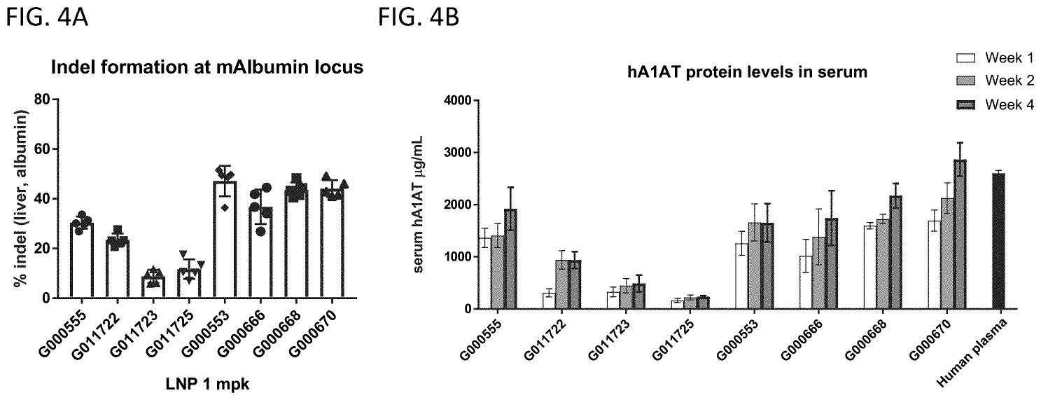

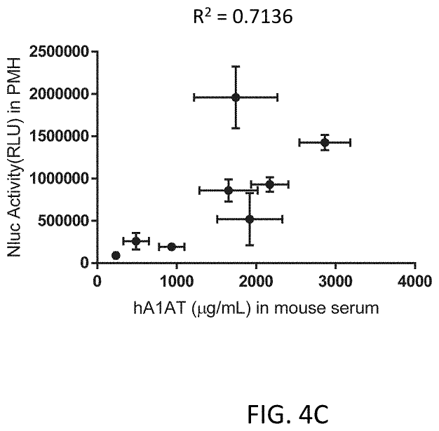

Eight different LNP formulations containing 8 different gRNA targeting intron 1 of albumin were delivered to mice along with ssAAV derived from P00450. The AAV and LNP were delivered at 1e12 vg/mouse and 1.0 mg/kg (with respect to total RNA cargo content), respectively. The gRNAs tested in this experiment are shown in FIG. 4A and FIG. 4B, using a shortened number for those listed in Table 11. Editing results at the mouse albumin locus are shown in FIG. 4A and Table 5. Serum hA1AT levels are shown in FIG. 4B and Table 6 at 1, 2, and 4 weeks post dose. FIG. 4C shows a correlation plot comparing the levels of expression as measured in RLU for a given guide from the in vitro experiment of Example 1 to the hA1AT transgene expression levels in vivo detected in this experiment using the same guide. The R² value of 0.71 demonstrated a positive correlation between the primary cell screening and the in vivo treatments.

TABLE 5
Editing at mouse albumin locus

| | Mean | | |
|---|---|---|---|
| Condition | % Indel | SD | Samples |
| G000555 | 30.4 | 2.4 | 5 |
| G011722 | 23.4 | 2.6 | 5 |
| G011723 | 8.8 | 2.6 | 5 |
| G011725 | 11.7 | 3.9 | 5 |
| G000553 | 47.1 | 6.1 | 5 |
| G000666 | 36.8 | 6.9 | 5 |
| G000668 | 43.6 | 3.0 | 5 |
| G000670 | 44.1 | 3.4 | 5 |

TABLE 6
hA1AT levels in serum

| | Week 1 | | | Week 2 | | | Week 4 | | |
|---|---|---|---|---|---|---|---|---|---|
| | Mean | | Samples | Mean | | Samples | Mean | | Samples |
| Condition | (μg/ml) | SD | (n) | (μg/ml) | SD | (n) | (μg/ml) | SD | (n) |
| G000555 | 1363 | 185 | 5 | 1409 | 230 | 5 | 1920 | 410 | 5 |
| G011722 | 308 | 78 | 5 | 941 | 178 | 5 | 937 | 160 | 5 |
| G011723 | 327 | 92 | 5 | 447 | 138 | 5 | 489 | 160 | 5 |
| G011725 | 163 | 40 | 5 | 220 | 47 | 5 | 234 | 20 | 5 |
| G000553 | 1259 | 230 | 5 | 1659 | 356 | 5 | 1652 | 367 | 5 |
| G000666 | 1019 | 317 | 5 | 1383 | 536 | 5 | 1743 | 525 | 5 |
| G000668 | 1596 | 60 | 5 | 1725 | 90 | 5 | 2172 | 234 | 5 |
| G000670 | 1694 | 202 | 5 | 2126 | 292 | 5 | 2866 | 320 | 5 |
| Human plasma | 2603 | 53 | 5 | | | | | | |

### Example 5

### In Vivo Knockdown of hSERPINA1 PiZ Transgene and Insertion of hSERPINA1 into mAlbumin Locus

In this example, a first round of editing to knock-down expression of the A1AT from the hSERPINA PiZ variant transgene (Stage 1) is followed by a second round of editing to insert hSERPINA1 into the mouse albumin locus (Stage 2). The ssAAV and LNPs tested in this Example were prepared and delivered to mice as described in Example 1 to male NSG-PiZ mice (Groups A, B, and C) and C57Bl/6 male mice (Group D) (Jackson Laboratory). NSG-PiZ mice are transgenic mice harboring copies of the human SERPINA1 PiZ variant (Glu342Lys) on the immunodeficient NOD scid gamma (NSG) background.

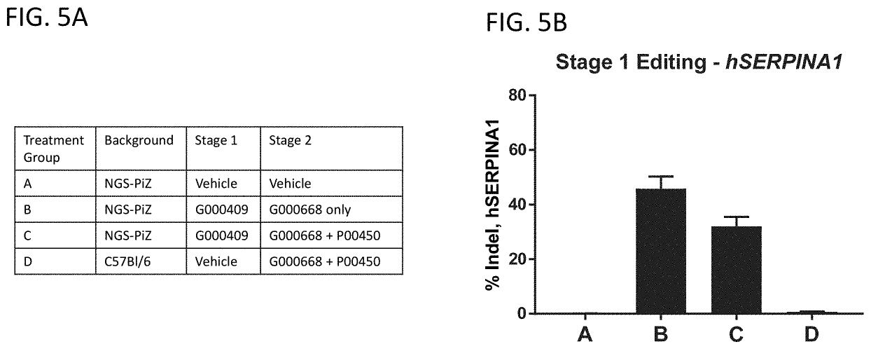

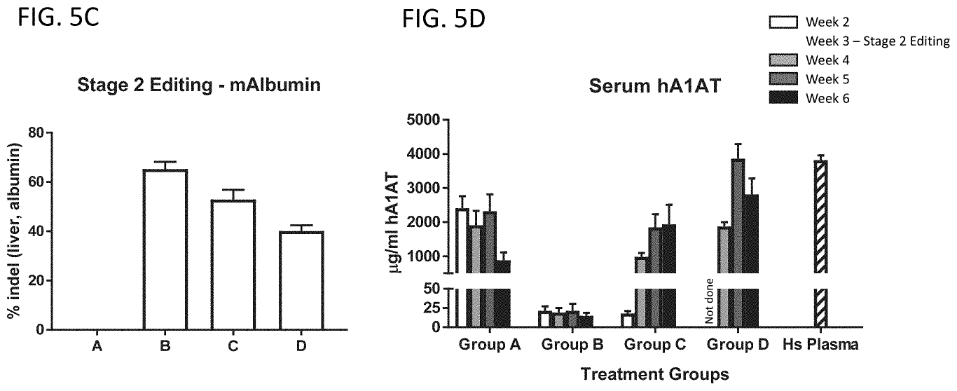

In Stage 1 of this experiment, mice were dosed with an LNP carrying Cas9 mRNA and sgRNA G000409 targeting the hSERPINA1 transgene at 0.3 mg/kg (with respect to total RNA cargo content). Two weeks after the Stage 1-dose, sera were collected to measure serum hA1AT levels. Stage 2 editing in this experiment was performed 3 weeks after the Stage 1 dosing. In Stage 2 dosing, mice from Stage 1 were dosed with 1 mg/kg (with respect to total RNA cargo content) LNP carrying Cas9 mRNA and sgRNA G000668 (targeting mouse albumin) along with ssAAV derived from P00450 at 1e12 vg/mouse. FIG. 5A outlines the editing conditions used for each test group in this experiment. Human A1AT levels in the serum were determined by ELISA (Aviva Biosystems, Cat# OKIA00048) at one, two and three weeks after Stage 2 dosing. Five weeks post Stage 2 dose, the animals were euthanized and liver tissue and sera were collected for editing and hA1AT serum expression, respectively.

FIG. 5B and Table 7 show indel formation in the hSERPINA1 PiZ variant that was targeted in Stage 1. FIG. 5C and Table 7 show indel formation in the albumin locus targeted in Stage 2. FIG. 5D and Table 8 shows hA1AT protein levels in serum at various time points as measured by ELISA, as well as hA1AT levels as measured in human plasma.

TABLE 7
Indel formation at hSERPINA1 and

| | hSERPINA1 | | | mAlbumin | | |
|---|---|---|---|---|---|---|
| Treatment | Mean | | Samples | Mean | | Samples |
| Group | Editing % | SD | (n) | Editing % | SD | (n) |
| A | 0.1 | 0.1 | 4 | 0.1 | 0.0 | 4 |
| B | 45.9 | 4.4 | 5 | 65.2 | 3.0 | 5 |
| C | 32.0 | 3.5 | 5 | 52.9 | 4.0 | 5 |
| D | 0.7 | 0.2 | 5 | 40.1 | 2.3 | 5 |

TABLE 8
hA1AT levels in serum

| Treatment Group | Data Type | Week 2 | Week 4 | Week 5 | Week 6 |
|---|---|---|---|---|---|
| Group A | Mean (μg/ml) | 2406.575 | 1903.775 | 2321.094 | 882.2813 |
| | SD | 348.6477 | 426.2463 | 491.8094 | 228.3315 |
| | Samples (n) | 4 | 4 | 4 | 4 |
| Group B | Mean (μg/ml) | 21.3279 | 18.94045 | 21.36955 | 14.82225 |
| | SD | 5.729652 | 6.187462 | 9.180648 | 3.996221 |
| | Samples (n) | 5 | 5 | 5 | 5 |
| Group C | Mean (μg/ml) | 17.8477 | 977.2608 | 1845.197 | 1938.48 |
| | SD | 3.154736 | 116.7203 | 385.4366 | 572.3113 |
| | Samples (n) | 5 | 5 | 5 | 5 |
| Group D | Mean (μg/ml) | N/A | 1871.738 | 3861.613 | 2815.315 |
| | SD | N/A | 126.3939 | 426.7188 | 463.9772 |
| | Samples (n) | N/A | 5 | 5 | 5 |
| Human Plasma | Mean (μg/ml) | 3816.88 | | | |
| | SD | 141.2429 | | | |
| | Samples (n) | 3 | | | |

### Example 6

### In Vitro Screening of a Bidirectional Construct Across Target Sites in Primary Mouse Hepatocytes Utilizing Various dgRNAs or sgRNAs

The experiment described in this Example tested the insertion of a bidirectional ssAAV construct (P00415) at a panel of target sites utilizing 41 different dual guide RNAs (dgRNA) or 4 single guide RNAs (sgRNA) targeting intron 1 of murine albumin in primary mouse hepatocytes (PMH).

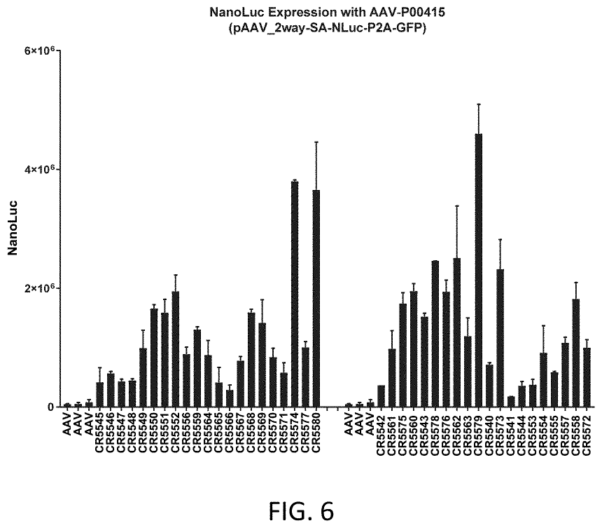

The ssAAV construct (P00415) and MessengerMAX or LNP delivery materials tested in this Example were prepared and delivered to PMH as described in Example 1, with the AAV at an MOI of 1e5. Following treatment, isolated genomic DNA and cell media was collected for editing and transgene expression analysis, respectively. The reporter vector comprised a NanoLuc ORF (in addition to GFP) that can be measured through luciferase-based fluorescence detection as described in Example 1, plotted in FIG. 6 as relative luciferase units (“RLU”). A schematic of the vector tested is provided in FIG. 1A. The dgRNAs tested are shown in FIG. 6, using a shortened number for those listed in Table 15 (e.g., where the leading zeros are omitted, for example where “CR5545” corresponds to “CR005545” in Table 15). Certain dgRNAs have a corresponding sgRNA listed in parentheses (e.g., G551 is a sgRNA which comprises the crRNA and trRNA of the dgRNA CR5542). The sgRNA construct is not tested in FIG. 6.

As shown in FIG. 6 and Table 32, varied levels of expression were detected.

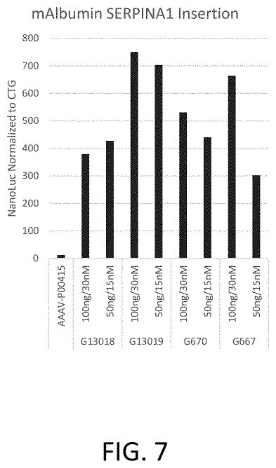

Certain dgRNAs which resulted in high transgene expression (e.g., CR5574, CR5580, CR5576, and CR5579) were tested as sgRNAs for their ability to insert hSERPINA1 in the murine albumin intron 1 site in PMH. Specifically, dgRNA CR5574, dgRNA CR5580, dgRNA CR5576, and dgRNA CR5579 correspond to sgRNA G013018, sgRNA G013018, sgRNA G667, and sgRNA G670, respectively. Either 50 ng or 100 ng Cas9 mRNA and 15 nM or 30 nM of each sgRNA was delivered to the PMH. The data in FIG. 7 are plotted as RLU normalized to CellTiter-Glo® (CTG). The sgRNAs tested are shown in FIG. 7, and are further described in Table 11. As shown in FIG. 7 and Table 33, varied levels of expression were detected.

TABLE 32
NanoLuc Expression with AAV-P00415

| | PMH AAV- P00415 | |
|---|---|---|
| | Average | St.Dev |
| | Luciferase | Luciferase |
| Guide | (RLU) | (RLU) |
| AAV | 47500 | 10170 |
| AAV | 52235 | 24895 |
| AAV | 80570 | 46130 |
| CR5545 | 415850 | 246450 |
| CR5546 | 565550 | 34150 |
| CR5547 | 432750 | 36050 |
| CR5548 | 445900 | 30100 |
| CR5549 | 993100 | 301900 |
| CR5550 | 1658000 | 68000 |
| CR5551 | 1587500 | 226500 |
| CR5552 | 1946000 | 277000 |
| CR5556 | 890600 | 118400 |
| CR5559 | 1302000 | 49000 |
| CR5564 | 874000 | 248000 |
| CR5565 | 411050 | 256550 |
| CR5566 | 286350 | 83650 |
| CR5567 | 778900 | 72000 |
| CR5568 | 1590500 | 56500 |
| CR5569 | 1417000 | 390000 |
| CR5570 | 838200 | 150900 |
| CR5571 | 577800 | 170200 |
| CR5574 | 3797000 | 25000 |
| CR5577 | 1001550 | 99450 |
| CR5580 | 3655500 | 803500 |
| AAV | 47500 | 10170 |
| AAV | 52200 | 24895 |
| AAV | 80600 | 46130 |
| CR5542 (G551) | 367000 | 300 |
| CR5561 (G552) | 982000 | 303350 |
| CR5575 (G553) | 1740000 | 186500 |
| CR5560 (G554) | 1950000 | 128500 |
| CR5543 (G555) | 1520000 | 59500 |
| CR5578 (G666) | 2460000 | 1000 |
| CR5576 (G667) | 1940000 | 197000 |
| CR5562 (G668) | 2510000 | 876000 |
| CR5563 (G669) | 1190000 | 310550 |
| CR5579 (G670) | 4600000 | 496500 |
| CR5540 | 712000 | 35550 |
| (G11722) | | |
| CR5573 | 2320000 | 500500 |
| (G11723) | | |
| CR5541 | 176000 | 1150 |
| (G11724) | | |
| CR5544 | 357000 | 72400 |
| (G11725) | | |
| CR5553 | 374000 | 93450 |
| (G11726) | | |
| CR5554 | 913000 | 455500 |
| (G11727) | | |
| CR5555 | 585000 | 13200 |
| (G11728) | | |
| CR5557 | 1080000 | 97200 |
| (G11729) | | |
| CR5558 | 1820000 | 277000 |
| (G11730) | | |
| CR5572 | 999000 | 135650 |
| (G11731) | | |

TABLE 33
mAlbumin SERPINA1 Insertion

| Guide | Concentration | NanoLuc/CTG |
|---|---|---|
| | AAAV-P00415 | 12.481248 |
| G013018 | 100 ng/30 nM | 379.68072 |
| | 50 ng/15 nM | 428.03358 |
| G013019 | 100 ng/30 nM | 750.48285 |
| | 50 ng/15 nM | 703.22584 |
| G000670 | 100 ng/30 nM | 531.21772 |
| | 50 ng/15 nM | 440.83152 |
| G000667 | 100 ng/30 nM | 664.4492 |
| | 50 ng/15 nM | 302.77669 |

TABLE 15
Mouse albumin guide dgRNA and modification
pattern

| Guide | Guide | SEQ | Full | SEQ | Genomic |
|---|---|---|---|---|---|
| ID | Sequence | ID | Sequence | ID | Coordinate |
| CR005540 | UGCUUG | 1133 | UGCUUGUA | 1153 | chr5: |
| | UAUUUU | | UUUUUCUA | | 90461039- |
| | UCUAGU | | GUAAGUUU | | 90461059 |
| | AA | | UAGAGCUA | | |
| | | | UGCUGUUU | | |
| | | | UG | | |
| CR005541 | UUUUUC | 1134 | UUUUUCUA | 1154 | chr5: |
| | UAGUAA | | GUAAUGGA | | 90461047- |
| | UGGAAG | | AGCCGUUU | | 90461067 |
| | CC | | UAGAGCUA | | |
| | | | UGCUGUUU | | |
| | | | UG | | |
| CR005542 | AUUUGC | 1135 | AUUUGCAU | 1155 | chr5: |
| | AUCUGA | | CUGAGAAC | | 90461148- |
| | GAACCC | | CCUUGUUU | | 90461168 |
| | UU | | UAGAGCUA | | |
| | | | UGCUGUUU | | |
| | | | UG | | |
| CR005543 | UGCAUC | 1136 | UGCAUCUG | 1156 | chr5: |
| | UGAGAA | | AGAACCCU | | 90461151- |
| | CCCUUA | | UAGGGUUU | | 90461171 |
| | GG | | UAGAGCUA | | |
| | | | UGCUGUUU | | |
| | | | UG | | |
| CR005544 | UUAUAU | 1137 | UUAUAUUA | 1157 | chr5: |
| | UAUUGA | | UUGAUAUA | | 90461174- |
| | UAUAUU | | UUUUGUUU | | 90461194 |
| | UU | | UAGAGCUA | | |
| | | | UGCUGUUU | | |
| | | | UG | | |
| CR005553 | GCACAG | 1138 | GCACAGAU | 1158 | chr5: |
| | AUAUAA | | AUAAACAC | | 90461480- |
| | ACACUU | | UUAAGUUU | | 90461500 |
| | AA | | UAGAGCUA | | |
| | | | UGCUGUUU | | |
| | | | UG | | |
| CR005554 | CACAGA | 1139 | CACAGAUA | 1159 | chr5: |
| | UAUAAA | | UAAACACU | | 90461481- |
| | CACUUA | | UAACGUUU | | 90461501 |
| | AC | | UAGAGCUA | | |
| | | | UGCUGUUU | | |
| | | | UG | | |
| CR005555 | GGUUUU | 1140 | GGUUUUAA | 1160 | chr5: |
| | AAAAAU | | AAAUAAUA | | 90461502- |
| | AAUAAU | | AUGUGUUU | | 90461522 |
| | GU | | UAGAGCUA | | |
| | | | UGCUGUUU | | |
| | | | UG | | |
| CR005557 | UCAGAU | 1141 | UCAGAUUU | 1161 | chr5: |
| | UUUCCU | | UCCUGUAA | | 90461572- |
| | GUAACG | | CGAUGUUU | | 90461592 |
| | AU | | UAGAGCUA | | |
| | | | UGCUGUUU | | |
| | | | UG | | |
| CR005558 | CAGAUU | 1142 | CAGAUUUU | 1162 | chr5: |
| | UUCCUG | | CCUGUAAC | | 90461573- |
| | UAACGA | | GAUCGUUU | | 90461593 |
| | UC | | UAGAGCUA | | |
| | | | UGCUGUUU | | |
| | | | UG | | |
| CR005560 | GAUCGG | 1143 | GAUCGGGA | 1163 | chr5: |
| | GAACUG | | ACUGGCAU | | 90461589- |
| | GCAUCU | | CUUCGUUU | | 90461609 |
| | UC | | UAGAGCUA | | |
| | | | UGCUGUUU | | |
| | | | UG | | |
| CR005561 | AUCGGG | 1144 | AUCGGGAA | 1164 | chr5: |
| | AACUGG | | CUGGCAUC | | 90461590- |
| | CAUCUU | | UUCAGUUU | | 90461610 |
| | CA | | UAGAGCUA | | |
| | | | UGCUGUUU | | |
| | | | UG | | |
| CR005562 | GCAUCU | 1145 | GCAUCUUC | 1165 | chr5: |
| | UCAGGG | | AGGGAGUA | | 90461601- |
| | AGUAGC | | GCUUGUUU | | 90461621 |
| | UU | | UAGAGCUA | | |
| | | | UGCUGUUU | | |
| | | | UG | | |
| CR005563 | CAAUCU | 1146 | CAAUCUUU | 1166 | chr5: |
| | UUAAAU | | AAAUAUGU | | 90461674- |
| | AUGUUG | | UGUGGUUU | | 90461694 |
| | UG | | UAGAGCUA | | |
| | | | UGCUGUUU | | |
| | | | UG | | |
| CR005572 | CAAUGG | 1147 | CAAUGGUA | 1167 | chr5: |
| | UAAAUA | | AAUAAGAA | | 90461408- |
| | AGAAAU | | AUAAGUUU | | 90461428 |
| | AA | | UAGAGCUA | | |
| | | | UGCUGUUU | | |
| | | | UG | | |
| CR005573 | GUAAAU | 1148 | GUAAAUAU | 1168 | chr5: |
| | AUCUAC | | CUACUAAG | | 90461425- |
| | UAAGAC | | ACAAGUUU | | 90461445 |
| | AA | | UAGAGCUA | | |
| | | | UGCUGUUU | | |
| | | | UG | | |
| CR005575 | GUUACA | 1149 | GUUACAGG | 1169 | chr5: |
| | GGAAAA | | AAAAUCUG | | 90461569- |
| | UCUGAA | | AAGGGUUU | | 90461589 |
| | GG | | UAGAGCUA | | |
| | | | UGCUGUUU | | |
| | | | UG | | |
| CR005576 | AUCGUU | 1150 | AUCGUUAC | 1170 | chr5: |
| | ACAGGA | | AGGAAAAU | | 90461572- |
| | AAAUCU | | CUGAGUUU | | 90461592 |
| | GA | | UAGAGCUA | | |
| | | | UGCUGUUU | | |
| | | | UG | | |
| CR005578 | CACUCU | 1151 | CACUCUUG | 1171 | chr5: |
| | UGUCUG | | UCUGUGGA | | 90461709- |
| | UGGAAA | | AACAGUUU | | 90461729 |
| | CA | | UAGAGCUA | | |
| | | | UGCUGUUU | | |
| | | | UG | | |
| CR005579 | UCACUC | 1152 | UCACUCUU | 1172 | chr5: |
| | UUGUCU | | GUCUGUGG | | 90461710- |
| | GUGGAA | | AAACGUUU | | 90461730 |
| | AC | | UAGAGCUA | | |
| | | | UGCUGUUU | | |
| | | | UG | | |

### Example 7

### In Vitro Screening of a Bidirectional Construct Across Target Sites in Primary Rat Hepatocytes

The experiment described in this Example tested the insertion of a bidirectional ssAAV construct (P00415) at a panel of target sites utilizing 32 different gRNAs targeting intron 1 of rat albumin in primary rat hepatocytes (PRH).

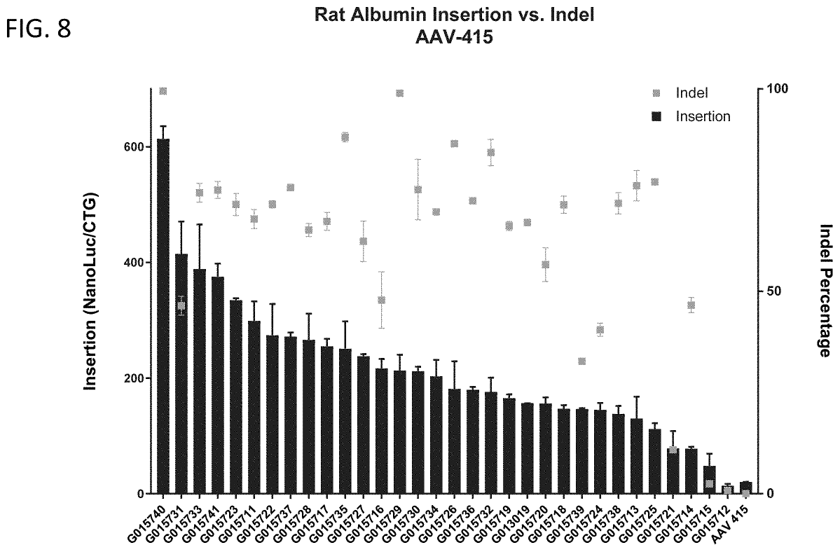

The ssAAV construct (P00415) and MessengerMAX materials tested in this Example were prepared and delivered to PRH as described in Example 1, with the AAV at an MOI of 1e6, 100 ng of Cas9 mRNA per sample, and sgRNA at a concentration of 25 nM. Following treatment, isolated genomic DNA and cell media was collected for editing and transgene expression analysis, respectively. The reporter vector comprised a NanoLuc ORF (in addition to GFP) that can be measured through luciferase-based fluorescence detection as described in Example 1. The data are plotted in FIG. 8 as relative luciferase units normalized to CellTiter-Glo® (“NanoLuc/CTG”). A schematic of the vector tested is provided in FIG. 1A.

As shown in FIG. 8, varied levels of editing (indel formation) were detected. However, high levels of editing did not necessarily result in more efficient expression of the transgenes.

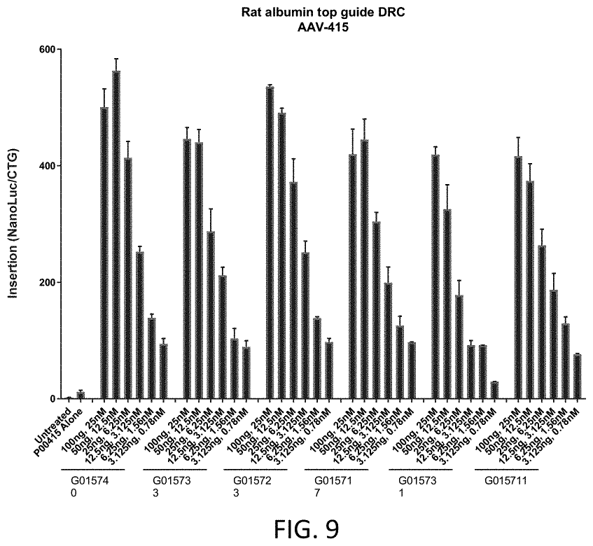

The insertion of the bidirectional ssAAV construct (P00415) at various target sites utilizing specific gRNAs tested in FIG. 8 were evaluated over a range of concentrations (Cas9: 3.125 ng, 6.25 ng, 12.5 ng, 25 ng, 5Ong, or 100 ng; sgRNA:0.78 nM, 1.56 nM, 3.125 nM, 6.25 nM, 12.5 nM, or 25 nM). As shown in FIG. 9, the insertion of the bidirectional ssAAV construct (P00415) at the rat albumin locus is dose dependent, that is, insertion rates are modulated with increasing Cas9/sgRNA dose.

TABLE 16
Rat albumin guide sgRNA and modification pattern

| | | SEQ | | SEQ |
|---|---|---|---|---|
| | | ID | Full | ID |
| Guide ID | Full Sequence | NO: | Sequence Modified | NO: |
| G015740 | ACUCUUGUCUGUGGGA | 1173 | mA*mC*mU*CUUGUCUGU | 1198 |
| | AUGGGUUUUAGAGCU | | GGGAAUGGGUUUUAGAm | |
| | AGAAAUAGCAAGUUA | | GmCmUmAmGmAmAmAm | |
| | AAGGCUAGUCCGUUAUC | | UmAmGmCAAGUUAAAAU | |
| | AAAUAAGGCUAGUCCG | | AmAmCmUmUmGmAmAm | |
| | UUAUCAACUUGAAAAA | | AmAmAmGmUmGmGmCm | |
| | GUGGCACCGAGUCGGU | | AmCmCmGmAmGmUmCmG | |
| | GCUUUU | | mGmUmGmCmU*mU*mU* | |
| | | | mU | |
| G015731 | UCCAAUCGCUACAGGA | 1174 | mU*mC*mC*AAUCGCUAC | 1199 |
| | AAAUGUUUUAGAGCU | | AGGAAAAUGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015733 | UUAGUAUAGCUAGGU | 1175 | mU*mU*mA*GUAUAGCUA | 1200 |
| | AGAGCGUUUUAGAGCU | | GGUAGAGCGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G017541 | ACUCCGAUGACAAUAA | 1176 | mA*mC*mU*CCGAUGACA | 1201 |
| | UGGGGUUUUAGAGCU | | AUAAUGGGGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015723 | GCGAUCUCACUCUUGU | 1177 | mG*mC*mG*AUCUCACUC | 1202 |
| | CUGUGUUUUAGAGCUA | | UUGUCUGUGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015711 | GAUUUUCCUGUAGCGA | 1178 | mG*mA*mU*UUUCCUGUA | 1203 |
| | UUGGGUUUUAGAGCU | | GCGAUUGGGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015722 | AUUCGUAAAAUUACUA | 1179 | mA*mU*mU*CGUAAAAUU | 1204 |
| | AUACGUUUUAGAGCUA | | ACUAAUACGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015737 | AGUCCCCCCAUUAUUG | 1180 | mA*mG*mU*CCCCCCAUU | 1205 |
| | UCAUGUUUUAGAGCUA | | AUUGUCAUGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015728 | GCUGAACUCACUAGUU | 1181 | mG*mC*mU*GAACUCACU | 1206 |
| | UCAAGUUUUAGAGCUA | | AGUUUCAAGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015717 | GAUUGGAGGCUGGGA | 1182 | mG*mA*mU*UGGAGGCUG | 1207 |
| | ACUUCGUUUUAGAGCU | | GGAACUUCGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015735 | UACAUUAAUAUAACCA | 1183 | mU*mA*mC*AUUAAUAUA | 1208 |
| | CAUAGUUUUAGAGCUA | | ACCACAUAGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015727 | AAACUCUGAAUGUAGU | 1184 | mA*mA*mA*CUCUGAAUG | 1209 |
| | CGAAGUUUUAGAGCUA | | UAGUCGAAGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015716 | AUGACUGAAACAUUCC | 1185 | mA*mU*mG*ACUGAAACA | 1210 |
| | GUCUGUUUUAGAGCUA | | UUCCGUCUGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015729 | GUGAUAUUAAUCAUCA | 1186 | mG*mU*mG*AUAUUAAUC | 1211 |
| | CCAAGUUUUAGAGCUA | | AUCACCAAGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015730 | AACUCCGAUGACAAUA | 1187 | mA*mA*mC*UCCGAUGAC | 1212 |
| | AUGGGUUUUAGAGCU | | AAUAAUGGGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015734 | CUUUGCAUUCAAACCC | 1188 | mC*mU*mU*UGCAUUCAA | 1213 |
| | AAGAGUUUUAGAGCU | | ACCCAAGAGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015726 | GAUAACUCCGAUGACA | 1189 | mG*mA*mU*AACUCCGAU | 1214 |
| | AUAAGUUUUAGAGCU | | GACAAUAAGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015736 | AUGUUUGUAAAUGUC | 1190 | mA*mU*mG*UUUGUAAAU | 1215 |
| | UACUAGUUUUAGAGCU | | GUCUACUAGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015732 | UGCAUCUGAGAAUCCU | 1191 | mU*mG*mC*AUCUGAGAA | 1216 |
| | UAUGGUUUUAGAGCU | | UCCUUAUGGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015719 | UGACUGAAACAUUCCG | 1192 | mU*mG*mA*CUGAAACAU | 1217 |
| | UCUUGUUUUAGAGCUA | | UCCGUCUUGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015720 | UCCUGUAGCGAUUGGA | 1193 | mU*mC*mC*UGUAGCGAU | 1218 |
| | GGCUGUUUUAGAGCUA | | UGGAGGCUGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015718 | UAUGCGUGUUUAGUA | 1194 | mU*mA*mU*GCGUGUUUA | 1219 |
| | UAGCUGUUUUAGAGCU | | GUAUAGCUGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015739 | ACUUUUAUUUUUCGUA | 1195 | mA*mC*mU*UUUAUUUUU | 1220 |
| | GUAAGUUUUAGAGCU | | CGUAGUAAGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015724 | UGUAUUAGUAAUUUU | 1196 | mU*mG*mU*AUUAGUAAU | 1221 |
| | ACGAAGUUUUAGAGCU | | UUUACGAAGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015738 | UUAAGUAAUUUUGAA | 1197 | mU*mU*mA*AGUAAUUUU | 1222 |
| | AUACCGUUUUAGAGCU | | GAAAUACCGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015713 | AUAACUCCGAUGACAA | 1229 | mA*mU*mA*ACUCCGAUG | 1223 |
| | UAAUGUUUUAGAGCU | | ACAAUAAUGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015725 | UAACUCCGAUGACAAU | 1230 | mU*mA*mA*CUCCGAUGA | 1224 |
| | AAUGGUUUUAGAGCU | | CAAUAAUGGUUUUAGAm | |
| | AGAAAUAGCAAGUUA | | GmCmUmAmGmAmAmAm | |
| | AAAUAAGGCUAGUCCG | | UmAmGmCAAGUUAAAAU | |
| | UUAUCAACUUGAAAAA | | AAGGCUAGUCCGUUAUC | |
| | GUGGCACCGAGUCGGU | | AmAmCmUmUmGmAmAm | |
| | GCUUUU | | AmAmAmGmUmGmGmCm | |
| | | | AmCmCmGmAmGmUmCmG | |
| | | | mGmUmGmCmU*mU*mU* | |
| | | | mU | |
| G015721 | UUUUCGUAGUAACGGA | 1231 | mU*mU*mU*UCGUAGUAA | 1225 |
| | AGCCGUUUUAGAGCUA | | CGGAAGCCGUUUUAGAm | |
| | GAAAUAGCAAGUUAA | | GmCmYmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015714 | UUCCUGUAGCGAUUGG | 1232 | mU*mU*mC*CUGUAGCGA | 1226 |
| | AGGCGUUUUAGAGCUA | | UUGGAGGCGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015715 | UCCCAGCCUCCAAUCG | 1233 | mU*mC*mC*CAGCCUCCA | 1227 |
| | CUACGUUUUAGAGCUA | | AUCGCUACGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |
| G015712 | UCCGAUUUUCCUGUAG | 1234 | mU*mC*mC*GAUUUUCCU | 1228 |
| | CGAUGUUUUAGAGCUA | | GUAGCGAUGUUUUAGAm | |
| | GAAAUAGCAAGUUAA | | GmCmUmAmGmAmAmAm | |
| | AAUAAGGCUAGUCCGU | | UmAmGmCAAGUUAAAAU | |
| | UAUCAACUUGAAAAAG | | AAGGCUAGUCCGUUAUC | |
| | UGGCACCGAGUCGGUG | | AmAmCmUmUmGmAmAm | |
| | CUUUU | | AmAmAmGmUmGmGmCm | |
| | | | AmCmCmGmAmGmUmCmG | |
| | | | mGmUmGmCmU*mU*mU* | |
| | | | mU | |

### Example 8

### In Vitro Screening of a Bidirectional Construct Across Target Sites in Primary Cynomolgus Hepatocytes Utilizing Various gRNAs

The experiment described in this Example tested the insertion of a bidirectional ssAAV construct (P00415) at a panel of target sites utilizing 34 different gRNAs targeting intron 1 of cynomolgus (“cyno”) albumin in primary cyno hepatocytes (PCH). The screen utilizing the 34 different gRNAs was performed twice to assess the variability between individual experiments.

The ssAAV and lipid packet delivery materials tested in this Example were prepared and delivered to PCH as described in Example 1. Following treatment, isolated genomic DNA and cell media was collected for editing and transgene expression analysis, respectively. Each of the vectors comprised a reporter that can be measured through luciferase-based fluorescence detection as described in Example 1 (derived from plasmid P00415). For this example, the AAV vectors contained the NanoLuc ORF (in addition to GFP). Each of the gRNAs tested and the corresponding editing data and expression of the transgenes are shown in Table 17 and Table 18. The expression of the transgene is measured as RLU normalized to CellTiter-Glo® (CTG).

As shown in Table 17, varied levels of editing were detected for each of the combinations tested. However, as shown in Table 18, high levels of editing did not necessarily result in more efficient expression of the transgenes, indicating little correlation between editing and insertion/expression of the bidirectional constructs in PCH.

TABLE 17
Albumin intron 1 editing data for sgRNAs
delivered to primary cyno hepatocytes

| | Exp. #1 | | Average |
|---|---|---|---|
| | Avg % | Exp. #2 Avg | of Exp. |
| GUIDE ID | Edit | % Edit | #1 and #2 |
| G009870 | 28.8 | 40.5 | 34.65 |
| G009848 | 41.9 | 35.2 | 38.55 |
| G009864 | 38.4 | 49.0 | 43.7 |
| G009874 | 30.2 | 39.6 | 34.9 |
| G009869 | 32.3 | 26.2 | 29.25 |
| G009860 | 42.0 | 30.8 | 36.4 |
| G009844 | 37.3 | 38.4 | 37.85 |
| G009849 | 48.8 | 51.2 | 50 |
| G009845 | 36.2 | 53.8 | 45 |
| G009859 | 36.1 | 29.2 | 32.65 |
| G009854 | 40.4 | 42.3 | 41.35 |
| G009862 | 34.9 | 32.3 | 33.6 |
| G009856 | 21.2 | 36.0 | 28.6 |
| G009846 | 43.8 | 41.7 | 42.75 |
| G009847 | 51.8 | 61.6 | 56.7 |
| G009855 | 39.9 | 39.4 | 39.65 |
| G009852 | 41.9 | 42.0 | 41.95 |
| G009851 | 40.9 | 23.6 | 32.25 |
| G009865 | 36.9 | 36.5 | 36.7 |
| G009872 | 36.2 | 34.6 | 35.4 |
| G009853 | 53.3 | 42.2 | 47.75 |
| G012766 | 45.7 | 21.0 | 33.35 |
| G009873 | 41.7 | 43.6 | 42.65 |
| G009868 | 27.4 | 29.2 | 28.3 |
| G009850 | 47.7 | 53.5 | 50.6 |
| G009861 | 32.9 | 27.2 | 30.05 |
| G009866 | 33.0 | 31.3 | 32.15 |
| G009876 | 34.1 | 29.2 | 31.65 |
| G009875 | 34.3 | 34.5 | 34.4 |
| G009863 | 59.5 | 51.7 | 55.6 |
| G009871 | 55.8 | 48.3 | 52.05 |
| G009857 | 21.4 | 22.1 | 21.75 |
| G009858 | 15.0 | 8.6 | 11.8 |
| G009867 | 48.2 | 31.3 | 39.75 |

TABLE 18
Albumin intron 1 expression data for constructs utilizing various sgRNAs
delivered to primary cyno hepatocytes

| | | | | | Average |
|---|---|---|---|---|---|
| | Exp. #1 | Exp. #1 Std | | Exp. #2 | of Exp. |
| | Avg | Dev | Exp. #2 Avg | Std Dev | #1 and |
| GUIDE ID | RLU/CTG | RLU/CTG | RLU/CTG | RLU/CTG | #2 |
| G009870 | 216.49 | 15.83 | 250.72 | 52.47 | 233.60 |
| G009848 | 320.06 | 90.30 | 241.93 | 58.04 | 281.00 |
| G009864 | 282.52 | 30.63 | 220.90 | 4.65 | 251.71 |
| G009874 | 209.18 | 46.68 | 217.59 | 17.69 | 213.38 |
| G009869 | 248.83 | 40.53 | 213.25 | 31.06 | 231.04 |
| G009860 | 200.75 | 2.50 | 197.89 | 74.55 | 199.32 |
| G009844 | 246.67 | 54.60 | 194.37 | 59.12 | 220.52 |
| G009849 | 240.66 | 47.60 | 193.26 | 17.24 | 216.96 |
| G009845 | 183.30 | 44.92 | 188.04 | 58.68 | 185.67 |
| G009859 | 215.47 | 21.27 | 187.92 | 38.90 | 201.70 |
| G009854 | 188.52 | 5.15 | 184.72 | 31.34 | 186.62 |
| G009862 | 225.87 | 4.24 | 182.45 | 4.39 | 204.16 |
| G009856 | 190.20 | 9.06 | 178.42 | 62.26 | 184.31 |
| G009846 | 223.93 | 27.92 | 177.23 | 51.96 | 200.58 |
| G009847 | 281.44 | 25.14 | 170.66 | 3.16 | 226.05 |
| G009855 | 256.16 | 40.14 | 169.57 | 8.00 | 212.87 |
| G009852 | 203.79 | 30.02 | 167.80 | 16.90 | 185.79 |
| G009851 | 200.23 | 26.64 | 167.48 | 46.06 | 183.85 |
| G009865 | 185.17 | 12.28 | 167.38 | 13.01 | 176.27 |
| G009872 | 115.79 | 15.80 | 144.73 | 11.44 | 130.26 |
| G009853 | 180.41 | 0.28 | 141.34 | 4.00 | 160.88 |
| G012766 | 131.47 | 15.22 | 139.08 | 59.22 | 135.27 |
| G009873 | 112.77 | 34.15 | 136.21 | 15.88 | 124.49 |
| G009868 | 210.02 | 9.35 | 133.32 | 18.70 | 171.67 |
| G009850 | 201.10 | 19.37 | 132.06 | 5.45 | 166.58 |
| G009861 | 169.03 | 5.39 | 124.62 | 20.05 | 146.83 |
| G009866 | 143.57 | 9.77 | 124.50 | 23.00 | 134.03 |
| G009876 | 132.26 | 31.77 | 120.94 | 30.00 | 126.60 |
| G009875 | 119.64 | 18.04 | 113.61 | 6.52 | 116.62 |
| G009863 | 160.89 | 5.30 | 108.83 | 24.78 | 134.86 |
| G009871 | 102.92 | 12.93 | 102.00 | 13.68 | 102.46 |
| G009857 | 54.05 | 11.70 | 63.37 | 4.00 | 58.71 |
| G009858 | 61.50 | 1.82 | 60.84 | 0.17 | 61.17 |
| G009867 | 14.14 | 2.65 | 12.16 | 0.25 | 13.15 |

### Example 9

### In Vivo Insertion of hSERPINA1 into the mAlbumin Locus Using a Bidirectional Construct Comprising a P2A Sequence

The effectiveness of various bidirectional constructs, with and without a P2A sequence, in facilitating the insertion of hSERPINA1 into the mouse albumin locus was tested. The ssAAV and LNPs tested in this Example were prepared and delivered to mice as described in Example 1 (n=5 per group). Sera collected at 1, 2, 4, and 5 weeks post-dose were taken to measure human alphal antitrypsin (hA1AT) serum expression.

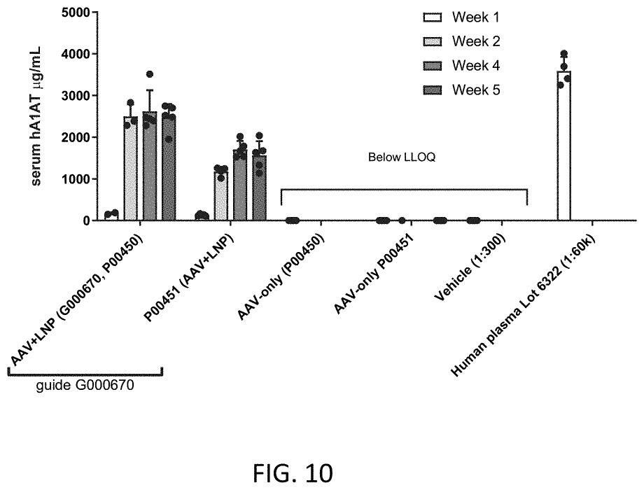

Two different constructs, P00450 and P00451, were delivered to mice along with an LNP formulation containing G000670 which targets intron 1 of albumin. The vector components and sequences for the P00450 and P00451 constructs are shown in Table 19. The AAV and LNP were delivered at 1e12 vg/mouse and 1.0 mg/kg (with respect to total RNA cargo content), respectively. Serum hA1AT levels are shown in FIG. 10 and Table 20 and Table 21 at 1, 2, 4, and 5 weeks post dose. As shown in Table 20 and Table 21, the inclusion of P2A did not necessarily result in more efficient expression of the transgene, indicating that hA1AT can be expressed with or without the inclusion of a 2A self cleaving peptide such as P2A.

TABLE 19
Vector Components and Sequences

| | Component | P00450 | P00451 |
|---|---|---|---|
| | 5′ITR | (SEQ ID NO: 263) | SEQ ID NO: 263 |
| 1^{st} Orientation | 2A | N/A | |
| | Splice | Mouse Albumin | SEQ ID NO: 264 |
| | Acceptor | Splice Acceptor | |
| | | (SEQ ID NO: 264) | |
| | Transgene | Human SERPINA1 | SEQ ID NO: 268 |
| | | (SEQ ID NO: 265) | |
| | Poly-A | SEQ ID NO: 266 | SEQ ID NO: 266 |
| 2^{nd} Orientation | Poly-A | SEQ ID NO: 267 | SEQ ID NO: 267 |
| | Transgene | Human SERPINA1 | |
| | | (SEQ ID NO: 268) | |
| | Splice | Mouse Albumin | SEQ ID NO: 269 |
| | Acceptor | Splice Acceptor | |
| | | (SEQ ID NO: 269) | |
| | 2A | N/A | |
| | 3′ITR | (SEQ ID NO: 270) | SEQ ID NO: 270 |

TABLE 20
hA1AT levels in serum (Weeks 1 and 2)

| | Week 1 | | | Week 2 | | |
|---|---|---|---|---|---|---|
| | Mean | | Samples | Mean | | Samples |
| Condition | (μg/ml) | SD | (n) | (μg/ml) | SD | (n) |
| AAV and LNP | 168.92 | 27.75 | 2 | 2499.05 | 289.49 | 3 |
| (P00450 + | | | | | | |
| G000670) | | | | | | |
| AAV and LNP | 130.90 | 22.22 | 5 | 1175.16 | 111.65 | 4 |
| (P00451 + | | | | | | |
| G000670) | | | | | | |
| AAV-only | 0 | 0 | 4 | 0 | 0 | 1 |
| (P00450) | | | | | | |
| AAV-only | 0 | 0 | 4 | 0 | 0 | 1 |
| (P00451) | | | | | | |
| Vehicle | 0 | 0 | 4 | 0 | 0 | 1 |
| Control | 3450.05 | 334.76 | 4 | | | |
| (Human | | | | | | |
| plasma lot) | | | | | | |

TABLE 21
hA1AT levels in serum (Weeks 3 and 4)

| | Week 3 | | | Week 4 | | |
|---|---|---|---|---|---|---|
| | Mean | | Samples | Mean | | Samples |
| Condition | (μg/ml) | SD | (n) | (μg/ml) | SD | (n) |
| AAV and LNP | 2620.77 | 504.64 | 5 | 2480.33 | 317.19 | 5 |
| (P00450 + | | | | | | |
| G000670) | | | | | | |
| AAV and LNP | 1705.83 | 203.36 | 5 | 1562.14 | 345.42 | 5 |
| (P00451 + | | | | | | |
| G000670) | | | | | | |
| AAV-only | | | | 0 | 0 | 5 |
| (P00450) | | | | | | |
| AAV-only | | | | 0 | 0 | 5 |
| (P00451) | | | | | | |
| Vehicle | | | | 0 | 0 | 5 |
| Control | | | | | | |
| (Human | | | | | | |
| plasma lot) | | | | | | |

### Example 10

### In Vivo Knockdown of hSERPINA1 PiZ Transgene and Insertion of hSERPINA1 into mAlbumin Locus

In this example, the ability to knock down the hSERPINA1 transgene and insert hSERPINA1 into the mouse albumin locus was evaluated. There are two stages to the experiment: (1) a first round of editing to knock-down expression of the A1AT from the hSERPINA PiZ variant transgene (Stage 1); and (2) a second round of editing to insert hSERPINA1 into the mouse albumin locus (Stage 2). The ssAAV and LNPs tested in this Example were prepared and delivered to male NSG-PiZ mice (Groups 1, 2, and 3) (Jackson Laboratory) as described in Example 1.

In Stage 1 of this experiment, mice were dosed with an LNP carrying Cas9 mRNA and sgRNA G000409 targeting the hSERPINA1 transgene at 0.3 mg/kg (with respect to total RNA cargo content) or a vehicle control. Two weeks after the Stage 1-dose, sera were collected to measure serum hA1AT levels. Stage 2 dosing in this experiment was performed 3 weeks after the Stage 1 dosing. In Stage 2 dosing, mice from Stage 1 were dosed with 1 mg/kg (with respect to total RNA cargo content) LNP carrying Cas9 mRNA and sgRNA G000668 (targeting mouse albumin) alone or with ssAAV derived from P00450 at 1e12 vg/mouse, and the control group received vehicle only. Table 22 outlines the editing conditions of each test group in this experiment. Human A1AT levels in the serum were determined by ELISA at one, two and three weeks after Stage 2 dosing. Five weeks post Stage 2 dose, the animals were euthanized and liver tissue and sera were collected to determine hA1AT serum expression levels.

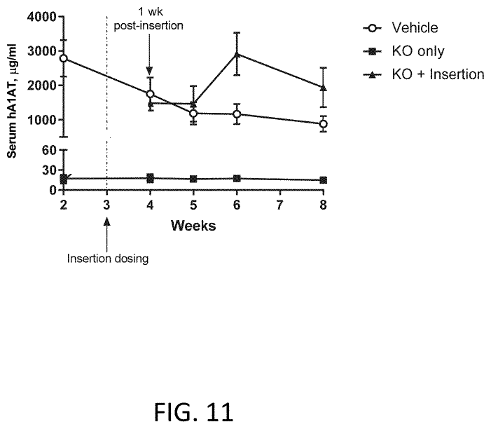

FIG. 11 and Table 23 shows hA1AT protein levels in serum at various time points as measured by ELISA.

TABLE 22
Editing conditions for each test group

| Treatment Group | Background | Stage 1 | Stage 2 |
|---|---|---|---|
| 1 | NGS-PiZ | Vehicle | Vehicle |
| 2 | NGS-PiZ | G000409 | G000668 only |
| 3 | NGS-PiZ | G000409 | G000668 + P00450 |

TABLE 23
hA1AT levels in serum

| Treatment | | Week | Week | Week | Week | Week |
|---|---|---|---|---|---|---|
| Group | Data Type | 2 | 4 | 5 | 6 | 8 |
| Group 1 | Mean | 2787.03 | 1752.09 | 1188.34 | 1165.73 | 882.28 |
| | (μg/ml) | | | | | |
| | SD | 530.59 | 479.90 | 325.73 | 288.97 | 228.33 |
| | Samples (n) | 4 | 4 | 4 | 4 | 4 |
| Group 2 | Mean | 17.08 | 17.71 | 16.58 | 17.25 | 14.82 |
| | (μg/ml) | | | | | |
| | SD | 5.86 | 6.08 | 4.88 | 4.89 | 3.99 |
| | Samples (n) | 5 | 5 | 5 | 5 | 5 |
| Group 3 | Mean | 13.99 | 1480.69 | 1465.54 | 2913.76 | 1938.48 |
| | (μg/ml) | | | | | |
| | SD | 3.20 | 215.35 | 519.08 | 619.28 | 572.31 |
| | Samples (n) | 5 | 5 | 5 | 5 | 5 |

### Example 11

### Durability of hA1AT Expression in Vivo Following Knockdown of hSERPINA1 PiZ Transgene and Insertion of hSERPINA1 into mAlbumin Locus

The durability of hA1AT expression over time in treated animals was assessed in this Example. To this end, hA1AT was measured in the serum of treated animals post-dose, as part of a 15-week durability study.

For this example, a first round of editing to knock-down expression of the A1AT from the hSERPINA PiZ variant transgene (Stage 1) is followed by a second round of editing to insert hSERPINA1 into the mouse albumin locus (Stage 2). The ssAAV, LNPs, and controls tested in this Example were prepared and delivered to mice as described in Example 1 to male NSG-PiZ mice (Groups 1, 2, 3, 4, 5, and 6).

In Stage 1 of this experiment, mice in Groups 2, 3, 4, and 6 were dosed with an LNP carrying Cas9 mRNA and sgRNA G000409 targeting the hSERPINA1 transgene at 0.3 mg/kg (with respect to total RNA cargo content). Stage 2 editing in this experiment was performed 3 weeks after the Stage 1 dosing. In Stage 2 dosing, mice from Groups 4, 5, and 6 were dosed with 1 mg/kg (with respect to total RNA cargo content) LNP carrying Cas9 mRNA and sgRNA G000666 or sgRNA G13019 (both targeting mouse albumin) along with ssAAV derived from P00450 at 1e12 vg/mouse. Table 24 outlines the editing conditions used for each test group in this experiment. Human A1AT levels in the serum were determined by ELISA at four, eight, and twelve weeks after Stage 2 dosing. Human A1AT (wild-type and mutant) levels in the serum were also determined by LC-MS/MS at one, two, four, eight, and twelve weeks after Stage 2 dosing. Twelve weeks post Stage 2 dose, the animals were euthanized and liver tissue and sera were collected for editing and hA1AT serum expression, respectively.

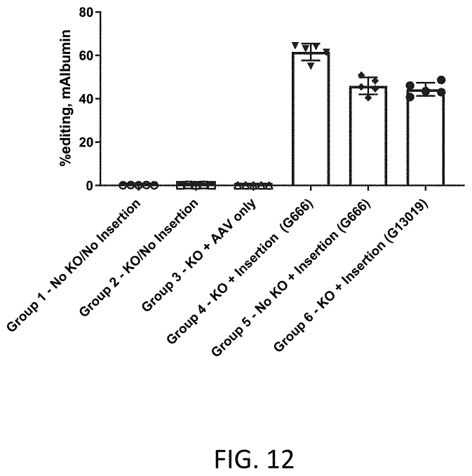

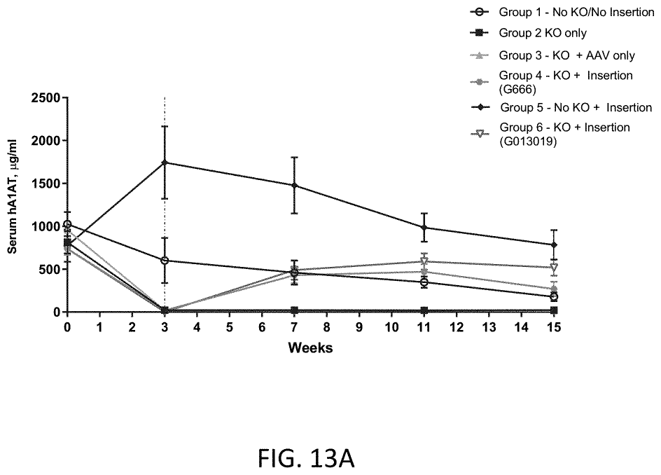

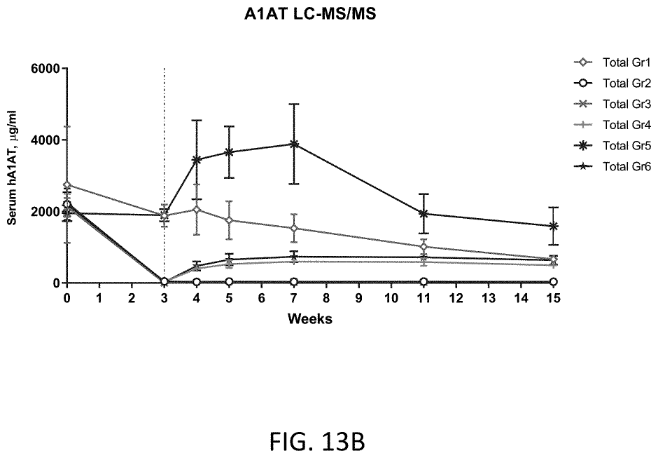

FIG. 12 and Table 25 show indel formation in the albumin locus targeted in Stage 2. FIG. 13A and Table 26 shows hA1AT protein levels in serum at various time points as measured by ELISA (Abcam, Cat # ab108799). As shown in FIG. 13A, hA1AT expression was sustained at each time point assessed for Groups 1, 4, 5, 6, and 7 out to 12 weeks after Stage 2 dosing. FIG. 13B and Table 29 shows hA1AT (wild-type and mutant) protein levels in serum at various time points as measured by LC-MS/MS. As shown in FIG. 13B, hA1AT levels decreased in each of the groups which were dosed with an LNP carrying Cas9 mRNA and sgRNA G000409 targeting the hSERPINA1 transgene (e.g., groups 2, 3, 4, and 6) during Stage 1 dosing. Meanwhile, each of the groups who were dosed with LNP carrying Cas9 mRNA and sgRNA along with ssAAV during Stage 2 dosing showed subsequent increases in serum hA1AT levels.

TABLE 24
Editing conditions for each test group

| Treatment | | | |
|---|---|---|---|
| Group | Background | Stage 1 | Stage 2 |
| 1 | NGS-PiZ | Vehicle (No KO) | Vehicle (No Insertion) |
| 2 | NGS-PiZ | G000409 (KO) | Vehicle (No Insertion) |
| 3 | NGS-PiZ | G000409 (KO) | P00450 (AAV only) |
| 4 | NGS-PiZ | G000409 (KO) | G000666 + P00450 |
| | | | (Insertion) |
| 5 | NGS-PiZ | Vehicle (No KO) | G000666 + P00450 |
| | | | (Insertion) |
| 6 | NGS-PiZ | G000409 (KO) | G13019 + P00450 |
| | | | (Insertion) |

TABLE 25
Indel formation at mAlbumin

| Treatment | mAlbumin | | |
|---|---|---|---|
| Group | Mean Editing % | SD | Samples (n) |
| 1 | 0.14 | 0.05 | 5 |
| 2 | 0.14 | 0.05 | 5 |
| 3 | 0.08 | 0.04 | 5 |
| 4 | 61.62 | 3.89 | 5 |
| 5 | 45.96 | 3.93 | 5 |
| 6 | 44.42 | 3.05 | 5 |

TABLE 26
hA1AT levels in serum as measured by ELISA

| Treatment | Data | Week | Week | Week | Week | Week |
|---|---|---|---|---|---|---|
| Group | Type | 0 | 3 | 7 | 11 | 15 |
| Group 1 | Mean | 1025.72 | 833.98 | 991.06 | 1216.32 | 1083.88 |
| | (μg/ml) | | | | | |
| | SD | 139.80 | 262.98 | 140.73 | 66.02 | 50.85 |
| | Samples (n) | 5 | 5 | 5 | 5 | 5 |
| Group 2 | Mean | 813.04 | 21.33 | 21.42 | 19.43 | 21.28 |
| | (μg/ml) | | | | | |
| | SD | 130.12 | 5.75 | 3.28 | 3.59 | 0 |
| | Samples (n) | 5 | 5 | 5 | 5 | 5 |
| Group 3 | Mean | 956.82 | 18.69 | 18.06 | 18.98 | 14.98 |
| | (μg/ml) | | | | | |
| | SD | 100.81 | 1.48 | 1.60 | 8.30 | 0 |
| | Samples (n) | 5 | 5 | 5 | 5 | 5 |
| Group 4 | Mean | 737.97 | 17.92 | 429.24 | 468.64 | 268.94 |
| | (μg/ml) | | | | | |
| | SD | 73.18 | 0 | 92.14 | 89.45 | 85.11 |
| | Samples (n) | 5 | 5 | 5 | 5 | 5 |
| Group 5 | Mean | 774.70 | 1743 | 1476.61 | 985.21 | 782.66 |
| | (μg/ml) | | | | | |
| | SD | 188.47 | 421.25 | 327.39 | 166.15 | 172.51 |
| | Samples (n) | 5 | 5 | 5 | 5 | 5 |
| Group 6 | Mean | 736.79 | 0 | 489.52 | 588.11 | 517.47 |
| | (μg/ml) | | | | | |
| | SD | 27.63 | 0 | 112.25 | 95.24 | 94.06 |
| | Samples (n) | 5 | 5 | 5 | 5 | 5 |

TABLE 29
hA1AT levels in serum as measured by LC-MS/MS

| Treatment | | Week | Week | Week | Week | Week | Week | Week |
|---|---|---|---|---|---|---|---|---|
| Group | Data Type | 0 | 3 | 4 | 5 | 7 | 11 | 15 |
| Group 1 | Mean (μg/ml) | 2746.67 | 1883.33 | 2050.00 | 1752.50 | 1532.00 | 1011.80 | 665.00 |
| | SD | 1624.85 | 310.05 | 704.66 | 528.04 | 390.22 | 207.33 | 85.20 |
| | Samples (n) | 3 | 3 | 5 | 4 | 5 | 5 | 5 |
| Group 2 | Mean (μg/ml) | 2202.00 | 40.50 | 32.28 | 36.70 | 34.25 | 39.90 | 33.25 |
| | SD | 438.94 | N = 1 | 8.60 | 10.61 | 9.11 | 20.25 | 14.04 |
| | Samples (n) | 5 | 1 | 5 | 2 | 4 | 3 | 4 |
| Group 3 | Mean (μg/ml) | 2127.50 | BQL | BQL | BQL | 25.00 | 27.60 | 2127.50 |
| | SD | 247.17 | | | | N = 1 | 0.46 | 247.17 |
| | Samples (n) | 4 | | | | 1 | 3 | 5 |
| Group 4 | Mean (μg/ml) | 2166.67 | 33.80 | 404.00 | 527.00 | 594.60 | 583.20 | 494.40 |
| | SD | 328.68 | N = 1 | 68.75 | 108.28 | 87.63 | 105.43 | 59.45 |
| | Samples (n) | 3 | 1 | 5 | 5 | 5 | 5 | 5 |
| Group 5 | Mean (μg/ml) | 1950.00 | 1895.00 | 3444.00 | 2655.00 | 3884.00 | 1936.00 | 1587.50 |
| | SD | 223.38 | 169.02 | 1105.05 | 722.84 | 1116.03 | 551.39 | 521.24 |
| | Samples (n) | 3 | 4 | 5 | 4 | 5 | 5 | 4 |
| Group 6 | Mean (μg/ml) | 2242.50 | 27.40 | 474.80 | 654.20 | 732.40 | 717.20 | 105.86 |
| | SD | 296.47 | N = 1 | 127.03 | 164.27 | 153.22 | 635.20 | 123.81 |
| | Samples (n) | 4 | 1 | 5 | 5 | 5 | 5 | 5 |

BQL = Below quantitation limit 25 μg/mL
BQL values ignored for average and standard deviation calculations

### Example 12

### Expression Level of hA1AT in Vivo with Various LNP or AAV Doses

The level of hA1AT expression in mice treated with various doses of LNP or AAV was assessed in this Example. The ssAAV and LNPs tested in this Example were prepared and delivered to mice as described in Example 1. Two weeks post dose, the animals were euthanized and liver tissue and sera were collected for editing and hA1AT serum expression, respectively.

Mice were dosed with (1) varying doses of LNP (e.g., 1 mg/kg, 0.3 mg/kg, or 0.1 mg/kg with respect to total RNA cargo content) carrying Cas9 mRNA and sgRNA G000666 (targeting mouse albumin) or (2) varying doses of ssAAV derived from P00450 (e.g., 3e12 vg/mouse, 1e12 vg/mouse, 3e11 vg/mouse, or Tell vg/mouse).

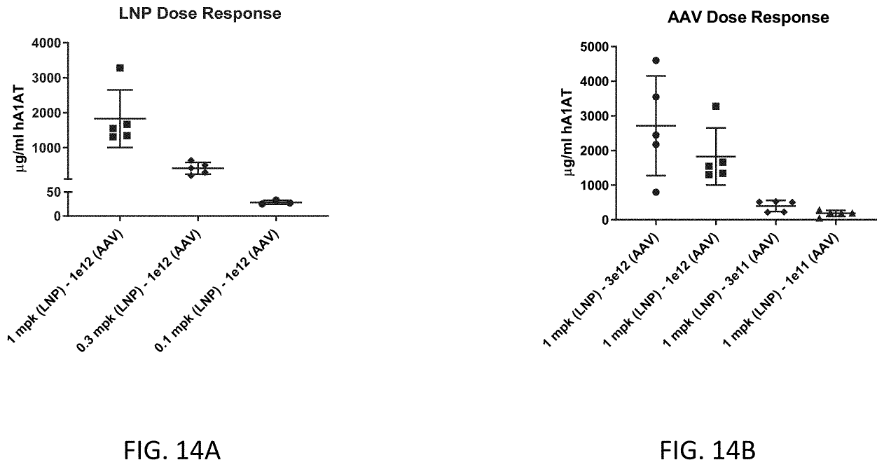

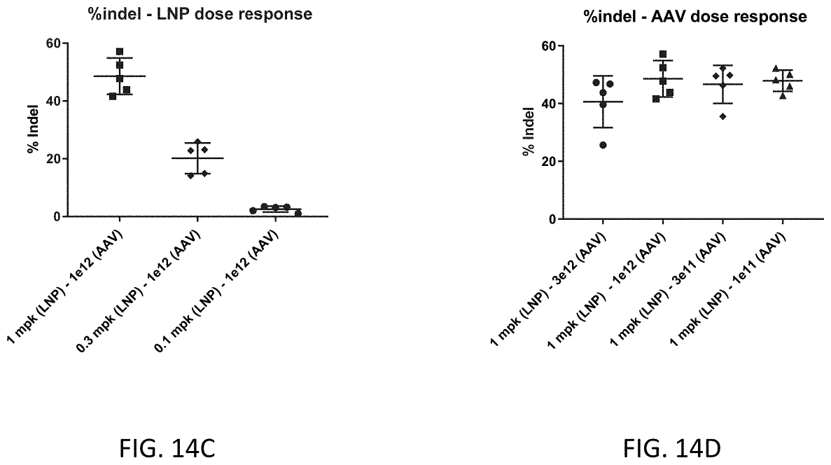

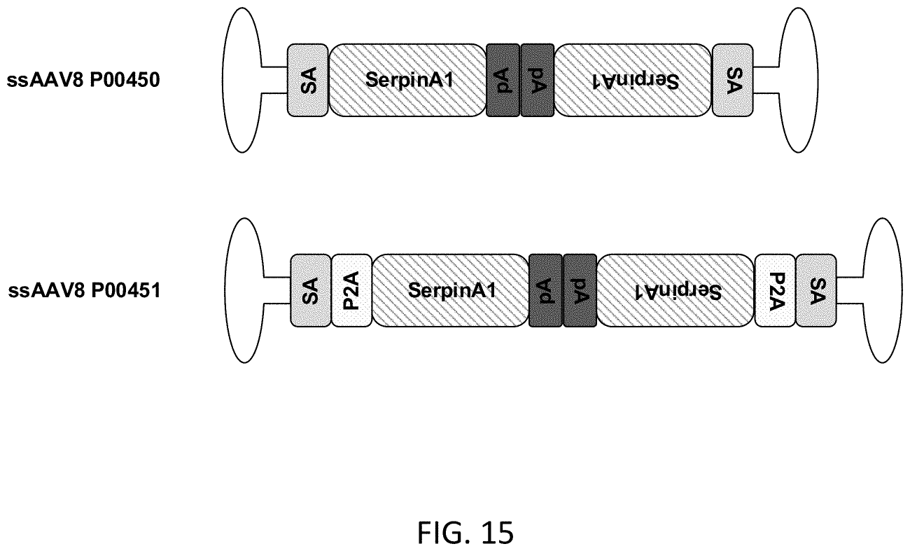

Human A1AT levels in the serum were determined by ELISA (Aviva Biosystems, Cat# OKIA00048) one week after dosing. FIG. 14A, FIG. 14B, and Table 27 show hA1AT protein levels in serum with various concentrations of LNP and AAV as measured by ELISA. As a reference, hA1AT levels in human plasma are approximately 3450.9 ug/ml. Editing results at the mouse albumin locus are shown in FIG. 14C, FIG. 14D, and Table 28. As shown in FIG. 14, FIG. 14B, FIG. 14C, and FIG. 14D hA1AT expression and indel formation increased in a dose dependent manner with increasing LNP or AAV dose, respectively. Furthermore, hSERPINA1 insertion in Wistar Rats using ssAAV and varying doses of LNP (e.g., 3 mg/kg, 1 mg/kg, or 0.3 mg/kg with respect to total RNA cargo content) carrying Cas9 mRNA and sgRNA G013019 (targeting rat albumin) showed that increasing doses of LNP results in increased expression of hA1AT in serum over 2 weeks (data not shown).

TABLE 27
hA1AT levels in serum

| | Mean | | Samples |
|---|---|---|---|
| Condition | (μg/ml) | SD | (n) |
| 1 mpk (LNP) and | 1827.13 | 824.68 | 5 |
| 1e12 (AAV) | | | |
| 0.3 mpk (LNP) and | 408.26 | 169.47 | 5 |
| 1e12 (AAV) | | | |
| 0.1 mpk (LNP) and | 28.54 | 4.10 | 5 |
| 1e12 (AAV) | | | |
| 1 mpk (LNP) and | 2714.41 | 1439.31 | 5 |
| 3e12 (AAV) | | | |
| 1 mpk (LNP) and | 1827.13 | 824.68 | 5 |
| 1e12 (AAV) | | | |
| 1 mpk (LNP) and | 399.09 | 160.19 | 5 |
| 3e11 (AAV) | | | |
| 1 mpk (LNP) and | 188.63 | 86.21 | 5 |
| 1e11 (AAV) | | | |
| Human plasma | | | |

TABLE 28
Editing at mouse albumin locus

| | Mean | | |
|---|---|---|---|
| | % | | |
| Condition | Indel | SD | Samples |
| 1 mpk (LNP) and | 48.56 | 6.29 | 5 |
| 1e12 (AAV) | | | |
| 0.3 mpk (LNP) | 20.16 | 5.31 | 5 |
| and 1e12 (AAV) | | | |
| 0.1 mpk (LNP) | 2.54 | 1.02 | 5 |
| and 1e12 (AAV) | | | |
| 1 mpk (LNP) and | 40.62 | 8.94 | 5 |
| 3e12 (AAV) | | | |
| 1 mpk (LNP) and | 48.56 | 6.29 | 5 |
| 1e12 (AAV) | | | |
| 1 mpk (LNP) and | 46.66 | 6.58 | 5 |
| 3e11 (AAV) | | | |
| 1 mpk (LNP) and | 47.88 | 3.67 | 5 |
| 1e11 (AAV) | | | |

### Example 13

### Off-Target Analysis of Albumin Human Guides

A biochemical method (See, e.g., Cameron et al., Nature Methods. 6, 600-606; 2017) was used to determine potential off-target genomic sites cleaved by Cas9 targeting Albumin. In this experiment, 13 sgRNA targeting human Albumin and two control guides with known off-target profiles were screened using isolated HEK293 genomic DNA. The number of potential off-target sites detected using a guide concentration of 16 nM in the biochemical assay were shown in Table 30. The assay identified potential off-target sites for the sgRNAs tested.

TABLE 30
Off-Target Analysis

| | | | Off-Target | |
|---|---|---|---|---|
| | | | Site Count | SEQ |
| | | | (Improved | ID |
| gRNA ID | Target | Guide Sequence | output) | NO: |
| G012753 | Albumin | GACUGAAACUUCACAGAAUA | 62 | 20 |
| G012761 | Albumin | AGUGCAAUGGAUAGGUCUUU | 75 | 28 |
| G012752 | Albumin | UGACUGAAACUUCACAGAAU | 223 | 19 |
| G012764 | Albumin | CCUCACUCUUGUCUGGGCAA | 3985 | 31 |
| G012763 | Albumin | UGGGCAAGGGAAGAAAAAAA | 5443 | 30 |
| G009857 | Albumin | AUUUAUGAGAUCAACAGCAC | 131 | 5 |
| G009859 | Albumin | UUAAAUAAAGCAUAGUGCAA | 91 | 7 |
| G009860 | Albumin | UAAAGCAUAGUGCAAUGGAU | 133 | 8 |
| G012762 | Albumin | UGAUUCCUACAGAAAAACUC | 68 | 29 |
| G009844 | Albumin | GAGCAACCUCACUCUUGUCU | 107 | 2 |
| G012765 | Albumin | ACCUCACUCUUGUCUGGGCA | 41 | 32 |
| G012766 | Albumin | UGAGCAACCUCACUCUUGUC | 78 | 33 |
| G009874 | Albumin | UAAUAAAAUUCAAACAUCCU | 53 | 13 |
| G000644 | EMX1 | GAGUCCGAGCAGAAGAAGAA | 304 | 2001 |
| G000645 | VEGFA | GACCCCCUCCACCCCGCCUC | 1641 | 2002 |

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

Human albumin intron 1:
(SEQ ID NO: 1)
GTAAGAAATCCATTTTTCTATTGTTCAACTTTTATTCTATTTTCCCAGTA
AAATAAAGTTTTAGTAAACTCTGCATCTTTAAAGAATTATTTTGGCATTT
ATTTCTAAAATGGCATAGTATTTTGTATTTGTGAAGTCTTACAAGGTTAT
CTTATTAATAAAATTCAAACATCCTAGGTAAAAAAAAAAAAAGGTCAGAA
TTGTTTAGTGACTGTAATTTTCTTTTGCGCACTAAGGAAAGTGCAAAGTA
ACTTAGAGTGACTGAAACTTCACAGAATAGGGTTGAAGATTGAATTCATA
ACTATCCCAAAGACCTATCCATTGCACTATGCTTTATTTAAAAACCACAA
AACCTGTGCTGTTGATCTCATAAATAGAACTTGTATTTATATTTATTTTC
ATTTTAGTCTGTCTTCTTGGTTGCTGTTGATAGACACTAAAAGAGTATTA
GATATTATCTAAGTTTGAATATAAGGCTATAAATATTTAATAATTTTTAA
AATAGTATTCTTGGTAATTGAATTATTCTTCTGTTTAAAGGCAGAAGAAA
TAATTGAACATCATCCTGAGTTTTTCTGTAGGAATCAGAGCCCAATATTT
TGAAACAAATGCATAATCTAAGTCAAATGGAAAGAAATATAAAAAGTAAC
ATTATTACTTCTTGTTTTCTTCAGTATTTAACAATCCTTTTTTTTCTTCC
CTTGCCCAG

TABLE 9
Human sgRNA and modification patterns

| | | SEQ | | SEQ |
|---|---|---|---|---|
| | | ID | | ID |
| Guide ID | Full Sequence | NO: | Full Sequence Modified | NO: |
| G009844 | GAGCAACCUCACUCUUGUCUGUUUU | 34 | mG*mA*mG*CAACCUCACUCUUGUCUGU | 66 |
| | AGAGCUAGAAAUAGCAAGUUAAAAU | | UUUAGAmGmCmUmAmGmAmAmAmUm | |
| | AAGGCUAGUCCGUUAUCAACUUGAA | | AmGmCAAGUUAAAAUAAGGCUAGUCC | |
| | AAAGUGGCACCGAGUCGGUGCUUUU | | GUUAUCAmAmCmUmUmGmAmAmAmAm | |
| | | | AmGmUmGmGmCmAmCmCmGmAmGmUm | |
| | | | CmGmGmUmGmCmU*mU*mU*mU | |
| G009851 | AUGCAUUUGUUUCAAAAUAUGUUUU | 35 | mA*mU*mG*CAUUUGUUUCAAAAUAUG | 67 |
| | AGAGCUAGAAAUAGCAAGUUAAAAU | | UUUUAGAmGmCmUmAmGmAmAmAmUm | |
| | AAGGCUAGUCCGUUAUCAACUUGAA | | AmGmCAAGUUAAAAUAAGGCUAGUCCG | |
| | AAAGUGGCACCGAGUCGGUGCUUUU | | UUAUCAmAmCmUmUmGmAmAmAmAmAm | |
| | | | GmUmGmGmCmAmCmCmGmAmGmUmCm | |
| | | | GmGmUmGmCmU*mU*mU*mU | |
| G009852 | UGCAUUUGUUUCAAAAUAUUGUUUU | 36 | mU*mG*mC*AUUUGUUUCAAAAUAUUGU | 68 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmUm | |
| | | | GmGmCmAmCmCmGmAmGmUmCmGmGmUm | |
| | | | GmCmU*mU*mU*mU | |
| G009857 | AUUUAUGAGAUCAACAGCACGUUUU | 37 | mA*mU*mU*UAUGAGAUCAACAGCACGU | 69 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGm | |
| | | | UmGmGmCmAmCmCmGmAmGmUmCmGmGm | |
| | | | UmGmCmU*mU*mU*mU | |
| G009858 | GAUCAACAGCACAGGUUUUGGUUUU | 38 | mG*mA*mU*CAACAGCACAGGUUUUGGU | 70 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGm | |
| | | | UmGmGmCmAmCmCmGmAmGmUmCmGm | |
| | | | GmUmGmCmU*mU*mU*mU | |
| G009859 | UUAAAUAAAGCAUAGUGCAAGUUUU | 39 | mU*mU*mA*AAUAAAGCAUAGUGCAAGUUU | 71 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G009860 | UAAAGCAUAGUGCAAUGGAUGUUUU | 40 | mU*mA*mA*AGCAUAGUGCAAUGGAUGUUU | 72 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G009861 | UAGUGCAAUGGAUAGGUCUUGUUUU | 41 | mU*mA*mG*UGCAAUGGAUAGGUCUUGUUU | 73 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G009866 | UACUAAAACUUUAUUUUACUGUUUU | 42 | mU*mA*mC*UAAAACUUUAUUUUACUGUUU | 74 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G009867 | AAAGUUGAACAAUAGAAAAAGUUUU | 43 | mA*mA*mA*GUUGAACAAUAGAAAAAGUUU | 75 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G009868 | AAUGCAUAAUCUAAGUCAAAGUUUU | 44 | mA*mA*mU*GCAUAAUCUAAGUCAAAGUUU | 76 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G009874 | UAAUAAAAUUCAAACAUCCUGUUUU | 45 | mU*mA*mA*UAAAAUUCAAACAUCCUGUUU | 77 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012747 | GCAUCUUUAAAGAAUUAUUUGUUUU | 46 | mG*mC*mA*UCUUUAAAGAAUUAUUUGUUU | 78 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012748 | UUUGGCAUUUAUUUCUAAAAGUUUU | 47 | mU*mU*mU*GGCAUUUAUUUCUAAAAGUUU | 79 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012749 | UGUAUUUGUGAAGUCUUACAGUUUU | 48 | mU*mG*mU*AUUUGUGAAGUCUUACAGUUU | 80 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012750 | UCCUAGGUAAAAAAAAAAAAGUUUU | 49 | mU*mC*mC*UAGGUAAAAAAAAAAAAGUUU | 81 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012751 | UAAUUUUCUUUUGCGCACUAGUUUU | 50 | mU*mA*mA*UUUUCUUUUGCGCACUAGUUU | 82 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012752 | UGACUGAAACUUCACAGAAUGUUUU | 51 | mU*mG*mA*CUGAAACUUCACAGAAUGUUU | 83 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012753 | GACUGAAACUUCACAGAAUAGUUUU | 52 | mG*mA*mC*UGAAACUUCACAGAAUAGUUU | 84 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012754 | UUCAUUUUAGUCUGUCUUCUGUUUU | 53 | mU*mU*mC*AUUUUAGUCUGUCUUCUGUUU | 85 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012755 | AUUAUCUAAGUUUGAAUAUAGUUUU | 54 | mA*mU*mU*AUCUAAGUUUGAAUAUAGUUU | 86 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012756 | AAUUUUUAAAAUAGUAUUCUGUUUU | 55 | mA*mA*mU*UUUUAAAAUAGUAUUCUGUUU | 87 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012757 | UGAAUUAUUCUUCUGUUUAAGUUUU | 56 | mU*mG*mA*AUUAUUCUUCUGUUUAAGUUU | 88 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012758 | AUCAUCCUGAGUUUUUCUGUGUUUU | 57 | mA*mU*mC*AUCCUGAGUUUUUCUGUGUUU | 89 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012759 | UUACUAAAACUUUAUUUUACGUUUU | 58 | mU*mU*mA*CUAAAACUUUAUUUUACGUUU | 90 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012760 | ACCUUUUUUUUUUUUUACCUGUUUU | 59 | mA*mC*mC*UUUUUUUUUUUUUACCUGUUU | 91 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012761 | AGUGCAAUGGAUAGGUCUUUGUUUU | 60 | mA*mG*mU*GCAAUGGAUAGGUCUUUGUUU | 92 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012762 | UGAUUCCUACAGAAAAACUCGUUUU | 61 | mU*mG*mA*UUCCUACAGAAAAACUCGUUU | 93 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012763 | UGGGCAAGGGAAGAAAAAAAGUUUU | 62 | mU*mG*mG*GCAAGGGAAGAAAAAAAGUUU | 94 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012764 | CCUCACUCUUGUCUGGGCAAGUUUU | 63 | mC*mC*mU*CACUCUUGUCUGGGCAAGUUU | 95 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012765 | ACCUCACUCUUGUCUGGGCAGUUUU | 64 | mA*mC*mC*UCACUCUUGUCUGGGCAGUUU | 96 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |
| G012766 | UGAGCAACCUCACUCUUGUCGUUUU | 65 | mU*mG*mA*GCAACCUCACUCUUGUCGUUU | 97 |
| | AGAGCUAGAAAUAGCAAGUUAAAAU | | UAGAmGmCmUmAmGmAmAmAmUmAmGmC | |
| | AAGGCUAGUCCGUUAUCAACUUGAA | | AAGUUAAAAUAAGGCUAGUCCGUUAUCAm | |
| | AAAGUGGCACCGAGUCGGUGCUUUU | | AmCmUmUmGmAmAmAmAmAmGmUmGmGm | |
| | | | CmAmCmCmGmAmGmUmCmGmGmUmGmCm | |
| | | | U*mU*mU*mU | |

TABLE 10
Mouse albumin guide RNA

| | | | SEQ |
|---|---|---|---|
| | | | ID |
| Guide ID | Guide Sequence | Mouse Genomic Coordinates (mm10) | NO: |
| G000551 | AUUUGCAUCUGAGAACCCUU | chr5: 90461148-90461168 | 98 |
| G000552 | AUCGGGAACUGGCAUCUUCA | chr5: 90461590-90461610 | 99 |
| G000553 | GUUACAGGAAAAUCUGAAGG | chr5: 90461569-90461589 | 100 |
| G000554 | GAUCGGGAACUGGCAUCUUC | chr5: 90461589-90461609 | 101 |
| G000555 | UGCAUCUGAGAACCCUUAGG | chr5: 90461151-90461171 | 102 |
| G000666 | CACUCUUGUCUGUGGAAACA | chr5: 90461709-90461729 | 103 |
| G000667 | AUCGUUACAGGAAAAUCUGA | chr5: 90461572-90461592 | 104 |
| G000668 | GCAUCUUCAGGGAGUAGCUU | chr5: 90461601-90461621 | 105 |
| G000669 | CAAUCUUUAAAUAUGUUGUG | chr5: 90461674-90461694 | 106 |
| G000670 | UCACUCUUGUCUGUGGAAAC | chr5: 90461710-90461730 | 107 |
| G011722 | UGCUUGUAUUUUUCUAGUAA | chr5: 90461039-90461059 | 108 |
| G011723 | GUAAAUAUCUACUAAGACAA | chr5: 90461425-90461445 | 109 |
| G011724 | UUUUUCUAGUAAUGGAAGCC | chr5: 90461047-90461067 | 110 |
| G011725 | UUAUAUUAUUGAUAUAUUUU | chr5: 90461174-90461194 | 111 |
| G011726 | GCACAGAUAUAAACACUUAA | chr5: 90461480-90461500 | 112 |
| G011727 | CACAGAUAUAAACACUUAAC | chr5: 90461481-90461501 | 113 |
| G011728 | GGUUUUAAAAAUAAUAAUGU | chr5: 90461502-90461522 | 114 |
| G011729 | UCAGAUUUUCCUGUAACGAU | chr5: 90461572-90461592 | 115 |
| G011730 | CAGAUUUUCCUGUAACGAUC | chr5: 90461573-90461593 | 116 |
| G011731 | CAAUGGUAAAUAAGAAAUAA | chr5: 90461408-90461428 | 117 |
| G013018 | GGAAAAUCUGAAGGUGGCAA | chr5: 90461563-90461583 | 118 |
| G013019 | GGCGAUCUCACUCUUGUCUG | chr5: 90461717-90461737 | 119 |

TABLE 11
Mouse albumin guide sRNA and modification pattern

| | | SEQ | | SEQ |
|---|---|---|---|---|
| | | ID | | ID |
| Guide ID | Full Sequence | NO: | Full Sequence Modified | NO: |
| G000551 | AUUUGCAUCUGAGAACCCUUGU | 120 | mA*mU*mU*UGCAUCUGAGAACCCUUGUUUUAGAm | 142 |
| | UUUAGAGCUAGAAAUAGCAAGU | | GmCmUmAmGmAmAmAmUmAmGmCAAGUUAAAAU | |
| | UAAAAUAAGGCUAGUCCGUUAU | | AAGGCUAGUCCGUUAUCAmAmCmUmUmGmAmAm | |
| | CAACUUGAAAAAGUGGCACCGA | | AmAmAmGmUmGmGmCmAmCmCmGmAmGmUmCmG | |
| | GUCGGUGCUUUU | | mGmUmGmCmU*mU*mU*mU | |
| G000552 | AUCGGGAACUGGCAUCUUCA | 121 | mA*mU*mC*GGGAACUGGCAUCUUCAGUUUUAGAm | 143 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |
| G000553 | GUUACAGGAAAAUCUGAAGG | 122 | mG*mU*mU*ACAGGAAAAUCUGAAGGGUUUUAGA | 144 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G000554 | GAUCGGGAACUGGCAUCUUC | 123 | mG*mA*mU*CGGGAACUGGCAUCUUCGUUUUAGAm | 145 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |
| G000555 | UGCAUCUGAGAACCCUUAGG | 124 | mU*mG*mC*AUCUGAGAACCCUUAGGGUUUUAGAm | 146 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |
| G000666 | CACUCUUGUCUGUGGAAACA | 125 | mC*mA*mC*UCUUGUCUGUGGAAACAGUUUUAGAm | 147 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |
| G000667 | AUCGUUACAGGAAAAUCUGA | 126 | mA*mU*mC*GUUACAGGAAAAUCUGAGUUUUAGA | 148 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G000668 | GCAUCUUCAGGGAGUAGCUU | 127 | mG*mC*mA*UCUUCAGGGAGUAGCUUGUUUUAGA | 149 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G000669 | CAAUCUUUAAAUAUGUUGUG | 128 | mC*mA*mA*UCUUUAAAUAUGUUGUGGUUUUAGA | 150 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G000670 | UCACUCUUGUCUGUGGAAAC | 129 | mU*mC*mA*CUCUUGUCUGUGGAAACGUUUUAGAm | 151 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |
| G011722 | UGCUUGUAUUUUUCUAGUAA | 130 | mU*mG*mC*UUGUAUUUUUCUAGUAAGUUUUAGA | 152 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G011723 | GUAAAUAUCUACUAAGACAA | 131 | mG*mU*mA*AAUAUCUACUAAGACAAGUUUUAGA | 153 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G011724 | UUUUUCUAGUAAUGGAAGCC | 132 | mU*mU*mU*UUCUAGUAAUGGAAGCCGUUUUAGA | 154 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G011725 | UUAUAUUAUUGAUAUAUUUU | 133 | mU*mU*mA*UAUUAUUGAUAUAUUUUGUUUUAGA | 155 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G011726 | GCACAGAUAUAAACACUUAA | 134 | mG*mC*mA*CAGAUAUAAACACUUAAGUUUUAGA | 156 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G011727 | CACAGAUAUAAACACUUAAC | 135 | mC*mA*mC*AGAUAUAAACACUUAACGUUUUAGAm | 157 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |
| G011728 | GGUUUUAAAAAUAAUAAUGU | 136 | mG*mG*mU*UUUAAAAAUAAUAAUGUGUUUUAGA | 158 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G011729 | UCAGAUUUUCCUGUAACGAU | 137 | mU*mC*mA*GAUUUUCCUGUAACGAUGUUUUAGA | 159 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G011730 | CAGAUUUUCCUGUAACGAUC | 138 | mC*mA*mG*AUUUUCCUGUAACGAUCGUUUUAGAm | 160 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |
| G011731 | CAAUGGUAAAUAAGAAAUAA | 139 | mC*mA*mA*UGGUAAAUAAGAAAUAAGUUUUAGA | 161 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G013018 | GGAAAAUCUGAAGGUGGCAA | 140 | mG*mG*mA*AAAUCUGAAGGUGGCAAGUUUUAGA | 162 |
| | GUUUUAGAGCUAGAAAUAGC | | mGmCmUmAmGmAmAmAmUmAmGmCAAGUUAAAA | |
| | AAGUUAAAAUAAGGCUAGUC | | UAAGGCUAGUCCGUUAUCAmAmCmUmUmGmAmA | |
| | CGUUAUCAACUUGAAAAAGU | | mAmAmAmGmUmGmGmCmAmCmCmGmAmGmUmCm | |
| | GGCACCGAGUCGGUGCUUUU | | GmGmUmGmCmU*mU*mU*mU | |
| G013019 | GGCGAUCUCACUCUUGUCUG | 141 | mG*mG*mC*GAUCUCACUCUUGUCUGGUUUUAGAm | 163 |
| | GUUUUAGAGCUAGAAAUAGC | | GmCmUmAmGmAmAmAmUmAmGmCAAGUUAAAAU | |
| | AAGUUAAAAUAAGGCUAGUC | | AAGGCUAGUCCGUUAUCAmAmCmUmUmGmAmAm | |
| | CGUUAUCAACUUGAAAAAGU | | AmAmAmGmUmGmGmCmAmCmCmGmAmGmUmCmG | |
| | GGCACCGAGUCGGUGCUUUU | | mGmUmGmCmU*mU*mU*mU | |

TABLE 12
Cyno albumin guide RNA

| | | | SEQ |
|---|---|---|---|
| | | | ID |
| Guide ID | Guide Sequence | Cyno Genomic Coordinates (mf5) | NO: |
| G009844 | GAGCAACCUCACUCUUGUCU | chr5: 61198711-61198731 | 2* |
| G009845 | AGCAACCUCACUCUUGUCUG | chr5: 61198712-61198732 | 165 |
| G009846 | ACCUCACUCUUGUCUGGGGA | chr5: 61198716-61198736 | 166 |
| G009847 | CCUCACUCUUGUCUGGGGAA | chr5: 61198717-61198737 | 167 |
| G009848 | CUCACUCUUGUCUGGGGAAG | chr5: 61198718-61198738 | 168 |
| G009849 | GGGGAAGGGGAGAAAAAAAA | chr5: 61198731-61198751 | 169 |
| G009850 | GGGAAGGGGAGAAAAAAAAA | chr5: 61198732-61198752 | 170 |
| G009851 | AUGCAUUUGUUUCAAAAUAU | chr5: 61198825-61198845 | 3* |
| G009852 | UGCAUUUGUUUCAAAAUAUU | chr5: 61198826-61198846 | 4* |
| G009853 | UGAUUCCUACAGAAAAAGUC | chr5: 61198852-61198872 | 173 |
| G009854 | UACAGAAAAAGUCAGGAUAA | chr5: 61198859-61198879 | 174 |
| G009855 | UUUCUUCUGCCUUUAAACAG | chr5: 61198889-61198909 | 175 |
| G009856 | UUAUAGUUUUAUAUUCAAAC | chr5: 61198957-61198977 | 176 |
| G009857 | AUUUAUGAGAUCAACAGCAC | chr5: 61199062-61199082 | 5* |
| G009858 | GAUCAACAGCACAGGUUUUG | chr5: 61199070-61199090 | 6* |
| G009859 | UUAAAUAAAGCAUAGUGCAA | chr5: 61199096-61199116 | 7* |
| G009860 | UAAAGCAUAGUGCAAUGGAU | chr5: 61199101-61199121 | 8* |
| G009861 | UAGUGCAAUGGAUAGGUCUU | chr5: 61199108-61199128 | 9* |
| G009862 | AGUGCAAUGGAUAGGUCUUA | chr5: 61199109-61199129 | 182 |
| G009863 | UUACUUUGCACUUUCCUUAG | chr5: 61199186-61199206 | 183 |
| G009864 | UACUUUGCACUUUCCUUAGU | chr5: 61199187-61199207 | 184 |
| G009865 | UCUGACCUUUUAUUUUACCU | chr5: 61199238-61199258 | 185 |
| G009866 | UACUAAAACUUUAUUUUACU | chr5: 61199367-61199387 | 10* |
| G009867 | AAAGUUGAACAAUAGAAAAA | chr5: 61199401-61199421 | 11* |
| G009868 | AAUGCAUAAUCUAAGUCAAA | chr5: 61198812-61198832 | 12* |
| G009869 | AUUAUCCUGACUUUUUCUGU | chr5: 61198860-61198880 | 189 |
| G009870 | UGAAUUAUUCCUCUGUUUAA | chr5: 61198901-61198921 | 190 |
| G009871 | UAAUUUUCUUUUGCCCACUA | chr5: 61199203-61199223 | 191 |
| G009872 | AAAAGGUCAGAAUUGUUUAG | chr5: 61199229-61199249 | 192 |
| G009873 | AACAUCCUAGGUAAAAUAAA | chr5: 61199246-61199266 | 193 |
| G009874 | UAAUAAAAUUCAAACAUCCU | chr5: 61199258-61199278 | 13 |
| G009875 | UUGUCAUGUAUUUCUAAAAU | chr5: 61199322-61199342 | 195 |
| G009876 | UUUGUCAUGUAUUUCUAAAA | chr5: 61199323-61199343 | 196 |

SEQ ID NOs marked with an “*” above indicate that the indicated gRNA is applicable to both cyno and human.

TABLE 13
Cyno sgRNA and modification patterns

| | | SEQ | | SEQ |
|---|---|---|---|---|
| | | ID | | ID |
| Guide ID | Full Sequence | NO: | Full Sequence Modified | NO: |
| G009844 | GAGCAACCUCACUCUUGUCU | 34* | mG*mA*mG*CAACCUCACUCUUGUCUGUUUUAG | 66* |
| | GUUUUAGAGCUAGAAAUAGC | | AmGmCmUmAmGmAmAmAmUmAmGmCAAGUUA | |
| | AAGUUAAAAUAAGGCUAGUC | | AAAUAAGGCUAGUCCGUUAUCAmAmCmUmUm | |
| | CGUUAUCAACUUGAAAAAGU | | GmAmAmAmAmAmGmUmGmGmCmAmCmCmGm | |
| | GGCACCGAGUCGGUGCUUUU | | AmGmUmCmGmGmUmGmCmU*mU*mU*mU | |
| G009845 | AGCAACCUCACUCUUGUCUG | 198 | mA*mG*mC*AACCUCACUCUUGUCUGGUUUUAG | 231 |
| | GUUUUAGAGCUAGAAAUAGC | | AmGmCmUmAmGmAmAmAmUmAmGmCAAGUUA | |
| | AAGUUAAAAUAAGGCUAGUC | | AAAUAAGGCUAGUCCGUUAUCAmAmCmUmUm | |
| | CGUUAUCAACUUGAAAAAGU | | GmAmAmAmAmAmGmUmGmGmCmAmCmCmGm | |
| | GGCACCGAGUCGGUGCUUUU | | AmGmUmCmGmGmUmGmCmU*mU*mU*mU | |
| G009846 | ACCUCACUCUUGUCUGGGGA | 199 | mA*mC*mC*UCACUCUUGUCUGGGGAGUUUU | 232 |
| | GUUUUAGAGCUAGAAAUAGC | | AGAmGmCmUmAmGmAmAmAmUmAmGmCAA | |
| | AAGUUAAAAUAAGGCUAGUC | | GUUAAAAUAAGGCUAGUCCGUUAUCAmAmCm | |
| | CGUUAUCAACUUGAAAAAGU | | UmUmGmAmAmAmAmAmGmUmGmGmCmAmCm | |
| | GGCACCGAGUCGGUGCUUUU | | CmGmAmGmUmCmGmGmUmGmCmU*mU*mU*mU | |
| G009847 | CCUCACUCUUGUCUGGGGAA | 200 | mC*mC*mU*CACUCUUGUCUGGGGAAGUUUUA | 233 |
| | GUUUUAGAGCUAGAAAUAGC | | GAmGmCmUmAmGmAmAmAmUmAmGmCAAGU | |
| | AAGUUAAAAUAAGGCUAGUC | | UAAAAUAAGGCUAGUCCGUUAUCAmAmCmUm | |
| | CGUUAUCAACUUGAAAAAGU | | UmGmAmAmAmAmAmGmUmGmGmCmAmCmCm | |
| | GGCACCGAGUCGGUGCUUUU | | GmAmGmUmCmGmGmUmGmCmU*mU*mU*mU | |
| G009848 | CUCACUCUUGUCUGGGGAAG | 201 | mC*mU*mC*ACUCUUGUCUGGGGAAGGUUUU | 234 |
| | GUUUUAGAGCUAGAAAUAGC | | AGAmGmCmUmAmGmAmAmAmUmAmGmCAA | |
| | AAGUUAAAAUAAGGCUAGUC | | GUUAAAAUAAGGCUAGUCCGUUAUCAmAmCm | |
| | CGUUAUCAACUUGAAAAAGU | | UmUmGmAmAmAmAmAmGmUmGmGmCmAmCm | |
| | GGCACCGAGUCGGUGCUUUU | | CmGmAmGmUmCmGmGmUmGmCmU*mU*mU*mU | |
| G009849 | GGGGAAGGGGAGAAAAAAAA | 202 | mG*mG*mG*GAAGGGGAGAAAAAAAAGUUUUAG | 235 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009850 | GGGAAGGGGAGAAAAAAAAA | 203 | mG*mG*mG*AAGGGGAGAAAAAAAAAGUUUUAG | 236 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009851 | AUGCAUUUGUUUCAAAAUAU | 35* | mA*mU*mG*CAUUUGUUUCAAAAUAUGUUUUAG | 67* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009852 | UGCAUUUGUUUCAAAAUAUU | 36* | mU*mG*mC*AUUUGUUUCAAAAUAUUGUUUUAG | 68* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009853 | UGAUUCCUACAGAAAAAGUC | 206 | mU*mG*mA*UUCCUACAGAAAAAGUCGUUUUAG | 239 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009854 | UACAGAAAAAGUCAGGAUAA | 207 | mU*mA*mC*AGAAAAAGUCAGGAUAAGUUUUAG | 240 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009855 | UUUCUUCUGCCUUUAAACAG | 208 | mU*mU*mU*CUUCUGCCUUUAAACAGGUUUUAG | 241 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009856 | UUAUAGUUUUAUAUUCAAAC | 209 | mU*mU*mA*UAGUUUUAUAUUCAAACGUUUUAG | 242 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009857 | AUUUAUGAGAUCAACAGCAC | 37* | mA*mU*mU*UAUGAGAUCAACAGCACGUUUUAG | 69* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009858 | GAUCAACAGCACAGGUUUUG | 38* | mG*mA*mU*CAACAGCACAGGUUUUGGUUUUAG | 70* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009859 | UUAAAUAAAGCAUAGUGCAA | 39* | mU*mU*mA*AAUAAAGCAUAGUGCAAGUUUUAG | 71* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009860 | UAAAGCAUAGUGCAAUGGAU | 40* | mU*mA*mA*AGCAUAGUGCAAUGGAUGUUUUAG | 72* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009861 | UAGUGCAAUGGAUAGGUCUU | 41* | mU*mA*mG*UGCAAUGGAUAGGUCUUGUUUUAG | 73* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009862 | AGUGCAAUGGAUAGGUCUUA | 215 | mA*mG*mU*GCAAUGGAUAGGUCUUAGUUUUAG | 248 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009863 | UUACUUUGCACUUUCCUUAG | 216 | mU*mU*mA*CUUUGCACUUUCCUUAGGUUUUAG | 249 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009864 | UACUUUGCACUUUCCUUAGU | 217 | mU*mA*mC*UUUGCACUUUCCUUAGUGUUUUAG | 250 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009865 | UCUGACCUUUUAUUUUACCU | 218 | mU*mC*mU*GACCUUUUAUUUUACCUGUUUUAG | 251 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009866 | UACUAAAACUUUAUUUUACU | 42* | mU*mA*mC*UAAAACUUUAUUUUACUGUUUUAG | 74* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009867 | AAAGUUGAACAAUAGAAAAA | 43* | mA*mA*mA*GUUGAACAAUAGAAAAAGUUUUAG | 75* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009868 | AAUGCAUAAUCUAAGUCAAA | 44* | mA*mA*mU*GCAUAAUCUAAGUCAAAGUUUUAG | 76* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009869 | AUUAUCCUGACUUUUUCUGU | 222 | mA*mU*mU*AUCCUGACUUUUUCUGUGUUUUAG | 255 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009870 | UGAAUUAUUCCUCUGUUUAA | 223 | mU*mG*mA*AUUAUUCCUCUGUUUAAGUUUUAG | 256 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009871 | UAAUUUUCUUUUGCCCACUA | 224 | mU*mA*mA*UUUUCUUUUGCCCACUAGUUUUAG | 257 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUm | |
| | CGUUAUCAACUUGAAAAAGU | | GmAmAmAmAmAmGmUmGmGmCmAmCmCmGm | |
| | GGCACCGAGUCGGUGCUUUU | | AmGmUmCmGmGmUmGmCmU*mU*mU*mU | |
| G009872 | AAAAGGUCAGAAUUGUUUAG | 225 | mA*mA*mA*AGGUCAGAAUUGUUUAGGUUUUAG | 258 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009873 | AACAUCCUAGGUAAAAUAAA | 226 | mA*mA*mC*AUCCUAGGUAAAAUAAAGUUUUAG | 259 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009874 | UAAUAAAAUUCAAACAUCCU | 45* | mU*mA*mA*UAAAAUUCAAACAUCCUGUUUUAG | 77* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009875 | UUGUCAUGUAUUUCUAAAAU | 228 | mU*mU*mG*UCAUGUAUUUCUAAAAUGUUUUAG | 261 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009876 | UUUGUCAUGUAUUUCUAAAA | 229 | mU*mU*mU*GUCAUGUAUUUCUAAAAGUUUUAG | 262 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |

SEQ ID NOs marked with an “*” above indicate that the indicated sgRNA is applicable to both cyno and human.

TABLE 14
Vector Components and Sequences

| | | 1^{st} orientation | | | 2^{nd} orientation | | | |
|---|---|---|---|---|---|---|---|---|
| Plasmid | 5′ | Splice | | | | | Splice | 3′ |
| ID | ITR | Acceptor | Transgene | Poly-A | Poly-A | Transgene | Acceptor | ITR |
| P00415 | (SEQ ID | Mouse Albumin | Nluc-P2A-GFP | SEQ ID | SEQ ID | Nluc-P2A-GFP | Mouse Albumin | (SEQ ID |
| | NO: 263) | Splice Acceptor | (SEQ ID | NO: 266 | NO: 267 | (SEQ ID | Splice Acceptor | NO: 270) |
| | | (SEQ ID | NO: 275) | | | NO: 276) | (SEQ ID | |
| | | NO: 264) | | | | | NO: 269) | |
| P00450 | (SEQ ID | Mouse Albumin | Human | SEQ ID | SEQ ID | Human | Mouse Albumin | (SEQ ID |
| | NO: 263) | Splice Acceptor | SERPINA1 | NO: 266 | NO: 267 | SERPINA1 | Splice Acceptor | NO: 270) |
| | | (SEQ ID | (SEQ ID | | | (SEQ ID | (SEQ ID | |
| | | NO: 264) | NO: 265) | | | NO: 268) | NO: 269) | |

5′ ITR Sequence (SEQ ID NO: 263):
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGC`GAGCGCGCAGAGA
GGGAGTGGCCAACTCCATCACTAGGGGTTCCT
Mouse Albumin Splice Acceptor (1^{st} orientation)(SEQ ID NO: 264):
TAGGTCAGTGAAGAGAAGAACAAAAAGCAGCATATTACAGTTAGTTGTCTTCATCA
ATCTTTAAATATGTTGTGTGGTTTTTCTCTCCCTGTTTCCACAG
Human SERPINA1, 1^{st} Orientation (SEQ ID NO: 265):
GAGGACCCCCAGGGCGACGCCGCCCAGAAGACCGACACCAGCCACCACGACCAGG
ACCACCCCACCTTCAACAAGATCACCCCCAACCTGGCCGAGTTCGCCTTCAGCCTGT
ACAGGCAGCTGGCCCACCAGAGCAACAGCACCAACATCTTCTTCAGCCCCGTGAGC
ATCGCCACCGCCTTCGCCATGCTGAGCCTGGGCACCAAGGCCGACACCCACGACGA
GATCCTGGAGGGCCTGAACTTCAACCTGACCGAGATCCCCGAGGCCCAGATCCACG
AGGGCTTCCAGGAGCTGCTGAGGACCCTGAACCAGCCCGACAGCCAGCTGCAGCTG
ACCACCGGCAACGGCCTGTTCCTGAGCGAGGGCCTGAAGCTGGTGGACAAGTTCCT
GGAGGACGTGAAGAAGCTGTACCACAGCGAGGCCTTCACCGTGAACTTCGGCGACA
CCGAGGAGGCCAAGAAGCAGATCAACGACTACGTGGAGAAGGGCACCCAGGGCAA
GATCGTGGACCTGGTGAAGGAGCTGGACAGGGACACCGTGTTCGCCCTGGTGAACT
ACATCTTCTTCAAGGGCAAGTGGGAGAGGCCCTTCGAGGTGAAGGACACCGAGGAG
GAGGACTTCCACGTGGACCAGGTGACCACCGTGAAGGTGCCCATGATGAAGAGGCT
GGGCATGTTCAACATCCAGCACTGCAAGAAGCTGAGCAGCTGGGTGCTGCTGATGA
AGTACCTGGGCAACGCCACCGCCATCTTCTTCCTGCCCGACGAGGGCAAGCTGCAGC
ACCTGGAGAACGAGCTGACCCACGACATCATCACCAAGTTCCTGGAGAACGAGGAC
AGGAGGAGCGCCAGCCTGCACCTGCCCAAGCTGAGCATCACCGGCACCTACGACCT
GAAGAGCGTGCTGGGCCAGCTGGGCATCACCAAGGTGTTCAGCAACGGCGCCGACC
TGAGCGGCGTGACCGAGGAGGCCCCCCTGAAGCTGAGCAAGGCCGTGCACAAGGCC
GTGCTGACCATCGACGAGAAGGGCACCGAGGCCGCCGGCGCCATGTTCCTGGAGGC
CATCCCCATGAGCATCCCCCCCGAGGTGAAGTTCAACAAGCCCTTCGTGTTCCTGAT
GATCGAGCAGAACACCAAGAGCCCCCTGTTCATGGGCAAGGTGGTGAACCCCACCC
AGAAGTAA
bGH Poly-A (1^{st} orientation)(SEQ ID NO: 266):
CCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAG
CAAGGGGGAGGATTGGGAAGACAATAGCAGGCATGCTGGGGATGCGGTGGGCTCTA
TGGCTTCTGAGGCGGAAAGAACCAGCTGGGGCTCTAGGGGGTATCCCC
SV40 Poly-A (2^{nd} orientation)(SEQ ID NO: 267):
AAAAAACCTCCCACACCTCCCCCTGAACCTGAAACATAAAATGAATGCAATTGTTGT
TGTTAACTTGTTTATTGCAGCTTATAATGGTTACAAATAAAGCAATAGCATCACAAA
TTTCACAAATAAAGCATTTTTTTCACTGCATTCTAGTTGTGGTTTGTCCAAACTCATC
AATGTATCTTATCATGTCTG
Human SERPINA1, 2^{nd} Orientation (SEQ ID NO: 268):
GAGGATCCCCAGGGAGATGCTGCCCAGAAGACAGATACATCCCACCATGATCAGGA
TCACCCAACCTTCAACAAGATCACCCCCAACCTGGCTGAGTTCGCCTTCAGCCTATA
CCGCCAGCTGGCACACCAGTCCAACAGCACCAATATCTTCTTCTCCCCAGTGAGCAT
CGCTACAGCCTTTGCAATGCTCTCCCTGGGGACCAAGGCTGACACTCACGATGAAAT
CCTGGAGGGCCTGAATTTCAACCTCACGGAGATTCCGGAGGCTCAGATCCATGAAG
GCTTCCAGGAACTCCTCCGTACCCTCAACCAGCCAGACAGCCAGCTCCAGCTGACCA
CCGGCAATGGCCTGTTCCTCAGCGAGGGCCTGAAGCTAGTGGATAAGTTTTTGGAGG
ATGTTAAAAAGTTGTACCACTCAGAAGCCTTCACTGTCAACTTCGGGGACACCGAAG
AGGCCAAGAAACAGATCAACGATTACGTGGAGAAGGGTACTCAAGGGAAAATTGTG
GATTTGGTCAAGGAGCTTGACAGAGACACAGTTTTTGCTCTGGTGAATTACATCTTC
TTTAAAGGCAAATGGGAGAGACCCTTTGAAGTCAAGGACACCGAGGAAGAGGACTT
CCACGTGGACCAGGTGACCACCGTGAAGGTGCCTATGATGAAGCGTTTAGGCATGTT
TAACATCCAGCACTGTAAGAAGCTGTCCAGCTGGGTGCTGCTGATGAAATACCTGGG
CAATGCCACCGCCATCTTCTTCCTGCCTGATGAGGGGAAACTACAGCACCTGGAAAA
TGAACTCACCCACGATATCATCACCAAGTTCCTGGAAAATGAAGACAGAAGGTCTG
CCAGCTTACATTTACCCAAACTGTCCATTACTGGAACCTATGATCTGAAGAGCGTCC
TGGGTCAACTGGGCATCACTAAGGTCTTCAGCAATGGGGCTGACCTCTCCGGGGTCA
CAGAGGAGGCACCCCTGAAGCTCTCCAAGGCCGTGCATAAGGCTGTGCTGACCATC
GACGAGAAAGGGACTGAAGCTGCTGGGGCCATGTTTTTAGAGGCCATACCCATGTC
TATCCCCCCCGAGGTCAAGTTCAACAAACCCTTTGTCTTCTTAATGATTGAACAAAA
TACCAAGTCTCCCCTCTTCATGGGAAAAGTGGTGAATCCCACCCAAAAAtaa
Mouse Albumin Splice Acceptor (2^{nd} orientation)(SEQ ID NO: 269):
CTGTGGAAACAGGGAGAGAAAAACCACACAACATATTTAAAGATTGATGAAGACAA
CTAACTGTAATATGCTGCTTTTTGTTCTTCTCTTCACTGACCTA
3′ ITR Sequence (SEQ ID NO: 270):
AGGAACCCCTAGTGATGGAGTTGGCCACTCCCTCTCTGCGCGCTCGCTCGCTCACTG
AGGCCGCCCGGGCAAAGCCCGGGCGTCGGGCGACCTTTGGTCGCCCGGCCTCAGTG
AGCGAGCGAGCGCGCAGAGAGGGAGTGGCCAA
Nluc-P2A-GFP (1^{st} Orientation)(SEQ ID NO: 275):
TTTCTTGATCATGAAAACGCCAACAAAATTCTGAATCGGCCAAAGAGGTATAATTCA
GGTAAATTGGAAGAGTTTGTTCAAGGGAACCTTGAGAGAGAATGTATGGAAGAAAA
GTGTAGTTTTGAAGAAGCAGTATTCACTTTGGAGGACTTTGTCGGTGACTGGAGGCA
AACCGCTGGTTATAATCTCGACCAAGTACTGGAACAGGGCGGGGTAAGTTCCCTCTT
TCAGAATTTGGGTGTAAGCGTCACACCAATCCAGCGGATTGTGTTGTCTGGAGAGAA
CGGACTCAAAATTGACATCCATGTTATCATTCCATATGAAGGTCTCAGTGGAGACCA
AATGGGGCAGATCGAGAAGATTTTCAAGGTAGTTTACCCAGTCGACGATCACCACTT
CAAAGTCATTCTCCACTATGGCACACTTGTTATCGACGGAGTAACTCCTAATATGAT
TGATTACTTTGGTCGCCCGTATGAGGGCATCGCAGTGTTTGATGGCAAAAAGATCAC
CGTAACAGGAACGTTGTGGAATGGGAACAAGATAATCGACGAGAGATTGATAAATC
CAGACGGGTCACTCCTGTTCAGGGTTACAATTAACGGCGTCACAGGATGGAGACTCT
GTGAACGAATACTGGCCACAAATTTTTCACTCCTGAAGCAGGCCGGAGACGTGGAG
GAAAACCCAGGGCCCGTGAGCAAGGGCGAGGAGCTGTTCACCGGGGTGGTGCCCAT
CCTGGTCGAGCTGGACGGCGACGTAAACGGCCACAAGTTCAGCGTGTCCGGCGAGG
GCGAGGGCGATGCCACCTACGGCAAGCTGACCCTGAAGTTCATCTGCACCACCGGC
AAGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCCC
GAAGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAAGG
GCATCGACTTCAAGGAGGACGGCAACATCCTGGGGCACAAGCTGGAGTACAACTAC
AACAGCCACAACGTCTATATCATGGCCGACAAGCAGAAGAACGGCATCAAGGTGAA
CTTCAAGATCCGCCACAACATCGAGGACGGCAGCGTGCAGCTCGCCGACCACTACC
AGCAGAACACCCCCATCGGCGACGGCCCCGTGCTGCTGCCCGACAACCACTACCTG
AGCACCCAGTCCGCCCTGAGCAAAGACCCCAACGAGAAGCGCGATCACATGGTCCT
GCTGGAGTTCGTGACCGCCGCCGGGATCACTCTCGGCATGGACGAGCTGTACAAGG
GAGGAGGAAGCCCGAAGAAGAAGAGAAAGGTCTAA
Nluc-P2A-GFP (2^{nd} Orientation)(SEQ ID NO: 276):
TTACACCTTCCTCTTCTTCTTGGGGCTGCCGCCGCCCTTGTACAGCTCGTCCATGCCC
AGGGTGATGCCGGCGGCGGTCACGAACTCCAGCAGCACCATGTGGTCCCTCTTCTCG
TTGGGGTCCTTGCTCAGGGCGCTCTGGGTGCTCAGGTAGTGGTTGTCGGGCAGCAGC
ACGGGGCCGTCGCCGATGGGGGTGTTCTGCTGGTAGTGGTCGGCCAGCTGCACGCTG
CCGTCCTCGATGTTGTGCCTGATCTTGAAGTTCACCTTGATGCCGTTCTTCTGCTTGT
CGGCCATGATGTACACGTTGTGGCTGTTGTAGTTGTACTCCAGCTTGTGGCCCAGGA
TGTTGCCGTCCTCCTTGAAGTCGATGCCCTTCAGCTCGATCCTGTTCACCAGGGTGTC
GCCCTCGAACTTCACCTCGGCCCTGGTCTTGTAGTTGCCGTCGTCCTTGAAGAAGAT
GGTCCTCTCCTGCACGTAGCCCTCGGGCATGGCGCTCTTGAAGAAGTCGTGCTGCTT
CATGTGGTCGGGGTACCTGCTGAAGCACTGCACGCCGTAGGTCAGGGTGGTCACCA
GGGTGGGCCAGGGCACGGGCAGCTTGCCGGTGGTGCAGATGAACTTCAGGGTCAGC
TTGCCGTAGGTGGCGTCGCCCTCGCCCTCGCCGCTCACGCTGAACTTGTGGCCGTTC
ACGTCGCCGTCCAGCTCCACCAGGATGGGCACCACGCCGGTGAACAGCTCCTCGCC
CTTGCTCACGGGGCCGGGGTTCTCCTCCACGTCGCCGGCCTGCTTCAGCAGGCTGAA
GTTGGTGGCCAGGATCCTCTCGCACAGCCTCCAGCCGGTCACGCCGTTGATGGTCAC
CCTGAACAGCAGGCTGCCGTCGGGGTTGATCAGCCTCTCGTCGATGATCTTGTTGCC
GTTCCACAGGGTGCCGGTCACGGTGATCTTCTTGCCGTCGAACACGGCGATGCCCTC
GTAGGGCCTGCCGAAGTAGTCGATCATGTTGGGGGTCACGCCGTCGATCACCAGGG
TGCCGTAGTGCAGGATCACCTTGAAGTGGTGGTCGTCCACGGGGTACACCACCTTGA
AAATCTTCTCGATCTGGCCCATCTGGTCGCCGCTCAGGCCCTCGTAGGGGATGATCA
CGTGGATGTCGATCTTCAGGCCGTTCTCGCCGCTCAGCACGATCCTCTGGATGGGGG
TCACGCTCACGCCCAGGTTCTGGAACAGGCTGCTCACGCCGCCCTGCTCCAGCACCT
GGTCCAGGTTGTAGCCGGCGGTCTGCCTCCAGTCGCCCACGAAGTCCTCCAGGGTGA
ACACGGCCTCCTCGAAGCTGCACTTCTCCTCCATGCACTCCCTCTCCAGGTTGCCCTG
CACGAACTCCTCCAGCTTGCCGCTGTTGTACCTCTTGGGCCTGTTCAGGATCTTGTTG
GCGTTCTCGTGGTCCAGGAA
P00415 full sequence (from ITR to ITR): (SEQ ID NO: 279)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTCTTAGGTCAGTGAAGAGA
AGAACAAAAAGCAGCATATTACAGTTAGTTGTCTTCATCAATCTTTAAATATGTTGT
GTGGTTTTTCTCTCCCTGTTTCCACAGTTTTTCTTGATCATGAAAACGCCAACAAAAT
TCTGAATCGGCCAAAGAGGTATAATTCAGGTAAATTGGAAGAGTTTGTTCAAGGGA
ACCTTGAGAGAGAATGTATGGAAGAAAAGTGTAGTTTTGAAGAAGCAGTATTCACT
TTGGAGGACTTTGTCGGTGACTGGAGGCAAACCGCTGGTTATAATCTCGACCAAGTA
CTGGAACAGGGCGGGGTAAGTTCCCTCTTTCAGAATTTGGGTGTAAGCGTCACACCA
ATCCAGCGGATTGTGTTGTCTGGAGAGAACGGACTCAAAATTGACATCCATGTTATC
ATTCCATATGAAGGTCTCAGTGGAGACCAAATGGGGCAGATCGAGAAGATTTTCAA
GGTAGTTTACCCAGTCGACGATCACCACTTCAAAGTCATTCTCCACTATGGCACACT
TGTTATCGACGGAGTAACTCCTAATATGATTGATTACTTTGGTCGCCCGTATGAGGG
CATCGCAGTGTTTGATGGCAAAAAGATCACCGTAACAGGAACGTTGTGGAATGGGA
ACAAGATAATCGACGAGAGATTGATAAATCCAGACGGGTCACTCCTGTTCAGGGTT
ACAATTAACGGCGTCACAGGATGGAGACTCTGTGAACGAATACTGGCCACAAATTT
TTCACTCCTGAAGCAGGCCGGAGACGTGGAGGAAAACCCAGGGCCCGTGAGCAAGG
GCGAGGAGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCT
CGTGACCACCCTGACCTACGGCGTGCAGTGCTTCAGCCGCTACCCCGACCACATGAA
GCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAA
CATCCTGGGGCACAAGCTGGAGTACAACTACAACAGCCACAACGTCTATATCATGG
CCGACAAGCAGAAGAACGGCATCAAGGTGAACTTCAAGATCCGCCACAACATCGAG
GACGGCAGCGTGCAGCTCGCCGACCACTACCAGCAGAACACCCCCATCGGCGACGG
CCCCGTGCTGCTGCCCGACAACCACTACCTGAGCACCCAGTCCGCCCTGAGCAAAG
ACCCCAACGAGAAGCGCGATCACATGGTCCTGCTGGAGTTCGTGACCGCCGCCGGG
ATCACTCTCGGCATGGACGAGCTGTACAAGGGAGGAGGAAGCCCGAAGAAGAAGA
GAAAGGTCTAACCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGG
GGCAGGACAGCAAGGGGGAGGATTGGGAAGACAATAGCAGGCATGCTGGGGATGC
GGTGGGCTCTATGGCTTCTGAGGCGGAAAGAACCAGCTGGGGCTCTAGGGGGTATC
CCCAAAAAACCTCCCACACCTCCCCCTGAACCTGAAACATAAAATGAATGCAATTGT
TGTTGTTAACTTGTTTATTGCAGCTTATAATGGTTACAAATAAAGCAATAGCATCAC
AAATTTCACAAATAAAGCATTTTTTTCACTGCATTCTAGTTGTGGTTTGTCCAAACTC
ATCAATGTATCTTATCATGTCTGTTACACCTTCCTCTTCTTCTTGGGGCTGCCGCCGC
CCTTGTACAGCTCGTCCATGCCCAGGGTGATGCCGGCGGCGGTCACGAACTCCAGCA
GCACCATGTGGTCCCTCTTCTCGTTGGGGTCCTTGCTCAGGGCGCTCTGGGTGCTCAG
GTAGTGGTTGTCGGGCAGCAGCACGGGGCCGTCGCCGATGGGGGTGTTCTGCTGGT
AGTGGTCGGCCAGCTGCACGCTGCCGTCCTCGATGTTGTGCCTGATCTTGAAGTTCA
CCTTGATGCCGTTCTTCTGCTTGTCGGCCATGATGTACACGTTGTGGCTGTTGTAGTT
GTACTCCAGCTTGTGGCCCAGGATGTTGCCGTCCTCCTTGAAGTCGATGCCCTTCAG
CTCGATCCTGTTCACCAGGGTGTCGCCCTCGAACTTCACCTCGGCCCTGGTCTTGTAG
TTGCCGTCGTCCTTGAAGAAGATGGTCCTCTCCTGCACGTAGCCCTCGGGCATGGCG
CTCTTGAAGAAGTCGTGCTGCTTCATGTGGTCGGGGTACCTGCTGAAGCACTGCACG
CCGTAGGTCAGGGTGGTCACCAGGGTGGGCCAGGGCACGGGCAGCTTGCCGGTGGT
GCAGATGAACTTCAGGGTCAGCTTGCCGTAGGTGGCGTCGCCCTCGCCCTCGCCGCT
CACGCTGAACTTGTGGCCGTTCACGTCGCCGTCCAGCTCCACCAGGATGGGCACCAC
GCCGGTGAACAGCTCCTCGCCCTTGCTCACGGGGCCGGGGTTCTCCTCCACGTCGCC
GGCCTGCTTCAGCAGGCTGAAGTTGGTGGCCAGGATCCTCTCGCACAGCCTCCAGCC
GGTCACGCCGTTGATGGTCACCCTGAACAGCAGGCTGCCGTCGGGGTTGATCAGCCT
CTCGTCGATGATCTTGTTGCCGTTCCACAGGGTGCCGGTCACGGTGATCTTCTTGCCG
TCGAACACGGCGATGCCCTCGTAGGGCCTGCCGAAGTAGTCGATCATGTTGGGGGTC
ACGCCGTCGATCACCAGGGTGCCGTAGTGCAGGATCACCTTGAAGTGGTGGTCGTCC
ACGGGGTACACCACCTTGAAAATCTTCTCGATCTGGCCCATCTGGTCGCCGCTCAGG
CCCTCGTAGGGGATGATCACGTGGATGTCGATCTTCAGGCCGTTCTCGCCGCTCAGC
ACGATCCTCTGGATGGGGGTCACGCTCACGCCCAGGTTCTGGAACAGGCTGCTCACG
CCGCCCTGCTCCAGCACCTGGTCCAGGTTGTAGCCGGCGGTCTGCCTCCAGTCGCCC
ACGAAGTCCTCCAGGGTGAACACGGCCTCCTCGAAGCTGCACTTCTCCTCCATGCAC
TCCCTCTCCAGGTTGCCCTGCACGAACTCCTCCAGCTTGCCGCTGTTGTACCTCTTGG
GCCTGTTCAGGATCTTGTTGGCGTTCTCGTGGTCCAGGAA
P00450 SEQ ID NO: 289
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTACTAGTtaggtcagtgaagagaagaa
caaaaagcagcatattacagttagttgtatcatcaatctttaaatatgttgtgtggtttttctctccctgtttccacagttGAGGACCCCC
AGGGCGACGCCGCCCAGAAGACCGACACCAGCCACCACGACCAGGACCACCCCACC
TTCAACAAGATCACCCCCAACCTGGCCGAGTTCGCCTTCAGCCTGTACAGGCAGCTG
GCCCACCAGAGCAACAGCACCAACATCTTCTTCAGCCCCGTGAGCATCGCCACCGCC
TTCGCCATGCTGAGCCTGGGCACCAAGGCCGACACCCACGACGAGATCCTGGAGGG
CCTGAACTTCAACCTGACCGAGATCCCCGAGGCCCAGATCCACGAGGGCTTCCAGG
AGCTGCTGAGGACCCTGAACCAGCCCGACAGCCAGCTGCAGCTGACCACCGGCAAC
GGCCTGTTCCTGAGCGAGGGCCTGAAGCTGGTGGACAAGTTCCTGGAGGACGTGAA
GAAGCTGTACCACAGCGAGGCCTTCACCGTGAACTTCGGCGACACCGAGGAGGCCA
AGAAGCAGATCAACGACTACGTGGAGAAGGGCACCCAGGGCAAGATCGTGGACCT
GGTGAAGGAGCTGGACAGGGACACCGTGTTCGCCCTGGTGAACTACATCTTCTTCAA
GGGCAAGTGGGAGAGGCCCTTCGAGGTGAAGGACACCGAGGAGGAGGACTTCCAC
GTGGACCAGGTGACCACCGTGAAGGTGCCCATGATGAAGAGGCTGGGCATGTTCAA
CATCCAGCACTGCAAGAAGCTGAGCAGCTGGGTGCTGCTGATGAAGTACCTGGGCA
ACGCCACCGCCATCTTCTTCCTGCCCGACGAGGGCAAGCTGCAGCACCTGGAGAAC
GAGCTGACCCACGACATCATCACCAAGTTCCTGGAGAACGAGGACAGGAGGAGCGC
CAGCCTGCACCTGCCCAAGCTGAGCATCACCGGCACCTACGACCTGAAGAGCGTGC
TGGGCCAGCTGGGCATCACCAAGGTGTTCAGCAACGGCGCCGACCTGAGCGGCGTG
ACCGAGGAGGCCCCCCTGAAGCTGAGCAAGGCCGTGCACAAGGCCGTGCTGACCAT
CGACGAGAAGGGCACCGAGGCCGCCGGCGCCATGTTCCTGGAGGCCATCCCCATGA
GCATCCCCCCCGAGGTGAAGTTCAACAAGCCCTTCGTGTTCCTGATGATCGAGCAGA
ACACCAAGAGCCCCCTGTTCATGGGCAAGGTGGTGAACCCCACCCAGAAGTAACAG
ACATGATAAGATACATTGATGAGTTTGGACAAACCACAACTAGAATGCAGTGAAAA
AAATGCTTTATTTGTGAAATTTGTGATGCTATTGCTTTATTTGTAACCATTATAAGCT
GCAATAAACAAGTTAACAACAACAATTGCATTCATTTTATGTTTCAGGTTCAGGGGG
AGGTGTGGGAGGTTTTTTggggataccccctagagccccagctggttctttccgcctcagaagccatagagcccaccgca
tccccagcatgcctgctattgtcttcccaatcctcccccttgctgtcctgccccaccccaccccccagaatagaatgacacctactcagacaat
gcgatgcaatttcctcattttattaggaaaggacagtgggagtggcaccttccagggtcaaggaaggcacgggggaggggcaaacaaca
gatggctggcaactagaaggcacagtcgaggttaTTTTTGGGTGGGATTCACCACTTTTCCCATGAAGAG
GGGAGACTTGGTATTTTGTTCAATCATTAAGAAGACAAAGGGTTTGTTGAACTTGAC
CTCGGGGGGGATAGACATGGGTATGGCCTCTAAAAACATGGCCCCAGCAGCTTCAG
TCCCTTTCTCGTCGATGGTCAGCACAGCCTTATGCACGGCCTTGGAGAGCTTCAGGG
GTGCCTCCTCTGTGACCCCGGAGAGGTCAGCCCCATTGCTGAAGACCTTAGTGATGC
CCAGTTGACCCAGGACGCTCTTCAGATCATAGGTTCCAGTAATGGACAGTTTGGGTA
AATGTAAGCTGGCAGACCTTCTGTCTTCATTTTCCAGGAACTTGGTGATGATATCGT
GGGTGAGTTCATTTTCCAGGTGCTGTAGTTTCCCCTCATCAGGCAGGAAGAAGATGG
CGGTGGCATTGCCCAGGTATTTCATCAGCAGCACCCAGCTGGACAGCTTCTTACAGT
GCTGGATGTTAAACATGCCTAAACGCTTCATCATAGGCACCTTCACGGTGGTCACCT
GGTCCACGTGGAAGTCCTCTTCCTCGGTGTCCTTGACTTCAAAGGGTCTCTCCCATTT
GCCTTTAAAGAAGATGTAATTCACCAGAGCAAAAACTGTGTCTCTGTCAAGCTCCTT
GACCAAATCCACAATTTTCCCTTGAGTACCCTTCTCCACGTAATCGTTGATCTGTTTC
TTGGCCTCTTCGGTGTCCCCGAAGTTGACAGTGAAGGCTTCTGAGTGGTACAACTTT
TTAACATCCTCCAAAAACTTATCCACTAGCTTCAGGCCCTCGCTGAGGAACAGGCCA
TTGCCGGTGGTCAGCTGGAGCTGGCTGTCTGGCTGGTTGAGGGTACGGAGGAGTTCC
TGGAAGCCTTCATGGATCTGAGCCTCCGGAATCTCCGTGAGGTTGAAATTCAGGCCC
TCCAGGATTTCATCGTGAGTGTCAGCCTTGGTCCCCAGGGAGAGCATTGCAAAGGCT
GTAGCGATGCTCACTGGGGAGAAGAAGATATTGGTGCTGTTGGACTGGTGTGCCAG
CTGGCGGTATAGGCTGAAGGCGAACTCAGCCAGGTTGGGGGTGATCTTGTTGAAGG
TTGGGTGATCCTGATCATGGTGGGATGTATCTGTCTTCTGGGCAGCATCTCCCTGGG
GATCCTCaactgtggaaacagggagagaaaaaccacacaacatatttaaagattgatgaagacaactaactgtaatatgctgctttttgt
tcttctcttcactgacctaACTAGTAGATCTAGGAACCCCTAGTGATGGAGTTGGCCACTCCCTC
TCTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
Albumin Signal Peptide Sequence SEQ ID NO: 2000
MKWVTFISLLFLFSSAYS

1. (canceled)

2. A method of expressing AAT in a subject in need thereof, comprising administering:
i) a nucleic acid construct comprising a heterologous AAT protein coding sequence;
ii) a RNA-guided DNA binding agent; and
iii) an albumin guide RNA (gRNA) comprising a sequence chosen from:
a) a sequence that is at least 95%, 90%, 85%, 80%, or 75% identical to a sequence selected from the group consisting of SEQ ID Nos: 2, 8, 13, 19, 28, 29, 31, 32, and 33;
b) a sequence that is at least 17, 18, 19, or 20 contiguous nucleotides of a sequence selected from the group consisting of SEQ ID NOs: 2, 8, 13, 19, 28, 29, 31, 32, and 33;
c) a sequence selected from the group consisting of SEQ ID NOs: 34, 40, 45, 51, 60, 61, 63, 64, 65, 66, 72, 77, 83, 92, 93, 95, 96, and 97;
d) a sequence that is at least 95%, 90%, 85%, 80%, or 75% identical to a sequence selected from the group consisting of SEQ ID NOs: 2-33;
e) at least 17, 18, 19, or 20 contiguous nucleotides of a sequence selected from the group consisting of SEQ ID NOs: 2-33;
f) a sequence selected from the group consisting of SEQ ID NOs: 34-97; and
g) a sequence that is complementary to 15 consecutive nucleotides +/−10 nucleotides of the genomic coordinates listed for SEQ ID NOs: 2-33,
thereby expressing AAT in a subject in need thereof.

3. A method of treating alpha-1 antitrypsin deficiency (AATD) in a subject in need of AAT protein, comprising administering:
i) a nucleic acid construct comprising a heterologous AAT protein coding sequence;
ii) a RNA-guided DNA binding agent; and
iii) an albumin guide RNA (gRNA) comprising a sequence chosen from:
a) a sequence that is at least 95%, 90%, 85%, 80%, or 75% identical to a sequence selected from the group consisting of SEQ ID Nos: 2, 8, 13, 19, 28, 29, 31, 32, and 33;
b) a sequence that is at least 17, 18, 19, or 20 contiguous nucleotides of a sequence selected from the group consisting of SEQ ID NOs: 2, 8, 13, 19, 28, 29, 31, 32, and 33;
c) a sequence selected from the group consisting of SEQ ID NOs: 34, 40, 45, 51, 60, 61, 63, 64, 65, 66, 72, 77, 83, 92, 93, 95, 96, and 97;
d) a sequence that is at least 95%, 90%, 85%, 80%, or 75% identical to a sequence selected from the group consisting of SEQ ID NOs: 2-33;
e) at least 17, 18, 19, or 20 contiguous nucleotides of a sequence selected from the group consisting of SEQ ID NOs: 2-33;
f) a sequence selected from the group consisting of SEQ ID NOs: 34-97; and
g) a sequence that is complementary to 15 consecutive nucleotides +/−10 nucleotides of the genomic coordinates listed for SEQ ID NOs: 2-33,
thereby treating AATD in the subject.

4. (canceled)

5. The method of claim 2, wherein the method further comprises inducing a double-stranded break (DSB) within the endogenous SERPINA1 gene.

6. The method of claim 2, wherein the method further comprises modifying the endogenous SERPINA1 gene.

7. The method of claim 5, wherein the DSB is induced within the endogenous SERPINA1 gene before or after administering the nucleic acid construct comprising a heterologous AAT protein coding sequence, the RNA-guided DNA binding agent, and the albumin gRNA.

8. The method of claim 2, wherein the method further comprises administering a SERPINA1 guide RNA that is at least partially complementary to a target sequence present in exon 2, 3, 4, or 5 of the endogenous human SERPINA1 gene.

9. The method of claim 8, wherein the SERPINA1 guide RNA comprises a guide sequence selected from SEQ ID NOs: 1000-1128 or a guide sequence that is at least 95%, 90%, 85%, 80%, or 75% identical to or is at least 17, 18, 19, and 20 consecutive nucleotides of a sequence chosen from SEQ ID NOs: 1000-1128.

10. The method of claim 8, wherein the method further comprises administering a RNA-guided DNA binding agent with the SERPINA1 guide RNA.

11. The method of claim 8, wherein non-homologous ending joining (NHEJ) leads to a change in the DNA selected from:
a) a mutation during repair of a DSB in the endogenous SERPINA1 gene; and
b) a deletion or insertion of a nucleotide(s) during repair of a DSB in the endogenous SERPINA1 gene.

12. (canceled)

13. The method of claim 11, wherein the deletion or insertion of a nucleotide(s) induces a frame shift or nonsense mutation in the endogenous SERPINA1 gene.

14. (canceled)

15. The method of claim 2, wherein the administration is in vivo.

16. (canceled)

17. The method of claim 2, wherein the nucleic acid construct is administered in a nucleic acid vector or a lipid nanoparticle.

18. The method of claim 2, wherein the RNA-guided DNA binding agent or albumin gRNA is administered in a nucleic acid vector and/or lipid nanoparticle.

19. The method of claim 8, wherein the RNA-guided DNA binding agent or SERPINA1 gRNA is administered in a nucleic acid vector or lipid nanoparticle.

20. The method of claim 17, wherein the nucleic acid vector is a viral vector.

21. The method of claim 20, wherein the viral vector is selected from the group consisting of an adeno associate viral (AAV) vector, adenovirus vector, retrovirus vector, and lentivirus vector.

22-26. (canceled)

27. The method of claim 2, wherein the RNA-guided DNA binding agent is a class 2 Cas nuclease.

28. The method of claim 27, wherein the Cas nuclease is a Cas9 nuclease.

29-31. (canceled)

32. The method of claim 2, wherein the nucleic acid construct is a bidirectional nucleic acid construct.

33-34. (canceled)

35. The method of claim 32, wherein the bidirectional nucleic acid construct does not comprise a promoter.

36. The method of claim 2, wherein the subject's level of functional AAT is increased to at least about 500 μg/ml.

37. The method of claim 2, wherein the subject's level of functional AAT is increased by at least about 10%, 20%, 30%, 40%, 50%, 60%, 70%, 80%, 90%, 100%, or more, as compared to the subject's level of functional AAT before administration.

38. The method of claim or 37, wherein the level of AAT is measured in serum, plasma, blood, cerebral spinal fluid, and/or sputum.

39-44. (canceled)

45. The method of claim 2, wherein the nucleic acid construct comprises a sequence that encodes a wild-type AAT protein, or a functional fragment thereof.

46-49. (canceled)

50. The method of claim 32, wherein the bidirectional nucleic acid construct comprises:
a) a first segment comprising a coding sequence for a heterologous AAT; and
b) a second segment comprising a reverse complement of a coding sequence of the heterologous AAT,
wherein the construct does not comprise a promoter that drives the expression of the heterologous AAT.

51-88. (canceled)

89. A method of treating alpha-1 antitrypsin deficiency (AATD) in a subject in need of AAT protein, comprising administering:
i) a gene editing system capable of reducing the endogenous expression of SERPINA1;
ii) a nucleic acid construct comprising a heterologous AAT protein coding sequence;
iii) a RNA-guided DNA binding agent; and
iv) an albumin guide RNA (gRNA) comprising a sequence chosen from:
a) a sequence that is at least 95%, 90%, 85%, 80%, or 75% identical to a sequence selected from the group consisting of SEQ ID Nos: 2, 8, 13, 19, 28, 29, 31, 32, and 33;
b) a sequence that is at least 17, 18, 19, or 20 contiguous nucleotides of a sequence selected from the group consisting of SEQ ID NOs: 2, 8, 13, 19, 28, 29, 31, 32, and 33;
c) a sequence selected from the group consisting of SEQ ID NOs: 34, 40, 45, 51, 60, 61, 63, 64, 65, 66, 72, 77, 83, 92, 93, 95, 96, and 97;
d) a sequence that is at least 95%, 90%, 85%, 80%, or 75% identical to a sequence selected from the group consisting of SEQ ID NOs: 2-33;
e) at least 17, 18, 19, or 20 contiguous nucleotides of a sequence selected from the group consisting of SEQ ID NOs: 2-33;
f) a sequence selected from the group consisting of SEQ ID NOs: 34-97; and
g) a sequence that is complementary to 15 consecutive nucleotides +/−10 nucleotides of the genomic coordinates listed for SEQ ID NOs: 2-33,
thereby treating AATD in the subject.

90. The method of claim 89, wherein the gene editing system comprises a SERPINA1 guide RNA comprising a guide sequence selected from SEQ ID NOs: 1000-1128 or a guide sequence that is at least 95%, 90%, 85%, 80%, or 75% identical to a sequence selected from SEQ ID NOs: 1000-1128.

91. A bidirectional nucleic acid construct comprising:
a) a first segment comprising a coding sequence for a AAT polypeptide; and
b) a second segment comprising a reverse complement of a coding sequence of the AAT polypeptide,
wherein the construct does not comprise a promoter that drives the expression of the AAT polypeptide.

92-122. (canceled)

123. The method of claim 2, wherein the gRNA comprises SEQ ID NO: 901.

124. The method of claim 3, wherein the treatment of AATD comprises alleviating liver and lung symptoms.

125. The method of claim 9, wherein the SERPINA1 guide RNA comprises a guide sequence selected from SEQ ID NOs: 1023, 1028, 1037, 1041, 1050, 1064, and 1106, or a guide sequence that is at least 95%, 90%, 85%, 80%, or 75% identical to or is at least 17, 18, 19, and 20 consecutive nucleotides of a sequence chosen from SEQ ID NOs: 1023, 1028, 1037, 1041, 1050, 1064, and 1106.


SEQUENCE LISTING (2002 sequences)

SEQ ID NO: 1 - DNA, 709 bp, Homo sapiens
gtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccag

SEQ ID NO: 2 - RNA, 20 nt, Artificial Sequence
  Description of Artificial Sequence: Synthetic oligonucleotide
gagcaaccucacucuugucu

SEQ ID NO: 3 - RNA, 20 nt, Artificial Sequence
  Description of Artificial Sequence: Synthetic oligonucleotide
augcauuuguuucaaaauau

SEQ ID NO: 4 - RNA, 20 nt, Artificial Sequence
  Description of Artificial Sequence: Synthetic oligonucleotide
ugcauuuguuucaaaauauu

SEQ ID NO: 5 - RNA, 20 nt, Artificial Sequence
  Description of Artificial Sequence: Synthetic oligonucleotide
auuuaugagaucaacagcac

SEQ ID NO: 6 - RNA, 20 nt, Artificial Sequence
  Description of Artificial Sequence: Synthetic oligonucleotide
gaucaacagcacagguuuug

SEQ ID NO: 7 - RNA, 20 nt, Artificial Sequence
  Description of Artificial Sequence: Synthetic oligonucleotide
uuaaauaaagcauagugcaa

SEQ ID NO: 8 - RNA, 20 nt, Artificial Sequence
  Description of Artificial Sequence: Synthetic oligonucleotide
uaaagcauagugcaauggau

SEQ ID NO: 9 - RNA, 20 nt, Artificial Sequence
  Description of Artificial Sequence: Synthetic oligonucleotide
uagugcaauggauaggucuu

SEQ ID NO: 10 - RNA, 20 nt, Artificial Sequence
  Description of Artificial Sequence: Synthetic oligonucleotide
uacuaaaacuuuauuuuacu

SEQ ID NO: 11 - RNA, 20 nt, Artificial Sequence
  Description of Artificial Sequence: Synthetic oligonucleotide
aaaguugaacaauagaaaaa

SEQ ID NO: 12 - RNA, 20 nt, Artificial Sequence
  Description of Artificial Sequence: Synthetic oligonucleotide
aaugcauaaucuaagucaaa

SEQ ID NO: 13 - RNA, 20 nt, Artificial Sequence
  Description of Artificial Sequence: Synthetic oligonucleotide
uaauaaaauucaaacauccu

SEQ ID NO: 14 - RNA, 20 nt, Artificial Sequence
  Description of Artificial Sequence: Synthetic oligonucleotide
gcaucuuuaaagaauuauuu

SEQ ID NO: 15 - RNA, 20 nt, Artificial Sequence
  Description of Artificial Sequence: Synthetic oligonucleotide
uuuggcauuuauuucuaaaa

SEQ ID NO: 16 - RNA, 20 nt, Artificial Sequence
  Description of Artificial Sequence: Synthetic oligonucleotide
uguauuugugaagucuuaca

SEQ ID NO: 17 - RNA, 20 nt, Artificial Sequence
  Description of Artificial Sequence: Synthetic oligonucleotide
uccuagguaaaaaaaaaaaa

SEQ ID NO: 18 - RNA, 20 nt, Artificial Sequence
  Description of Artificial Sequence: Synthetic oligonucleotide
uaauuuucuuuugcgcacua

SEQ ID NO: 19 - RNA, 20 nt, Artificial Sequence
  Description of Artificial Sequence: Synthetic oligonucleotide
ugacugaaacuucacagaau

SEQ ID NO: 20 - RNA, 20 nt, Artificial Sequence
  Description of Artificial Sequence: Synthetic oligonucleotide
gacugaaacuucacagaaua

SEQ ID NO: 21 - RNA, 20 nt, Artificial Sequence
  Description of Artificial Sequence: Synthetic oligonucleotide
uucauuuuagucugucuucu

SEQ ID NO: 22 - RNA, 20 nt, Artificial Sequence
  Description of Artificial Sequence: Synthetic oligonucleotide
auuaucuaaguuugaauaua

SEQ ID NO: 23 - RNA, 20 nt, Artificial Sequence
  Description of Artificial Sequence: Synthetic oligonucleotide
aauuuuuaaaauaguauucu

SEQ ID NO: 24 - RNA, 20 nt, Artificial Sequence
  Description of Artificial Sequence: Synthetic oligonucleotide
ugaauuauucuucuguuuaa

SEQ ID NO: 25 - RNA, 20 nt, Artificial Sequence
  Description of Artificial Sequence: Synthetic oligonucleotide
aucauccugaguuuuucugu

SEQ ID NO: 26 - RNA, 20 nt, Artificial Sequence
  Description of Artificial Sequence: Synthetic oligonucleotide
uuacuaaaacuuuauuuuac

SEQ ID NO: 27 - RNA, 20 nt, Artificial Sequence
  Description of Artificial Sequence: Synthetic oligonucleotide
accuuuuuuuuuuuuuaccu

SEQ ID NO: 28 - RNA, 20 nt, Artificial Sequence
  Description of Artificial Sequence: Synthetic oligonucleotide
agugcaauggauaggucuuu

SEQ ID NO: 29 - RNA, 20 nt, Artificial Sequence
  Description of Artificial Sequence: Synthetic oligonucleotide
ugauuccuacagaaaaacuc

SEQ ID NO: 30 - RNA, 20 nt, Artificial Sequence
  Description of Artificial Sequence: Synthetic oligonucleotide
ugggcaagggaagaaaaaaa

SEQ ID NO: 31 - RNA, 20 nt, Artificial Sequence
  Description of Artificial Sequence: Synthetic oligonucleotide
ccucacucuugucugggcaa

SEQ ID NO: 32 - RNA, 20 nt, Artificial Sequence
  Description of Artificial Sequence: Synthetic oligonucleotide
accucacucuugucugggca

SEQ ID NO: 33 - RNA, 20 nt, Artificial Sequence
  Description of Artificial Sequence: Synthetic oligonucleotide
ugagcaaccucacucuuguc

SEQ ID NO: 34 - RNA, 100 nt, Artificial Sequence
  Description of Artificial Sequence: Synthetic polynucleotide
gagcaaccucacucuugucuguuuuagagcuagaaauagcaaguuaaaauaaggcuaguccguuaucaacuugaaaaaguggcaccgagucggugcuuuu

SEQ ID NO: 35 - RNA, 100 nt, Artificial Sequence
  Description of Artificial Sequence: Synthetic polynucleotide
augcauuuguuucaaaauauguuuuagagcuagaaauagcaaguuaaaauaaggcuaguccguuaucaacuugaaaaaguggcaccgagucggugcuuuu

SEQ ID NO: 36 - RNA, 100 nt, Artificial Sequence
  Description of Artificial Sequence: Synthetic polynucleotide
ugcauuuguuucaaaauauuguuuuagagcuagaaauagcaaguuaaaauaaggcuaguccguuaucaacuugaaaaaguggcaccgagucggugcuuuu

SEQ ID NO: 37 - RNA, 100 nt, Artificial Sequence
  Description of Artificial Sequence: Synthetic polynucleotide
auuuaugagaucaacagcacguuuuagagcuagaaauagcaaguuaaaauaaggcuaguccguuaucaacuugaaaaaguggcaccgagucggugcuuuu

SEQ ID NO: 38 - RNA, 100 nt, Artificial Sequence
  Description of Artificial Sequence: Synthetic polynucleotide
gaucaacagcacagguuuugguuuuagagcuagaaauagcaaguuaaaauaaggcuaguccguuaucaacuugaaaaaguggcaccgagucggugcuuuu

SEQ ID NO: 39 - RNA, 100 nt, Artificial Sequence
  Description of Artificial Sequence: Synthetic polynucleotide
uuaaauaaagcauagugcaaguuuuagagcuagaaauagcaaguuaaaauaaggcuaguccguuaucaacuugaaaaaguggcaccgagucggugcuuuu

SEQ ID NO: 40 - RNA, 100 nt, Artificial Sequence
  Description of Artificial Sequence: Synthetic polynucleotide
uaaagcauagugcaauggauguuuuagagcuagaaauagcaaguuaaaauaaggcuaguccguuaucaacuugaaaaaguggcaccgagucggugcuuuu

SEQ ID NO: 41 - RNA, 100 nt, Artificial Sequence
  Description of Artificial Sequence: Synthetic polynucleotide
uagugcaauggauaggucuuguuuuagagcuagaaauagcaaguuaaaauaaggcuaguccguuaucaacuugaaaaaguggcaccgagucggugcuuuu

SEQ ID NO: 42 - RNA, 100 nt, Artificial Sequence
  Description of Artificial Sequence: Synthetic polynucleotide
uacuaaaacuuuauuuuacuguuuuagagcuagaaauagcaaguuaaaauaaggcuaguccguuaucaacuugaaaaaguggcaccgagucggugcuuuu

SEQ ID NO: 43 - RNA, 100 nt, Artificial Sequence
  Description of Artificial Sequence: Synthetic polynucleotide
aaaguugaacaauagaaaaaguuuuagagcuagaaauagcaaguuaaaauaaggcuaguccguuaucaacuugaaaaaguggcaccgagucggugcuuuu

SEQ ID NO: 44 - RNA, 100 nt, Artificial Sequence
  Description of Artificial Sequence: Synthetic polynucleotide
aaugcauaaucuaagucaaaguuuuagagcuagaaauagcaaguuaaaauaaggcuaguccguuaucaacuugaaaaaguggcaccgagucggugcuuuu

SEQ ID NO: 45 - RNA, 100 nt, Artificial Sequence
  Description of Artificial Sequence: Synthetic polynucleotide
uaauaaaauucaaacauccuguuuuagagcuagaaauagcaaguuaaaauaaggcuaguccguuaucaacuugaaaaaguggcaccgagucggugcuuuu

SEQ ID NO: 46 - RNA, 100 nt, Artificial Sequence
  Description of Artificial Sequence: Synthetic polynucleotide
gcaucuuuaaagaauuauuuguuuuagagcuagaaauagcaaguuaaaauaaggcuaguccguuaucaacuugaaaaaguggcaccgagucggugcuuuu

SEQ ID NO: 47 - RNA, 100 nt, Artificial Sequence
  Description of Artificial Sequence: Synthetic polynucleotide
uuuggcauuuauuucuaaaaguuuuagagcuagaaauagcaaguuaaaauaaggcuaguccguuaucaacuugaaaaaguggcaccgagucggugcuuuu

SEQ ID NO: 48 - RNA, 100 nt, Artificial Sequence
  Description of Artificial Sequence: Synthetic polynucleotide
uguauuugugaagucuuacaguuuuagagcuagaaauagcaaguuaaaauaaggcuaguccguuaucaacuugaaaaaguggcaccgagucggugcuuuu

SEQ ID NO: 49 - RNA, 100 nt, Artificial Sequence
  Description of Artificial Sequence: Synthetic polynucleotide
uccuagguaaaaaaaaaaaaguuuuagagcuagaaauagcaaguuaaaauaaggcuaguccguuaucaacuugaaaaaguggcaccgagucggugcuuuu

SEQ ID NO: 50 - RNA, 100 nt, Artificial Sequence
  Description of Artificial Sequence: Synthetic polynucleotide
uaauuuucuuuugcgcacuaguuuuagagcuagaaauagcaaguuaaaauaaggcuaguccguuaucaacuugaaaaaguggcaccgagucggugcuuuu

SEQ ID NO: 51 - RNA, 100 nt, Artificial Sequence
  Description of Artificial Sequence: Synthetic polynucleotide
ugacugaaacuucacagaauguuuuagagcuagaaauagcaaguuaaaauaaggcuaguccguuaucaacuugaaaaaguggcaccgagucggugcuuuu

SEQ ID NO: 52 - RNA, 100 nt, Artificial Sequence
  Description of Artificial Sequence: Synthetic polynucleotide
gacugaaacuucacagaauaguuuuagagcuagaaauagcaaguuaaaauaaggcuaguccguuaucaacuugaaaaaguggcaccgagucggugcuuuu

SEQ ID NO: 53 - RNA, 100 nt, Artificial Sequence
  Description of Artificial Sequence: Synthetic polynucleotide
uucauuuuagucugucuucuguuuuagagcuagaaauagcaaguuaaaauaaggcuaguccguuaucaacuugaaaaaguggcaccgagucggugcuuuu

SEQ ID NO: 54 - RNA, 100 nt, Artificial Sequence
  Description of Artificial Sequence: Synthetic polynucleotide
auuaucuaaguuugaauauaguuuuagagcuagaaauagcaaguuaaaauaaggcuaguccguuaucaacuugaaaaaguggcaccgagucggugcuuuu

SEQ ID NO: 55 - RNA, 100 nt, Artificial Sequence
  Description of Artificial Sequence: Synthetic polynucleotide
aauuuuuaaaauaguauucuguuuuagagcuagaaauagcaaguuaaaauaaggcuaguccguuaucaacuugaaaaaguggcaccgagucggugcuuuu

SEQ ID NO: 56 - RNA, 100 nt, Artificial Sequence
  Description of Artificial Sequence: Synthetic polynucleotide
ugaauuauucuucuguuuaaguuuuagagcuagaaauagcaaguuaaaauaaggcuaguccguuaucaacuugaaaaaguggcaccgagucggugcuuuu

SEQ ID NO: 57 - RNA, 100 nt, Artificial Sequence
  Description of Artificial Sequence: Synthetic polynucleotide
aucauccugaguuuuucuguguuuuagagcuagaaauagcaaguuaaaauaaggcuaguccguuaucaacuugaaaaaguggcaccgagucggugcuuuu

SEQ ID NO: 58 - RNA, 100 nt, Artificial Sequence
  Description of Artificial Sequence: Synthetic polynucleotide
uuacuaaaacuuuauuuuacguuuuagagcuagaaauagcaaguuaaaauaaggcuaguccguuaucaacuugaaaaaguggcaccgagucggugcuuuu

SEQ ID NO: 59 - RNA, 100 nt, Artificial Sequence
  Description of Artificial Sequence: Synthetic polynucleotide
accuuuuuuuuuuuuuaccuguuuuagagcuagaaauagcaaguuaaaauaaggcuaguccguuaucaacuugaaaaaguggcaccgagucggugcuuuu

SEQ ID NO: 60 - RNA, 100 nt, Artificial Sequence
  Description of Artificial Sequence: Synthetic polynucleotide
agugcaauggauaggucuuuguuuuagagcuagaaauagcaaguuaaaauaaggcuaguccguuaucaacuugaaaaaguggcaccgagucggugcuuuu

SEQ ID NO: 61 - RNA, 100 nt, Artificial Sequence
  Description of Artificial Sequence: Synthetic polynucleotide
ugauuccuacagaaaaacucguuuuagagcuagaaauagcaaguuaaaauaaggcuaguccguuaucaacuugaaaaaguggcaccgagucggugcuuuu

SEQ ID NO: 62 - RNA, 100 nt, Artificial Sequence
  Description of Artificial Sequence: Synthetic polynucleotide
ugggcaagggaagaaaaaaaguuuuagagcuagaaauagcaaguuaaaauaaggcuaguccguuaucaacuugaaaaaguggcaccgagucggugcuuuu

SEQ ID NO: 63 - RNA, 100 nt, Artificial Sequence
  Description of Artificial Sequence: Synthetic polynucleotide
ccucacucuugucugggcaaguuuuagagcuagaaauagcaaguuaaaauaaggcuaguccguuaucaacuugaaaaaguggcaccgagucggugcuuuu

SEQ ID NO: 64 - RNA, 100 nt, Artificial Sequence
  Description of Artificial Sequence: Synthetic polynucleotide
accucacucuugucugggcaguuuuagagcuagaaauagcaaguuaaaauaaggcuaguccguuaucaacuugaaaaaguggcaccgagucggugcuuuu

SEQ ID NO: 65 - RNA, 100 nt, Artificial Sequence
  Description of Artificial Sequence: Synthetic polynucleotide
ugagcaaccucacucuugucguuuuagagcuagaaauagcaaguuaaaauaaggcuaguccguuaucaacuugaaaaaguggcaccgagucggugcuuuu

SEQ ID NO: 66 - RNA, 100 nt, Artificial Sequence; identical to SEQ ID NO: 34
  Description of Artificial Sequence: Synthetic polynucleotide

SEQ ID NO: 67 - RNA, 100 nt, Artificial Sequence; identical to SEQ ID NO: 35
  Description of Artificial Sequence: Synthetic polynucleotide

SEQ ID NO: 68 - RNA, 100 nt, Artificial Sequence; identical to SEQ ID NO: 36
  Description of Artificial Sequence: Synthetic polynucleotide

SEQ ID NO: 69 - RNA, 100 nt, Artificial Sequence; identical to SEQ ID NO: 37
  Description of Artificial Sequence: Synthetic polynucleotide

SEQ ID NO: 70 - RNA, 100 nt, Artificial Sequence; identical to SEQ ID NO: 38
  Description of Artificial Sequence: Synthetic polynucleotide

SEQ ID NO: 71 - RNA, 100 nt, Artificial Sequence; identical to SEQ ID NO: 39
  Description of Artificial Sequence: Synthetic polynucleotide

SEQ ID NO: 72 - RNA, 100 nt, Artificial Sequence; identical to SEQ ID NO: 40
  Description of Artificial Sequence: Synthetic polynucleotide

SEQ ID NO: 73 - RNA, 100 nt, Artificial Sequence; identical to SEQ ID NO: 41
  Description of Artificial Sequence: Synthetic polynucleotide

SEQ ID NO: 74 - RNA, 100 nt, Artificial Sequence; identical to SEQ ID NO: 42
  Description of Artificial Sequence: Synthetic polynucleotide

SEQ ID NO: 75 - RNA, 100 nt, Artificial Sequence; identical to SEQ ID NO: 43
  Description of Artificial Sequence: Synthetic polynucleotide

SEQ ID NO: 76 - RNA, 100 nt, Artificial Sequence; identical to SEQ ID NO: 44
  Description of Artificial Sequence: Synthetic polynucleotide

SEQ ID NO: 77 - RNA, 100 nt, Artificial Sequence; identical to SEQ ID NO: 45
  Description of Artificial Sequence: Synthetic polynucleotide

SEQ ID NO: 78 - RNA, 100 nt, Artificial Sequence; identical to SEQ ID NO: 46
  Description of Artificial Sequence: Synthetic polynucleotide

SEQ ID NO: 79 - RNA, 100 nt, Artificial Sequence; identical to SEQ ID NO: 47
  Description of Artificial Sequence: Synthetic polynucleotide

SEQ ID NO: 80 - RNA, 100 nt, Artificial Sequence; identical to SEQ ID NO: 48
  Description of Artificial Sequence: Synthetic polynucleotide

SEQ ID NO: 81 - RNA, 100 nt, Artificial Sequence; identical to SEQ ID NO: 49
  Description of Artificial Sequence: Synthetic polynucleotide

SEQ ID NO: 82 - RNA, 100 nt, Artificial Sequence; identical to SEQ ID NO: 50
  Description of Artificial Sequence: Synthetic polynucleotide

SEQ ID NO: 83 - RNA, 100 nt, Artificial Sequence; identical to SEQ ID NO: 51
  Description of Artificial Sequence: Synthetic polynucleotide

SEQ ID NO: 84 - RNA, 100 nt, Artificial Sequence; identical to SEQ ID NO: 52
  Description of Artificial Sequence: Synthetic polynucleotide

SEQ ID NO: 85 - RNA, 100 nt, Artificial Sequence; identical to SEQ ID NO: 53
  Description of Artificial Sequence: Synthetic polynucleotide

SEQ ID NO: 86 - RNA, 100 nt, Artificial Sequence; identical to SEQ ID NO: 54
  Description of Artificial Sequence: Synthetic polynucleotide

SEQ ID NO: 87 - RNA, 100 nt, Artificial Sequence; identical to SEQ ID NO: 55
  Description of Artificial Sequence: Synthetic polynucleotide

SEQ ID NO: 88 - RNA, 100 nt, Artificial Sequence; identical to SEQ ID NO: 56
  Description of Artificial Sequence: Synthetic polynucleotide

SEQ ID NO: 89 - RNA, 100 nt, Artificial Sequence; identical to SEQ ID NO: 57
  Description of Artificial Sequence: Synthetic polynucleotide

SEQ ID NO: 90 - RNA, 100 nt, Artificial Sequence; identical to SEQ ID NO: 58
  Description of Artificial Sequence: Synthetic polynucleotide

SEQ ID NO: 91 - RNA, 100 nt, Artificial Sequence; identical to SEQ ID NO: 59
  Description of Artificial Sequence: Synthetic polynucleotide

SEQ ID NO: 92 - RNA, 100 nt, Artificial Sequence; identical to SEQ ID NO: 60
  Description of Artificial Sequence: Synthetic polynucleotide

SEQ ID NO: 93 - RNA, 100 nt, Artificial Sequence; identical to SEQ ID NO: 61
  Description of Artificial Sequence: Synthetic polynucleotide

SEQ ID NO: 94 - RNA, 100 nt, Artificial Sequence; identical to SEQ ID NO: 62
  Description of Artificial Sequence: Synthetic polynucleotide

SEQ ID NO: 95 - RNA, 100 nt, Artificial Sequence; identical to SEQ ID NO: 63
  Description of Artificial Sequence: Synthetic polynucleotide

SEQ ID NO: 96 - RNA, 100 nt, Artificial Sequence; identical to SEQ ID NO: 64
  Description of Artificial Sequence: Synthetic polynucleotide

SEQ ID NO: 97 - RNA, 100 nt, Artificial Sequence; identical to SEQ ID NO: 65
  Description of Artificial Sequence: Synthetic polynucleotide

SEQ ID NO: 98 - RNA, 20 nt, Artificial Sequence
  Description of Artificial Sequence: Synthetic oligonucleotide
auuugcaucugagaacccuu

SEQ ID NO: 99 - RNA, 20 nt, Artificial Sequence
  Description of Artificial Sequence: Synthetic oligonucleotide
aucgggaacuggcaucuuca

SEQ ID NO: 100 - RNA, 20 nt, Artificial Sequence
  Description of Artificial Sequence: Synthetic oligonucleotide
guuacaggaaaaucugaagg

SEQ ID NO: 101 - RNA, 20 nt, Artificial Sequence
  Description of Artificial Sequence: Synthetic oligonucleotide
gaucgggaacuggcaucuuc

SEQ ID NO: 102 - RNA, 20 nt, Artificial Sequence
  Description of Artificial Sequence: Synthetic oligonucleotide
ugcaucugagaacccuuagg

SEQ ID NO: 103 - RNA, 20 nt, Artificial Sequence
  Description of Artificial Sequence: Synthetic oligonucleotide
cacucuugucuguggaaaca

SEQ ID NO: 104 - RNA, 20 nt, Artificial Sequence
  Description of Artificial Sequence: Synthetic oligonucleotide
aucguuacaggaaaaucuga

SEQ ID NO: 105 - RNA, 20 nt, Artificial Sequence
  Description of Artificial Sequence: Synthetic oligonucleotide
gcaucuucagggaguagcuu

SEQ ID NO: 106 - RNA, 20 nt, Artificial Sequence
  Description of Artificial Sequence: Synthetic oligonucleotide
caaucuuuaaauauguugug

SEQ ID NO: 107 - RNA, 20 nt, Artificial Sequence
  Description of Artificial Sequence: Synthetic oligonucleotide
ucacucuugucuguggaaac

SEQ ID NO: 108 - RNA, 20 nt, Artificial Sequence
  Description of Artificial Sequence: Synthetic oligonucleotide
ugcuuguauuuuucuaguaa

SEQ ID NO: 109 - RNA, 20 nt, Artificial Sequence
  Description of Artificial Sequence: Synthetic oligonucleotide
guaaauaucuacuaagacaa

SEQ ID NO: 110 - RNA, 20 nt, Artificial Sequence
  Description of Artificial Sequence: Synthetic oligonucleotide
uuuuucuaguaauggaagcc

SEQ ID NO: 111 - RNA, 20 nt, Artificial Sequence
  Description of Artificial Sequence: Synthetic oligonucleotide
uuauauuauugauauauuuu

SEQ ID NO: 112 - RNA, 20 nt, Artificial Sequence
  Description of Artificial Sequence: Synthetic oligonucleotide
gcacagauauaaacacuuaa

SEQ ID NO: 113 - RNA, 20 nt, Artificial Sequence
  Description of Artificial Sequence: Synthetic oligonucleotide
cacagauauaaacacuuaac

SEQ ID NO: 114 - RNA, 20 nt, Artificial Sequence
  Description of Artificial Sequence: Synthetic oligonucleotide
gguuuuaaaaauaauaaugu

SEQ ID NO: 115 - RNA, 20 nt, Artificial Sequence
  Description of Artificial Sequence: Synthetic oligonucleotide
ucagauuuuccuguaacgau

SEQ ID NO: 116 - RNA, 20 nt, Artificial Sequence
  Description of Artificial Sequence: Synthetic oligonucleotide
cagauuuuccuguaacgauc

SEQ ID NO: 117 - RNA, 20 nt, Artificial Sequence
  Description of Artificial Sequence: Synthetic oligonucleotide
caaugguaaauaagaaauaa

SEQ ID NO: 118 - RNA, 20 nt, Artificial Sequence
  Description of Artificial Sequence: Synthetic oligonucleotide
ggaaaaucugaagguggcaa

SEQ ID NO: 119 - RNA, 20 nt, Artificial Sequence
  Description of Artificial Sequence: Synthetic oligonucleotide
ggcgaucucacucuugucug

SEQ ID NO: 120 - RNA, 100 nt, Artificial Sequence
  Description of Artificial Sequence: Synthetic polynucleotide
auuugcaucugagaacccuuguuuuagagcuagaaauagcaaguuaaaauaaggcuaguccguuaucaacuugaaaaaguggcaccgagucggugcuuuu

SEQ ID NO: 121 - RNA, 100 nt, Artificial Sequence
  Description of Artificial Sequence: Synthetic polynucleotide
aucgggaacuggcaucuucaguuuuagagcuagaaauagcaaguuaaaauaaggcuaguccguuaucaacuugaaaaaguggcaccgagucggugcuuuu

SEQ ID NO: 122 - RNA, 100 nt, Artificial Sequence
  Description of Artificial Sequence: Synthetic polynucleotide
guuacaggaaaaucugaaggguuuuagagcuagaaauagcaaguuaaaauaaggcuaguccguuaucaacuugaaaaaguggcaccgagucggugcuuuu

SEQ ID NO: 123 - RNA, 100 nt, Artificial Sequence
  Description of Artificial Sequence: Synthetic polynucleotide
gaucgggaacuggcaucuucguuuuagagcuagaaauagcaaguuaaaauaaggcuaguccguuaucaacuugaaaaaguggcaccgagucggugcuuuu

SEQ ID NO: 124 - RNA, 100 nt, Artificial Sequence
  Description of Artificial Sequence: Synthetic polynucleotide
ugcaucugagaacccuuaggguuuuagagcuagaaauagcaaguuaaaauaaggcuaguccguuaucaacuugaaaaaguggcaccgagucggugcuuuu

SEQ ID NO: 125 - RNA, 100 nt, Artificial Sequence
  Description of Artificial Sequence: Synthetic polynucleotide
cacucuugucuguggaaacaguuuuagagcuagaaauagcaaguuaaaauaaggcuaguccguuaucaacuugaaaaaguggcaccgagucggugcuuuu

SEQ ID NO: 126 - RNA, 100 nt, Artificial Sequence
  Description of Artificial Sequence: Synthetic polynucleotide
aucguuacaggaaaaucugaguuuuagagcuagaaauagcaaguuaaaauaaggcuaguccguuaucaacuugaaaaaguggcaccgagucggugcuuuu

SEQ ID NO: 127 - RNA, 100 nt, Artificial Sequence
  Description of Artificial Sequence: Synthetic polynucleotide
gcaucuucagggaguagcuuguuuuagagcuagaaauagcaaguuaaaauaaggcuaguccguuaucaacuugaaaaaguggcaccgagucggugcuuuu

SEQ ID NO: 128 - RNA, 100 nt, Artificial Sequence
  Description of Artificial Sequence: Synthetic polynucleotide
caaucuuuaaauauguugugguuuuagagcuagaaauagcaaguuaaaauaaggcuaguccguuaucaacuugaaaaaguggcaccgagucggugcuuuu

SEQ ID NO: 129 - RNA, 100 nt, Artificial Sequence
  Description of Artificial Sequence: Synthetic polynucleotide
ucacucuugucuguggaaacguuuuagagcuagaaauagcaaguuaaaauaaggcuaguccguuaucaacuugaaaaaguggcaccgagucggugcuuuu

SEQ ID NO: 130 - RNA, 100 nt, Artificial Sequence
  Description of Artificial Sequence: Synthetic polynucleotide
ugcuuguauuuuucuaguaaguuuuagagcuagaaauagcaaguuaaaauaaggcuaguccguuaucaacuugaaaaaguggcaccgagucggugcuuuu

SEQ ID NO: 131 - RNA, 100 nt, Artificial Sequence
  Description of Artificial Sequence: Synthetic polynucleotide
guaaauaucuacuaagacaaguuuuagagcuagaaauagcaaguuaaaauaaggcuaguccguuaucaacuugaaaaaguggcaccgagucggugcuuuu

SEQ ID NO: 132 - RNA, 100 nt, Artificial Sequence
  Description of Artificial Sequence: Synthetic polynucleotide
uuuuucuaguaauggaagccguuuuagagcuagaaauagcaaguuaaaauaaggcuaguccguuaucaacuugaaaaaguggcaccgagucggugcuuuu

SEQ ID NO: 133 - RNA, 100 nt, Artificial Sequence
  Description of Artificial Sequence: Synthetic polynucleotide
uuauauuauugauauauuuuguuuuagagcuagaaauagcaaguuaaaauaaggcuaguccguuaucaacuugaaaaaguggcaccgagucggugcuuuu

SEQ ID NO: 134 - RNA, 100 nt, Artificial Sequence
  Description of Artificial Sequence: Synthetic polynucleotide
gcacagauauaaacacuuaaguuuuagagcuagaaauagcaaguuaaaauaaggcuaguccguuaucaacuugaaaaaguggcaccgagucggugcuuuu

SEQ ID NO: 135 - RNA, 100 nt, Artificial Sequence
  Description of Artificial Sequence: Synthetic polynucleotide
cacagauauaaacacuuaacguuuuagagcuagaaauagcaaguuaaaauaaggcuaguccguuaucaacuugaaaaaguggcaccgagucggugcuuuu

SEQ ID NO: 136 - RNA, 100 nt, Artificial Sequence
  Description of Artificial Sequence: Synthetic polynucleotide
gguuuuaaaaauaauaauguguuuuagagcuagaaauagcaaguuaaaauaaggcuaguccguuaucaacuugaaaaaguggcaccgagucggugcuuuu

SEQ ID NO: 137 - RNA, 100 nt, Artificial Sequence
  Description of Artificial Sequence: Synthetic polynucleotide
ucagauuuuccuguaacgauguuuuagagcuagaaauagcaaguuaaaauaaggcuaguccguuaucaacuugaaaaaguggcaccgagucggugcuuuu

SEQ ID NO: 138 - RNA, 100 nt, Artificial Sequence
  Description of Artificial Sequence: Synthetic polynucleotide
cagauuuuccuguaacgaucguuuuagagcuagaaauagcaaguuaaaauaaggcuaguccguuaucaacuugaaaaaguggcaccgagucggugcuuuu

SEQ ID NO: 139 - RNA, 100 nt, Artificial Sequence
  Description of Artificial Sequence: Synthetic polynucleotide
caaugguaaauaagaaauaaguuuuagagcuagaaauagcaaguuaaaauaaggcuaguccguuaucaacuugaaaaaguggcaccgagucggugcuuuu

SEQ ID NO: 140 - RNA, 100 nt, Artificial Sequence
  Description of Artificial Sequence: Synthetic polynucleotide
ggaaaaucugaagguggcaaguuuuagagcuagaaauagcaaguuaaaauaaggcuaguccguuaucaacuugaaaaaguggcaccgagucggugcuuuu

SEQ ID NO: 141 - RNA, 100 nt, Artificial Sequence
  Description of Artificial Sequence: Synthetic polynucleotide
ggcgaucucacucuugucugguuuuagagcuagaaauagcaaguuaaaauaaggcuaguccguuaucaacuugaaaaaguggcaccgagucggugcuuuu

SEQ ID NO: 142 - RNA, 100 nt, Artificial Sequence; identical to SEQ ID NO: 120
  Description of Artificial Sequence: Synthetic polynucleotide

SEQ ID NO: 143 - RNA, 100 nt, Artificial Sequence; identical to SEQ ID NO: 121
  Description of Artificial Sequence: Synthetic polynucleotide

SEQ ID NO: 144 - RNA, 100 nt, Artificial Sequence; identical to SEQ ID NO: 122
  Description of Artificial Sequence: Synthetic polynucleotide

SEQ ID NO: 145 - RNA, 100 nt, Artificial Sequence; identical to SEQ ID NO: 123
  Description of Artificial Sequence: Synthetic polynucleotide

SEQ ID NO: 146 - RNA, 100 nt, Artificial Sequence; identical to SEQ ID NO: 124
  Description of Artificial Sequence: Synthetic polynucleotide

SEQ ID NO: 147 - RNA, 100 nt, Artificial Sequence; identical to SEQ ID NO: 125
  Description of Artificial Sequence: Synthetic polynucleotide

SEQ ID NO: 148 - RNA, 100 nt, Artificial Sequence; identical to SEQ ID NO: 126
  Description of Artificial Sequence: Synthetic polynucleotide

SEQ ID NO: 149 - RNA, 100 nt, Artificial Sequence; identical to SEQ ID NO: 127
  Description of Artificial Sequence: Synthetic polynucleotide

SEQ ID NO: 150 - RNA, 100 nt, Artificial Sequence; identical to SEQ ID NO: 128
  Description of Artificial Sequence: Synthetic polynucleotide

SEQ ID NO: 151 - RNA, 100 nt, Artificial Sequence; identical to SEQ ID NO: 129
  Description of Artificial Sequence: Synthetic polynucleotide

SEQ ID NO: 152 - RNA, 100 nt, Artificial Sequence; identical to SEQ ID NO: 130
  Description of Artificial Sequence: Synthetic polynucleotide

SEQ ID NO: 153 - RNA, 100 nt, Artificial Sequence; identical to SEQ ID NO: 131
  Description of Artificial Sequence: Synthetic polynucleotide

SEQ ID NO: 154 - RNA, 100 nt, Artificial Sequence; identical to SEQ ID NO: 132
  Description of Artificial Sequence: Synthetic polynucleotide

SEQ ID NO: 155 - RNA, 100 nt, Artificial Sequence; identical to SEQ ID NO: 133
  Description of Artificial Sequence: Synthetic polynucleotide

SEQ ID NO: 156 - RNA, 100 nt, Artificial Sequence; identical to SEQ ID NO: 134
  Description of Artificial Sequence: Synthetic polynucleotide

SEQ ID NO: 157 - RNA, 100 nt, Artificial Sequence; identical to SEQ ID NO: 135
  Description of Artificial Sequence: Synthetic polynucleotide

SEQ ID NO: 158 - RNA, 100 nt, Artificial Sequence; identical to SEQ ID NO: 136
  Description of Artificial Sequence: Synthetic polynucleotide

SEQ ID NO: 159 - RNA, 100 nt, Artificial Sequence; identical to SEQ ID NO: 137
  Description of Artificial Sequence: Synthetic polynucleotide

SEQ ID NO: 160 - RNA, 100 nt, Artificial Sequence; identical to SEQ ID NO: 138
  Description of Artificial Sequence: Synthetic polynucleotide

SEQ ID NO: 161 - RNA, 100 nt, Artificial Sequence; identical to SEQ ID NO: 139
  Description of Artificial Sequence: Synthetic polynucleotide

SEQ ID NO: 162 - RNA, 100 nt, Artificial Sequence; identical to SEQ ID NO: 140
  Description of Artificial Sequence: Synthetic polynucleotide

SEQ ID NO: 163 - RNA, 100 nt, Artificial Sequence; identical to SEQ ID NO: 141
  Description of Artificial Sequence: Synthetic polynucleotide

SEQ ID NO: 164 - skipped

SEQ ID NO: 165 - RNA, 20 nt, Artificial Sequence
  Description of Artificial Sequence: Synthetic oligonucleotide
agcaaccucacucuugucug

SEQ ID NO: 166 - RNA, 20 nt, Artificial Sequence
  Description of Artificial Sequence: Synthetic oligonucleotide
accucacucuugucugggga

SEQ ID NO: 167 - RNA, 20 nt, Artificial Sequence
  Description of Artificial Sequence: Synthetic oligonucleotide
ccucacucuugucuggggaa

SEQ ID NO: 168 - RNA, 20 nt, Artificial Sequence
  Description of Artificial Sequence: Synthetic oligonucleotide
cucacucuugucuggggaag

SEQ ID NO: 169 - RNA, 20 nt, Artificial Sequence
  Description of Artificial Sequence: Synthetic oligonucleotide
ggggaaggggagaaaaaaaa

SEQ ID NO: 170 - RNA, 20 nt, Artificial Sequence
  Description of Artificial Sequence: Synthetic oligonucleotide
gggaaggggagaaaaaaaaa

SEQ ID NO: 171-172 - skipped (2 entries)

SEQ ID NO: 173 - RNA, 20 nt, Artificial Sequence
  Description of Artificial Sequence: Synthetic oligonucleotide
ugauuccuacagaaaaaguc

SEQ ID NO: 174 - RNA, 20 nt, Artificial Sequence
  Description of Artificial Sequence: Synthetic oligonucleotide
uacagaaaaagucaggauaa

SEQ ID NO: 175 - RNA, 20 nt, Artificial Sequence
  Description of Artificial Sequence: Synthetic oligonucleotide
uuucuucugccuuuaaacag

SEQ ID NO: 176 - RNA, 20 nt, Artificial Sequence
  Description of Artificial Sequence: Synthetic oligonucleotide
uuauaguuuuauauucaaac

SEQ ID NO: 177-181 - skipped (5 entries)

SEQ ID NO: 182 - RNA, 20 nt, Artificial Sequence
  Description of Artificial Sequence: Synthetic oligonucleotide
agugcaauggauaggucuua

SEQ ID NO: 183 - RNA, 20 nt, Artificial Sequence
  Description of Artificial Sequence: Synthetic oligonucleotide
uuacuuugcacuuuccuuag

SEQ ID NO: 184 - RNA, 20 nt, Artificial Sequence
  Description of Artificial Sequence: Synthetic oligonucleotide
uacuuugcacuuuccuuagu

SEQ ID NO: 185 - RNA, 20 nt, Artificial Sequence
  Description of Artificial Sequence: Synthetic oligonucleotide
ucugaccuuuuauuuuaccu

SEQ ID NO: 186-188 - skipped (3 entries)

SEQ ID NO: 189 - RNA, 20 nt, Artificial Sequence
  Description of Artificial Sequence: Synthetic oligonucleotide
auuauccugacuuuuucugu

SEQ ID NO: 190 - RNA, 20 nt, Artificial Sequence
  Description of Artificial Sequence: Synthetic oligonucleotide
ugaauuauuccucuguuuaa

SEQ ID NO: 191 - RNA, 20 nt, Artificial Sequence
  Description of Artificial Sequence: Synthetic oligonucleotide
uaauuuucuuuugcccacua

SEQ ID NO: 192 - RNA, 20 nt, Artificial Sequence
  Description of Artificial Sequence: Synthetic oligonucleotide
aaaaggucagaauuguuuag

SEQ ID NO: 193 - RNA, 20 nt, Artificial Sequence
  Description of Artificial Sequence: Synthetic oligonucleotide
aacauccuagguaaaauaaa

SEQ ID NO: 194 - skipped

SEQ ID NO: 195 - RNA, 20 nt, Artificial Sequence
  Description of Artificial Sequence: Synthetic oligonucleotide
uugucauguauuucuaaaau

SEQ ID NO: 196 - RNA, 20 nt, Artificial Sequence
  Description of Artificial Sequence: Synthetic oligonucleotide
uuugucauguauuucuaaaa

SEQ ID NO: 197 - skipped

SEQ ID NO: 198 - RNA, 100 nt, Artificial Sequence
  Description of Artificial Sequence: Synthetic polynucleotide
agcaaccucacucuugucugguuuuagagcuagaaauagcaaguuaaaauaaggcuaguccguuaucaacuugaaaaaguggcaccgagucggugcuuuu

SEQ ID NO: 199 - RNA, 100 nt, Artificial Sequence
  Description of Artificial Sequence: Synthetic polynucleotide
accucacucuugucuggggaguuuuagagcuagaaauagcaaguuaaaauaaggcuaguccguuaucaacuugaaaaaguggcaccgagucggugcuuuu

SEQ ID NO: 200 - RNA, 100 nt, Artificial Sequence
  Description of Artificial Sequence: Synthetic polynucleotide
ccucacucuugucuggggaaguuuuagagcuagaaauagcaaguuaaaauaaggcuaguccguuaucaacuugaaaaaguggcaccgagucggugcuuuu

SEQ ID NO: 201 - RNA, 100 nt, Artificial Sequence
  Description of Artificial Sequence: Synthetic polynucleotide
cucacucuugucuggggaagguuuuagagcuagaaauagcaaguuaaaauaaggcuaguccguuaucaacuugaaaaaguggcaccgagucggugcuuuu

SEQ ID NO: 202 - RNA, 100 nt, Artificial Sequence
  Description of Artificial Sequence: Synthetic polynucleotide
ggggaaggggagaaaaaaaaguuuuagagcuagaaauagcaaguuaaaauaaggcuaguccguuaucaacuugaaaaaguggcaccgagucggugcuuuu

SEQ ID NO: 203 - RNA, 100 nt, Artificial Sequence
  Description of Artificial Sequence: Synthetic polynucleotide
gggaaggggagaaaaaaaaaguuuuagagcuagaaauagcaaguuaaaauaaggcuaguccguuaucaacuugaaaaaguggcaccgagucggugcuuuu

SEQ ID NO: 204-205 - skipped (2 entries)

SEQ ID NO: 206 - RNA, 100 nt, Artificial Sequence
  Description of Artificial Sequence: Synthetic polynucleotide
ugauuccuacagaaaaagucguuuuagagcuagaaauagcaaguuaaaauaaggcuaguccguuaucaacuugaaaaaguggcaccgagucggugcuuuu

SEQ ID NO: 207 - RNA, 100 nt, Artificial Sequence
  Description of Artificial Sequence: Synthetic polynucleotide
uacagaaaaagucaggauaaguuuuagagcuagaaauagcaaguuaaaauaaggcuaguccguuaucaacuugaaaaaguggcaccgagucggugcuuuu

SEQ ID NO: 208 - RNA, 100 nt, Artificial Sequence
  Description of Artificial Sequence: Synthetic polynucleotide
uuucuucugccuuuaaacagguuuuagagcuagaaauagcaaguuaaaauaaggcuaguccguuaucaacuugaaaaaguggcaccgagucggugcuuuu

SEQ ID NO: 209 - RNA, 100 nt, Artificial Sequence
  Description of Artificial Sequence: Synthetic polynucleotide
uuauaguuuuauauucaaacguuuuagagcuagaaauagcaaguuaaaauaaggcuaguccguuaucaacuugaaaaaguggcaccgagucggugcuuuu

SEQ ID NO: 210-214 - skipped (5 entries)

SEQ ID NO: 215 - RNA, 100 nt, Artificial Sequence
  Description of Artificial Sequence: Synthetic polynucleotide
agugcaauggauaggucuuaguuuuagagcuagaaauagcaaguuaaaauaaggcuaguccguuaucaacuugaaaaaguggcaccgagucggugcuuuu

SEQ ID NO: 216 - RNA, 100 nt, Artificial Sequence
  Description of Artificial Sequence: Synthetic polynucleotide
uuacuuugcacuuuccuuagguuuuagagcuagaaauagcaaguuaaaauaaggcuaguccguuaucaacuugaaaaaguggcaccgagucggugcuuuu

SEQ ID NO: 217 - RNA, 100 nt, Artificial Sequence
  Description of Artificial Sequence: Synthetic polynucleotide
uacuuugcacuuuccuuaguguuuuagagcuagaaauagcaaguuaaaauaaggcuaguccguuaucaacuugaaaaaguggcaccgagucggugcuuuu

SEQ ID NO: 218 - RNA, 100 nt, Artificial Sequence
  Description of Artificial Sequence: Synthetic polynucleotide
ucugaccuuuuauuuuaccuguuuuagagcuagaaauagcaaguuaaaauaaggcuaguccguuaucaacuugaaaaaguggcaccgagucggugcuuuu

SEQ ID NO: 219-221 - skipped (3 entries)

SEQ ID NO: 222 - RNA, 100 nt, Artificial Sequence
  Description of Artificial Sequence: Synthetic polynucleotide
auuauccugacuuuuucuguguuuuagagcuagaaauagcaaguuaaaauaaggcuaguccguuaucaacuugaaaaaguggcaccgagucggugcuuuu

SEQ ID NO: 223 - RNA, 100 nt, Artificial Sequence
  Description of Artificial Sequence: Synthetic polynucleotide
ugaauuauuccucuguuuaaguuuuagagcuagaaauagcaaguuaaaauaaggcuaguccguuaucaacuugaaaaaguggcaccgagucggugcuuuu

SEQ ID NO: 224 - RNA, 100 nt, Artificial Sequence
  Description of Artificial Sequence: Synthetic polynucleotide
uaauuuucuuuugcccacuaguuuuagagcuagaaauagcaaguuaaaauaaggcuaguccguuaucaacuugaaaaaguggcaccgagucggugcuuuu

SEQ ID NO: 225 - RNA, 100 nt, Artificial Sequence
  Description of Artificial Sequence: Synthetic polynucleotide
aaaaggucagaauuguuuagguuuuagagcuagaaauagcaaguuaaaauaaggcuaguccguuaucaacuugaaaaaguggcaccgagucggugcuuuu

SEQ ID NO: 226 - RNA, 100 nt, Artificial Sequence
  Description of Artificial Sequence: Synthetic polynucleotide
aacauccuagguaaaauaaaguuuuagagcuagaaauagcaaguuaaaauaaggcuaguccguuaucaacuugaaaaaguggcaccgagucggugcuuuu

SEQ ID NO: 227 - skipped

SEQ ID NO: 228 - RNA, 100 nt, Artificial Sequence
  Description of Artificial Sequence: Synthetic polynucleotide
uugucauguauuucuaaaauguuuuagagcuagaaauagcaaguuaaaauaaggcuaguccguuaucaacuugaaaaaguggcaccgagucggugcuuuu

SEQ ID NO: 229 - RNA, 100 nt, Artificial Sequence
  Description of Artificial Sequence: Synthetic polynucleotide
uuugucauguauuucuaaaaguuuuagagcuagaaauagcaaguuaaaauaaggcuaguccguuaucaacuugaaaaaguggcaccgagucggugcuuuu

SEQ ID NO: 230 - skipped

SEQ ID NO: 231 - RNA, 100 nt, Artificial Sequence; identical to SEQ ID NO: 198
  Description of Artificial Sequence: Synthetic polynucleotide

SEQ ID NO: 232 - RNA, 100 nt, Artificial Sequence; identical to SEQ ID NO: 199
  Description of Artificial Sequence: Synthetic polynucleotide

SEQ ID NO: 233 - RNA, 100 nt, Artificial Sequence; identical to SEQ ID NO: 200
  Description of Artificial Sequence: Synthetic polynucleotide

SEQ ID NO: 234 - RNA, 100 nt, Artificial Sequence; identical to SEQ ID NO: 201
  Description of Artificial Sequence: Synthetic polynucleotide

SEQ ID NO: 235 - RNA, 100 nt, Artificial Sequence; identical to SEQ ID NO: 202
  Description of Artificial Sequence: Synthetic polynucleotide

SEQ ID NO: 236 - RNA, 100 nt, Artificial Sequence; identical to SEQ ID NO: 203
  Description of Artificial Sequence: Synthetic polynucleotide

SEQ ID NO: 237-238 - skipped (2 entries)

SEQ ID NO: 239 - RNA, 100 nt, Artificial Sequence; identical to SEQ ID NO: 206
  Description of Artificial Sequence: Synthetic polynucleotide

SEQ ID NO: 240 - RNA, 100 nt, Artificial Sequence; identical to SEQ ID NO: 207
  Description of Artificial Sequence: Synthetic polynucleotide

SEQ ID NO: 241 - RNA, 100 nt, Artificial Sequence; identical to SEQ ID NO: 208
  Description of Artificial Sequence: Synthetic polynucleotide

SEQ ID NO: 242 - RNA, 100 nt, Artificial Sequence; identical to SEQ ID NO: 209
  Description of Artificial Sequence: Synthetic polynucleotide

SEQ ID NO: 243-247 - skipped (5 entries)

SEQ ID NO: 248 - RNA, 100 nt, Artificial Sequence; identical to SEQ ID NO: 215
  Description of Artificial Sequence: Synthetic polynucleotide

SEQ ID NO: 249 - RNA, 100 nt, Artificial Sequence; identical to SEQ ID NO: 216
  Description of Artificial Sequence: Synthetic polynucleotide

SEQ ID NO: 250 - RNA, 100 nt, Artificial Sequence; identical to SEQ ID NO: 217
  Description of Artificial Sequence: Synthetic polynucleotide

SEQ ID NO: 251 - RNA, 100 nt, Artificial Sequence; identical to SEQ ID NO: 218
  Description of Artificial Sequence: Synthetic polynucleotide

SEQ ID NO: 252-254 - skipped (3 entries)

SEQ ID NO: 255 - RNA, 100 nt, Artificial Sequence; identical to SEQ ID NO: 222
  Description of Artificial Sequence: Synthetic polynucleotide

SEQ ID NO: 256 - RNA, 100 nt, Artificial Sequence; identical to SEQ ID NO: 223
  Description of Artificial Sequence: Synthetic polynucleotide

SEQ ID NO: 257 - RNA, 100 nt, Artificial Sequence; identical to SEQ ID NO: 224
  Description of Artificial Sequence: Synthetic polynucleotide

SEQ ID NO: 258 - RNA, 100 nt, Artificial Sequence; identical to SEQ ID NO: 225
  Description of Artificial Sequence: Synthetic polynucleotide

SEQ ID NO: 259 - RNA, 100 nt, Artificial Sequence; identical to SEQ ID NO: 226
  Description of Artificial Sequence: Synthetic polynucleotide

SEQ ID NO: 260 - skipped

SEQ ID NO: 261 - RNA, 100 nt, Artificial Sequence; identical to SEQ ID NO: 228
  Description of Artificial Sequence: Synthetic polynucleotide

SEQ ID NO: 262 - RNA, 100 nt, Artificial Sequence; identical to SEQ ID NO: 229
  Description of Artificial Sequence: Synthetic polynucleotide

SEQ ID NO: 263 - DNA, 145 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 264 - DNA, 100 bp, Artificial Sequence
  Description of Artificial Sequence: Synthetic polynucleotide
taggtcagtgaagagaagaacaaaaagcagcatattacagttagttgtcttcatcaatctttaaatatgttgtgtggtttttctctccctgtttccacag

SEQ ID NO: 265 - DNA, 1185 bp, Artificial Sequence
  Description of Artificial Sequence: Synthetic polynucleotide
gaggacccccagggcgacgccgcccagaagaccgacaccagccaccacgaccaggaccaccccaccttcaacaagatcacccccaacctggccgagttcgccttcagcctgtacaggcagctggcccaccagagcaacagcaccaacatcttcttcagccccgtgagcatcgccaccgccttcgccatgctgagcctgggcaccaaggccgacacccacgacgagatcctggagggcctgaacttcaacctgaccgagatccccgaggcccagatccacgagggcttccaggagctgctgaggaccctgaaccagcccgacagccagctgcagctgaccaccggcaacggcctgttcctgagcgagggcctgaagctggtggacaagttcctggaggacgtgaagaagctgtaccacagcgaggccttcaccgtgaacttcggcgacaccgaggaggccaagaagcagatcaacgactacgtggagaagggcacccagggcaagatcgtggacctggtgaaggagctggacagggacaccgtgttcgccctggtgaactacatcttcttcaagggcaagtgggagaggcccttcgaggtgaaggacaccgaggaggaggacttccacgtggaccaggtgaccaccgtgaaggtgcccatgatgaagaggctgggcatgttcaacatccagcactgcaagaagctgagcagctgggtgctgctgatgaagtacctgggcaacgccaccgccatcttcttcctgcccgacgagggcaagctgcagcacctggagaacgagctgacccacgacatcatcaccaagttcctggagaacgaggacaggaggagcgccagcctgcacctgcccaagctgagcatcaccggcacctacgacctgaagagcgtgctgggccagctgggcatcaccaaggtgttcagcaacggcgccgacctgagcggcgtgaccgaggaggcccccctgaagctgagcaaggccgtgcacaaggccgtgctgaccatcgacgagaagggcaccgaggccgccggcgccatgttcctggaggccatccccatgagcatcccccccgaggtgaagttcaacaagcccttcgtgttcctgatgatcgagcagaacaccaagagccccctgttcatgggcaaggtggtgaaccccacccagaagtaa

SEQ ID NO: 266 - DNA, 276 bp, Artificial Sequence
  Description of Artificial Sequence: Synthetic polynucleotide
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccc

SEQ ID NO: 267 - DNA, 192 bp, Artificial Sequence
  Description of Artificial Sequence: Synthetic polynucleotide
aaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 268 - DNA, 1185 bp, Artificial Sequence
  Description of Artificial Sequence: Synthetic polynucleotide
gaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 269 - DNA, 100 bp, Artificial Sequence
  Description of Artificial Sequence: Synthetic polynucleotide
ctgtggaaacagggagagaaaaaccacacaacatatttaaagattgatgaagacaactaactgtaatatgctgctttttgttcttctcttcactgaccta

SEQ ID NO: 270 - DNA, 145 bp, Artificial Sequence
  Description of Artificial Sequence: Synthetic polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 271-274 - skipped (4 entries)

SEQ ID NO: 275 - DNA, 1446 bp, Artificial Sequence
  Description of Artificial Sequence: Synthetic polynucleotide
tttcttgatcatgaaaacgccaacaaaattctgaatcggccaaagaggtataattcaggtaaattggaagagtttgttcaagggaaccttgagagagaatgtatggaagaaaagtgtagttttgaagaagcagtattcactttggaggactttgtcggtgactggaggcaaaccgctggttataatctcgaccaagtactggaacagggcggggtaagttccctctttcagaatttgggtgtaagcgtcacaccaatccagcggattgtgttgtctggagagaacggactcaaaattgacatccatgttatcattccatatgaaggtctcagtggagaccaaatggggcagatcgagaagattttcaaggtagtttacccagtcgacgatcaccacttcaaagtcattctccactatggcacacttgttatcgacggagtaactcctaatatgattgattactttggtcgcccgtatgagggcatcgcagtgtttgatggcaaaaagatcaccgtaacaggaacgttgtggaatgggaacaagataatcgacgagagattgataaatccagacgggtcactcctgttcagggttacaattaacggcgtcacaggatggagactctgtgaacgaatactggccacaaatttttcactcctgaagcaggccggagacgtggaggaaaacccagggcccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagcccgaagaagaagagaaaggtctaa

SEQ ID NO: 276 - DNA, 1446 bp, Artificial Sequence
  Description of Artificial Sequence: Synthetic polynucleotide
ttacaccttcctcttcttcttggggctgccgccgcccttgtacagctcgtccatgcccagggtgatgccggcggcggtcacgaactccagcagcaccatgtggtccctcttctcgttggggtccttgctcagggcgctctgggtgctcaggtagtggttgtcgggcagcagcacggggccgtcgccgatgggggtgttctgctggtagtggtcggccagctgcacgctgccgtcctcgatgttgtgcctgatcttgaagttcaccttgatgccgttcttctgcttgtcggccatgatgtacacgttgtggctgttgtagttgtactccagcttgtggcccaggatgttgccgtcctccttgaagtcgatgcccttcagctcgatcctgttcaccagggtgtcgccctcgaacttcacctcggccctggtcttgtagttgccgtcgtccttgaagaagatggtcctctcctgcacgtagccctcgggcatggcgctcttgaagaagtcgtgctgcttcatgtggtcggggtacctgctgaagcactgcacgccgtaggtcagggtggtcaccagggtgggccagggcacgggcagcttgccggtggtgcagatgaacttcagggtcagcttgccgtaggtggcgtcgccctcgccctcgccgctcacgctgaacttgtggccgttcacgtcgccgtccagctccaccaggatgggcaccacgccggtgaacagctcctcgcccttgctcacggggccggggttctcctccacgtcgccggcctgcttcagcaggctgaagttggtggccaggatcctctcgcacagcctccagccggtcacgccgttgatggtcaccctgaacagcaggctgccgtcggggttgatcagcctctcgtcgatgatcttgttgccgttccacagggtgccggtcacggtgatcttcttgccgtcgaacacggcgatgccctcgtagggcctgccgaagtagtcgatcatgttgggggtcacgccgtcgatcaccagggtgccgtagtgcaggatcaccttgaagtggtggtcgtccacggggtacaccaccttgaaaatcttctcgatctggcccatctggtcgccgctcaggccctcgtaggggatgatcacgtggatgtcgatcttcaggccgttctcgccgctcagcacgatcctctggatgggggtcacgctcacgcccaggttctggaacaggctgctcacgccgccctgctccagcacctggtccaggttgtagccggcggtctgcctccagtcgcccacgaagtcctccagggtgaacacggcctcctcgaagctgcacttctcctccatgcactccctctccaggttgccctgcacgaactcctccagcttgccgctgttgtacctcttgggcctgttcaggatcttgttggcgttctcgtggtccaggaa

SEQ ID NO: 277-278 - skipped (2 entries)

SEQ ID NO: 279 - DNA, 3615 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcagtattcactttggaggactttgtcggtgactggaggcaaaccgctggttataatctcgaccaagtactggaacagggcggggtaagttccctctttcagaatttgggtgtaagcgtcacaccaatccagcggattgtgttgtctggagagaacggactcaaaattgacatccatgttatcattccatatgaaggtctcagtggagaccaaatggggcagatcgagaagattttcaaggtagtttacccagtcgacgatcaccacttcaaagtcattctccactatggcacacttgttatcgacggagtaactcctaatatgattgattactttggtcgcccgtatgagggcatcgcagtgtttgatggcaaaaagatcaccgtaacaggaacgttgtggaatgggaacaagataatcgacgagagattgataaatccagacgggtcactcctgttcagggttacaattaacggcgtcacaggatggagactctgtgaacgaatactggccacaaatttttcactcctgaagcaggccggagacgtggaggaaaacccagggcccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagcccgaagaagaagagaaaggtctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacaccttcctcttcttcttggggctgccgccgcccttgtacagctcgtccatgcccagggtgatgccggcggcggtcacgaactccagcagcaccatgtggtccctcttctcgttggggtccttgctcagggcgctctgggtgctcaggtagtggttgtcgggcagcagcacggggccgtcgccgatgggggtgttctgctggtagtggtcggccagctgcacgctgccgtcctcgatgttgtgcctgatcttgaagttcaccttgatgccgttcttctgcttgtcggccatgatgtacacgttgtggctgttgtagttgtactccagcttgtggcccaggatgttgccgtcctccttgaagtcgatgcccttcagctcgatcctgttcaccagggtgtcgccctcgaacttcacctcggccctggtcttgtagttgccgtcgtccttgaagaagatggtcctctcctgcacgtagccctcgggcatggcgctcttgaagaagtcgtgctgcttcatgtggtcggggtacctgctgaagcactgcacgccgtaggtcagggtggtcaccagggtgggccagggcacgggcagcttgccggtggtgcagatgaacttcagggtcagcttgccgtaggtggcgtcgccctcgccctcgccgctcacgctgaacttgtggccgttcacgtcgccgtccagctccaccaggatgggcaccacgccggtgaacagctcctcgcccttgctcacggggccggggttctcctccacgtcgccggcctgcttcagcaggctgaagttggtggccaggatcctctcgcacagcctccagccggtcacgccgttgatggtcaccctgaacagcaggctgccgtcggggttgatcagcctctcgtcgatgatcttgttgccgttccacagggtgccggtcacggtgatcttcttgccgtcgaacacggcgatgccctcgtagggcctgccgaagtagtcgatcatgttgggggtcacgccgtcgatcaccagggtgccgtagtgcaggatcaccttgaagtggtggtcgtccacggggtacaccaccttgaaaatcttctcgatctggcccatctggtcgccgctcaggccctcgtaggggatgatcacgtggatgtcgatcttcaggccgttctcgccgctcagcacgatcctctggatgggggtcacgctcacgcccaggttctggaacaggctgctcacgccgccctgctccagcacctggtccaggttgtagccggcggtctgcctccagtcgcccacgaagtcctccagggtgaacacggcctcctcgaagctgcacttctcctccatgcactccctctccaggttgccctgcacgaactcctccagcttgccgctgttgtacctcttgggcctgttcaggatcttgttggcgttctcgtggtccaggaa

SEQ ID NO: 280-287 - skipped (8 entries)

SEQ ID NO: 288 - DNA, 4104 bp, Artificial Sequence
  Description of Artificial Sequence: Synthetic polynucleotide
atggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgat

SEQ ID NO: 289 - DNA, 3356 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctactagttaggtcagtgaagagaagaacaaaaagcagcatattacagttagttgtcttcatcaatctttaaatatgttgtgtggtttttctctccctgtttccacagttgaggacccccagggcgacgccgcccagaagaccgacaccagccaccacgaccaggaccaccccaccttcaacaagatcacccccaacctggccgagttcgccttcagcctgtacaggcagctggcccaccagagcaacagcaccaacatcttcttcagccccgtgagcatcgccaccgccttcgccatgctgagcctgggcaccaaggccgacacccacgacgagatcctggagggcctgaacttcaacctgaccgagatccccgaggcccagatccacgagggcttccaggagctgctgaggaccctgaaccagcccgacagccagctgcagctgaccaccggcaacggcctgttcctgagcgagggcctgaagctggtggacaagttcctggaggacgtgaagaagctgtaccacagcgaggccttcaccgtgaacttcggcgacaccgaggaggccaagaagcagatcaacgactacgtggagaagggcacccagggcaagatcgtggacctggtgaaggagctggacagggacaccgtgttcgccctggtgaactacatcttcttcaagggcaagtgggagaggcccttcgaggtgaaggacaccgaggaggaggacttccacgtggaccaggtgaccaccgtgaaggtgcccatgatgaagaggctgggcatgttcaacatccagcactgcaagaagctgagcagctgggtgctgctgatgaagtacctgggcaacgccaccgccatcttcttcctgcccgacgagggcaagctgcagcacctggagaacgagctgacccacgacatcatcaccaagttcctggagaacgaggacaggaggagcgccagcctgcacctgcccaagctgagcatcaccggcacctacgacctgaagagcgtgctgggccagctgggcatcaccaaggtgttcagcaacggcgccgacctgagcggcgtgaccgaggaggcccccctgaagctgagcaaggccgtgcacaaggccgtgctgaccatcgacgagaagggcaccgaggccgccggcgccatgttcctggaggccatccccatgagcatcccccccgaggtgaagttcaacaagcccttcgtgttcctgatgatcgagcagaacaccaagagccccctgttcatgggcaaggtggtgaaccccacccagaagtaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcaactgtggaaacagggagagaaaaaccacacaacatatttaaagattgatgaagacaactaactgtaatatgctgctttttgttcttctcttcactgacctaactagtagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 290-299 - skipped (10 entries)

SEQ ID NO: 300 - RNA, 100 nt, Artificial Sequence
  Description of Artificial Sequence: Synthetic polynucleotide
  modified_base (1)..(20): a, c, u, g, unknown or other
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 301-499 - skipped (199 entries)

SEQ ID NO: 500 - DNA, 9 bp, Artificial Sequence
  Description of Artificial Sequence: Synthetic oligonucleotide
ctggaccga

SEQ ID NO: 501 - DNA, 9 bp, Artificial Sequence
  Description of Artificial Sequence: Synthetic oligonucleotide
tcggtccag

SEQ ID NO: 502 - DNA, 9 bp, Artificial Sequence
  Description of Artificial Sequence: Synthetic oligonucleotide
tcggtccag

SEQ ID NO: 503-599 - skipped (97 entries)

SEQ ID NO: 600 - protein, 7 aa, Simian virus 40
PKKKRKV

SEQ ID NO: 601 - protein, 7 aa, Simian virus 40
PKKKRRV

SEQ ID NO: 602 - protein, 16 aa, Unknown
  Description of Unknown: Nucleoplasmin bipartite NLS sequence
KRPAATKKAGQAKKKK

SEQ ID NO: 603-699 - skipped (97 entries)

SEQ ID NO: 700 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 701 - DNA, 3220 bp, Homo sapiens
acaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 702 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 703-799 - skipped (97 entries)

SEQ ID NO: 800 - RNA, 6 nt, Artificial Sequence
  Description of Artificial Sequence: Synthetic oligonucleotide
aauaaa

SEQ ID NO: 801 - RNA, 6 nt, Artificial Sequence
  Description of Artificial Sequence: Synthetic oligonucleotide
uauaaa

SEQ ID NO: 802 - RNA, 6 nt, Artificial Sequence
  Description of Artificial Sequence: Synthetic oligonucleotide
awuaaa

SEQ ID NO: 803-899 - skipped (97 entries)

SEQ ID NO: 900 - RNA, 22 nt, Artificial Sequence
  Description of Artificial Sequence: Synthetic oligonucleotide
guuuuagagcuaugcuguuuug

SEQ ID NO: 901 - RNA, 80 nt, Artificial Sequence
  Description of Artificial Sequence: Synthetic oligonucleotide
guuuuagagcuagaaauagcaaguuaaaauaaggcuaguccguuaucaacuugaaaaaguggcaccgagucggugcuuuu

SEQ ID NO: 902-999 - skipped (98 entries)

SEQ ID NO: 1000 - RNA, 20 nt, Artificial Sequence
  Description of Artificial Sequence: Synthetic oligonucleotide
gccagacuccaaguucugcc

SEQ ID NO: 1001 - RNA, 20 nt, Artificial Sequence
  Description of Artificial Sequence: Synthetic oligonucleotide
uaaggccaguggaaagaauu

SEQ ID NO: 1002 - RNA, 20 nt, Artificial Sequence
  Description of Artificial Sequence: Synthetic oligonucleotide
ggcagcgaggaguccacagu

SEQ ID NO: 1003 - RNA, 20 nt, Artificial Sequence
  Description of Artificial Sequence: Synthetic oligonucleotide
ucuuuccacuggccuuaacc

SEQ ID NO: 1004 - RNA, 20 nt, Artificial Sequence
  Description of Artificial Sequence: Synthetic oligonucleotide
caaugccgucuucugucucg

SEQ ID NO: 1005 - RNA, 20 nt, Artificial Sequence
  Description of Artificial Sequence: Synthetic oligonucleotide
aaugccgucuucugucucgu

SEQ ID NO: 1006 - RNA, 20 nt, Artificial Sequence
  Description of Artificial Sequence: Synthetic oligonucleotide
augccgucuucugucucgug

SEQ ID NO: 1007 - RNA, 20 nt, Artificial Sequence
  Description of Artificial Sequence: Synthetic oligonucleotide
augccccacgagacagaaga

SEQ ID NO: 1008 - RNA, 20 nt, Artificial Sequence
  Description of Artificial Sequence: Synthetic oligonucleotide
cucguggggcauccuccugc

SEQ ID NO: 1009 - RNA, 20 nt, Artificial Sequence
  Description of Artificial Sequence: Synthetic oligonucleotide
ggauccucagccagggagac

SEQ ID NO: 1010 - RNA, 20 nt, Artificial Sequence
  Description of Artificial Sequence: Synthetic oligonucleotide
ucccuggcugaggaucccca

SEQ ID NO: 1011 - RNA, 20 nt, Artificial Sequence
  Description of Artificial Sequence: Synthetic oligonucleotide
ucccuggggauccucagcca

SEQ ID NO: 1012 - RNA, 20 nt, Artificial Sequence
  Description of Artificial Sequence: Synthetic oligonucleotide
cucccuggggauccucagcc

SEQ ID NO: 1013 - RNA, 20 nt, Artificial Sequence
  Description of Artificial Sequence: Synthetic oligonucleotide
gugggauguaucugucuucu

SEQ ID NO: 1014 - RNA, 20 nt, Artificial Sequence
  Description of Artificial Sequence: Synthetic oligonucleotide
ggugggauguaucugucuuc

SEQ ID NO: 1015 - RNA, 20 nt, Artificial Sequence
  Description of Artificial Sequence: Synthetic oligonucleotide
agauacaucccaccaugauc

SEQ ID NO: 1016 - RNA, 20 nt, Artificial Sequence
  Description of Artificial Sequence: Synthetic oligonucleotide
ugggugauccugaucauggu

SEQ ID NO: 1017 - RNA, 20 nt, Artificial Sequence
  Description of Artificial Sequence: Synthetic oligonucleotide
uugggugauccugaucaugg

SEQ ID NO: 1018 - RNA, 20 nt, Artificial Sequence
  Description of Artificial Sequence: Synthetic oligonucleotide
agguugggugauccugauca

SEQ ID NO: 1019 - RNA, 20 nt, Artificial Sequence
  Description of Artificial Sequence: Synthetic oligonucleotide
gggugaucuuguugaagguu

SEQ ID NO: 1020 - RNA, 20 nt, Artificial Sequence
  Description of Artificial Sequence: Synthetic oligonucleotide
ggggugaucuuguugaaggu

SEQ ID NO: 1021 - RNA, 20 nt, Artificial Sequence
  Description of Artificial Sequence: Synthetic oligonucleotide
caacaagaucacccccaacc

SEQ ID NO: 1022 - RNA, 20 nt, Artificial Sequence
  Description of Artificial Sequence: Synthetic oligonucleotide
aggcgaacucagccagguug

SEQ ID NO: 1023 - RNA, 20 nt, Artificial Sequence
  Description of Artificial Sequence: Synthetic oligonucleotide
gaaggcgaacucagccaggu

SEQ ID NO: 1024 - RNA, 20 nt, Artificial Sequence
  Description of Artificial Sequence: Synthetic oligonucleotide
ggcugaaggcgaacucagcc

SEQ ID NO: 1025 - RNA, 20 nt, Artificial Sequence
  Description of Artificial Sequence: Synthetic oligonucleotide
cagcuggcgguauaggcuga

SEQ ID NO: 1026 - RNA, 20 nt, Artificial Sequence
  Description of Artificial Sequence: Synthetic oligonucleotide
cuucagccuauaccgccagc

SEQ ID NO: 1027 - RNA, 20 nt, Artificial Sequence
  Description of Artificial Sequence: Synthetic oligonucleotide
ggugugccagcuggcgguau

SEQ ID NO: 1028 - RNA, 20 nt, Artificial Sequence
  Description of Artificial Sequence: Synthetic oligonucleotide
uguuggacuggugugccagc

SEQ ID NO: 1029 - RNA, 20 nt, Artificial Sequence
  Description of Artificial Sequence: Synthetic oligonucleotide
agauauuggugcuguuggac

SEQ ID NO: 1030 - RNA, 20 nt, Artificial Sequence
  Description of Artificial Sequence: Synthetic oligonucleotide
gaagaagauauuggugcugu

SEQ ID NO: 1031 - RNA, 20 nt, Artificial Sequence
  Description of Artificial Sequence: Synthetic oligonucleotide
cacuggggagaagaagauau

SEQ ID NO: 1032 - RNA, 20 nt, Artificial Sequence
  Description of Artificial Sequence: Synthetic oligonucleotide
ggcuguagcgaugcucacug

SEQ ID NO: 1033 - RNA, 20 nt, Artificial Sequence
  Description of Artificial Sequence: Synthetic oligonucleotide
aggcuguagcgaugcucacu

SEQ ID NO: 1034 - RNA, 20 nt, Artificial Sequence
  Description of Artificial Sequence: Synthetic oligonucleotide
aaggcuguagcgaugcucac

SEQ ID NO: 1035 - RNA, 20 nt, Artificial Sequence
  Description of Artificial Sequence: Synthetic oligonucleotide
ugacacucacgaugaaaucc

SEQ ID NO: 1036 - RNA, 20 nt, Artificial Sequence
  Description of Artificial Sequence: Synthetic oligonucleotide
cacucacgaugaaauccugg

SEQ ID NO: 1037 - RNA, 20 nt, Artificial Sequence
  Description of Artificial Sequence: Synthetic oligonucleotide
acucacgaugaaauccugga

SEQ ID NO: 1038 - RNA, 20 nt, Artificial Sequence
  Description of Artificial Sequence: Synthetic oligonucleotide
gguugaaauucaggcccucc

SEQ ID NO: 1039 - RNA, 20 nt, Artificial Sequence
  Description of Artificial Sequence: Synthetic oligonucleotide
gggccugaauuucaaccuca

SEQ ID NO: 1040 - RNA, 20 nt, Artificial Sequence
  Description of Artificial Sequence: Synthetic oligonucleotide
uuucaaccucacggagauuc

SEQ ID NO: 1041 - RNA, 20 nt, Artificial Sequence
  Description of Artificial Sequence: Synthetic oligonucleotide
caaccucacggagauuccgg

SEQ ID NO: 1042 - RNA, 20 nt, Artificial Sequence
  Description of Artificial Sequence: Synthetic oligonucleotide
gagccuccggaaucuccgug

SEQ ID NO: 1043 - RNA, 20 nt, Artificial Sequence
  Description of Artificial Sequence: Synthetic oligonucleotide
ccggaggcucagauccauga

SEQ ID NO: 1044 - RNA, 20 nt, Artificial Sequence
  Description of Artificial Sequence: Synthetic oligonucleotide
ugaggguacggaggaguucc

SEQ ID NO: 1045 - RNA, 20 nt, Artificial Sequence
  Description of Artificial Sequence: Synthetic oligonucleotide
cuggcugguugaggguacgg

SEQ ID NO: 1046 - RNA, 20 nt, Artificial Sequence
  Description of Artificial Sequence: Synthetic oligonucleotide
cuggcugucuggcugguuga

SEQ ID NO: 1047 - RNA, 20 nt, Artificial Sequence
  Description of Artificial Sequence: Synthetic oligonucleotide
cuccagcugaccaccggcaa

SEQ ID NO: 1048 - RNA, 20 nt, Artificial Sequence
  Description of Artificial Sequence: Synthetic oligonucleotide
ggccauugccgguggucagc

SEQ ID NO: 1049 - RNA, 20 nt, Artificial Sequence
  Description of Artificial Sequence: Synthetic oligonucleotide
gaggaacaggccauugccgg

SEQ ID NO: 1050 - RNA, 20 nt, Artificial Sequence
  Description of Artificial Sequence: Synthetic oligonucleotide
gcugaggaacaggccauugc

SEQ ID NO: 1051 - RNA, 20 nt, Artificial Sequence
  Description of Artificial Sequence: Synthetic oligonucleotide
caauggccuguuccucagcg

SEQ ID NO: 1052 - RNA, 20 nt, Artificial Sequence
  Description of Artificial Sequence: Synthetic oligonucleotide
aauggccuguuccucagcga

SEQ ID NO: 1053 - RNA, 20 nt, Artificial Sequence
  Description of Artificial Sequence: Synthetic oligonucleotide
ucaggcccucgcugaggaac

SEQ ID NO: 1054 - RNA, 20 nt, Artificial Sequence
  Description of Artificial Sequence: Synthetic oligonucleotide
cuagcuucaggcccucgcug

SEQ ID NO: 1055 - RNA, 20 nt, Artificial Sequence
  Description of Artificial Sequence: Synthetic oligonucleotide
cagcgagggccugaagcuag

SEQ ID NO: 1056 - RNA, 20 nt, Artificial Sequence
  Description of Artificial Sequence: Synthetic oligonucleotide
aaaacuuauccacuagcuuc

SEQ ID NO: 1057 - RNA, 20 nt, Artificial Sequence
  Description of Artificial Sequence: Synthetic oligonucleotide
gaagcuaguggauaaguuuu

SEQ ID NO: 1058 - RNA, 20 nt, Artificial Sequence
  Description of Artificial Sequence: Synthetic oligonucleotide
gcuaguggauaaguuuuugg

SEQ ID NO: 1059 - RNA, 20 nt, Artificial Sequence
  Description of Artificial Sequence: Synthetic oligonucleotide
ugacagugaaggcuucugag

SEQ ID NO: 1060 - RNA, 20 nt, Artificial Sequence
  Description of Artificial Sequence: Synthetic oligonucleotide
aagccuucacugucaacuuc

SEQ ID NO: 1061 - RNA, 20 nt, Artificial Sequence
  Description of Artificial Sequence: Synthetic oligonucleotide
agccuucacugucaacuucg

SEQ ID NO: 1062 - RNA, 20 nt, Artificial Sequence
  Description of Artificial Sequence: Synthetic oligonucleotide
guccccgaaguugacaguga

SEQ ID NO: 1063 - RNA, 20 nt, Artificial Sequence
  Description of Artificial Sequence: Synthetic oligonucleotide
caacuucggggacaccgaag

SEQ ID NO: 1064 - RNA, 20 nt, Artificial Sequence
  Description of Artificial Sequence: Synthetic oligonucleotide
gaucuguuucuuggccucuu

SEQ ID NO: 1065 - RNA, 20 nt, Artificial Sequence
  Description of Artificial Sequence: Synthetic oligonucleotide
guaaucguugaucuguuucu

SEQ ID NO: 1066 - RNA, 20 nt, Artificial Sequence
  Description of Artificial Sequence: Synthetic oligonucleotide
gaaacagaucaacgauuacg

SEQ ID NO: 1067 - RNA, 20 nt, Artificial Sequence
  Description of Artificial Sequence: Synthetic oligonucleotide
gaucaacgauuacguggaga

SEQ ID NO: 1068 - RNA, 20 nt, Artificial Sequence
  Description of Artificial Sequence: Synthetic oligonucleotide
aucaacgauuacguggagaa

SEQ ID NO: 1069 - RNA, 20 nt, Artificial Sequence
  Description of Artificial Sequence: Synthetic oligonucleotide
uacguggagaaggguacuca

SEQ ID NO: 1070 - RNA, 20 nt, Artificial Sequence
  Description of Artificial Sequence: Synthetic oligonucleotide
acguggagaaggguacucaa

SEQ ID NO: 1071 - RNA, 20 nt, Artificial Sequence
  Description of Artificial Sequence: Synthetic oligonucleotide
ucaagggaaaauuguggauu

SEQ ID NO: 1072 - RNA, 20 nt, Artificial Sequence
  Description of Artificial Sequence: Synthetic oligonucleotide
gaaaauuguggauuugguca

SEQ ID NO: 1073 - RNA, 20 nt, Artificial Sequence
  Description of Artificial Sequence: Synthetic oligonucleotide
cagagacacaguuuuugcuc

SEQ ID NO: 1074 - RNA, 20 nt, Artificial Sequence
  Description of Artificial Sequence: Synthetic oligonucleotide
uccccucucuccaggcaaau

SEQ ID NO: 1075 - RNA, 20 nt, Artificial Sequence
  Description of Artificial Sequence: Synthetic oligonucleotide
cucgguguccuugacuucaa

SEQ ID NO: 1076 - RNA, 20 nt, Artificial Sequence
  Description of Artificial Sequence: Synthetic oligonucleotide
cuuugaagucaaggacaccg

SEQ ID NO: 1077 - RNA, 20 nt, Artificial Sequence
  Description of Artificial Sequence: Synthetic oligonucleotide
cacguggaaguccucuuccu

SEQ ID NO: 1078 - RNA, 20 nt, Artificial Sequence
  Description of Artificial Sequence: Synthetic oligonucleotide
cgaggaagaggacuuccacg

SEQ ID NO: 1079 - RNA, 20 nt, Artificial Sequence
  Description of Artificial Sequence: Synthetic oligonucleotide
agaggacuuccacguggacc

SEQ ID NO: 1080 - RNA, 20 nt, Artificial Sequence
  Description of Artificial Sequence: Synthetic oligonucleotide
cgguggucaccugguccacg

SEQ ID NO: 1081 - RNA, 20 nt, Artificial Sequence
  Description of Artificial Sequence: Synthetic oligonucleotide
ggaccaggugaccaccguga

SEQ ID NO: 1082 - RNA, 20 nt, Artificial Sequence
  Description of Artificial Sequence: Synthetic oligonucleotide
gcaccuucacgguggucacc

SEQ ID NO: 1083 - RNA, 20 nt, Artificial Sequence
  Description of Artificial Sequence: Synthetic oligonucleotide
caucauaggcaccuucacgg

SEQ ID NO: 1084 - RNA, 20 nt, Artificial Sequence
  Description of Artificial Sequence: Synthetic oligonucleotide
gugccuaugaugaagcguuu

SEQ ID NO: 1085 - RNA, 20 nt, Artificial Sequence
  Description of Artificial Sequence: Synthetic oligonucleotide
augccuaaacgcuucaucau

SEQ ID NO: 1086 - RNA, 20 nt, Artificial Sequence
  Description of Artificial Sequence: Synthetic oligonucleotide
uggacagcuucuuacagugc

SEQ ID NO: 1087 - RNA, 20 nt, Artificial Sequence
  Description of Artificial Sequence: Synthetic oligonucleotide
cuguaagaagcuguccagcu

SEQ ID NO: 1088 - RNA, 20 nt, Artificial Sequence
  Description of Artificial Sequence: Synthetic oligonucleotide
ggugcugcugaugaaauacc

SEQ ID NO: 1089 - RNA, 20 nt, Artificial Sequence
  Description of Artificial Sequence: Synthetic oligonucleotide
gugcugcugaugaaauaccu

SEQ ID NO: 1090 - RNA, 20 nt, Artificial Sequence
  Description of Artificial Sequence: Synthetic oligonucleotide
agauggcgguggcauugccc

SEQ ID NO: 1091 - RNA, 20 nt, Artificial Sequence
  Description of Artificial Sequence: Synthetic oligonucleotide
aggcaggaagaagauggcgg

SEQ ID NO: 1092 - RNA, 20 nt, Artificial Sequence
  Description of Artificial Sequence: Synthetic oligonucleotide
ggucagcacagccuuaugca

SEQ ID NO: 1093 - RNA, 20 nt, Artificial Sequence
  Description of Artificial Sequence: Synthetic oligonucleotide
agaaagggacugaagcugcu

SEQ ID NO: 1094 - RNA, 20 nt, Artificial Sequence
  Description of Artificial Sequence: Synthetic oligonucleotide
gaaagggacugaagcugcug

SEQ ID NO: 1095 - RNA, 20 nt, Artificial Sequence
  Description of Artificial Sequence: Synthetic oligonucleotide
ugcuggggccauguuuuuag

SEQ ID NO: 1096 - RNA, 20 nt, Artificial Sequence
  Description of Artificial Sequence: Synthetic oligonucleotide
ggguauggccucuaaaaaca

SEQ ID NO: 1097 - RNA, 20 nt, Artificial Sequence
  Description of Artificial Sequence: Synthetic oligonucleotide
uguugaacuugaccucgggg

SEQ ID NO: 1098 - RNA, 20 nt, Artificial Sequence
  Description of Artificial Sequence: Synthetic oligonucleotide
ggguuuguugaacuugaccu

SEQ ID NO: 1099 - RNA, 20 nt, Artificial Sequence
  Description of Artificial Sequence: Synthetic oligonucleotide
uucugggcagcaucucccug

SEQ ID NO: 1100 - RNA, 20 nt, Artificial Sequence
  Description of Artificial Sequence: Synthetic oligonucleotide
ucuucugggcagcaucuccc

SEQ ID NO: 1101 - RNA, 20 nt, Artificial Sequence
  Description of Artificial Sequence: Synthetic oligonucleotide
uggacuggugugccagcugg

SEQ ID NO: 1102 - RNA, 20 nt, Artificial Sequence
  Description of Artificial Sequence: Synthetic oligonucleotide
agccuuugcaaugcucuccc

SEQ ID NO: 1103 - RNA, 20 nt, Artificial Sequence
  Description of Artificial Sequence: Synthetic oligonucleotide
uucaucgugagugucagccu

SEQ ID NO: 1104 - RNA, 20 nt, Artificial Sequence
  Description of Artificial Sequence: Synthetic oligonucleotide
ucuccgugagguugaaauuc

SEQ ID NO: 1105 - RNA, 20 nt, Artificial Sequence
  Description of Artificial Sequence: Synthetic oligonucleotide
gucagcuggagcuggcuguc

SEQ ID NO: 1106 - RNA, 20 nt, Artificial Sequence
  Description of Artificial Sequence: Synthetic oligonucleotide
agccagcuccagcugaccac

SEQ ID NO: 1107 - RNA, 20 nt, Artificial Sequence
  Description of Artificial Sequence: Synthetic oligonucleotide
aucaggcaggaagaagaugg

SEQ ID NO: 1108 - RNA, 20 nt, Artificial Sequence
  Description of Artificial Sequence: Synthetic oligonucleotide
caucuucuuccugccugaug

SEQ ID NO: 1109 - RNA, 20 nt, Artificial Sequence
  Description of Artificial Sequence: Synthetic oligonucleotide
aucuucuuccugccugauga

SEQ ID NO: 1110 - RNA, 20 nt, Artificial Sequence
  Description of Artificial Sequence: Synthetic oligonucleotide
cgauaucaucaccaaguucc

SEQ ID NO: 1111 - RNA, 20 nt, Artificial Sequence
  Description of Artificial Sequence: Synthetic oligonucleotide
cagaucauagguuccaguaa

SEQ ID NO: 1112 - RNA, 20 nt, Artificial Sequence
  Description of Artificial Sequence: Synthetic oligonucleotide
aucacuaaggucuucagcaa

SEQ ID NO: 1113 - RNA, 20 nt, Artificial Sequence
  Description of Artificial Sequence: Synthetic oligonucleotide
ucacuaaggucuucagcaau

SEQ ID NO: 1114 - RNA, 20 nt, Artificial Sequence
  Description of Artificial Sequence: Synthetic oligonucleotide
cacuaaggucuucagcaaug

SEQ ID NO: 1115 - RNA, 20 nt, Artificial Sequence
  Description of Artificial Sequence: Synthetic oligonucleotide
cucaccuuggagagcuucag

SEQ ID NO: 1116 - RNA, 20 nt, Artificial Sequence
  Description of Artificial Sequence: Synthetic oligonucleotide
ucucaccuuggagagcuuca

SEQ ID NO: 1117 - RNA, 20 nt, Artificial Sequence
  Description of Artificial Sequence: Synthetic oligonucleotide
aucucaccuuggagagcuuc

SEQ ID NO: 1118 - RNA, 20 nt, Artificial Sequence
  Description of Artificial Sequence: Synthetic oligonucleotide
uuguugaacuugaccucggg

SEQ ID NO: 1119 - RNA, 20 nt, Artificial Sequence
  Description of Artificial Sequence: Synthetic oligonucleotide
uuuguugaacuugaccucgg

SEQ ID NO: 1120 - RNA, 20 nt, Artificial Sequence
  Description of Artificial Sequence: Synthetic oligonucleotide
guuuguugaacuugaccucg

SEQ ID NO: 1121 - RNA, 20 nt, Artificial Sequence
  Description of Artificial Sequence: Synthetic oligonucleotide
gguuuguugaacuugaccuc

SEQ ID NO: 1122 - RNA, 20 nt, Artificial Sequence
  Description of Artificial Sequence: Synthetic oligonucleotide
ucaaucauuaagaagacaaa

SEQ ID NO: 1123 - RNA, 20 nt, Artificial Sequence
  Description of Artificial Sequence: Synthetic oligonucleotide
uucaaucauuaagaagacaa

SEQ ID NO: 1124 - RNA, 20 nt, Artificial Sequence
  Description of Artificial Sequence: Synthetic oligonucleotide
uaccaagucuccccucuuca

SEQ ID NO: 1125 - RNA, 20 nt, Artificial Sequence
  Description of Artificial Sequence: Synthetic oligonucleotide
accaagucuccccucuucau

SEQ ID NO: 1126 - RNA, 20 nt, Artificial Sequence
  Description of Artificial Sequence: Synthetic oligonucleotide
uccccucuucaugggaaaag

SEQ ID NO: 1127 - RNA, 20 nt, Artificial Sequence
  Description of Artificial Sequence: Synthetic oligonucleotide
caccacuuuucccaugaaga

SEQ ID NO: 1128 - RNA, 20 nt, Artificial Sequence
  Description of Artificial Sequence: Synthetic oligonucleotide
ucaccacuuuucccaugaag

SEQ ID NO: 1129 - protein, 24 aa, Homo sapiens
MPSSVSWGILLLAGLCCLVPVSLA

SEQ ID NO: 1130 - protein, 7 aa, Artificial Sequence
  Description of Artificial Sequence: Synthetic peptide
AVLTIDK

SEQ ID NO: 1131 - protein, 8 aa, Artificial Sequence
  Description of Artificial Sequence: Synthetic peptide
AVLTIDEK

SEQ ID NO: 1132 - protein, 8 aa, Artificial Sequence
  Description of Artificial Sequence: Synthetic peptide
SASLHLPK

SEQ ID NO: 1133 - RNA, 20 nt, Artificial Sequence
  Description of Artificial Sequence: Synthetic oligonucleotide
ugcuuguauuuuucuaguaa

SEQ ID NO: 1134 - RNA, 20 nt, Artificial Sequence
  Description of Artificial Sequence: Synthetic oligonucleotide
uuuuucuaguaauggaagcc

SEQ ID NO: 1135 - RNA, 20 nt, Artificial Sequence
  Description of Artificial Sequence: Synthetic oligonucleotide
auuugcaucugagaacccuu

SEQ ID NO: 1136 - RNA, 20 nt, Artificial Sequence
  Description of Artificial Sequence: Synthetic oligonucleotide
ugcaucugagaacccuuagg

SEQ ID NO: 1137 - RNA, 20 nt, Artificial Sequence
  Description of Artificial Sequence: Synthetic oligonucleotide
uuauauuauugauauauuuu

SEQ ID NO: 1138 - RNA, 20 nt, Artificial Sequence
  Description of Artificial Sequence: Synthetic oligonucleotide
gcacagauauaaacacuuaa

SEQ ID NO: 1139 - RNA, 20 nt, Artificial Sequence
  Description of Artificial Sequence: Synthetic oligonucleotide
cacagauauaaacacuuaac

SEQ ID NO: 1140 - RNA, 20 nt, Artificial Sequence
  Description of Artificial Sequence: Synthetic oligonucleotide
gguuuuaaaaauaauaaugu

SEQ ID NO: 1141 - RNA, 20 nt, Artificial Sequence
  Description of Artificial Sequence: Synthetic oligonucleotide
ucagauuuuccuguaacgau

SEQ ID NO: 1142 - RNA, 20 nt, Artificial Sequence
  Description of Artificial Sequence: Synthetic oligonucleotide
cagauuuuccuguaacgauc

SEQ ID NO: 1143 - RNA, 20 nt, Artificial Sequence
  Description of Artificial Sequence: Synthetic oligonucleotide
gaucgggaacuggcaucuuc

SEQ ID NO: 1144 - RNA, 20 nt, Artificial Sequence
  Description of Artificial Sequence: Synthetic oligonucleotide
aucgggaacuggcaucuuca

SEQ ID NO: 1145 - RNA, 20 nt, Artificial Sequence
  Description of Artificial Sequence: Synthetic oligonucleotide
gcaucuucagggaguagcuu

SEQ ID NO: 1146 - RNA, 20 nt, Artificial Sequence
  Description of Artificial Sequence: Synthetic oligonucleotide
caaucuuuaaauauguugug

SEQ ID NO: 1147 - RNA, 20 nt, Artificial Sequence
  Description of Artificial Sequence: Synthetic oligonucleotide
caaugguaaauaagaaauaa

SEQ ID NO: 1148 - RNA, 20 nt, Artificial Sequence
  Description of Artificial Sequence: Synthetic oligonucleotide
guaaauaucuacuaagacaa

SEQ ID NO: 1149 - RNA, 20 nt, Artificial Sequence
  Description of Artificial Sequence: Synthetic oligonucleotide
guuacaggaaaaucugaagg

SEQ ID NO: 1150 - RNA, 20 nt, Artificial Sequence
  Description of Artificial Sequence: Synthetic oligonucleotide
aucguuacaggaaaaucuga

SEQ ID NO: 1151 - RNA, 20 nt, Artificial Sequence
  Description of Artificial Sequence: Synthetic oligonucleotide
cacucuugucuguggaaaca

SEQ ID NO: 1152 - RNA, 20 nt, Artificial Sequence
  Description of Artificial Sequence: Synthetic oligonucleotide
ucacucuugucuguggaaac

SEQ ID NO: 1153 - RNA, 42 nt, Artificial Sequence
  Description of Artificial Sequence: Synthetic oligonucleotide
ugcuuguauuuuucuaguaaguuuuagagcuaugcuguuuug

SEQ ID NO: 1154 - RNA, 42 nt, Artificial Sequence
  Description of Artificial Sequence: Synthetic oligonucleotide
uuuuucuaguaauggaagccguuuuagagcuaugcuguuuug

SEQ ID NO: 1155 - RNA, 42 nt, Artificial Sequence
  Description of Artificial Sequence: Synthetic oligonucleotide
auuugcaucugagaacccuuguuuuagagcuaugcuguuuug

SEQ ID NO: 1156 - RNA, 42 nt, Artificial Sequence
  Description of Artificial Sequence: Synthetic oligonucleotide
ugcaucugagaacccuuaggguuuuagagcuaugcuguuuug

SEQ ID NO: 1157 - RNA, 42 nt, Artificial Sequence
  Description of Artificial Sequence: Synthetic oligonucleotide
uuauauuauugauauauuuuguuuuagagcuaugcuguuuug

SEQ ID NO: 1158 - RNA, 42 nt, Artificial Sequence
  Description of Artificial Sequence: Synthetic oligonucleotide
gcacagauauaaacacuuaaguuuuagagcuaugcuguuuug

SEQ ID NO: 1159 - RNA, 42 nt, Artificial Sequence
  Description of Artificial Sequence: Synthetic oligonucleotide
cacagauauaaacacuuaacguuuuagagcuaugcuguuuug

SEQ ID NO: 1160 - RNA, 42 nt, Artificial Sequence
  Description of Artificial Sequence: Synthetic oligonucleotide
gguuuuaaaaauaauaauguguuuuagagcuaugcuguuuug

SEQ ID NO: 1161 - RNA, 42 nt, Artificial Sequence
  Description of Artificial Sequence: Synthetic oligonucleotide
ucagauuuuccuguaacgauguuuuagagcuaugcuguuuug

SEQ ID NO: 1162 - RNA, 42 nt, Artificial Sequence
  Description of Artificial Sequence: Synthetic oligonucleotide
cagauuuuccuguaacgaucguuuuagagcuaugcuguuuug

SEQ ID NO: 1163 - RNA, 42 nt, Artificial Sequence
  Description of Artificial Sequence: Synthetic oligonucleotide
gaucgggaacuggcaucuucguuuuagagcuaugcuguuuug

SEQ ID NO: 1164 - RNA, 42 nt, Artificial Sequence
  Description of Artificial Sequence: Synthetic oligonucleotide
aucgggaacuggcaucuucaguuuuagagcuaugcuguuuug

SEQ ID NO: 1165 - RNA, 42 nt, Artificial Sequence
  Description of Artificial Sequence: Synthetic oligonucleotide
gcaucuucagggaguagcuuguuuuagagcuaugcuguuuug

SEQ ID NO: 1166 - RNA, 42 nt, Artificial Sequence
  Description of Artificial Sequence: Synthetic oligonucleotide
caaucuuuaaauauguugugguuuuagagcuaugcuguuuug

SEQ ID NO: 1167 - RNA, 42 nt, Artificial Sequence
  Description of Artificial Sequence: Synthetic oligonucleotide
caaugguaaauaagaaauaaguuuuagagcuaugcuguuuug

SEQ ID NO: 1168 - RNA, 42 nt, Artificial Sequence
  Description of Artificial Sequence: Synthetic oligonucleotide
guaaauaucuacuaagacaaguuuuagagcuaugcuguuuug

SEQ ID NO: 1169 - RNA, 42 nt, Artificial Sequence
  Description of Artificial Sequence: Synthetic oligonucleotide
guuacaggaaaaucugaaggguuuuagagcuaugcuguuuug

SEQ ID NO: 1170 - RNA, 42 nt, Artificial Sequence
  Description of Artificial Sequence: Synthetic oligonucleotide
aucguuacaggaaaaucugaguuuuagagcuaugcuguuuug

SEQ ID NO: 1171 - RNA, 42 nt, Artificial Sequence
  Description of Artificial Sequence: Synthetic oligonucleotide
cacucuugucuguggaaacaguuuuagagcuaugcuguuuug

SEQ ID NO: 1172 - RNA, 42 nt, Artificial Sequence
  Description of Artificial Sequence: Synthetic oligonucleotide
ucacucuugucuguggaaacguuuuagagcuaugcuguuuug

SEQ ID NO: 1173 - RNA, 100 nt, Artificial Sequence
  Description of Artificial Sequence: Synthetic polynucleotide
acucuugucugugggaauggguuuuagagcuagaaauagcaaguuaaaauaaggcuaguccguuaucaacuugaaaaaguggcaccgagucggugcuuuu

SEQ ID NO: 1174 - RNA, 100 nt, Artificial Sequence
  Description of Artificial Sequence: Synthetic polynucleotide
uccaaucgcuacaggaaaauguuuuagagcuagaaauagcaaguuaaaauaaggcuaguccguuaucaacuugaaaaaguggcaccgagucggugcuuuu

SEQ ID NO: 1175 - RNA, 100 nt, Artificial Sequence
  Description of Artificial Sequence: Synthetic polynucleotide
uuaguauagcuagguagagcguuuuagagcuagaaauagcaaguuaaaauaaggcuaguccguuaucaacuugaaaaaguggcaccgagucggugcuuuu

SEQ ID NO: 1176 - RNA, 100 nt, Artificial Sequence
  Description of Artificial Sequence: Synthetic polynucleotide
acuccgaugacaauaaugggguuuuagagcuagaaauagcaaguuaaaauaaggcuaguccguuaucaacuugaaaaaguggcaccgagucggugcuuuu

SEQ ID NO: 1177 - RNA, 100 nt, Artificial Sequence
  Description of Artificial Sequence: Synthetic polynucleotide
gcgaucucacucuugucuguguuuuagagcuagaaauagcaaguuaaaauaaggcuaguccguuaucaacuugaaaaaguggcaccgagucggugcuuuu

SEQ ID NO: 1178 - RNA, 100 nt, Artificial Sequence
  Description of Artificial Sequence: Synthetic polynucleotide
gauuuuccuguagcgauuggguuuuagagcuagaaauagcaaguuaaaauaaggcuaguccguuaucaacuugaaaaaguggcaccgagucggugcuuuu

SEQ ID NO: 1179 - RNA, 100 nt, Artificial Sequence
  Description of Artificial Sequence: Synthetic polynucleotide
auucguaaaauuacuaauacguuuuagagcuagaaauagcaaguuaaaauaaggcuaguccguuaucaacuugaaaaaguggcaccgagucggugcuuuu

SEQ ID NO: 1180 - RNA, 100 nt, Artificial Sequence
  Description of Artificial Sequence: Synthetic polynucleotide
aguccccccauuauugucauguuuuagagcuagaaauagcaaguuaaaauaaggcuaguccguuaucaacuugaaaaaguggcaccgagucggugcuuuu

SEQ ID NO: 1181 - RNA, 100 nt, Artificial Sequence
  Description of Artificial Sequence: Synthetic polynucleotide
gcugaacucacuaguuucaaguuuuagagcuagaaauagcaaguuaaaauaaggcuaguccguuaucaacuugaaaaaguggcaccgagucggugcuuuu

SEQ ID NO: 1182 - RNA, 100 nt, Artificial Sequence
  Description of Artificial Sequence: Synthetic polynucleotide
gauuggaggcugggaacuucguuuuagagcuagaaauagcaaguuaaaauaaggcuaguccguuaucaacuugaaaaaguggcaccgagucggugcuuuu

SEQ ID NO: 1183 - RNA, 100 nt, Artificial Sequence
  Description of Artificial Sequence: Synthetic polynucleotide
uacauuaauauaaccacauaguuuuagagcuagaaauagcaaguuaaaauaaggcuaguccguuaucaacuugaaaaaguggcaccgagucggugcuuuu

SEQ ID NO: 1184 - RNA, 100 nt, Artificial Sequence
  Description of Artificial Sequence: Synthetic polynucleotide
aaacucugaauguagucgaaguuuuagagcuagaaauagcaaguuaaaauaaggcuaguccguuaucaacuugaaaaaguggcaccgagucggugcuuuu

SEQ ID NO: 1185 - RNA, 100 nt, Artificial Sequence
  Description of Artificial Sequence: Synthetic polynucleotide
augacugaaacauuccgucuguuuuagagcuagaaauagcaaguuaaaauaaggcuaguccguuaucaacuugaaaaaguggcaccgagucggugcuuuu

SEQ ID NO: 1186 - RNA, 100 nt, Artificial Sequence
  Description of Artificial Sequence: Synthetic polynucleotide
gugauauuaaucaucaccaaguuuuagagcuagaaauagcaaguuaaaauaaggcuaguccguuaucaacuugaaaaaguggcaccgagucggugcuuuu

SEQ ID NO: 1187 - RNA, 100 nt, Artificial Sequence
  Description of Artificial Sequence: Synthetic polynucleotide
aacuccgaugacaauaauggguuuuagagcuagaaauagcaaguuaaaauaaggcuaguccguuaucaacuugaaaaaguggcaccgagucggugcuuuu

SEQ ID NO: 1188 - RNA, 100 nt, Artificial Sequence
  Description of Artificial Sequence: Synthetic polynucleotide
cuuugcauucaaacccaagaguuuuagagcuagaaauagcaaguuaaaauaaggcuaguccguuaucaacuugaaaaaguggcaccgagucggugcuuuu

SEQ ID NO: 1189 - RNA, 100 nt, Artificial Sequence
  Description of Artificial Sequence: Synthetic polynucleotide
gauaacuccgaugacaauaaguuuuagagcuagaaauagcaaguuaaaauaaggcuaguccguuaucaacuugaaaaaguggcaccgagucggugcuuuu

SEQ ID NO: 1190 - RNA, 100 nt, Artificial Sequence
  Description of Artificial Sequence: Synthetic polynucleotide
auguuuguaaaugucuacuaguuuuagagcuagaaauagcaaguuaaaauaaggcuaguccguuaucaacuugaaaaaguggcaccgagucggugcuuuu

SEQ ID NO: 1191 - RNA, 100 nt, Artificial Sequence
  Description of Artificial Sequence: Synthetic polynucleotide
ugcaucugagaauccuuaugguuuuagagcuagaaauagcaaguuaaaauaaggcuaguccguuaucaacuugaaaaaguggcaccgagucggugcuuuu

SEQ ID NO: 1192 - RNA, 100 nt, Artificial Sequence
  Description of Artificial Sequence: Synthetic polynucleotide
ugacugaaacauuccgucuuguuuuagagcuagaaauagcaaguuaaaauaaggcuaguccguuaucaacuugaaaaaguggcaccgagucggugcuuuu

SEQ ID NO: 1193 - RNA, 100 nt, Artificial Sequence
  Description of Artificial Sequence: Synthetic polynucleotide
uccuguagcgauuggaggcuguuuuagagcuagaaauagcaaguuaaaauaaggcuaguccguuaucaacuugaaaaaguggcaccgagucggugcuuuu

SEQ ID NO: 1194 - RNA, 100 nt, Artificial Sequence
  Description of Artificial Sequence: Synthetic polynucleotide
uaugcguguuuaguauagcuguuuuagagcuagaaauagcaaguuaaaauaaggcuaguccguuaucaacuugaaaaaguggcaccgagucggugcuuuu

SEQ ID NO: 1195 - RNA, 100 nt, Artificial Sequence
  Description of Artificial Sequence: Synthetic polynucleotide
acuuuuauuuuucguaguaaguuuuagagcuagaaauagcaaguuaaaauaaggcuaguccguuaucaacuugaaaaaguggcaccgagucggugcuuuu

SEQ ID NO: 1196 - RNA, 100 nt, Artificial Sequence
  Description of Artificial Sequence: Synthetic polynucleotide
uguauuaguaauuuuacgaaguuuuagagcuagaaauagcaaguuaaaauaaggcuaguccguuaucaacuugaaaaaguggcaccgagucggugcuuuu

SEQ ID NO: 1197 - RNA, 100 nt, Artificial Sequence
  Description of Artificial Sequence: Synthetic polynucleotide
uuaaguaauuuugaaauaccguuuuagagcuagaaauagcaaguuaaaauaaggcuaguccguuaucaacuugaaaaaguggcaccgagucggugcuuuu

SEQ ID NO: 1198 - RNA, 100 nt, Artificial Sequence; identical to SEQ ID NO: 1173
  Description of Artificial Sequence: Synthetic polynucleotide

SEQ ID NO: 1199 - RNA, 100 nt, Artificial Sequence; identical to SEQ ID NO: 1174
  Description of Artificial Sequence: Synthetic polynucleotide

SEQ ID NO: 1200 - RNA, 100 nt, Artificial Sequence; identical to SEQ ID NO: 1175
  Description of Artificial Sequence: Synthetic polynucleotide

SEQ ID NO: 1201 - RNA, 100 nt, Artificial Sequence; identical to SEQ ID NO: 1176
  Description of Artificial Sequence: Synthetic polynucleotide

SEQ ID NO: 1202 - RNA, 100 nt, Artificial Sequence; identical to SEQ ID NO: 1177
  Description of Artificial Sequence: Synthetic polynucleotide

SEQ ID NO: 1203 - RNA, 100 nt, Artificial Sequence; identical to SEQ ID NO: 1178
  Description of Artificial Sequence: Synthetic polynucleotide

SEQ ID NO: 1204 - RNA, 100 nt, Artificial Sequence; identical to SEQ ID NO: 1179
  Description of Artificial Sequence: Synthetic polynucleotide

SEQ ID NO: 1205 - RNA, 100 nt, Artificial Sequence; identical to SEQ ID NO: 1180
  Description of Artificial Sequence: Synthetic polynucleotide

SEQ ID NO: 1206 - RNA, 100 nt, Artificial Sequence; identical to SEQ ID NO: 1181
  Description of Artificial Sequence: Synthetic polynucleotide

SEQ ID NO: 1207 - RNA, 100 nt, Artificial Sequence; identical to SEQ ID NO: 1182
  Description of Artificial Sequence: Synthetic polynucleotide

SEQ ID NO: 1208 - RNA, 100 nt, Artificial Sequence; identical to SEQ ID NO: 1183
  Description of Artificial Sequence: Synthetic polynucleotide

SEQ ID NO: 1209 - RNA, 100 nt, Artificial Sequence; identical to SEQ ID NO: 1184
  Description of Artificial Sequence: Synthetic polynucleotide

SEQ ID NO: 1210 - RNA, 100 nt, Artificial Sequence; identical to SEQ ID NO: 1185
  Description of Artificial Sequence: Synthetic polynucleotide

SEQ ID NO: 1211 - RNA, 100 nt, Artificial Sequence; identical to SEQ ID NO: 1186
  Description of Artificial Sequence: Synthetic polynucleotide

SEQ ID NO: 1212 - RNA, 100 nt, Artificial Sequence; identical to SEQ ID NO: 1187
  Description of Artificial Sequence: Synthetic polynucleotide

SEQ ID NO: 1213 - RNA, 100 nt, Artificial Sequence; identical to SEQ ID NO: 1188
  Description of Artificial Sequence: Synthetic polynucleotide

SEQ ID NO: 1214 - RNA, 100 nt, Artificial Sequence; identical to SEQ ID NO: 1189
  Description of Artificial Sequence: Synthetic polynucleotide

SEQ ID NO: 1215 - RNA, 100 nt, Artificial Sequence; identical to SEQ ID NO: 1190
  Description of Artificial Sequence: Synthetic polynucleotide

SEQ ID NO: 1216 - RNA, 100 nt, Artificial Sequence; identical to SEQ ID NO: 1191
  Description of Artificial Sequence: Synthetic polynucleotide

SEQ ID NO: 1217 - RNA, 100 nt, Artificial Sequence; identical to SEQ ID NO: 1192
  Description of Artificial Sequence: Synthetic polynucleotide

SEQ ID NO: 1218 - RNA, 100 nt, Artificial Sequence; identical to SEQ ID NO: 1193
  Description of Artificial Sequence: Synthetic polynucleotide

SEQ ID NO: 1219 - RNA, 100 nt, Artificial Sequence; identical to SEQ ID NO: 1194
  Description of Artificial Sequence: Synthetic polynucleotide

SEQ ID NO: 1220 - RNA, 100 nt, Artificial Sequence; identical to SEQ ID NO: 1195
  Description of Artificial Sequence: Synthetic polynucleotide

SEQ ID NO: 1221 - RNA, 100 nt, Artificial Sequence; identical to SEQ ID NO: 1196
  Description of Artificial Sequence: Synthetic polynucleotide

SEQ ID NO: 1222 - RNA, 100 nt, Artificial Sequence; identical to SEQ ID NO: 1197
  Description of Artificial Sequence: Synthetic polynucleotide

SEQ ID NO: 1223 - RNA, 100 nt, Artificial Sequence
  Description of Artificial Sequence: Synthetic polynucleotide
auaacuccgaugacaauaauguuuuagagcuagaaauagcaaguuaaaauaaggcuaguccguuaucaacuugaaaaaguggcaccgagucggugcuuuu

SEQ ID NO: 1224 - RNA, 100 nt, Artificial Sequence
  Description of Artificial Sequence: Synthetic polynucleotide
uaacuccgaugacaauaaugguuuuagagcuagaaauagcaaguuaaaauaaggcuaguccguuaucaacuugaaaaaguggcaccgagucggugcuuuu

SEQ ID NO: 1225 - RNA, 100 nt, Artificial Sequence
  Description of Artificial Sequence: Synthetic polynucleotide
uuuucguaguaacggaagccguuuuagagcuagaaauagcaaguuaaaauaaggcuaguccguuaucaacuugaaaaaguggcaccgagucggugcuuuu

SEQ ID NO: 1226 - RNA, 100 nt, Artificial Sequence
  Description of Artificial Sequence: Synthetic polynucleotide
uuccuguagcgauuggaggcguuuuagagcuagaaauagcaaguuaaaauaaggcuaguccguuaucaacuugaaaaaguggcaccgagucggugcuuuu

SEQ ID NO: 1227 - RNA, 100 nt, Artificial Sequence
  Description of Artificial Sequence: Synthetic polynucleotide
ucccagccuccaaucgcuacguuuuagagcuagaaauagcaaguuaaaauaaggcuaguccguuaucaacuugaaaaaguggcaccgagucggugcuuuu

SEQ ID NO: 1228 - RNA, 100 nt, Artificial Sequence
  Description of Artificial Sequence: Synthetic polynucleotide
uccgauuuuccuguagcgauguuuuagagcuagaaauagcaaguuaaaauaaggcuaguccguuaucaacuugaaaaaguggcaccgagucggugcuuuu

SEQ ID NO: 1229 - RNA, 100 nt, Artificial Sequence; identical to SEQ ID NO: 1223
  Description of Artificial Sequence: Synthetic polynucleotide

SEQ ID NO: 1230 - RNA, 100 nt, Artificial Sequence; identical to SEQ ID NO: 1224
  Description of Artificial Sequence: Synthetic polynucleotide

SEQ ID NO: 1231 - RNA, 100 nt, Artificial Sequence; identical to SEQ ID NO: 1225
  Description of Artificial Sequence: Synthetic polynucleotide

SEQ ID NO: 1232 - RNA, 100 nt, Artificial Sequence; identical to SEQ ID NO: 1226
  Description of Artificial Sequence: Synthetic polynucleotide

SEQ ID NO: 1233 - RNA, 100 nt, Artificial Sequence; identical to SEQ ID NO: 1227
  Description of Artificial Sequence: Synthetic polynucleotide

SEQ ID NO: 1234 - RNA, 100 nt, Artificial Sequence; identical to SEQ ID NO: 1228
  Description of Artificial Sequence: Synthetic polynucleotide

SEQ ID NO: 1235-1999 - skipped (765 entries)

SEQ ID NO: 2000 - protein, 18 aa, Artificial Sequence
  Description of Artificial Sequence: Synthetic peptide
MKWVTFISLLFLFSSAYS

SEQ ID NO: 2001 - RNA, 20 nt, Artificial Sequence
  Description of Artificial Sequence: Synthetic oligonucleotide
gaguccgagcagaagaagaa

SEQ ID NO: 2002 - RNA, 20 nt, Artificial Sequence
  Description of Artificial Sequence: Synthetic oligonucleotide
gacccccuccaccccgccuc